(19) United States
(12) Patent Application Publication
(10) Pub. No.: US 2025/0059159 A1
(43) Pub. Date: Feb. 20, 2025

(54) PLPRO INHIBITORS

(71) Applicants: SHANGHAI SYNERGY PHARMACEUTICAL SCIENCES CO., LTD., Shanghai (CN); ZHEJIANG HUAHAI PHARMACEUTICAL CO., LTD., Taizhou (CN)

(72) Inventors: Jian Wu, Chester Springs, PA (US); Tasneem Murtuza Vaid, Aurora, IL (US); Hoshin Kim, New Providence, NJ (US); Jun Lu, Princeton, NJ (US); Min Li, Ringoes, NJ (US); Leslie Wo-mei Fung, Winnetka, IL (US); Michael Evart Johnson, Winnetka, IL (US)

(21) Appl. No.: 18/720,330

(22) PCT Filed: Dec. 15, 2022
(86) PCT No.: PCT/US2022/052978

§ 371 Date: Jun. 14, 2024

Related U.S. Application Data
(60) Provisional application No. 63/290,337, filed on Dec. 16, 2021.

(51) Int. Cl. C07D 401/12; A61K 31/4525; A61K 31/4535; A61K 31/454; A61K 31/4709; A61K 31/4725; A61K 31/496; A61K 45/06; A61P 31/14; C07D 211/58; C07D 405/12; C07D 405/14; C07D 409/10; C07D 413/04; C07D 413/12; C07D 417/04; C07D 487/10

(52) U.S. Cl. CPC C07D 401/12; A61K 31/4525; A61K 31/4535; A61K 31/454; A61K 31/4709; A61K 31/4725; A61K 31/496; A61K 45/06; A61P 31/14; C07D 211/58; C07D 405/12; C07D 405/14; C07D 409/10; C07D 413/04; C07D 413/12; C07D 417/04; C07D 487/10

(57) ABSTRACT

Provided herein are novel compounds (e.g., Formula X or Y), pharmaceutical compositions, and methods of using the same. The compounds herein can typically inhibit PLpro activities. The compounds herein can also be used for treating a variety of diseases or disorders, such as viral infection caused by a coronavirus such as SARS-CoV-2.

### CROSS-REFERENCE TO RELATED APPLICATIONS

This application claims the benefit of U.S. Provisional Application No. 63/290,337, filed Dec. 16, 2021, the content of which is herein incorporated by reference in its entirety.

### FIELD OF THE DISCLOSURE

This disclosure generally relates to PLpro inhibitors and compositions, methods of preparation and methods of use, such as in treating viral infections.

### BACKGROUND

In the past two decades, there have been two major coronavirus outbreaks, the SARS-CoV (2002) and the MERS (2012). The recent coronavirus outbreak is known as the 2019-nCoV outbreak, recently renamed as SARS-CoV-2. The coronaviruses infecting humans (HCoVs) belong to two genera (alpha coronaviruses and beta coronaviruses). The alpha coronaviruses infecting humans are HCoV-229E and HCoV-NL63, and the beta coronaviruses infecting humans are HCoV-HKU1, HCoV-OC43, Middle East respiratory syndrome coronavirus (MERS-CoV), the severe acute respiratory syndrome coronavirus (SARS-CoV), and SARS-CoV-2.

Coronavirus encodes for two large polyproteins that are further processed by virally encoded cysteine proteases, namely, the papain-like protease (PLpro) and the 3-chymotrypsin-like protease (3CLpro, also known as the main protease-Mpro). The processing of the viral polyproteins is essential for maturation and infectivity of the virus. The SARS-CoV-2 PLpro is responsible for processing three cleavage sites of the viral polyprotein to release mature non-structural proteins 1, 2 and 3. Apart from proteolytic processing, PLpro also has a deubiquitinase and deISGylating activity [1-4]. Because of the crucial roles these two proteases play in the viral life-cycle, they are important targets for antiviral drug design.

Studies conducted using SARS CoV proteases have shown that PLpro is essential for viral protein production and infectivity. Small molecule inhibitors of SARS CoV PLpro protease have been shown to possess anti-viral activities against SARS CoV as well as SARS CoV-2 [5-7]. PLpro is required for viral polyprotein maturation and deconjugates ubiquitin from various substrates involved in maintaining host cell immunity, for example, by producing interferon [8]. The net effect of these different functions helps SARS-CoV antagonize the establishment of an antiviral state in the host [2-4]. PLpro inhibits host immune responses by suppressing interferon (IFN) signaling, a major anti-viral response pathway [9].

Previous attempts to design inhibitors against SARS-CoV PLpro yielded some promising results for a family of naphthalene-based inhibitors [10, 11] although no PLpro inhibitors have been approved as an anti-viral drug. With SARS-CoV outbreak effectively contained in 2003 with no reemergence, these potential anti-CoV therapeutics were slowly abandoned.

Coronavirus infections, such as the COVID-19 pandemic, have had a huge medical, social and economic impact [12, 13]. Thus, there is an urgent need to develop effective therapies to treat such infections.

### SUMMARY

The present disclosure is based, in part, on the discovery of a class of novel small molecule PLpro inhibitors. In various embodiments, the present disclosure generally relates to compounds and compositions useful for treating viral infections, such as SARS-CoV-2 infections. Without wishing to be bound by theories, it is believed that compounds and compositions of the present disclosure can inhibit PLpro activities of a virus and therefore inhibits reproduction of the virus. In some embodiments, compounds of the present disclosure can be characterized as an irreversible inhibitor, which can provide advantages over a reversible inhibitor, such advantages include a long duration of action after only a short exposure to their target. As such, if the irreversible inhibitor has a short t1/2, the possibility of off-target toxicity will also be minimized.

In some embodiments, the present disclosure provides a compound of Formula X or Y, or a pharmaceutically acceptable salt thereof,

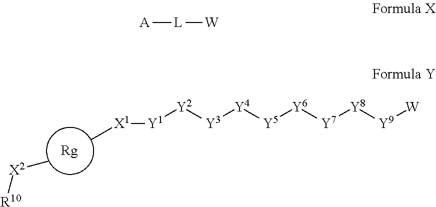

wherein the variables A, L, W, X¹, X², Y¹, Y², Y³, Y⁴, Y⁵, Y⁶, Y⁷, Y⁸, Y⁹, Rg, and R¹⁰ are defined herein for the respective formula. In some embodiments, the compound can be characterized as having a subformula according to Formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as defined herein. In some embodiments, the present disclosure also provides a compound according to Formula I, II, III, IV, V, as defined herein, or a pharmaceutically acceptable salt thereof. In some embodiments, the present disclosure provides a compound selected from compounds shown in Table A herein, or a pharmaceutically acceptable salt thereof. In some embodiments, the present disclosure provides a compound selected from Compound Nos. 1-177, or a pharmaceutically acceptable salt thereof.

In an embodiment, the present disclosure provides a pharmaceutical composition comprising a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) and optionally a pharmaceutically acceptable carrier, additive or excipient and optionally an additional bioactive agent. In some embodiments, the additional bioactive agent is an antiviral agent. In some embodiments, the pharmaceutical composition is in a form selected from tablet, powder, microparticle, nanoparticle, granule, capsule, liquid, aqueous solution, suspension, or dispersion.

In an embodiment, the present disclosure provides a method for treating coronavirus infections and associated conditions in a subject in need thereof, comprising administering to the subject a therapeutically effective amount of a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) or a pharmaceutical composition comprising the compound of the present disclosure. In some embodiments, the coronavirus infection is caused by a virus selected from SARS-CoV, HCoV-229E, HCoV-OC43, HCoV-NL63, MERS-CoV, HCoV-HKU1, and SARS-CoV-2. In some embodiments, the coronavirus infection is caused by SARS-CoV-2. In some embodiments, the compound of the present disclosure is administered in an amount effect to modulate the amount or activity of a viral protein encoded by the coronavirus in the subject, wherein the viral protein is responsible for viral reproduction. In some embodiments, the viral protein comprises Papain-like protease (PLpro) encoded by the coronavirus selected from SARS-CoV, HCoV-229E, HCoV-OC43, HCoV-NL63, MERS-CoV, HCoV-HKU1 and SARS-CoV-2. In some embodiments, the viral protein comprises PLpro encoded by SARS-CoV-2.

In some embodiments, the present disclosure provides a method of inhibiting papain-like protease (PLpro), the method comprising administering to a subject in need thereof an effective amount of a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) or a pharmaceutical composition comprising the compound of the present disclosure. In some embodiments, the subject suffers from viral infection caused by a virus selected from SARS-CoV, HCoV-229E, HCoV-OC43, HCoV-NL63, MERS-CoV, HCoV-HKU1, and SARS-CoV-2. In some preferred embodiments, the subject suffers from SARS-CoV-2 infection. In some embodiments, the Papain-like protease (PLpro) is encoded by a virus selected from SARS-CoV, HCoV-229E, HCoV-OC43, HCoV-NL63, MERS-CoV, HCoV-HKU1 and SARS-CoV-2, such as by SARS-CoV-2.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally or parenterally. In some embodiments, the administering can be an injection, infusion, oral delivery, or inhalation.

Compounds of the present disclosure can be used as a monotherapy or in a combination therapy. For example, a compound of the present disclosure can be used in a combination therapy along with one or more additional other antiviral agents, such as those described herein.

It is to be understood that both the foregoing summary and the following detailed description are exemplary and explanatory only, and are not restrictive of the invention herein.

### DETAILED DESCRIPTION

Coronavirus (CoV) genomes encode polyproteins consisting of structural proteins, replicase proteins, and proteases [13]. When compared with SARS-CoV and MERS-CoV, the SARS-CoV-2 showed less genetic similarity: 79% with SARS-CoV and 50% with MERS-CoV. However, the coding regions of SARS-CoV-2 had a similar genomic organization as that of the bat coronaviruses and SARS-CoV. The only significant difference was the S protein in SARS-CoV-2 was longer as compared to the S proteins encoded by the bat coronavirus, SARS-CoV, and MERS-CoV. Although phylogenetic analysis placed SARS-CoV-2 and SARS-CoV in different clades, the two viruses had around 50 conserved amino acids in the Si domain of the S protein. The receptor-binding domain (S1) of SARS-CoV-2 was closely similar to the Si domain of SARS-CoV. SARS-CoV-2 shared more similarities in terms of structure and pathogenicity with SARS-CoV than MERS-CoV. Both the CoVs use the same spike (S) protein for binding to the host cells, and both the CoVs utilize similar cellular protease for activating the S protein [15].

Recently, crystal structures of SARS-CoV PLpro in complex with ubiquitin (Ub) were determined. The PLpro-Ub complex crystal structures may provide a snapshot of the transition of PLpro from the “FQ” to the “E+Q” states revealing once again the high plasticity of the BL2 loop and its interactions with substrates, intermediates, products and inhibitors.

PLpro deconjugates ubiquitin and the ubiquitin-like protein ISG15 from host-cell proteins, enabling coronaviruses to evade the host innate immune responses. The host defense mechanism is characterized by the production of pro-inflammatory cytokines, including type I b-interferon (IFN-b) and chemokines such as CCL5 and CXCL10 [16]. A recent review [17] listed the known effects of SARS-CoV on host cell defense mechanisms as reported in various cell studies, specifically, the actions of PLpro that block the expression of proinflammatory cytokines such as IFN-b and the establishment of the antiviral state. SARS-CoV PLpro interferes with the activation of transcription factors IRF3 [18] and NF-kB [17]. Specifically, SARS-CoV blocks IRF3 phosphorylation, homodimerization, and consequently nuclear translocation, thereby inhibiting cytokine expression. The protease stabilizes NF-kB and IkBa to inhibit the activation of the NF-kB signaling pathway [20]. Finally, SARS-CoV PLpro decreases endogenous levels of proinflammatory cytokines and chemokines in activated cells [16]. Equivalent effects are observed for SARS-CoV-2 PLpro [2-4].

For this reason, antiviral drugs that are targeted against PLpro may exert a dual mechanism of action, not only inhibiting viral replication, but also blocking virus-induced cell signaling events that can compromise the host defense [2, 17].

In a broad aspect, the present disclosure provides compounds and compositions useful for inhibiting PLpro activities, such as for selective, covalent inhibition of the PLpro enzymes which can overcome problems associated with certain existing non-covalent enzyme inhibitor drugs. In some embodiments, the present disclosure also provides methods of using the compounds and compositions herein for treating virus infections such as SARS-CoV-2 infection. In some embodiments, the compounds and compositions herein can be an irreversible inhibitor that inhibits PLpro enzyme activities. The irreversible inhibition can lead to improved therapeutic efficacy in comparison with reversible inhibition of non-covalent inhibitors.

As used herein, an “irreversible inhibitor” is an inhibitor that can covalently bind to a target protein and inhibit the activity of the target protein for a period that is longer than the functional life of the target protein. Irreversible inhibitors are usually characterized by time dependency. Recovery of target protein activity when inhibited by an irreversible inhibitor is dependent upon new protein synthesis. Suitable methods for determining if a compound is an irreversible inhibitor are well-known in the art through kinetic analysis. As used herein, a “reversible inhibitor” is a compound that reversibly binds a target protein and inhibits the activity of the target protein. Recovery of target protein activity when inhibited by a reversible inhibitor can occur by dissociation of the reversible inhibitor from the target protein.

### Formula X

As discussed herein, in some embodiments, compounds of the present disclosure are typically designed such that a PLpro inhibitor can be covalently linked to a “warhead” which can form a covalent bond with PLpro through a proper linker. The PLpro inhibitor, linker, and warhead suitable for this design are not particularly limited and are exemplified herein.

In some embodiments, the present disclosure provides a compound of Formula X, or a pharmaceutically acceptable salt thereof,

A-L-W Formula X,
wherein:
A represents a residue of a papain-like protease (PLpro) inhibitor,
W represents a group capable of forming a covalent bond with PLpro, preferably, capable of forming a covalent bond with an SH group of a cysteine residue of PLpro, and
L represents a linker that covalently links A and W.

In some embodiments, A can be characterized as having a structure according to

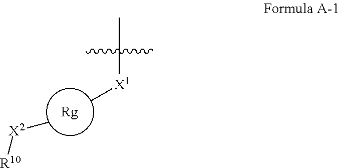

wherein:
X¹ is absent, CR¹¹R¹², NR¹³C(O), or —C(O)N(R¹³)—,
X² is absent, optionally substituted C₁₋₄ alkylene, or optionally substituted C₁₋₄ heteroalkylene,
Rg is an optionally substituted ring structure having 4-10 ring atoms, preferably, an optionally substituted 4-10 membered heterocyclic ring, and wherein:
R¹⁰ is an optionally substituted aryl or an optionally substituted heteroaryl,
R¹¹ and R¹² are each independently hydrogen, halogen, OH, NH₂, COOH, CONH₂, SO₂NH₂, G^{A}, OG^{A}, NH(G^{A}), N(G^{A})(G^{A}), COOG^{A}, CONH(G^{A}), CON(G^{A})(G^{A}), SO₂NH(G^{A}), SO₂N(G^{A})(G^{A}), NHCOG^{A}, N(G^{A})COG^{A}, NHSO₂(G^{A}), N(G^{A})SO₂(G^{A}), COG^{A}, or SO₂(G^{A}), or R¹¹ and R¹² are joined to form a 3-8 membered ring structure; R¹³ is hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G^{A}),
wherein G^{A} at each occurrence is independently an optionally substituted C₁₋₆ alkyl, optionally substituted C₂₋₆ alkenyl, optionally substituted C₂₋₆ alkynyl, an optionally substituted C₁₋₆ heteroalkyl, or an optionally substituted ring structure having 4-10 ring atoms. As used herein, unless specified or otherwise contrary from context, a “ring structure” includes both carbocyclic and heterocyclic ring, which can be saturated, partially unsaturated, or aromatic, and the ring structure can be monocyclic or contain two or more rings. For example, as used herein an “optionally substituted ring structure having 4-10 ring atoms” includes both C₄₋₁₀ carbocyclic ring and 4-10 membered heterocyclic ring, which can be aromatic or nonaromatic, which can be optionally substituted with one or more suitable substituents as described herein. A divalent ring structure herein should be understood as bonding with the remainder of the molecule through ring atom(s), which can be carbon or heteroatom such as nitrogen. For example, it should be understood that ring Rg is bonded with X¹ and X² through one or two, typically two ring atoms (e.g., ring nitrogen or ring carbon atom, etc.) of Rg.

Typically, in the moiety of Formula A-1, Rg is optionally substituted 4-10 membered heterocyclic ring. More preferably, Rg is an optionally substituted 4-7 membered monocyclic heterocyclic ring having one or two ring heteroatoms, each independently O, S, or N, for example, a saturated 4-7 membered monocyclic heterocyclic ring, such as piperidine, piperazine, pyrrolidine ring, etc. In preferred embodiments, Rg is a non-aromatic ring. In some embodiments, Rg can be an unsubstituted 4-10 membered heterocyclic ring. When substituted, Rg can be typically substituted with one or more such as 1-3 substituents independently selected from F, Cl, —OH, protected hydroxyl, oxo (as applicable), NH₂, protected amino, NH(C₁₋₄ alkyl) or a protected derivative thereof, N(C₁₋₄ alkyl)(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy.

For example, in some embodiments, the moiety of Formula A-1 can be characterized as having a structure according to Formula A-2:

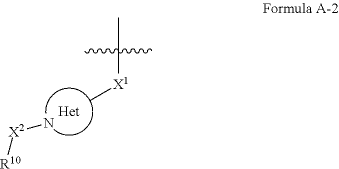

wherein Het represents an optionally substituted 4-10 membered heterocyclic ring, which is bonded with X² through a ring nitrogen atom as drawn, and is bonded with X¹ through another ring atom, wherein the 4-10 membered heterocyclic ring has 0-3 ring heteroatoms each independently O, S, or N, in addition to the ring nitrogen atom as drawn. In preferred embodiments, Het is a non-aromatic ring, such as a 4-7 membered saturated monocyclic heterocyclic ring having 0 or 1 ring heteroatom in addition to the nitrogen atom as shown in Formula A-2. In some embodiments, Het is unsubstituted. In some embodiments, Het can also be substituted with one or more such as 1-3 substituents. Suitable substituents are described herein, such as those described in connection with Rg.

In some specific embodiments, the moiety of Formula A-1 can be characterized as having a structure according to Formula A-3:

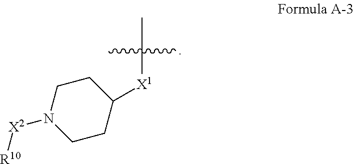

X² in Formula A-1 (including sub-formula A-2 or A-3) is typically an optionally substituted C₁₋₄ alkylene. For example, in some embodiments, X² can be a linear or branched unsubstituted C₁₋₄ alkylene (e.g., CH₂, CH(CH₃), CH(C₂H₅), etc.). When substituted, the C₁₋₄ alkylene can be typically substituted with one or more, such as 1-3, substituents independently selected from F, Cl, —OH, protected hydroxyl, oxo (as applicable), NH₂, protected amino, NH(C₁₋₄ alkyl) or a protected derivative thereof, N(C₁₋₄ alkyl)(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, two or more substituents of the C₁₋₄ alkylene can also be joined to form a ring structure, such as a 3-7 membered ring structure, for example,

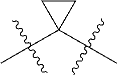 [SMILES: *C1(*)CC1]

can viewed as a methylene, with two gem substituents joined to form a 3-membered ring structure.

In some embodiments, X² in Formula A-1 (including sub-formula A-2 or A-3) can also be absent. In other words, R¹⁰ is attached to Rg directly.

In some more specific embodiments, X² in Formula A-1 (including sub-formula A-2 or A-3) can be represented by CR¹⁴R¹⁵, wherein R¹⁴ and R¹⁵ are each independently hydrogen, an optionally substituted C₁₋₄ alkyl, optionally substituted C₂₋₄ alkenyl, optionally substituted C₂₋₄ alkynyl, or an optionally substituted ring structure having 4-10 ring atoms. In some more specific embodiments, X² in Formula A-1 (including sub-formula A-2 or A-3) can be represented by CR¹⁴R¹⁵, wherein one of R¹⁴ and R¹⁵ is COOH, or an ester or amide thereof, and the other of R¹⁴ and R¹⁵ is as defined in the foregoing. In some embodiments, X² in Formula A-1 (including sub-formula A-2 or A-3) can be represented by CR¹⁴R¹⁵, wherein R¹⁴ and R¹⁵ together with the carbon they are both attached to form a 3-6 membered ring which is optionally substituted. For example, in some embodiments, one of R¹⁴ and R¹⁵ is hydrogen, and the other of R¹⁴ and R¹⁵ is hydrogen, C₁₋₄ alkyl optionally substituted with 1-3 G^{S1}, C₃₋₆ cycloalkyl optionally substituted with 1-3 G^{S1}, or phenyl optionally substituted with 1-3 G^{S1}, wherein G^{S1} at each occurrence is independently halogen (preferably F), NH₂, OH, C₁₋₄ alkyl, or C₁₋₄ alkoxy. In some embodiments, one of R¹⁴ and R¹⁵ is hydrogen, and the other of R¹⁴ and R¹⁵ is hydrogen, methyl, or phenyl. In some embodiments, one of R¹⁴ and R¹⁵ is hydrogen, and the other of R¹⁴ and R¹⁵ is hydrogen, methyl, ethyl, hydroxymethyl, 2-hydroxyethyl, aminomethyl or 2-aminoethyl. In some embodiments, one of R¹⁴ and R¹⁵ is hydrogen, and the other of R¹⁴ and R¹⁵ is CH₂F, CH₂OH, CHF₂, CD₃, CF₃, ethyl, CN, cyclopropyl, COOH, or COOCH₃. In some embodiments, R¹⁴ and R¹⁵ together with the carbon they are both attached to form a cyclopropyl.

R¹⁰ in Formula A-1 (including sub-formula A-2 or A-3) is typically an optionally substituted aryl. For example, in some embodiments, R¹⁰ in Formula A-1 (including sub-formula A-2 or A-3) can be an optionally substituted phenyl or optionally substituted naphthyl. In some embodiments, the phenyl or naphthyl is unsubstituted. When substituted, the phenyl or naphthyl can be typically substituted with one or more substituents (e.g., 1-3 substituents) independently selected from F, Cl, Br, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

In more specific embodiments, R¹⁰ in Formula A-1 (including sub-formula A-2 or A-3) can be an

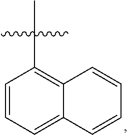 [SMILES: *c1cccc2ccccc12]

which is optionally substituted, such as with 1-3 G^{S1}, wherein G^{S1} at each occurrence is independently halogen (preferably F or Cl), OH, C₁₋₄ alkyl, or C₁₋₄ alkoxy (e.g., methoxy). In more specific embodiments, R¹⁰ in Formula A-1 (including sub-formula A-2 or A-3) can be an

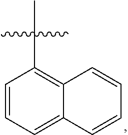 [SMILES: *c1cccc2ccccc12]

which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring. In more specific embodiments, R¹⁰ in Formula A-1 (including sub-formula A-2 or A-3) can be an

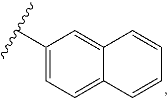 [SMILES: *c1ccc2ccccc2c1]

which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy, OCF₃); or an optionally substituted 3-7 membered ring.

In more specific embodiments, R¹⁰ in Formula A-1 (including sub-formula A-2 or A-3) can be an optionally substituted phenyl,

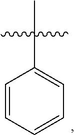 [SMILES: *c1ccccc1]

which is optionally substituted, such as with 1-3 G^{S1B}, wherein G^{S1}s at each occurrence is independently halogen (e.g., F, Cl, or Br), OH, C₁₋₄ alkyl optionally substituted with 1-3 F (e.g., methyl, CF₃), C₁₋₄ alkoxy optionally substituted with 1-3 F (e.g., methoxy), or an optionally substituted 3-7 membered ring (e.g., phenyl, thienyl, etc.).

In some preferred embodiments, X²—R¹⁰ in Formula A-1 (including sub-formula A-2 or A-3) can be

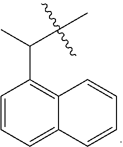 [SMILES: *C(C)c1cccc2ccccc12]

In some embodiments, X²—R¹⁰ in Formula A-1 (including sub-formula A-2 or A-3) can be

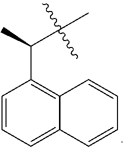 [SMILES: *[C@H](C)c1cccc2ccccc12]

In some embodiments, X²—R¹⁰ in Formula A-1 (including sub-formula A-2 or A-3) can be

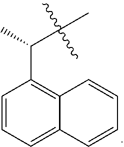 [SMILES: *[C@@H](C)c1cccc2ccccc12]

As used herein, when a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s), for example, the compound can have an enantiomeric excess (ee) of at least 60%, preferably, at least 80%, at least 90%, at least 95%, at least 98%, at least 99%, etc. The presence and/or amounts of stereoisomers can be readily determined by a person of ordinary skill in the art in view of the present disclosure, including through the use of a chiral HPLC or any other suitable methods.

In some embodiments, R¹⁰ in Formula A-1 (including sub-formula A-2 or A-3) can also be an optionally substituted heteroaryl such as quinolinyl, isoquinolinyl or quinazolinyl. In some embodiments, R¹⁰ in Formula A-1 (including sub-formula A-2 or A-3) can be a heteroaryl ring, such as a bicyclic heteroaryl having 9 or 10 ring atoms with 1-3 ring heteroatoms, each independently N, O, or S, such as quinoline, isoquinoline, benzo[d]thiazole, etc. For example, in some embodiments, R¹⁰ in Formula A-1 (including sub-formula A-2 or A-3) can be

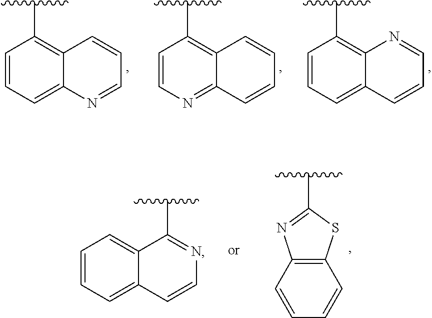

which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy, OCF₃); or an optionally substituted 3-7 membered ring.

In some embodiments, X¹ in Formula A-1 (including sub-formula A-2 or A-3) can be NR¹³, wherein R¹³ is defined herein. In some preferred embodiments, R¹³ can be hydrogen, optionally substituted C₁₋₆ alkyl, COG^{A} or SO₂G^{A}, wherein G^{A} includes any of those described herein. In some embodiments, R¹³ is an optionally substituted C₁₋₆ alkyl, optionally substituted C₂₋₆ alkenyl or optionally substituted C₂₋₆ alkynyl, e.g., substituted with COG^{A} or SO₂G^{A}, such as COG^{A1}, COG^{A2}, COG^{A3}, SO₂G^{A1}, SO₂G^{A4}, or SO₂G^{A5}, as defined herein, for example, R¹³ can be or

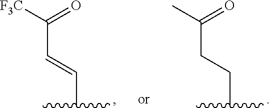

For example, in some embodiments, X¹ in Formula A-1 (including sub-formula A-2 or A-3) can be NR¹³ wherein R¹³ is COG^{A1}, wherein G^{A1} is an optionally substituted C₁₋₆ alkyl, optionally substituted C₃₋₆ cycloalkyl, optionally substituted phenyl, optionally substituted naphthyl, or an optionally substituted ring structure having 4-10 ring atoms with 1-3 ring heteroatoms. In some embodiments, R¹³ can be COG^{A2}, wherein G^{A2} is a C₁₋₆ alkyl, which is optionally substituted with 1-3 G^{S2},
wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure. In some embodiments, R¹³ can be COG^{A}2, wherein G^{A2} is a C₁₋₆ alkyl, which is optionally substituted with 1-3 G^{S2A}, wherein G^{S2A} at each occurrence is independently halogen (preferably F), OH, COOH, CONH₂, G^{S2B}, CONHG^{S2B}, CONG^{S2B} G^{S2B}, SO₂NH₂, SO₂NHG^{S2B}, SO₂NG^{S2B}G^{S2B}, COG^{S2B}, CO₂G^{S2B}, or SO₂G^{S2B}, wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, wherein G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure. In some embodiments, R¹³ can be COG^{A3}, wherein G^{A3} is a C₃₋₆ cycloalkyl, which is optionally substituted with 1-5 G^{S3}, wherein
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure; and G^{S1} at each occurrence is independently halogen (preferably F), OH, C₁₋₄ alkyl, or C₁₋₄ alkoxy. In any of the embodiments described herein, unless specified or otherwise contrary from context, R¹³ can be selected from:

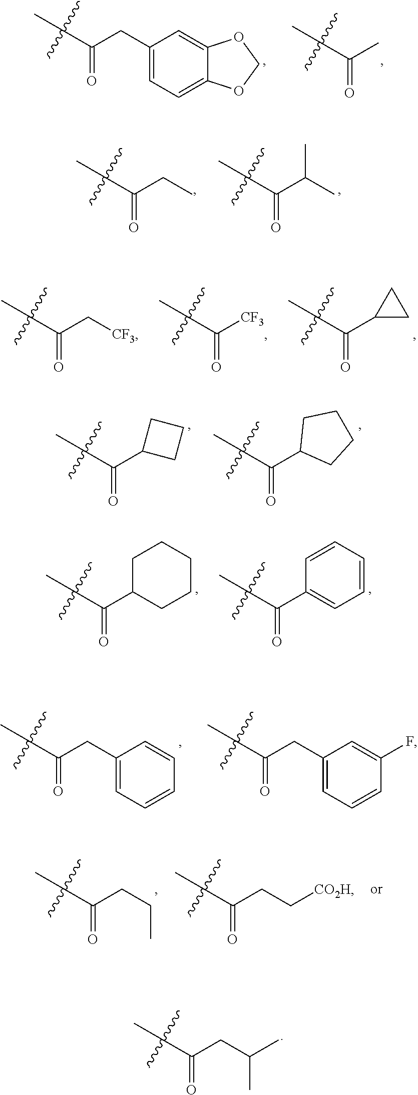

In some embodiments, X¹ in Formula A-1 (including sub-formula A-2 or A-3) can be NR¹, wherein R¹³ is SO₂G^{A1}, wherein G^{A1} is an optionally substituted C₁₋₆ alkyl, optionally substituted C₃₋₆ cycloalkyl, optionally substituted phenyl, optionally substituted naphthyl, or an optionally substituted ring structure having 4-10 ring atoms with 1-3 ring heteroatoms. In some embodiments, R¹³ can be SO₂G^{A4}, and G^{A4} is a C₁₋₆ alkyl, which is optionally substituted with 1-3 G^{S2}, wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3} wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure. In some embodiments, R¹³ can be SO₂G^{A4}, and G^{A4} is a C₁₋₆ alkyl, optionally substituted with 1-3 G^{S2A}, wherein G^{S2A} at each occurrence is independently halogen (preferably F), OH, COOH, CONH₂, G^{S2B}, CONHG^{S2B} , CONG^{S2B} G^{S2B}, SO₂NH₂, SO₂NHG^{S2B}, SO₂NG^{S2B}G^{S2B}, COG^{S2B}, CO₂G^{S2B}, or SO₂G^{S2B}, wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure. In some embodiments, R¹³ can be SO₂G^{A5}, wherein G^{A5} is a C₃₋₆ cycloalkyl or phenyl, which is optionally substituted with 1-5 G^{S3}, wherein
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure; and G^{S1} at each occurrence is independently halogen (preferably F), OH, C₁₋₄ alkyl, or C₁₋₄ alkoxy. In some embodiments, R¹³ can be SO₂G^{A5}, wherein G^{A5} is a 4-7 membered heterocyclic ring having 1 or 2 ring heteroatoms, which are independently N, O, or S, such as pyrrolidine, piperidine, or piperazine, etc., wherein the 4-7 membered heterocyclic ring is optionally substituted with 1-3 substituents independently selected from G^{S3A} (C₁₋₄ alkylene)-G^{S3A}, OG^{S3A}, NHG^{S3A}, NG^{S3A}G^{S3A}, or (C₁₋₄ heteroalkylene)-G^{S3A} G^{S3A} at each occurrence is independently halogen (preferably F), OH, COOH, CONH₂, G^{S2B}, CONHG^{S2B}, CONG^{S2B} G^{S2B}, SO₂NH₂, SO₂NHG^{S2B}, SO₂NG^{S2B}G^{S2B}, COG^{S2B}, CO₂G^{S2B}, or SO₂G^{S2B}, wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure. In some embodiments, X¹ in Formula A-1 (including sub-formula A-2 or A-3) can be NR¹³ wherein R¹³ can be COOG^{A}, CONH₂, CONH(G^{A}), CON(G^{A})(G^{A}), SO₂NH₂, SO₂NH(G^{A}), or SO₂N(G^{A})(G^{A}), wherein G^{A} is defined herein. In any of the embodiments described herein, unless specified or otherwise contrary from context, R¹³ can be selected from:

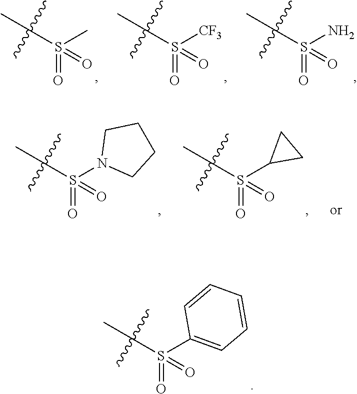

In any of the embodiments described herein, unless specified or otherwise contrary from context, R¹³ can be selected from:

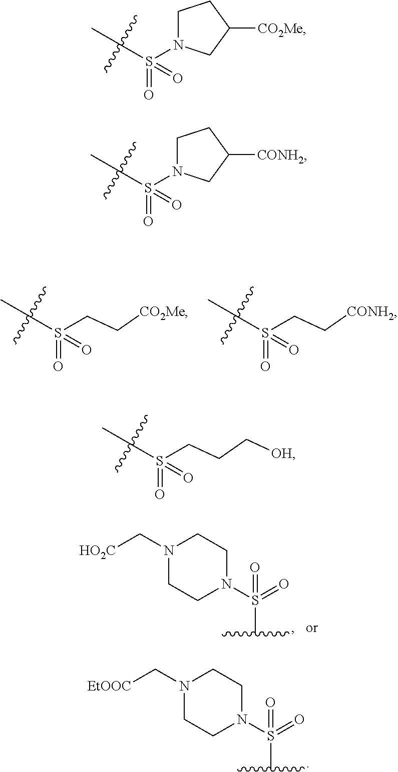

In some embodiments, X¹ in Formula A-1 (including sub-formula A-2 or A-3) can be CR¹¹R¹², wherein R¹¹ and R¹² are defined herein. For example, in some embodiments, one of R¹¹ and R¹² is hydrogen, and the other of R¹¹ and R¹² can be hydrogen, NH₂, NH(G^{A}), N(G^{A})(G^{A}), NHCO(G^{A}), or N(G^{A})CO(G^{A}), wherein G^{A} is defined herein. In some embodiments, one of R¹¹ and R¹² is hydrogen, and the other of R¹¹ and R¹² can be hydrogen, NH₂, NH(G^{A6}), or N(G^{A6})(G^{A6}), wherein G^{A6} at each occurrence is independently a C₁₋₆ alkyl, which is optionally substituted with 1-3 G^{S2},
wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure.

In some embodiments, L in Formula X can be characterized as having a structure according to Formula L-1 (A and W are shown to show direction of attachment):

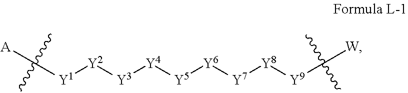

wherein:
Y¹ and Y⁹ are each independently absent, an optionally substituted C₁₋₄ alkylene, optionally substituted C₁₋₄ heteroalkylene, or optionally substituted 3-8 membered ring structure,
Y², Y³, Y⁴, Y₅, Y⁶, Y⁷, and Y⁸ are each independently absent, CR²⁰R²¹, C(O), C(S), O, NR²², S, SO, or SO₂, wherein up to 4 of Y², Y³, Y⁴, Y⁵, Y⁶, Y⁷, and Y⁸ can be absent, wherein
   (i) two adjacent CR²⁰R²¹ units can optionally form a double bond to form —C(R²⁰)═C(R²¹)— or —C≡C—;
   (ii) two adjacent CR²⁰R²¹ and NR²² units can optionally form a double bond to form —C(R²⁰)=N—; or
   (iii) two adjacent NR²² units can optionally form a double bond to form —N═N—; or three or four consecutive Y², Y³, Y⁴, Y⁵, Y⁶, Y⁷, and Y⁸ together (e.g., Y⁴, Y⁵, and Y⁶; Y⁵, Y⁶, and Y⁷, or Y⁶, Y⁷, and Y⁸, etc.) represent a 3-10 membered ring structure, and the remaining of Y², Y³, Y⁴, Y⁵, Y⁶, Y⁷, and Y⁸ are as defined above, wherein:
R²⁰ and R²¹ at each occurrence are each independently hydrogen, halogen, OH, NH₂, COOH, CONH₂, SO₂NH₂, G^{A}, OG^{A}, NH(G^{A}), N(G^{A})(G^{A}), COOG^{A}, CONH(G^{A}), CON(G^{A})(G^{A}), SO₂NH(G^{A}), SO₂N(G^{A})(G^{A}), NHCOG^{A}, N(G^{A})COG^{A}, NHSO₂(G^{A}), N(G^{A})SO₂(G^{A}), COG^{A}, or SO₂(G^{A}),
R²² at each occurrence is independently hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G^{A}),
wherein G^{A} at each occurrence is independently an optionally substituted C₁₋₆ alkyl, optionally substituted C₂₋₆ alkenyl, optionally substituted C₂₋₆ alkynyl, optionally substituted C₁₋₆ heteroalkyl, or an optionally substituted ring structure having 4-10 ring atoms;
or two or more instances of R²⁰, R²¹ and R²² (e.g., R²⁰ and R²¹ on the same carbon, R²⁰ and R²⁰ on two different carbons, R²⁰ and R²², etc.), can be joined to form a 3-8 membered ring structure.

In some embodiments, Y¹ can be absent. In some embodiments, Y¹ is an optionally substituted C₁₋₄ alkylene, such as CH₂ or CD₂. In some embodiments, Y¹ is an optionally substituted 4-8 membered heterocyclic ring having 1-3 ring heteroatoms independently O, S, or N, such as

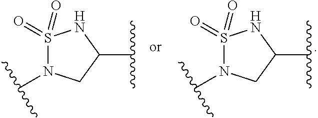 [SMILES: *C1CN(*)S(=O)(=O)N1 ; *C1CN(*)S(=O)(=O)N1]

In some embodiments, Y⁹ can be absent, an optionally substituted C₁₋₄ alkylene, such as CH₂, CD₂ or CF₂, or an optionally substituted 4-8 membered heterocyclic ring having 1 or 2 ring heteroatoms independent y O, S, or N. In some embodiments, Y⁹ can be a C₃₋₆ membered carbocyclic ring, such as

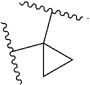 [SMILES: *C1(*)CC1]

In some embodiments, Y⁹ can be a C₁₋₄ heteroalkylene (e.g., —NH—CH₂—, —CH₂—NH—CH₂—, etc.), which is optionally substituted, such as with oxo, F, CF₃, etc. Typically, the C₁₋₄ heteroalkylene herein has 1 or 2 heteroatoms, such as one oxygen, one nitrogen, two oxygen, two nitrogen, or one oxygen and one nitrogen, not considering the substituents. In some embodiments, the C₁₋₄ heteroalkylene can be substituted with an oxo and can have a structure such as —C(O)—NH—CH₂—, etc.

In some embodiments, Y² is CH₂ or CH(C₁₋₄ alkyl), such as CH(CH₃), wherein the C₁₋₄ alkyl is optionally substituted, such as with substituents independently selected from F, OH, and NH₂. In some embodiments, Y² is absent. In some embodiments, Y² is CH(CH₃), C(CH₃)₂, CD₂ or

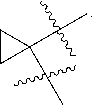 [SMILES: *C1(*)CC1]

In some embodiments, Y³ is C(O). In some embodiments, Y³ is absent. In some embodiments, Y³ is CH(OH) or CH(C₁₋₄ alkyl), wherein the C₁₋₄ alkyl is optionally substituted, such as with substituents independently selected from F, OH, and NH₂, for example, Y³ is CH(CF₃).

In some embodiments, Y⁴ is NH, CH₂ or CH(C₁₋₄ alkyl). In some embodiments, Y⁴ is absent. In some embodiments, Y⁴ is N(C₁₋₄ alkyl), such as NCH₃. Each of the C₁₋₄ alkyl in this paragraph should be understood as can be optionally substituted, such as with substituents independently selected from F, OH, and NH₂.

In some embodiments, Y⁵ is NH, CH₂, CH(C₁₋₄ alkyl), C(C₁₋₄ alkyl)(C₁₋₄ alkyl), or

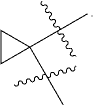 [SMILES: *C1(*)CC1]

In some embodiments, Y⁵ is absent. In some embodiments, Y⁵ is CH(CH₃), C(CH₃)₂, CD₂ or

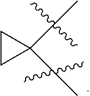 [SMILES: *C1(*)CC1]

In some embodiments, Y⁶ is C(O), CH₂ or CH(C₁₋₄ alkyl). In some embodiments, Y⁶ is absent. In some embodiments, Y⁶ is CH(OH) or CH(C₁₋₄ alkyl), wherein the C₁₋₄ alkyl is optionally substituted, such as with substituents independently selected from F, OH, and NH₂, for example, Y⁶ is CH(CF₃).

In some embodiments, Y⁷ is NH, CH₂ or CH(C₁₋₄ alkyl). In some embodiments, Y⁷ is absent. In some embodiments, Y⁷ is N(C₁₋₄ alkyl), such as NCH₃, CH(CH₃), C(CH₃)₂, or CD₂. Each of the C₁₋₄ alkyl in this paragraph should be understood as can be optionally substituted, such as with substituents independently selected from F, OH, and NH₂.

In some embodiments, Y⁸ is NH. In some embodiments, ^{Y} is O or N(C₁₋₄ alkyl), such as NCH₃, wherein the C₁₋₄ alkyl is optionally substituted, such as with substituents independently selected from F, OH, and NH₂. In some embodiments, Y⁸ is CH₂ or CH(C₁₋₄ alkyl). In some embodiments, Y⁸ is CO. In some embodiments, ^{Y} is CH(CH₃), C(CH₃)₂, CD₂ or

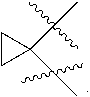 [SMILES: *C1(*)CC1]

In some embodiments, Y⁸ can also be absent.

In some embodiments, the linker of Formula L-1 can be characterized by one or more of the following features:
(i) Y² is CH₂ or CH(C₁₋₄ alkyl);
(ii) Y³ is C(O);
(iii) Y⁴ is NH, CH₂ or CH(C₁₋₄ alkyl);
(iv) Y⁵ is NH, CH₂, CH(C₁₋₄ alkyl), C(C₁₋₄ alkyl)(C₁₋₄ alkyl), or

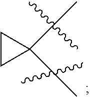 [SMILES: *C1(*)CC1]

(v) Y⁶ is C(O), CH₂ or CH(C₁₋₄ alkyl);
(vi) Y⁷ is absent, C(O), NH, CH₂ or CH(C₁₋₄ alkyl); and/or
(vii) Y⁸ is NH, CH₂ or CH(C₁₋₄ alkyl), or absent.
It should be understood that the two C₁₋₄ alkyl in “C(C₁₋₄ alkyl)(C₁₋₄ alkyl)” are independently selected and can be the same or different. Other similar expressions should be understood similarly.

In some embodiments, the linker of Formula L-1 can be characterized in that two of Y², Y³, Y⁴, Y⁵, Y⁶, Y⁷, and Y⁸ are C(O), for example, Y³ and Y⁶ are C(O). In some embodiments, the linker of Formula L-1 can be characterized in that one of Y², Y³, Y⁴, Y⁵, Y⁶, Y⁷, and Y⁸ is C(O). In some embodiments, the linker of Formula L-1 can be characterized in that one of Y², Y³, Y⁴, Y⁵, Y⁶, Y⁷, and Y⁸ is SO₂. In some embodiments, the linker of Formula L-1 can be characterized in that up to four (e.g., 1, 2, 3, or 4) of Y², Y³, Y⁴, Y⁵, Y⁶, Y⁷, and Y⁸ can be NR²². In some embodiments, the linker of Formula L-1 can be characterized in that Y³ is C(O) and Y⁴ is NR²².

In some embodiments, the linker of Formula L-1 can be characterized in that Y⁴, Y⁵ and Y⁶ are joined to form a 3-10 membered ring, such as a 4, 5, or 6-membered heterocyclic ring having 1-3 ring heteroatoms independently selected from N, O, and S, such as

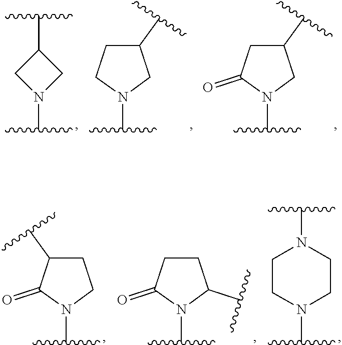

or a spiro bicyclic ring, such as

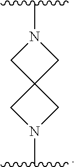 [SMILES: *N1CC2(C1)CN(*)C2]

In some embodiments, the linker of Formula L-1 can be characterized in that Y⁵, Y⁶ and Y⁷ are joined to form a 3-6 membered ring, such as a 5-membered heteroaryl ring having 1-3 ring heteroatoms independently selected from N, O, and S, such as

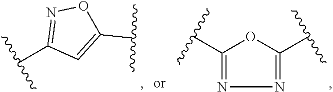 [SMILES: *c1cc(*)on1 ; *c1nnc(*)o1]

or a 4 or 5-membered heterocyclic ring having 1-3 ring heteroatoms independently selected from N, O, and S, such as

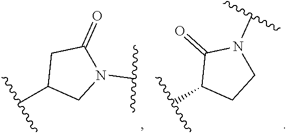

In some embodiments, the linker of Formula L-1 can be characterized in that Y⁶, Y⁷ and Y⁸ are joined to form a 3-6 membered ring, such as a 4 or 5-membered heterocyclic ring having 1-3 ring heteroatoms independently selected from N, O, and S.

In some embodiments, the linker of Formula L-1 can be characterized in that Y⁶ is C(O) and Y⁷ is NR²². In some embodiments, the linker of Formula L-1 can be characterized in that Y⁷ is C(O) and Y⁸ is NR²². In some embodiments, the linker of Formula L-1 can be characterized in that Y⁸ is absent, CH₂ or CH(C₁₋₄ alkyl).

Combinations of features of Formula L-1 are not particularly limited and include those exemplified in the specific compounds shown in Table A or Compound Nos. 1-177 herein. For example, in some embodiments, Formula A has a structure according to Formula A-1, and the linker of Formula L-1 can be characterized as having Formula L-2, L-2A, L-3, L-3A, L-4, L-4A, L-5 or L-6 (X¹ and W are shown to show direction of attachment of the linker to A and W):

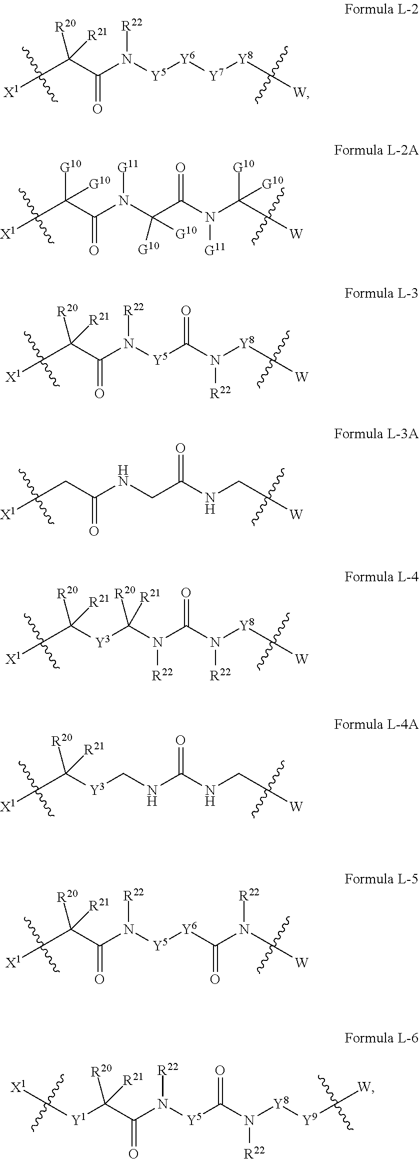

wherein in Formula L-2A, each G¹⁰ is independently hydrogen, deuterium, methyl, or two G¹⁰ together with the carbon they are both attached to form a cyclopropylene,

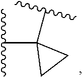 [SMILES: *C1(*)CC1]

and each G¹¹ is hydrogen or methyl, provided that out of all instances of G¹⁰ and G¹¹ combined, at most four instances are not hydrogen or deuterium, preferably, at most three instances are not hydrogen or deuterium, more preferably, only one or two instances are not hydrogen or deuterium,
wherein the remaining variables are as defined for the respective variable herein.

In some embodiments, R²⁰ and R²¹ at each occurrence are each independently hydrogen or optionally substituted C₁₋₄ alkyl, (e.g., CF₃). In some embodiments, R²⁰ and R²¹ at each occurrence are each independently hydrogen, deuterium, OH, or optionally substituted C₁₋₄ alkyl (e.g., CF₃). In some embodiments, R²² at each occurrence is independently hydrogen or optionally substituted C₁₋₄ alkyl. Other definitions of R²⁰, R²¹, and R²² are described herein.

In some embodiments, the linker of Formula L-1 (e.g., L-2, L-3, L-4, or L-5) can be characterized as having one or more of the following applicable features:
i) Y¹ is absent;
ii) Y² is CH₂ or CH(C₁₋₄ alkyl);
iii) Y³ is C(O);
iv) Y⁴ is NH, CH₂ or CH(C₁₋₄ alkyl);
v) Y⁵ is NH, CH₂, CH(C₁₋₄ alkyl), C(C₁₋₄ alkyl)(C₁₋₄ alkyl), or

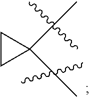 [SMILES: *C1(*)CC1]

vi) Y⁶ is C(O), CH₂ or CH(C₁₋₄ alkyl);
vii) Y⁷ is NH, CH₂ or CH(C₁₋₄ alkyl); or Y⁷ is C(O);
viii) Y⁸ is NH; or Y⁸ is CH₂ or CH(C₁₋₄ alkyl); or Y⁸ is absent; and
ix) Y⁹ is absent, an optionally substituted C₁₋₄ alkylene, or an optionally substituted 4-8 membered heterocyclic ring having 1 or 2 ring heteroatoms independently O, S, or N.

In some embodiments, the linker L in Formula X can have a structure of

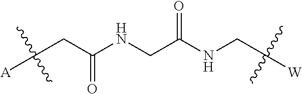

(A and W are shown to show direction of attachment). In some embodiments, the linker L in Formula X can have a structure of

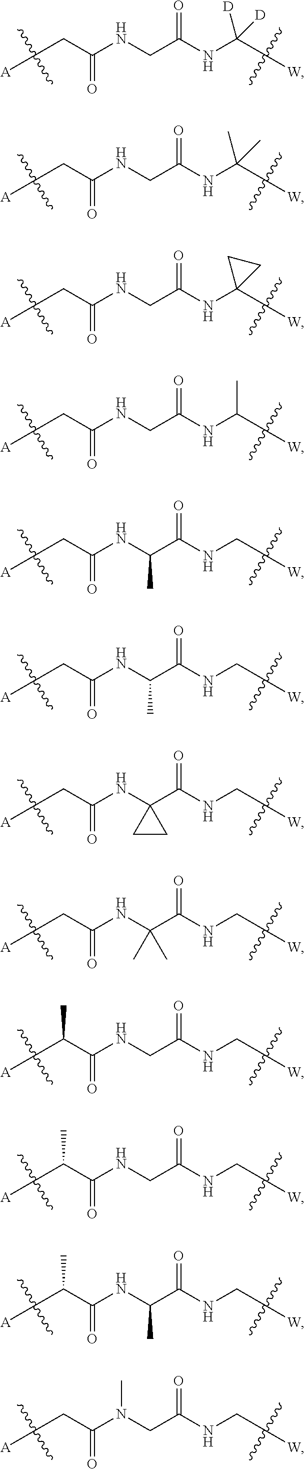

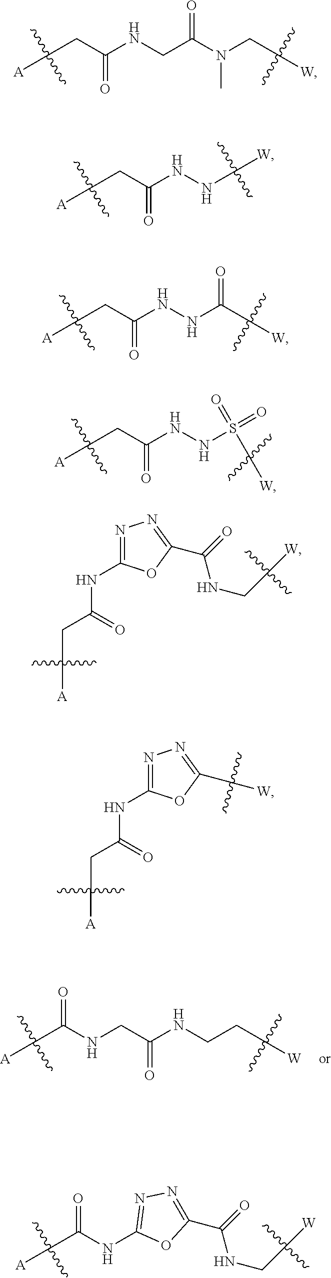

(A and W are shown to show direction of attachment). In some embodiments, the linker L in Formula X can have a structure of

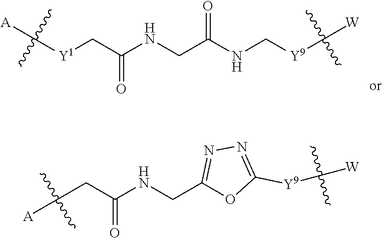

(A and W are shown to show direction of attachment). In some embodiments, Y¹ is absent. In some embodiments, Y⁹ is absent. In some embodiments, Y⁹ is a C₁₋₄ alkylene or C₁₋₄ heteroalkylene, each of which can be branched or linear.

In some embodiments, W in Formula X can be characterized as having one or more of the following moiety:

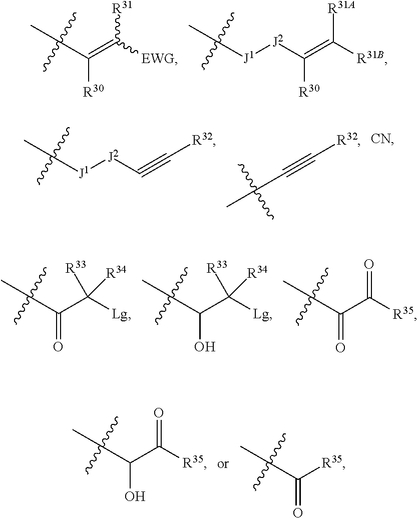

or a protected or masked derivative thereof,
wherein:
EWG is an electron withdrawing group, e.g., an aldehyde, ester, amide, sulfone, sulfonamide, CN, etc.,
Lg is OH, CN, or a leaving group, e.g., F, Cl, etc.
R³⁰ and R³¹ are each independently G^{B} or an electron withdrawing group, or R³⁰ and R³¹ or EWG are joined to form a non-aromatic ring structure, or R³¹ and EWG are joined to form a non-aromatic ring structure;
R^{31A} and R^{31B} are each independently G^{B} or an electron withdrawing group, or R³⁰ and R^{31B} are joined to form a non-aromatic ring structure, or R^{31A} and R^{31B} are joined to form a non-aromatic ring structure;
J¹ is O, NR³⁶, C₁₋₄ alkylene, C₁₋₄ heteroalkylene, or absent;
J² is absent, C(O), SO, or SO₂;
R³² is G^{B} or an electron withdrawing group,
R³³ and R³⁴ are independently hydrogen or optionally substituted C₁₋₄ alkyl;
R³⁵ is G^{B}, OG^{B}, NH(G^{B}), N(G^{B})(G^{B}), and
R³⁶ is hydrogen or optionally substituted C₁₋₄ alkyl or an electron withdrawing group, wherein G^{B} at each occurrence is independently hydrogen, an optionally substituted C₁₋₆ alkyl, optionally substituted C₂₋₆ alkenyl, optionally substituted C₂₋₆ alkynyl, or an optionally substituted ring structure having 4-10 ring atoms.

For example, in some embodiments, W can be selected from the following:

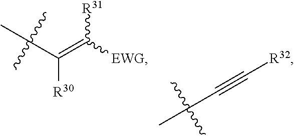

wherein:
EWG is selected from COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G^{A}),
wherein G^{A} at each occurrence is independently an optionally substituted C₁₋₆ alkyl, optionally substituted C₂₋₆ alkenyl, optionally substituted C₂₋₆ alkynyl, an optionally substituted C₁₋₆ heteroalkyl, or an optionally substituted ring structure having 4-10 ring atoms. For example, in some embodiments, R³⁰ and R³¹ are both hydrogen. In some embodiments, R³² is hydrogen or a C₁₋₄ alkyl. In some embodiments, W can be

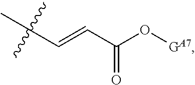

C(O)R³⁵, or

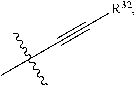

wherein G^{A7} is an optionally substituted C₁₋₆ alkyl, R³⁵ is hydrogen or a C₁₋₄ alkyl, and R³² is hydrogen or a C₁₋₄ alkyl. In some embodiments, Y⁹—W can be —(C₁₋₄ alkylene)-CN, CN,

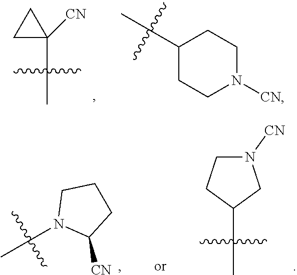

In some embodiments, Y⁹—W can be

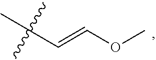 [SMILES: */C=C/OC]

which may be viewed as a masked CH₂CHO group. In some embodiments, W can be C(O)H,

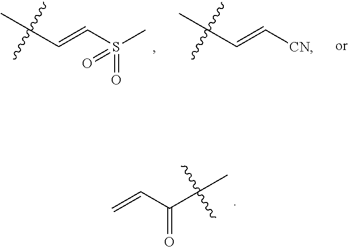

In some embodiments, W can be

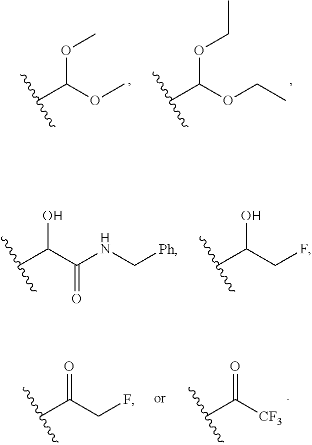

In some embodiments, W can be

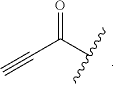 [SMILES: *C(=O)C#C]

In some preferred embodiments, W can be

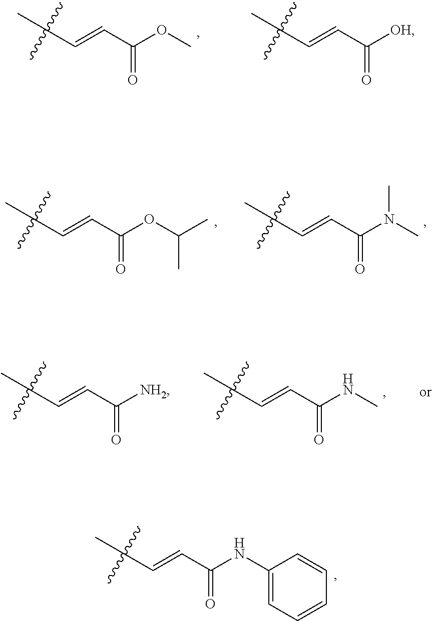

In some embodiments, W can be

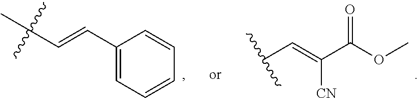

In some preferred embodiments, W can be or

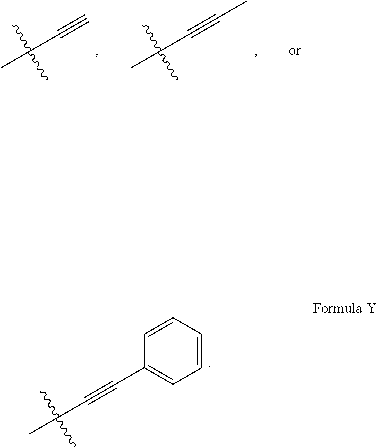

In some embodiments, the present disclosure provides a compound of Formula Y, or a pharmaceutically acceptable salt thereof,

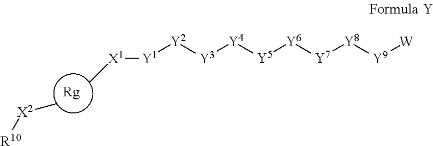

wherein:
X¹ is absent, CR¹¹R¹², NR¹³C(O), or —C(O)N(R¹³)—,
X² is absent, optionally substituted C₁₋₄ alkylene, or optionally substituted C₁₋₄ heteroalkylene,
Rg is an optionally substituted ring structure having 4-10 ring atoms, preferably, an optionally substituted 4-10 membered heterocyclic ring,
Y¹ and Y⁹ are each independently absent, an optionally substituted C₁₋₄ alkylene, optionally substituted C₁₋₄ heteroalkylene, or optionally substituted 3-8 membered ring structure,
Y², Y³, Y⁴, Y⁵, Y⁶, Y⁷, and Y⁸ are each independently absent, CR²⁰R²¹, C(O), O, NR²², S, SO, or SO₂, wherein up to 4 (e.g., 0, 1, 2, 3, or 4) of Y², Y³, Y⁴, Y⁵, Y⁶, Y⁷, and Y⁸ can be absent, wherein
(i) two adjacent CR²⁰R²¹ units can optionally form a double bond to form —C(R²⁰)═C(R²¹)— or —C≡C—;
(ii) two adjacent CR²⁰R²¹ and NR²² units can optionally form a double bond to form —C(R²⁰)═N—; or
(iii) two adjacent NR²² units can optionally form a double bond to form —N═N—; or three or four consecutive Y², Y³, Y⁴, Y⁵, Y⁶, Y⁷, and Y⁸ together (e.g., Y⁴, Y⁵, and Y⁶; or Y⁵, Y⁶, and Y⁷, etc.) represent a 3-10 membered ring structure, and the remaining of Y², Y³, Y⁴, Y⁵, Y⁶, Y⁷, and Y⁸ are as defined above,
W is'

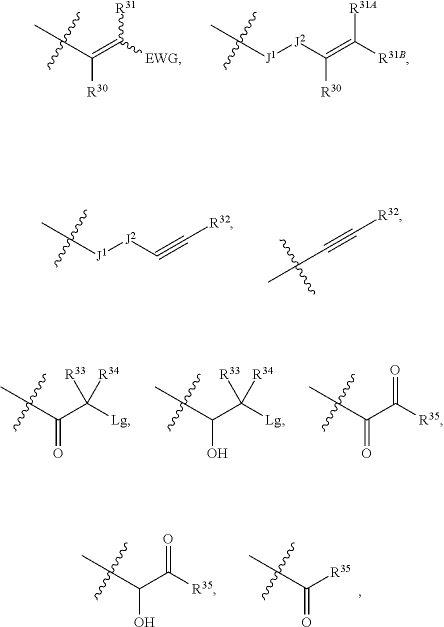

or CN, or a protected or masked derivative thereof,
R¹⁰ is an optionally substituted aryl or an optionally substituted heteroaryl,
wherein:
R¹¹ and R¹² are each independently hydrogen, halogen, OH, NH₂, COOH, CONH₂, SO₂NH₂, G^{A}, OG^{A}, NH(G^{A}), N(G^{A})(G^{A}), COOG^{A}, CONH(G^{A}), CON(G^{A})(G^{A}), SO₂NH(G^{A}), SO₂N(G^{A})(G^{A}), NHCOG^{A}, N(G^{A})COG^{A}, NHSO₂(G^{A}), N(G^{A})SO₂(G^{A}),
COG^{A}, or SO₂(G^{A}), or R¹¹ and R¹² are joined to form a 3-8 membered ring structure; R¹³ is hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G),
R²⁰ and R²¹ at each occurrence are each independently hydrogen, halogen, OH, NH₂, COOH, CONH₂, G^{A}, OG^{A}, NH(G^{A}), N(G^{A})(G^{A}), COOG^{A}, CONH₂, CONH(G^{A}), CON(G^{A})(G^{A}), SO₂NH₂, SO₂NH(G^{A}), SO₂N(G^{A})(G^{A}), NHCOG^{A}, N(G^{A})COG^{A}, NHSO₂(G^{A}), N(G^{A})SO₂(G^{A}), COG^{A}, or SO₂(G^{A}),
R²² at each occurrence is independently hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G^{A}),
or two or more instances of R²⁰, R²¹ and R²² (e.g., R²⁰ and R²¹ on the same carbon, R²⁰ and R²⁰ on two different carbons, R²⁰ and R²², etc.), can be joined to form a 3-8 membered ring structure;
EWG is an electron withdrawing group, e.g., an aldehyde, ester, amide, sulfone, sulfonamide, CN, etc.,
R³⁰ and R³¹ are each independently hydrogen, G^{A}, or an electron withdrawing group, or R³⁰ and R³¹ or EWG are joined to form a non-aromatic ring structure, or R³¹ and EWG are joined to form a non-aromatic ring structure;
R^{31A} and R^{31B} are each independently hydrogen, G^{A}, or an electron withdrawing group, or R³⁰ and R^{31B} are joined to form a non-aromatic ring structure, or R^{31A} and R^{31B} are joined to form a non-aromatic ring structure;
J¹ is O, NR³⁶, C₁₋₄ alkylene, C₁₋₄ heteroalkylene, or absent, wherein R³⁶ is hydrogen, optionally substituted C₁₋₄ alkyl, or an electron withdrawing group;
J² is absent, C(O), SO, or SO₂;
R³² is hydrogen, G^{A}, or an electron withdrawing group,
Lg is OH, CN, or a leaving group, e.g., F, Cl, etc.
R³³ and R³⁴ are independently hydrogen or optionally substituted C₁₋₄ alkyl;
R³⁵ is G^{B}, OG^{B}, NH(G^{B}), N(G^{B})(G^{B}), and
R³⁶ is hydrogen or optionally substituted C₁₋₄ alkyl or an electron withdrawing group,
wherein G^{B} at each occurrence is independently hydrogen, an optionally substituted C₁₋₆ alkyl, optionally substituted C₂₋₆ alkenyl, optionally substituted C₂₋₆ alkynyl, or an optionally substituted ring structure having 4-10 ring atoms; and
wherein G^{A} at each occurrence is independently an optionally substituted C₁₋₆ alkyl, optionally substituted C₂₋₆ alkenyl, optionally substituted C₂₋₆ alkynyl, optionally substituted C₁₋₆ heteroalkyl, or an optionally substituted ring structure having 4-10 ring atoms.

In some embodiments, the compound of Formula Y can be characterized as having a formula according to Formula Y-1, Y-2, Y-2-E, Y-2-F, Y-3, Y-3A, Y-3B, Y-3C, Y-4, Y-5, Y-6, or Y-7:

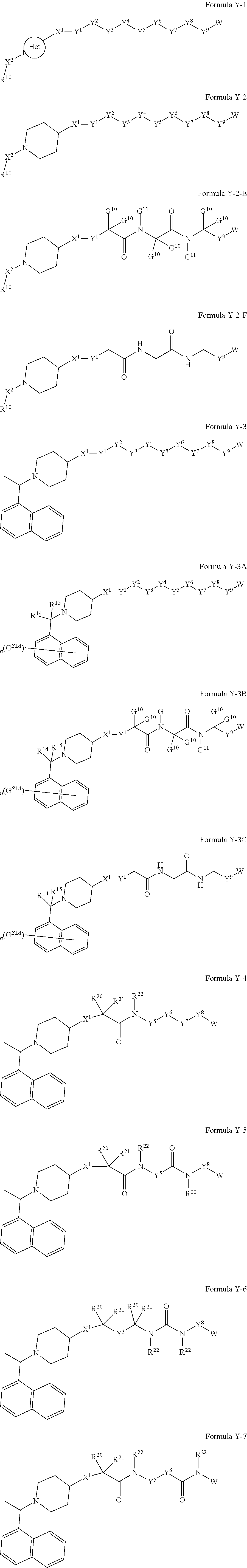

wherein Het represents an optionally substituted 4-10 membered heterocyclic ring, which is bonded with X² through the ring nitrogen atom as drawn, and is bonded with X¹ through another ring atom, wherein the 4-10 membered heterocyclic ring has 0-3 ring heteroatoms each independently O, S, or N, in addition to the as drawn ring nitrogen atom,
wherein in Formula Y-2-E or Y-3B, each G¹⁰ is independently hydrogen, deuterium, methyl, or two G¹⁰ together with the carbon they are both attached to form a cyclopropylene,

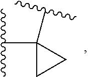 [SMILES: *C1(*)CC1]

and each G¹¹ is hydrogen or methyl, provided that out of all instances of G¹⁰ and G¹¹ combined, at most four instances are not hydrogen or deuterium, preferably, at most three instances are not hydrogen or deuterium, more preferably, only one or two instances are not hydrogen or deuterium;
wherein in Formula Y-3A, Y-3B or Y-3C, (i) R¹⁴ and R¹⁵ are each independently hydrogen, an optionally substituted C₁₋₄ alkyl, optionally substituted C₂₋₄ alkenyl, optionally substituted C₂₋₄ alkynyl, or an optionally substituted ring structure having 4-10 ring atoms; (ii) one of R¹⁴ and R¹⁵ is COOH, or an ester or amide thereof, and the other of R¹⁴ and R¹⁵ is defined in (i); or (iii) R¹⁴ and R¹⁵ together with the carbon they are both attached to form a 3-6 membered ring which is optionally substituted;
wherein n is 0, 1, 2, 3, or 4, preferably, 0, 1, or 2, and
G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring. To be clear, it should be understood that each G^{S1A} in Formula Y-3A, Y-3B, or Y-3C can attach to any available position of the naphthyl ring.

The variables in Formula Y such as X¹, X², R⁹, R¹⁰, Y¹, Y², Y³, Y⁴, Y Y⁶, Y⁷, Y⁸, Y⁹, and W can include any of those respective definitions described herein in connection with Formula X or its subformulae or substructures. The variables in Formula Y such as X¹, X², R⁹, R¹⁰, Y¹, Y², Y³, Y⁴, Y⁵, Y⁶, Y⁷, Y⁸, Y⁹, and W also include any of those respective definitions shown in the specific compounds in Table A herein or in the Examples section, e.g., Compounds. 1-177.

In some preferred embodiments, X² in Formula Y (e.g., any of the subformulae) can be CR¹⁴R¹⁵ as defined herein. For example, in some embodiments, one of R¹⁴ and R¹⁵ is hydrogen, and the other of R¹⁴ and R¹⁵ is CH₂F, CH₂OH, CHF₂, CD₃, CF₃, ethyl, CN, cyclopropyl, COOH, or COOCH₃.

In some preferred embodiments, R¹⁰ in Formula Y (e.g., any of the subformulae) can be an optionally substituted phenyl or optionally substituted naphthyl as defined herein. For example, in some embodiments, R¹⁰ can be

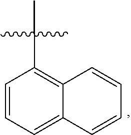 [SMILES: *c1cccc2ccccc12]

which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring. In some embodiments, R¹⁰ can be

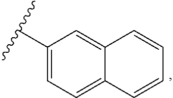 [SMILES: *c1ccc2ccccc2c1]

which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring. In more embodiments, R¹⁰ can be an optionally substituted phenyl,

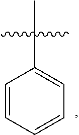 [SMILES: *c1ccccc1]

which is optionally substituted, such as with 1-3 G^{S1B}, wherein G^{S1B} at each occurrence is independently halogen (e.g., F, Cl, or Br), OH, C₁₋₄ alkyl optionally substituted with 1-3 F (e.g., methyl, CF₃), C₁₋₄ alkoxy optionally substituted with 1-3 F (e.g., methoxy), or an optionally substituted 3-7 membered ring (e.g., phenyl, thienyl, etc.), for example, R¹⁰ can be

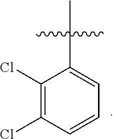 [SMILES: *c1cccc(Cl)c1Cl]

In some preferred embodiments, X²—R¹⁰ in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7) can be

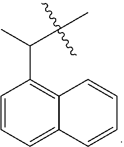 [SMILES: *C(C)c1cccc2ccccc12]

In some embodiments, X²—R¹⁰ in Formula Y (including sub-formula Y-1, Y-1-A, Y-1-B, Y-1-C, Y-1-D, Y-2, Y-2-A, Y-2-B, Y-2-C Y-2-D, Y-2-E, Y-2-F, Y-3, Y-3A, Y-3B, Y-3C, Y-4, Y-5, Y-6, or Y-7) can be

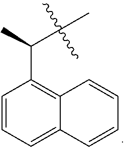 [SMILES: *[C@H](C)c1cccc2ccccc12]

In some embodiments, X²—R¹⁰ in Formula Y (including sub-formula Y-1, Y-1-A, Y-1-B, Y-1-C, Y-1-D, Y-2, Y-2-A, Y-2-B Y-2-C, Y-2-D Y-2-E, Y-2-F, Y-3, Y-3A, Y-3B, Y-3C, Y-4, Y-5, Y-6, or Y-7) can be

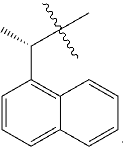 [SMILES: *[C@@H](C)c1cccc2ccccc12]

In some embodiments, X¹ in Formula Y (including sub-formula Y-1, Y-2, Y-2-E, Y-2-F, Y-3, Y-3A, Y-3B, Y-3C, Y-4, Y-5, Y-6, or Y-7) can be NR¹³, wherein R¹³ is defined herein. For example, in some embodiments, R¹³ can be hydrogen, optionally substituted C₁₋₆ alkyl, COG^{A} or SO₂G^{A}, wherein G^{A} is defined herein. In some embodiments, R¹³ can be an optionally substituted C₁₋₆ alkyl, optionally substituted C₂₋₆ alkenyl or optionally substituted C₂₋₆ alkynyl, e.g., as described herein, such as

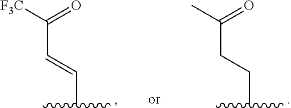

In some embodiments, R¹³ can be COG^{A1}, wherein G^{A1} is an optionally substituted C₁₋₆ alkyl, optionally substituted C₃₋₆ cycloalkyl, optionally substituted phenyl, optionally substituted naphthyl, or an optionally substituted ring structure having 4-10 ring atoms with 1-3 ring heteroatoms. In some embodiments, R¹³ can be COG^{A2}, wherein (i) G^{A2} is a C₁₋₆ alkyl, which is optionally substituted with 1-3 G^{S2}
wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, or (ii) G^{A2} is a C₁₋₆ alkyl, which is optionally substituted with 1-3 G^{S2A}, wherein G^{S2A} at each occurrence is independently halogen (preferably F), OH, COOH, CONH₂, G^{S2B}, CONHG^{S2B}CONG^{S2B} G^{S2B}, SO₂NH₂, SO₂NHG^{S2B}, SO₂NG^{S2B}G^{S2B}, COG^{S2B}, CO₂G^{S2B}, or SO₂G^{S2B} wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}
   wherein
   G^{S1} at each occurrence is independently halogen (preferably F), OH, C₁₋₄ alkyl, or C₁₋₄ alkoxy and
   G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure. In some embodiments, R¹³ can be COG^{A3}, wherein G^{A3} is a C₃₋₆ cycloalkyl, which is optionally substituted with 1-5 G^{S3}, wherein
   G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure; and G^{S1} at each occurrence is independently halogen (preferably F), OH, C₁₋₄ alkyl, or C₁₋₄ alkoxy. In any of the embodiments described herein, unless specified or otherwise contrary from context, R¹³ can be selected from:

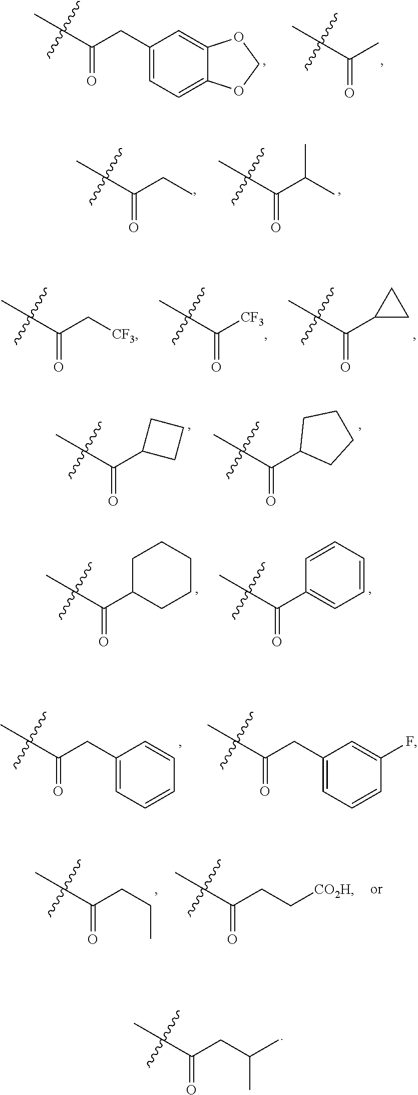

In some embodiments, X¹ in Formula Y (including sub-formula Y-1, Y-2, Y-2-E, Y-2-F, Y-3, Y-3A, Y-3B, Y-3C, Y-4, Y-5, Y-6, or Y-7) can be NR³, wherein R¹³ is SO₂G^{A1} wherein G^{A1} is an optionally substituted C₁₋₆ alkyl, optionally substituted C₃₋₆ cycloalkyl, optionally substituted phenyl, optionally substituted naphthyl, or an optionally substituted ring structure having 4-10 ring atoms with 1-3 ring heteroatoms. In some embodiments, R¹³ can be SO₂G^{A4}, and (i) G^{A4} is a C₁₋₆ alkyl, which is optionally substituted with 1-3 G^{S2}, wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}; or (ii) G^{A4} is a C₁₋₆ alkyl, optionally substituted with 1-3 G^{S2A}, wherein G^{S2A} at each occurrence is independently halogen (preferably F), OH, COOH, CONH₂, G^{S2B}, CONHG^{S2B}, CONG^{S2B} G^{S2B}, SO₂NH₂, SO₂NHG^{S2B}, SO₂NG^{S2B}G^{S2B}, COG^{S2B}, CO₂G^{S2B}, or SO₂G^{S2B}, wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure. In some embodiments, R¹³ can be SO₂G^{A5}, wherein G^{A5} is a C₃₋₆ cycloalkyl or phenyl, which is optionally substituted with 1-5 G^{S3}, wherein
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure; and G^{S1} at each occurrence is independently halogen (preferably F), OH, C₁₋₄ alkyl, or C₁₋₄ alkoxy. In some embodiments, R¹³ can be SO₂G^{A5}, wherein G^{A5} is a 4-7 membered heterocyclic ring having 1 or 2 ring heteroatoms, which are independently N, O, or S, such as pyrrolidine, piperidine, or piperazine, etc., wherein the 4-7 membered heterocyclic ring is optionally substituted with 1-3 substituents independently selected from G^{S3A}, (C₁₋₄ alkylene)-G^{S3A}, OG^{S3A}, NHG^{S3A}, NG^{S3A}G^{S3A}, or (C₁₋₄ heteroalkylene)-G^{S3A}, G^{S3A} at each occurrence is independently halogen (preferably F), OH, COOH, CONH₂, G^{S2B} , CONHG^{S2B} , CONG^{S2B} G^{S2B}, SO₂NH₂, SO₂NHG^{S2B}, SO₂NG^{S2B}G^{S2B} COG^{S2B}, CO₂G^{S2B}, or SO₂G^{S2B}, wherein G^{S2B} at each occurrence is independently as defined hereinabove. In some embodiments, X¹ in Formula A-1 (including sub-formula A-2 or A-3) can be NR¹³, wherein R¹³ can be COOG^{A}, CONH₂, CONH(G^{A}), CON(G^{A})(G^{A}), SO₂NH₂, SO₂NH(G^{A}), or SO₂N(G^{A})(G^{A}), wherein G^{A} is defined herein. In any of the embodiments described herein, unless specified or otherwise contrary from context R¹³ can be selected from:

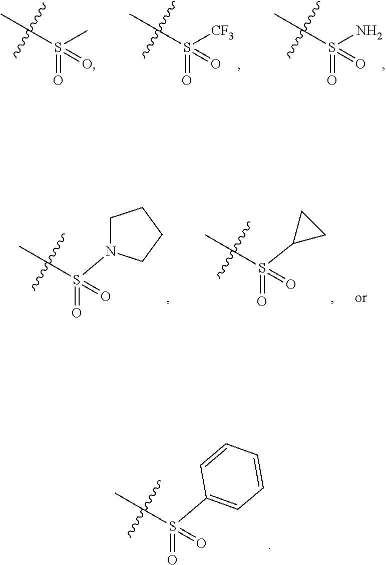

In any of the embodiments described herein, unless specified or otherwise contrary from context, R¹³ can be selected from:

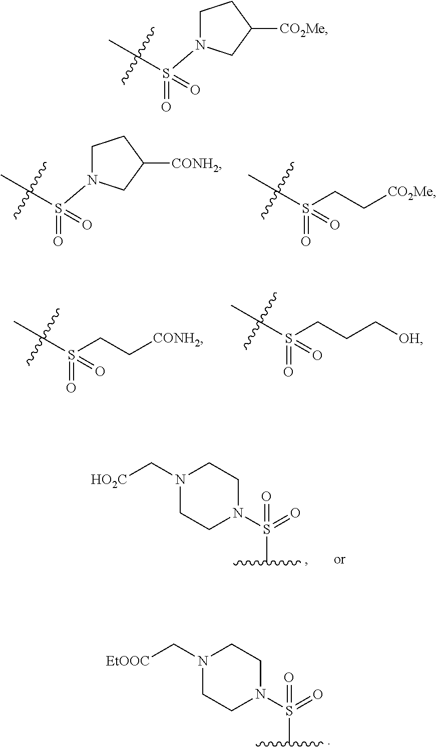

In some embodiments, the compound of Formula Y-1 can have a structure according to Formula Y-1-A, Y-1-B, Y-1-C, or Y-1-D:

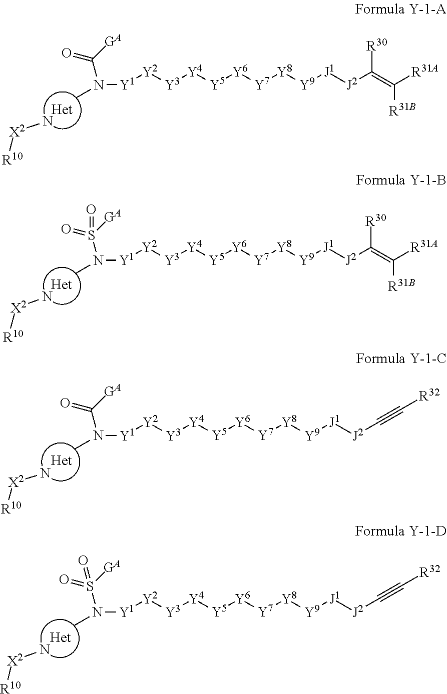

wherein the variables X², Het, R¹⁰, Y¹, Y², Y³, Y⁴, Y⁵, Y⁶, Y⁷, Y⁸, Y⁸, G^{A}, J¹, J², R³⁰, R^{31A}, R^{31B}, and R³² include any of those respective definitions as described herein in connection with Formula Y, or an applicable subformula, in any combinations.

In some embodiments, the linker of

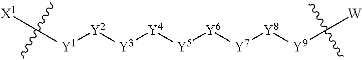

in Formula Y and any applicable subformulae can have any of the definitions described herein for Formula L-1 (e.g., Formula L-2, L-2A, L-3, L-3A, L-4, L-4A, L-5 or L-6) in connection with Formula X. In some embodiments, Y¹ to Y⁹ in Formula Y (including sub-formula Y-1 or Y-2) can be such that the linker of

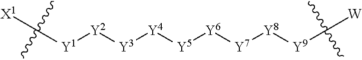

in the formula can be characterized as having a structure according to

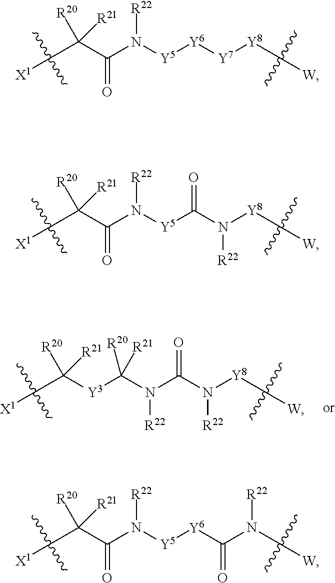

wherein the variables R²⁰, R²¹, R²², Y³, Y⁵, Y⁶, Y⁷, and Y⁸ include any of those described herein in any combinations, for example, as described in connection with Formula Y-2-E, Y-2-F, Y-3, Y-3A, Y-3B, Y-3C, Y-4, Y-5, or Y-6.

In some embodiments, Y¹ to Y⁹ in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable) can be characterized as having one or more (e.g., 2, 3, 4, 5, 6, 7, 8, or all) of the following applicable features in any combinations:
i) Y¹ is absent;
ii) Y² is CH₂ or CH(C₁₋₄ alkyl);
iii) Y³ is C(O);
iv) Y⁴ is NH, CH₂ or CH(C₁₋₄ alkyl);
v) Y⁵ is NH, CH₂, CH(C₁₋₄ alkyl), C(C₁₋₄ alkyl)(C₁₋₄ alkyl), or

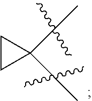 [SMILES: *C1(*)CC1]

vi) Y⁶ is C(O), CH₂ or CH(C₁₋₄ alkyl);
vii) Y⁷ is NH, CH₂ or CH(C₁₋₄ alkyl); or Y⁷ is C(O);
viii) Y⁸ is NH; or Y⁸ is CH₂ or CH(C₁₋₄ alkyl); or Y⁸ is absent; and
ix) Y⁹ is absent, an optionally substituted C₁₋₄ alkylene, or an optionally substituted 4-8 membered heterocyclic ring having 1 or 2 ring heteroatoms independently 0, S, or N.

In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), none of Y², Y³, Y⁴, Y⁵, Y⁶, Y⁷, and Y⁸ is absent. In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), one or two of Y², Y³, Y⁴, Y⁵, Y⁶, Y⁷, and Y⁸ is absent. In some embodiments, Y² is absent. In some embodiments, Y³ is absent. In some embodiments, Y⁴ is absent. In some embodiments, Y⁵ is absent. In some embodiments, Y⁶ is absent. In some embodiments, Y⁷ is absent. In some embodiments, Y⁸ is absent.

In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), two of Y², Y³, Y⁴, Y⁵, Y⁶, Y⁷, and Y⁸ are C(O). In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), one of Y², Y³, Y⁴, Y⁵, Y⁶, Y⁷, and Y⁸ is C(O). In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), one of Y², Y³, Y⁴, Y⁵, Y⁶, Y⁷, and Y⁸ is SO₂. In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), up to four of Y², Y³, Y⁴, Y⁵s Y⁶ 7 and Y⁸ can be NR²². In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y³ is C(O) and Y⁴ is NR²². In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y⁴, Y⁵ and Y⁶ are joined to form a 3-6 membered ring, such as a 4 or 5-membered heterocyclic ring having 1-3 ring heteroatoms independently selected from N, O, and S. In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y⁵, Y⁶ and Y⁷ are joined to form a 3-6 membered ring, such as a 5-membered heteroaryl ring having 1-3 ring heteroatoms independently selected from N, O, and S. In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y⁶, Y⁷ and Y¹ are joined to form a 3-6 membered ring, such as a 4 or 5-membered heterocyclic ring having 1-3 ring heteroatoms independently selected from N, O, and S. In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y⁶ is C(O) and Y⁷ is NR²². In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y⁷ is C(O) and Y⁸ is NR²². In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), Y⁸ is absent, CH₂ or CH(C₁₋₄ alkyl). Other definitions of Y² to Y⁸ include any of those respective definitions described herein for Formula L-1 (e.g., Formula L-2, L-2A, L-3, L-3A, L-4, L-4A, L-5 or L-6) in connection with Formula X.

In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), R²⁰ and R²¹ at each occurrence are each independently hydrogen or optionally substituted C₁₋₄ alkyl. In some embodiments, in Formula Y (including sub-formula Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, as applicable), R²² at each occurrence is independently hydrogen or optionally substituted C₁₋₄ alkyl. Other definitions of R²⁰, R²¹, and R²² are described herein.

In some embodiments, the moiety of

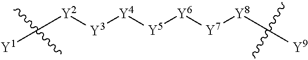

in Formula Y and any applicable subformulae can have a structure of

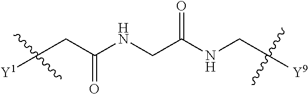

(Y¹ and Y⁹ are shown to show direction of attachment). In some embodiments, Y¹ is absent. In some embodiments, Y⁹ is absent. In some embodiments, Y⁹ is a C₁₋₄ alkylene or C₁₋₄ heteroalkylene. In some embodiments, the linker of

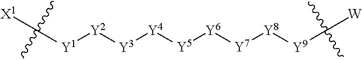

in Formula Y and any applicable subformulae can have a structure of

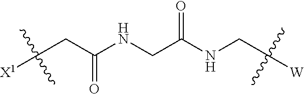

(X¹ and W are shown to show direction of attachment). In some embodiments, the linker of

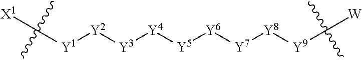

in Formula Y and any applicable subformulae can have a structure of

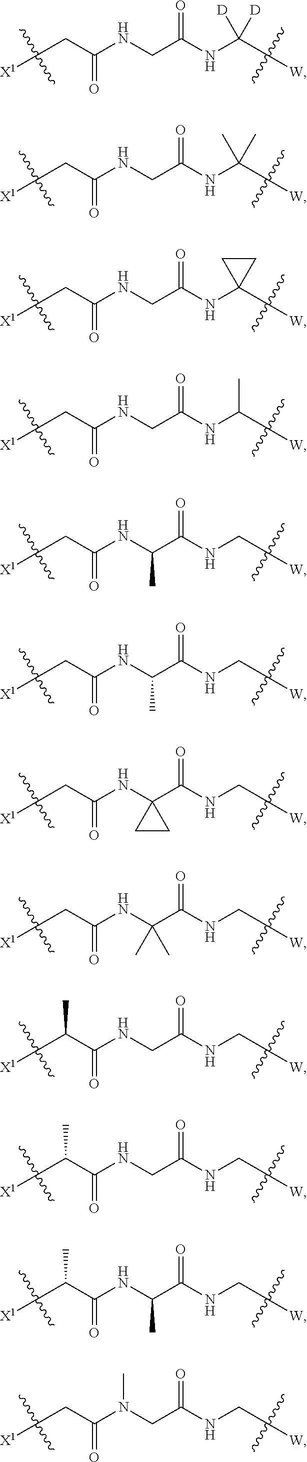

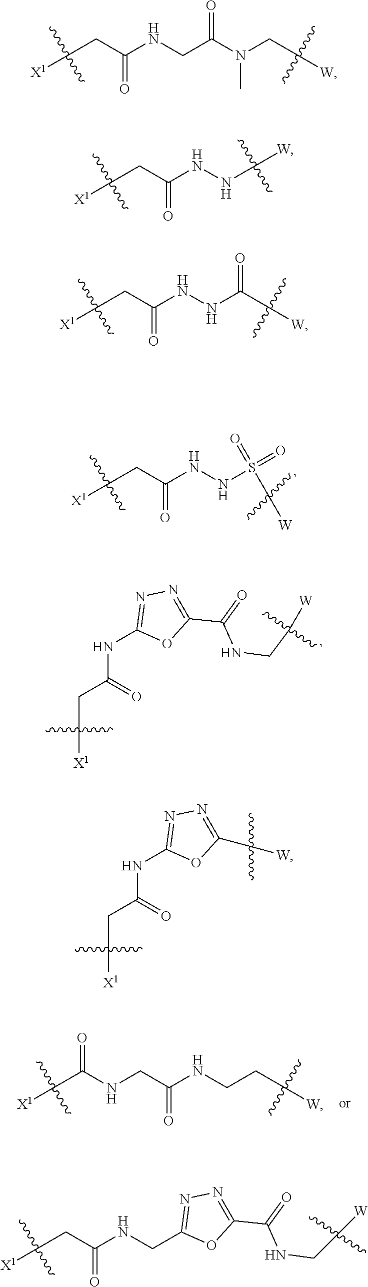

(X¹ and W are shown to show direction of attachment). In some embodiments, the linker of

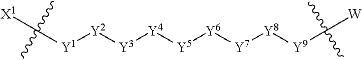

in Formula Y and any applicable subformulae can have a structure of

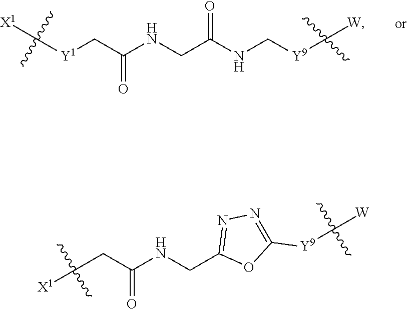

(X¹ and W are shown to show direction of attachment). In some embodiments, Y¹ is absent. In some embodiments, Y⁹ is absent. In some embodiments, Y⁹ is a C₁₋₄ alkylene or C₁₋₄ heteroalkylene, each of which can be branched or linear. In some embodiments, the linker of

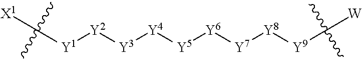

in Formula Y and any applicable subformulae can have a structure of the Y₁₀-Linker-Y₁₀ as defined in connection with Formula I, II, III, IV, or V in any of Embodiments 1-14 herein, or any of those described herein in the specific compounds shown in Table A herein or the Examples section.

Suitable W groups for Formula Y are not particularly limited and include any of those described herein in connection with Formula X.

For example, in some embodiments, the compound of Formula Y-2 can have a structure according to Formula Y-2-A, Y-2-B, Y-2-C, or Y-2-D:

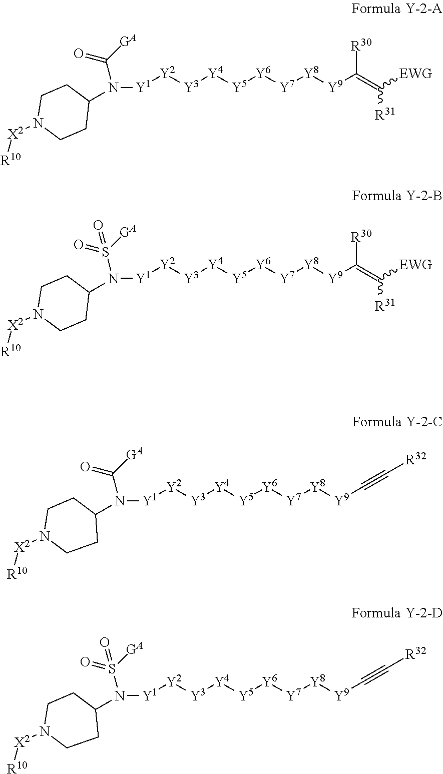

wherein the variables X², R¹⁰, Y¹, Y², Y³, Y⁴, Y⁵, Y⁶, Y⁷, Y⁸, Y⁹, G^{A}, R³⁰ , R³¹, EWG, and R³² include any of those respective definitions as described herein in connection with Formula Y, or an applicable subformula, in any combinations. For example, in some embodiments, X²—R¹⁰ in Formula Y-2-A, Y-2-B, Y-2-C, or Y-2-D can be

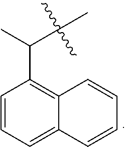 [SMILES: *C(C)c1cccc2ccccc12]

In some embodiments, X²—R¹⁰ in Formula Y-2-A, Y-2-B, Y-2-C, or Y-2-D can be

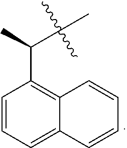 [SMILES: *[C@H](C)c1cccc2ccccc12]

In some embodiments, X²—R¹⁰ in Formula Y-2-A, Y-2-B, Y-2-C, or Y-2-D can be

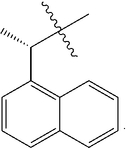 [SMILES: *[C@@H](C)c1cccc2ccccc12]

In some embodiments, R³⁰ and R³¹ are both hydrogen, and EWG is COOH, COO(C₁₋₄ alkyl), CONH₂, CONH(C₁₋₄ alkyl), CON(C₁₋₄ alkyl)(C₁₋₄ alkyl), CN, or SO₂(C₁₋₄ alkyl). In some embodiments, R³² is hydrogen or a C₁₋₄ alkyl. In some embodiments,

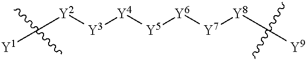

in Formula Y-2-A, Y-2-B, Y-2-C, or Y-2-D can have a structure of

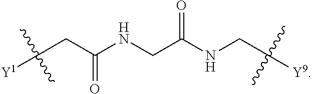

In some embodiments, Y¹ is absent. In some embodiments, Y⁹ is absent. In some embodiments, Y⁹ is a C₁₋₄ alkylene or C₁₋₄ heteroalkylene. In some embodiments, G^{A} is a C₁₋₆ alkyl, which is optionally substituted with 1-3 G^{S2}, wherein G^{S2} is defined herein. In some embodiments, G^{A} is a C₃₋₆ cycloalkyl, which is optionally substituted with 1-5 G^{S3}, wherein G^{S3} is defined herein. In some embodiments, G^{A} is phenyl which is optionally substituted with 1-5 G^{S3}, wherein G^{S3} is defined herein.

In some embodiments, W in Formula Y (including sub-formula Y-1, Y-1-A, Y-1-B, Y-1-C, Y-1-D, Y-2, Y-2-A, Y-2-B, Y-2-C, Y-2-D Y-2-E Y-2-F, Y-3, Y-3A, Y-3B, Y-3C, Y-4, Y-5, Y-6, or Y-7, as applicable) can be

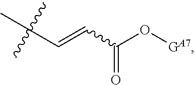

### C(O)R³⁵, or

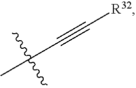

wherein G^{A7} is an optionally substituted C₁₋₆ alkyl, R³⁵ is hydrogen or a C₁₋₄ alkyl, and R³² is hydrogen or a C₁₋₄ alkyl.

In some embodiments, in Formula Y (including sub-formula Y-1, Y-2, Y-3, Y-3A, Y-3B, Y-3C, Y-4 Y-5 Y-6, or Y-7, as applicable), Y⁹—W can be —(C₁₋₄ alkylene)-CN, CN,

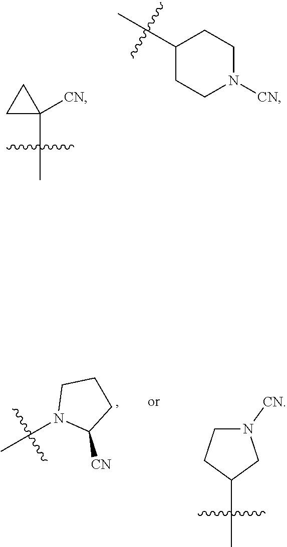 [SMILES: *C1(C#N)CC1 ; *C1CCN(C#N)CC1 ; *N1CCC[C@H]1C#N ; *C1CCN(C#N)C1]

In some embodiments, Y⁹—W can be

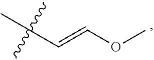 [SMILES: */C=C/OC]

which may be viewed as a masked CH₂CHO group. In some embodiments, W in Formula Y (including sub-formula Y-1, Y-2, Y-3, Y-3A, Y-3B, Y-3C, Y-4, Y-5, Y-6, or Y-7, as applicable) can be C(O)H,

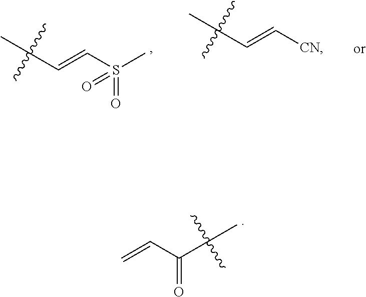

In some embodiments, W can be

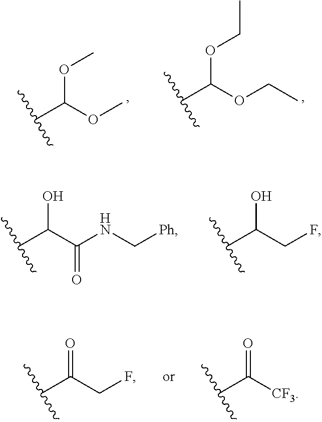

In some embodiments, W can be

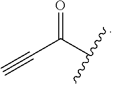 [SMILES: *C(=O)C#C]

In some preferred embodiments, W in Formula Y (including sub-formula Y-1, Y-2, Y-3, Y-3A, Y-3B, Y-3C, Y-4, Y-5, Y-6, or Y-7, as applicable) can be

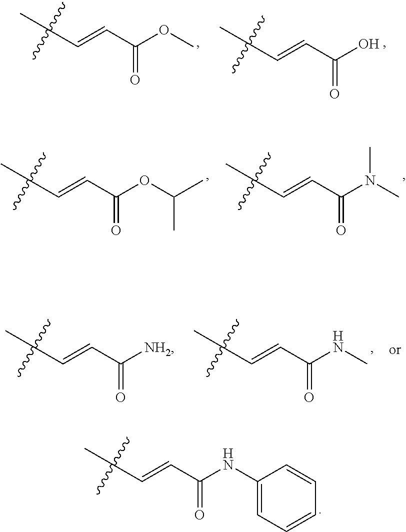

In some embodiments, W can be

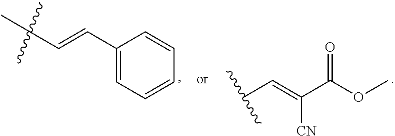

In some preferred embodiments, W in Formula Y (including sub-formula Y-1, Y-2, Y-3, Y-3A, Y-3B, Y-3C, Y-4, Y-5, Y-6, or Y-7, as applicable) can be

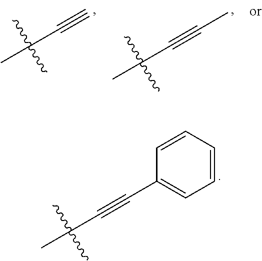

### Formula I, II, III, IV, or V

In some embodiments, the present disclosure also provides a compound of formula I, II, III, IV, or V. Particularly preferred compounds of the present disclosure include those described in the Table A.

In some embodiments, the present disclosure provides the following exemplary embodiments:

Embodiment 1. A compound according to Formula I, or a pharmaceutically acceptable salt thereof:

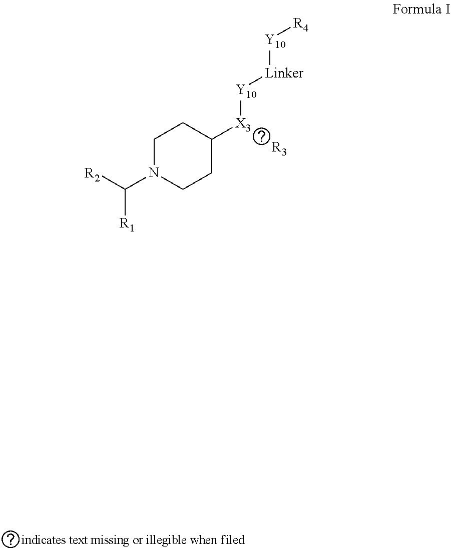

wherein R₁ is selected from aryl or heteroaryl, each of which is optionally substituted; and R₂ is selected from hydrogen, alkyl, haloalkyl, heteroalkyl, hydroxyl, alkoxy or a pro-drug moiety, each of which is optionally substituted;
wherein R₃ is selected from hydrogen, —C(═O)—R_{5A1}, —SO₂—R_{5A1}, —NHC(═O)—R_{5A1}, —NHSO₂—R_{5A1}, —C(═O)—(CH₂)ₙ—R_{5A1}, —NHCH₂—R_{5A1}, —(CH₂)ₙ—C(═O)—R_{5A1}, —(CH₂)ₙ—SO₂—R_{5A1}, —SO₂—(CH₂)ₙ—R_{5A1}, —C(═O)—CH₂—R_{5A1}, —C(═O)—NH—R_{5A1}, —SO₂—NH—R_{5A1}, or —SO₂—CH₂—R_{5A1};
wherein X₃ is CH or N; wherein each Y₁₀ is independently absent or selected from —C(═O)—, —CHR_{5A2}—, —CH₂CHR_{5A2}—, —NR_{5A2}—, —CHR_{5A2}—NH—;
wherein Linker is selected from —(C═O)—NR_{5A2}—(CH₂)ₙ—(C═O)—, —(C═O)—NR_{5A2}—(CH₂)ₙ—(C═O)-heteroalkyl-, —(C═O)—NR_{5A2}—(CH₂)ₙ—(C═O)-heteroaryl, —(C═O)—NR_{5A2}—, —SO₂—NR_{5A2}—(CH₂)ₙ—(C═O)—, —(C═O)—NR_{5A2}—(CH₂)ₙ—SO₂—, —SO₂—NR_{5A2}—(CH₂)ₙ—SO₂—, —NR_{5A2}—, —(C═O)—NR_{5A2}—(CH₂)ₙ-heteroaryl-, —(C═O)—NR_{5A2}—(CH₂)ₙ-heterocycloalkyl-, —(OCH₂CH₂)ₙ—(PEG), alkyl linker, aminoalkyl linker; cycloalkyl linker, heterocycloalkyl linker, aryl linker, heteroaryl linker, and wherein n is an integer between 0 and 7;
and wherein R₄ is selected from a functional group for reversible or irreversible covalent interactions, including but not limited to —CR_{5A3}—C≡C—R₆, —C≡C—R₆, —CR_{5A3}—CH═CH—C(═O)—R₆, —CH═CH—C(═O)—R₆, —CR_{5A3}—CH═CH—C(═O)—Z—R₆, —CH═CH—C(═O)—Z—R₆, —CR_{5A3}—CH═CH—SO₂—R₆, —CH═CH—SO₂—R₆, —CR_{5A3}—CH═CH—SO₃—R₆, —CH═CH—SO₃—R₆, —CR_{5A3}—C≡N, or —C≡N;
wherein Z is selected from NH, N(R_{5B}), O or S;

In some preferred embodiments R₃ is —C(═O)—R_{5A1} or —SO₂—R_{5A1};wherein each of R_{5A1}—R_{5A3} is independently selected from hydrogen, alkyl, cycloalkyl, heterocycloalkyl, aryl, heteroaryl, halogen, —CH₂N(R₆)₂, —CH₂NHR₆, or —C≡N; In some preferred embodiments, each of R_{5A1}—R_{5A3} is independently hydrogen, methyl, ethyl, propyl, cyclopropyl, cyclobutyl, cyclopentyl or phenyl, each of which is optionally substituted. In some preferred embodiments, X₃ is N. In some preferred embodiments, Y₁₀ is —CHR_{5A2}; wherein R_{5A2} is hydrogen, methyl, ethyl, cyclopropyl or halogen (preferably F). In some preferred embodiments, Linker is —(C═O)—NR_{5A2}—(CH₂)ₙ—(C═O)—; wherein R_{5A2} is hydrogen, methyl, ethyl, cyclopropyl and wherein n is an integer between 0 and 3, wherein R₆ is selected from hydrogen, halogen, alkyl, alkenyl, acyl, aryl, or heteroaryl. In some preferred embodiments, R₄ is —C≡C—R₆ or —CH═CH—C(═O)—Z—R₆; wherein R₆ is hydrogen, methyl, ethyl, cyclopropyl or phenyl, each of which is optionally substituted; wherein Z is O or NH.

Embodiment 2. The compound of embodiment 1, or a pharmaceutically acceptable salt thereof, wherein R₁, and R₂ are not the same.

Embodiment 3. The compound of embodiment 1, or a pharmaceutically acceptable salt thereof, wherein R₁, and R₂ are the same.

Embodiment 4. The compound of any of embodiments 1-3, or a pharmaceutically acceptable salt thereof, wherein R₁ is an aryl. In some preferred embodiments, R₁ is naphthyl or phenyl, each of which is optionally substituted.

Embodiment 5. The compound of any of embodiments 1-3, or a pharmaceutically acceptable salt thereof, wherein R₁ is a heteroaryl. In some preferred embodiments, R₁ is quinolinyl, isoquinolinyl or quinazolinyl each of which is optionally substituted.

Embodiment 6. The compound of any of embodiments 1-5, or a pharmaceutically acceptable salt thereof, wherein R₂ is an alkyl. In some preferred embodiments, R₂ is hydrogen, methyl, ethyl, hydroxymethyl, 2-hydroxyethyl, aminomethyl or 2-aminomethyl.

Embodiment 7. The compound of any of embodiments 1-5, or a pharmaceutically acceptable salt thereof, wherein R₂ is (R)-configuration.

Embodiment 8. The compound of embodiment 1, or a pharmaceutically acceptable salt thereof, wherein R₁ is an aryl and R₂ is an alkyl. In some preferred embodiments, R₁ is naphthyl and R₂ is methyl, each of which is optionally substituted.

Embodiment 9. The compound of any of embodiments 1-8, or a pharmaceutically acceptable salt thereof, wherein R₃ is —C(═O)—R_{5A1}.

Embodiment 10. The compound of e any of embodiments 1-8, or a pharmaceutically acceptable salt thereof, wherein R₃ is —SO₂—R_{5A1}.

Embodiment 11. The compound of embodiment 1, or a pharmaceutically acceptable salt thereof, having a structure according to Formula II:

### Formula II

wherein each of R_{5B1}—R_{5B4} is independently selected from hydrogen, alkyl, cycloalkyl, heterocycloalkyl, aryl, heteroaryl, halogen, —CH₂N(R₆)₂, —CH₂NHR₆, or —C≡N; In some preferred embodiments, R_{5B1} and R_{5B3} are independently selected from hydrogen, methyl, halogen (preferably F), hydroxymethyl, 2-hydroxymethyl, aminomethyl or 2-aminomethyl. In some preferred embodiments, R_{5B2} and R_{5B4} are independently selected from hydrogen, methyl, ethyl, propyl, cyclopropyl, cyclobutyl or cyclopentyl.

Embodiment 12. The compound of embodiment 1, or a pharmaceutically acceptable salt thereof, having a structure according to Formula III:

### Formula III

wherein each of R_{5C1}—R_{5C4} is independently selected from hydrogen, alkyl, cycloalkyl, heterocycloalkyl, aryl, heteroaryl, halogen, —CH₂N(R₆)₂, —CH₂NHR₆, or —C≡N; In some preferred embodiments, R_{5C1} and R^{5C2} are independently selected from hydrogen, methyl, halogen (preferably F), hydroxymethyl, 2-hydroxymehyl, aminomethyl or 2-aminomethyl. In some preferred embodiments, R_{5C3} and R_{5C4} are independently selected from hydrogen, methyl, ethyl, propyl, cyclopropyl, cyclobutyl or cyclopentyl.

Embodiment 13. The compound of embodiment 1, or a pharmaceutically acceptable salt thereof, having a structure according to Formula IV:

### Formula IV

wherein each of R_{5D1}—R_{5D5} is independently selected from hydrogen, alkyl, cycloalkyl, heterocycloalkyl, aryl, heteroaryl, halogen, —CH₂N(R₆)₂, —CH₂NHR₆, or —C—N; In some preferred embodiments, R_{5D1}, R_{5D3}, and R_{5D4} are independently selected from hydrogen, methyl, halogen (preferably F), hydroxymethyl, 2-hydroxymehyl, aminomethyl or 2-aminomethyl. In some preferred embodiments, R_{5D2} and R_{5D5} are independently selected from hydrogen, methyl, ethyl, propyl, cyclopropyl, cyclobutyl or cyclopentyl.

Embodiment 14. The compound of embodiment 1, or a pharmaceutically acceptable salt thereof, having a structure according to Formula V:

### Formula V

wherein each of Y₁₁-Y₁₄ is absent or independently selected from —C(═O)—, —C(═O)NH—, —CHR_{5A2}—, —CH₂CHR_{5A2}—, —NR_{5A2}—, —CHR_{5A2}—NH—; in some preferred embodiments, Y₁₁ is —CHR_{5A2}— and Y₁₂ is —C(═O)NH—, in some preferred embodiments, Y₁₃ is —NR_{5A2}—, Y₁₄ is —C(═O)—, —C(═O)NH—, or CHR_{5A2}—; in some preferred embodiments, R_{5A2} is hydrogen, methyl, ethyl, propyl, cyclopropyl, cylobutyl, cyclopentyl or phenyl, each of which is optionally substituted, wherein R₇ is selected from alkyl, cycloalkyl, heterocycloalkyl, aryl, or heteroaryl, in some preferred embodiments, R₇ is 3-8 membered heterocyclic ring. In some preferred embodiments, R₇ is

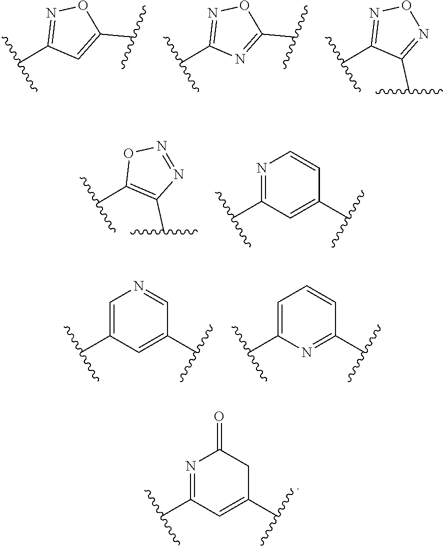

Embodiment 15. A pharmaceutical composition comprising the compound according to any of Embodiments 1-14 and pharmaceutically acceptable carrier, additive or excipient and optionally an additional bioactive agent.

Embodiment 16. The pharmaceutical composition according to Embodiment 15, wherein the additional bioactive agent is an antiviral agent.

Embodiment 17. The pharmaceutical composition according to any one of Embodiments 15 and 16, wherein the pharmaceutical composition is in a form selected from tablet, powder, microparticle, nanoparticle, granule, capsule, liquid, aqueous solution, suspension, or dispersion.

Embodiment 18. A method for treating coronavirus infection in a subject in need thereof comprising administering to the subject a therapeutically effective amount of the compound according to any one of Embodiments 1-14.

Embodiment 19. The method according to embodiment 18, wherein the method comprising administering to the subject an effective amount of the compound, wherein the dysregulated viral protein encoded by the coronavirus is responsible for the infection, and wherein the compound modulates the activity of the viral protein in the subject.

Embodiment 20. The method according to any of Embodiments 18 and 19, wherein the coronavirus infection selected from the group of SARS-CoV, HCoV-229E, HCoV-OC43, HCoV-NL63, MERS-CoV, HCoV-HKU1, and SARS-CoV-2.

Embodiment 21. The method according to any of Embodiments 18-20, wherein the coronavirus infection is SARS-CoV-2.

Embodiment 22. The method according to any of Embodiments 19-21, wherein the viral protein comprises Papain-like protease (PLpro) encoded by the coronavirus selected from the group of SARS-CoV, HCoV-229E, HCoV-OC43, HCoV-NL63, MERS-CoV, HCoV-HKU1 and SARS-CoV-2.

Embodiment 23. The method according to Embodiment 22, wherein the viral protein comprises PLpro encoded by SARS-CoV-2.

Embodiment 24. The method according to any of Embodiments 18-23, wherein the compound is administered to the subject by injection, infusion, oral delivery, or inhalation.

### Exemplary Compounds and Embodiments

In some embodiments, the present disclosure also provides a compound according to the following exemplary embodiments:

EM1. A compound according to Formula Y, Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7, or a pharmaceutically acceptable salt thereof, wherein the variables are defined herein.

EM2. The compound according to EM1, or a pharmaceutically acceptable salt thereof, wherein X¹ is NR¹³, preferably, R¹³ is hydrogen, optionally substituted C₁₋₆ alkyl, COG^{A} or SO₂G^{A}.

EM3. The compound according to EM1 or EM2, or a pharmaceutically acceptable salt thereof wherein X¹ is NR¹³ and R¹³ is selected from:

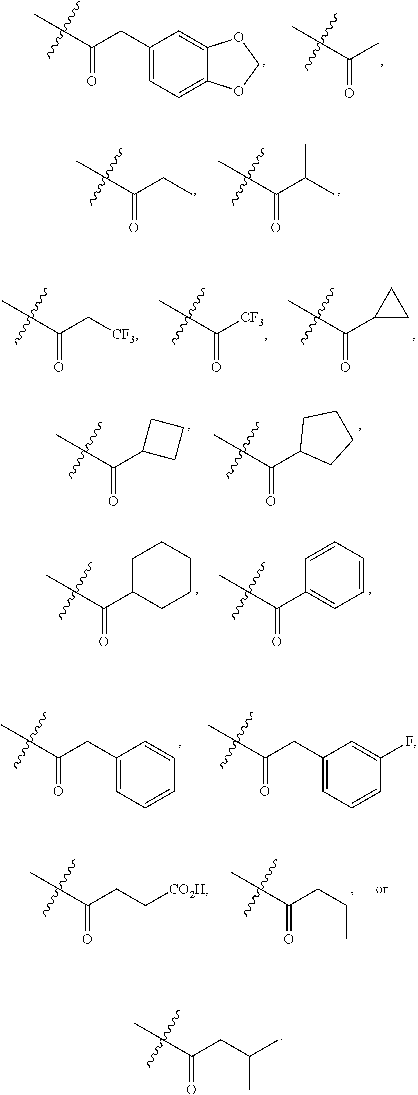

### or R¹³ is

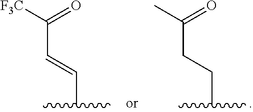

EM4. The compound according to EM1 or EM2, or a pharmaceutically acceptable salt thereof, wherein X¹ is NR¹³, and R¹³ is selected from:

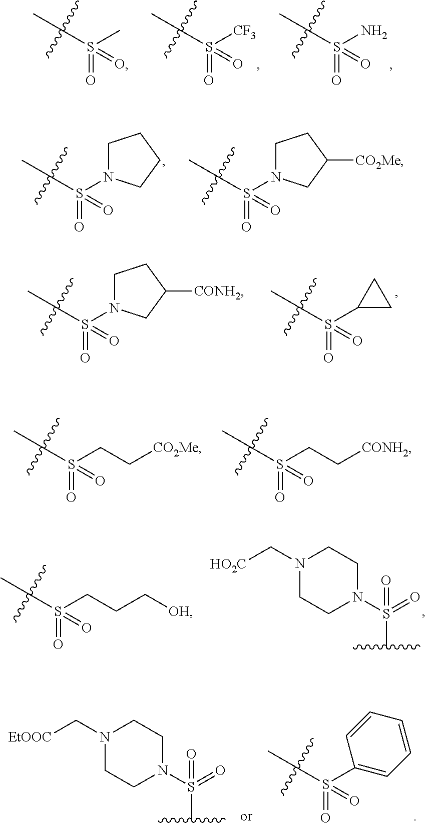

EM5. The compound according to any of EM1-EM4, or a pharmaceutically acceptable salt thereof, wherein (i) Y¹ is absent, (ii) Y¹ is an optionally substituted C₁₋₄ alkylene, such as CH₂ or CD₂, or (iii) Y¹ is an optionally substituted 4-8 membered heterocyclic ring having 1-3 ring heteroatoms independently O, S, or N, such as

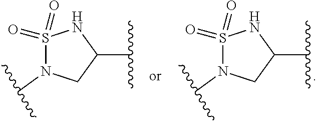 [SMILES: *C1CN(*)S(=O)(=O)N1 ; *C1CN(*)S(=O)(=O)N1]

EM6. The compound according to any of EM1-EM5, or a pharmaceutically acceptable salt thereof, wherein (i) Y² is CH₂ or CH(C₁₋₄ alkyl), such as CH(CH₃), wherein the C₁₋₄ alkyl is optionally substituted, such as with substituents independently selected from F, OH, and NH₂, (ii) Y² is absent, or (iii) Y² is CH(CH₃), C(CH₃)₂, CD₂ or

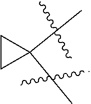 [SMILES: *C1(*)CC1]

EM7. The compound according to any of EM1-EM6, or a pharmaceutically acceptable salt thereof, wherein (i) Y³ is C(O), (ii) Y³ is absent, or (iii) Y³ is CH(OH) or CH(C₁₋₄ alkyl), wherein the C₁₋₄ alkyl is optionally substituted, such as with substituents independently selected from F, OH, and NH₂, for example, Y³ is CH(CF₃).

EM8. The compound according to any of EM1-EM7, or a pharmaceutically acceptable salt thereof, wherein (i) Y⁴ is NH, CH₂ or CH(C₁₋₄ alkyl), (ii) Y⁴ is absent, or (iii) Y⁴ is N(C₁₋₄ alkyl), such as NCH₃, wherein each of the C₁₋₄ alkyl in (i) and (iii) is optionally substituted, such as with substituents independently selected from F, OH, and NH₂.

EM9. The compound according to any of EM1-EM8, or a pharmaceutically acceptable salt thereof, wherein (i) Y⁵ is NH, CH₂, CH(C₁₋₄ alkyl), C(C₁₋₄ alkyl)(C₁₋₄ alkyl), or

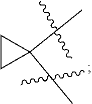 [SMILES: *C1(*)CC1]

(ii) Y⁵ is absent, or (iii) Y⁵ is CH(CH₃), C(CH₃)₂, CD₂ or

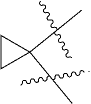 [SMILES: *C1(*)CC1]

EM10. The compound according to any of EM1-EM9, or a pharmaceutically acceptable salt thereof, wherein (i) Y⁶ is C(O), CH₂ or CH(C₁₋₄ alkyl); (ii) Y⁶ is absent, or (iii) Y⁶ is CH(OH) or CH(C₁₋₄ alkyl), wherein the C₁₋₄ alkyl is optionally substituted, such as with substituents independently selected from F, OH, and NH₂, for example, Y⁶ is CH(CF₃).

EM11. The compound according to any of EM1-EM10, or a pharmaceutically acceptable salt thereof, wherein (i) Y⁷ is NH, CH₂ or CH(C₁₋₄ alkyl); (ii) Y⁷ is absent, or (iii) Y⁷ is N(C₁₋₄ alkyl), such as NCH₃, CH(CH₃), C(CH₃)₂, or CD₂, wherein each of the C₁₋₄ alkyl in (i) and (iii) is optionally substituted, such as with substituents independently selected from F, OH, and NH₂.

EM12. The compound according to any of EM1-EM10, or a pharmaceutically acceptable salt thereof, wherein Y⁷ is C(O).

EM13. The compound according to any of EM1-EM12, or a pharmaceutically acceptable salt thereof, wherein Y⁸ is NH or Y⁸ is O or N(C₁₋₄ alkyl), such as NCH₃, wherein the C₁₋₄ alkyl is optionally substituted, such as with substituents independently selected from F, OH, and NH₂.

EM14. The compound according to any of EM1-EM12, or a pharmaceutically acceptable salt thereof, wherein i Y⁸ is CH₂ or CH(C₁₋₄ alkyl), (ii) Y⁸ is CO; or (iii) Y⁸ is CH(CH₃), C(CH₃)₂, CD₂ or

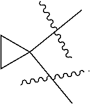 [SMILES: *C1(*)CC1]

EM15. The compound according to any of EM1-EM12, or a pharmaceutically acceptable salt thereof, wherein Y⁸ is absent.

EM16. The compound according to any of EM1-EM15, or a pharmaceutically acceptable salt thereof, wherein (i) Y⁹ is absent, an optionally substituted C₁₋₄ alkylene, such as CH₂ or CD₂, or an optionally substituted 4-8 membered heterocyclic ring having 1 or 2 ring heteroatoms independently O, S, or N, (ii) Y⁹ is a C₃-6 membered carbocyclic ring, such as

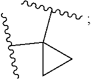 [SMILES: *C1(*)CC1]

or (iii) Y⁹ is a C₁₋₄ heteroalkylene, which is optionally substituted, such as with oxo, F, CF₃, etc.

EM17. The compound according to any of EM1-EM16, or a pharmaceutically acceptable salt thereof, wherein the linker of

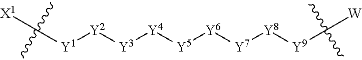

in Formula Y has a structure of

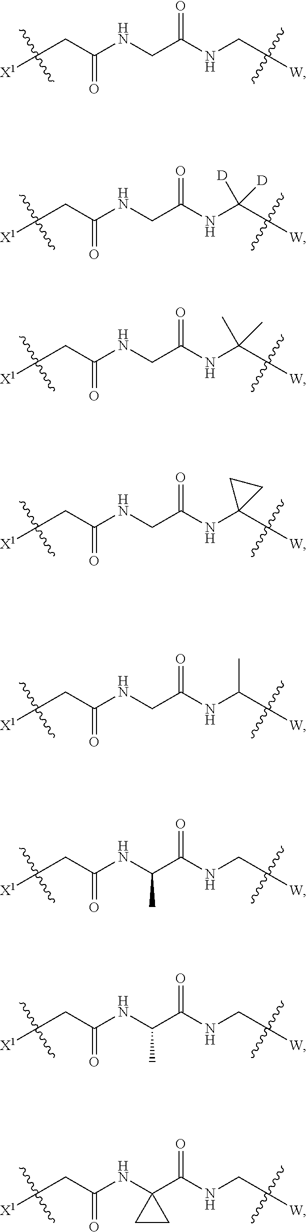

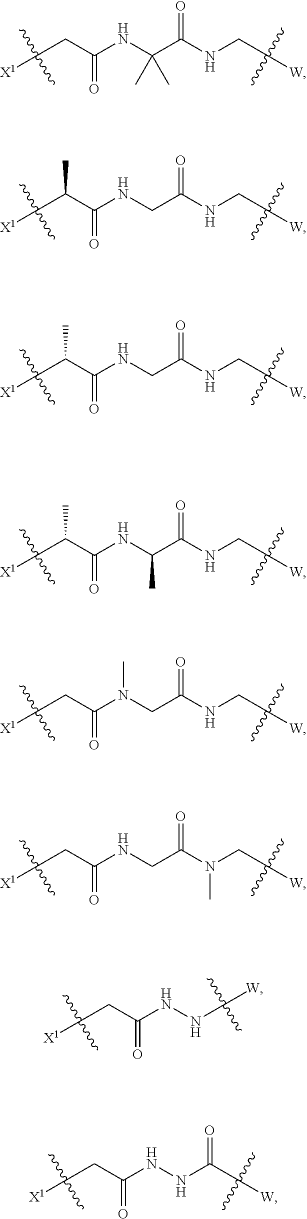

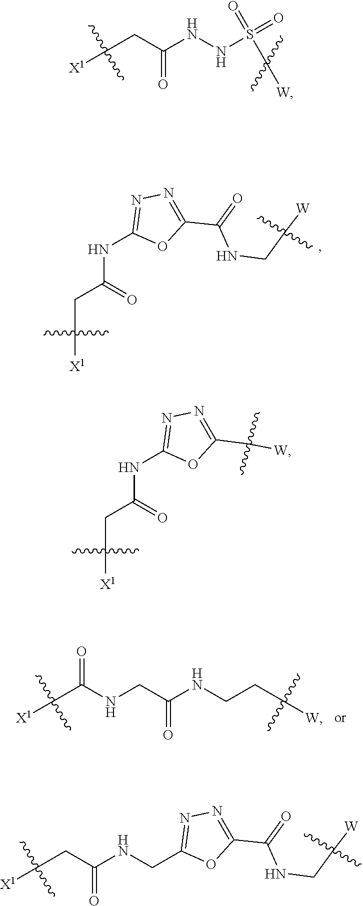

and W are shown to show direction of attachment).

EM18. The compound according to any of EM1-EM17, or a pharmaceutically acceptable salt thereof, wherein W is

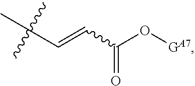

### C(O)R³⁵, or

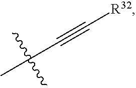

wherein G^{A7} is an optionally substituted C₁₋₆ alkyl, R³⁵ is hydrogen or a C₁₋₄ alkyl, and R³² is hydrogen or a C₁₋₄ alkyl.

EM19. The compound according to any of EM1-EM17, or a pharmaceutically acceptable salt thereof, wherein W is

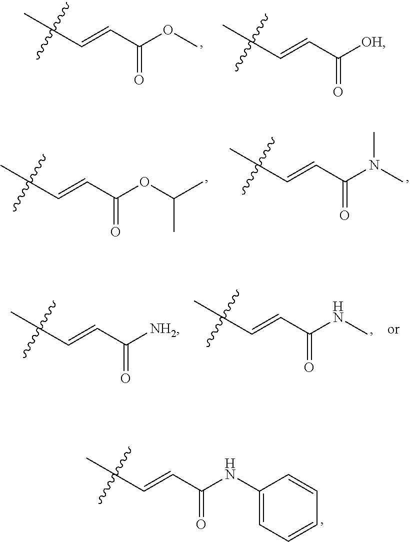

### or W is

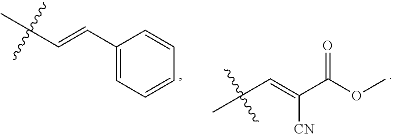

EM20. The compound according to any of EM1-EM17, or a pharmaceutically acceptable salt thereof, wherein W is

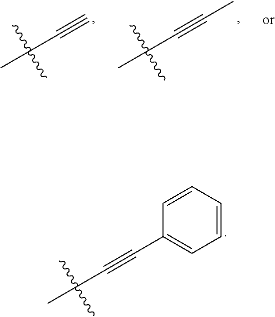

EM21. The compound according to any of EM1-EM17, or a pharmaceutically acceptable salt thereof, wherein Y⁹—W is —(C₁₋₄ alkylene)-CN, CN,

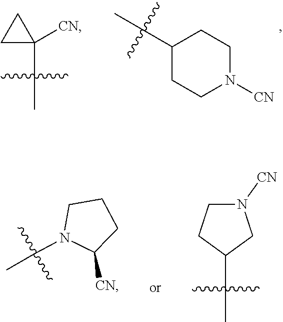 [SMILES: *C1(C#N)CC1 ; *C1CCN(C#N)CC1 ; "," ; *N1CCC[C@H]1C#N ; *C1CCN(C#N)C1 ; "or"]

### or Y⁹—W is

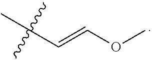 [SMILES: */C=C/OC]

EM22. The compound according to any of EM1-EM17, or a pharmaceutically acceptable salt thereof, wherein W is C(O)H,

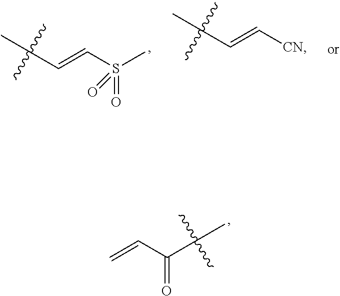

### or W is

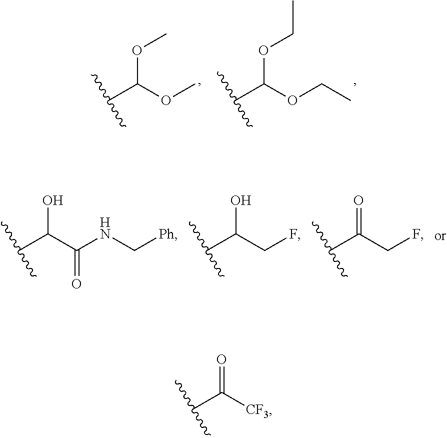

### or W is

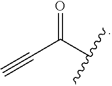 [SMILES: *C(=O)C#C]

In some embodiments, the present disclosure also provides a compound selected from those described in Table A, or a pharmaceutically acceptable salt thereof:

TABLE A
List of Exemplary Compounds

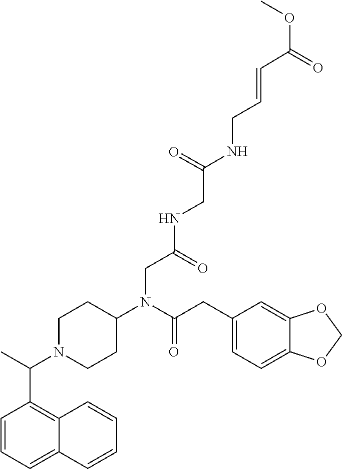 [SMILES: COC(=O)/C=C/CNC(=O)CNC(=O)CN(C(=O)Cc1ccc2c(c1)OCO2)C1CCN(C(C)c2cccc3ccccc23)CC1]

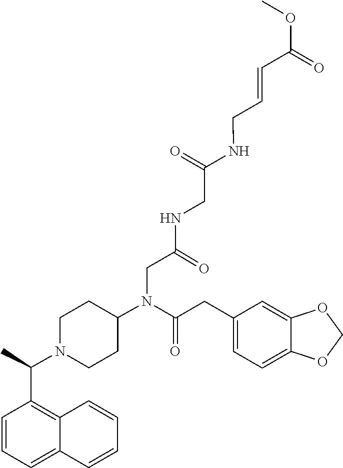 [SMILES: COC(=O)/C=C/CNC(=O)CNC(=O)CN(C(=O)Cc1ccc2c(c1)OCO2)C1CCN([C@H](C)c2cccc3ccccc23)CC1]

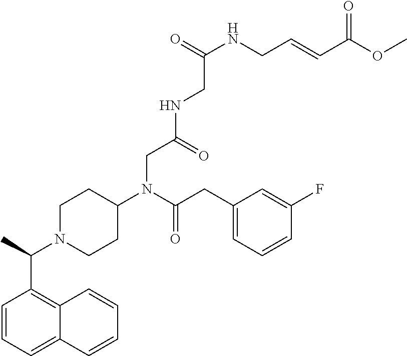 [SMILES: COC(=O)/C=C/CNC(=O)CNC(=O)CN(C(=O)Cc1cccc(F)c1)C1CCN([C@H](C)c2cccc3ccccc23)CC1]

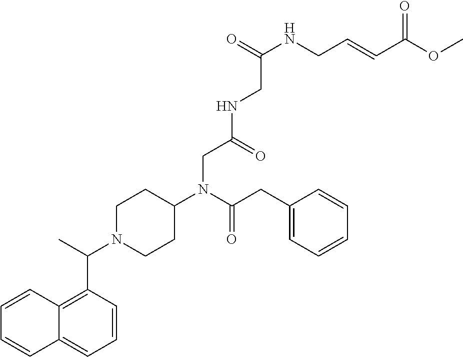 [SMILES: COC(=O)/C=C/CNC(=O)CNC(=O)CN(C(=O)Cc1ccccc1)C1CCN(C(C)c2cccc3ccccc23)CC1]

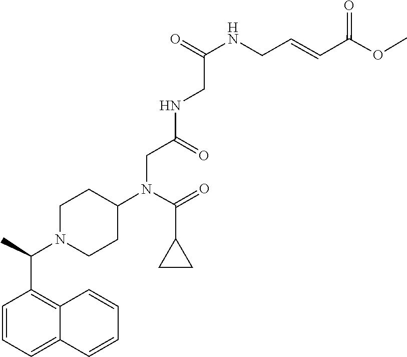 [SMILES: COC(=O)/C=C/CNC(=O)CNC(=O)CN(C(=O)C1CC1)C1CCN([C@H](C)c2cccc3ccccc23)CC1]

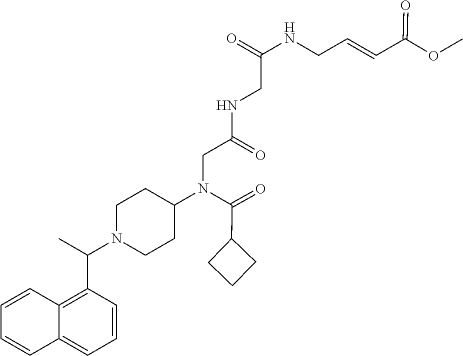 [SMILES: COC(=O)/C=C/CNC(=O)CNC(=O)CN(C(=O)C1CCC1)C1CCN(C(C)c2cccc3ccccc23)CC1]

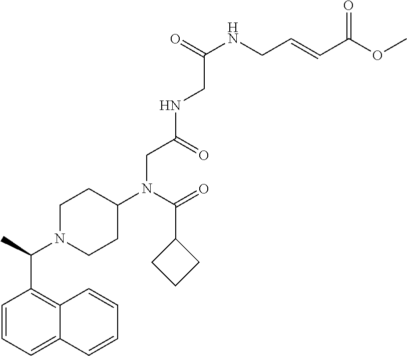 [SMILES: COC(=O)/C=C/CNC(=O)CNC(=O)CN(C(=O)C1CCC1)C1CCN([C@H](C)c2cccc3ccccc23)CC1]

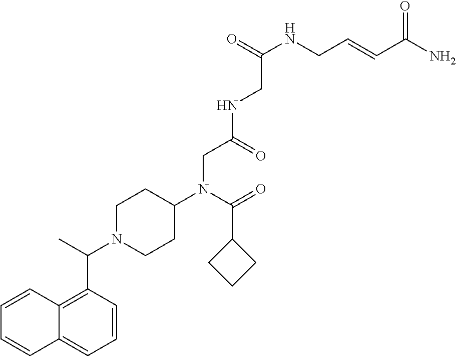 [SMILES: CC(c1cccc2ccccc12)N1CCC(N(CC(=O)NCC(=O)NC/C=C/C(N)=O)C(=O)C2CCC2)CC1]

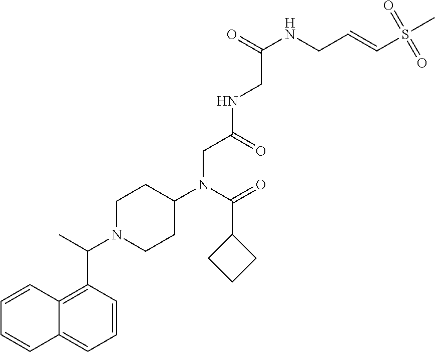 [SMILES: CC(c1cccc2ccccc12)N1CCC(N(CC(=O)NCC(=O)NC/C=C/S(C)(=O)=O)C(=O)C2CCC2)CC1]

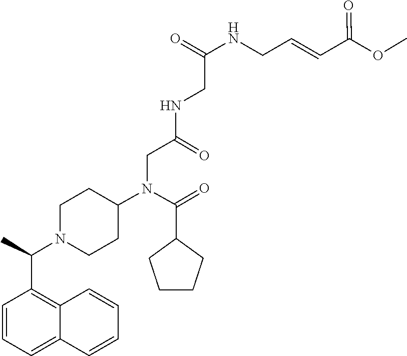 [SMILES: COC(=O)/C=C/CNC(=O)CNC(=O)CN(C(=O)C1CCCC1)C1CCN([C@H](C)c2cccc3ccccc23)CC1]

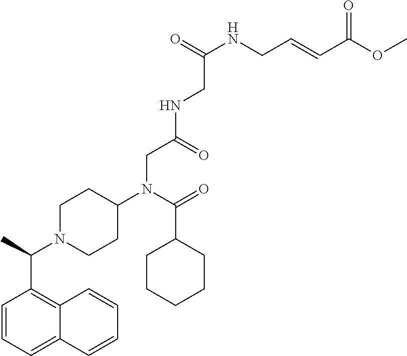 [SMILES: COC(=O)/C=C/CNC(=O)CNC(=O)CN(C(=O)C1CCCCC1)C1CCN([C@H](C)c2cccc3ccccc23)CC1]

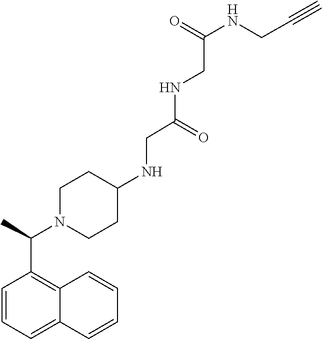 [SMILES: C#CCNC(=O)CNC(=O)CNC1CCN([C@H](C)c2cccc3ccccc23)CC1]

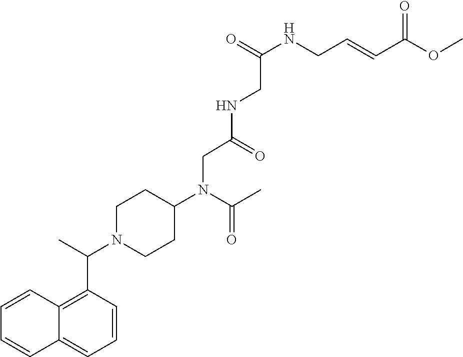 [SMILES: COC(=O)/C=C/CNC(=O)CNC(=O)CN(C(C)=O)C1CCN(C(C)c2cccc3ccccc23)CC1]

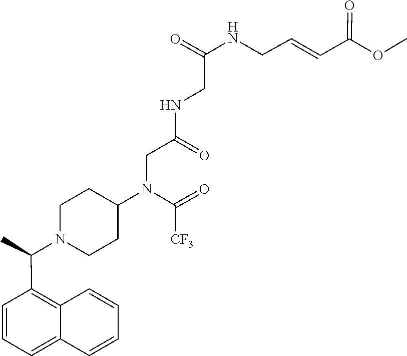 [SMILES: COC(=O)/C=C/CNC(=O)CNC(=O)CN(C(=O)C(F)(F)F)C1CCN([C@H](C)c2cccc3ccccc23)CC1]

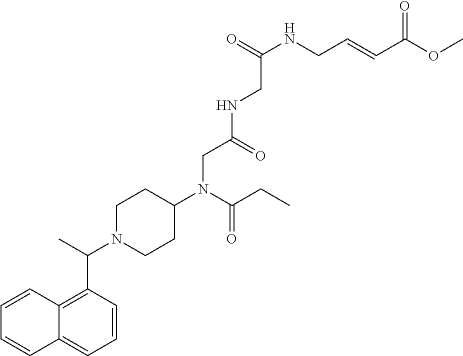 [SMILES: CCC(=O)N(CC(=O)NCC(=O)NC/C=C/C(=O)OC)C1CCN(C(C)c2cccc3ccccc23)CC1]

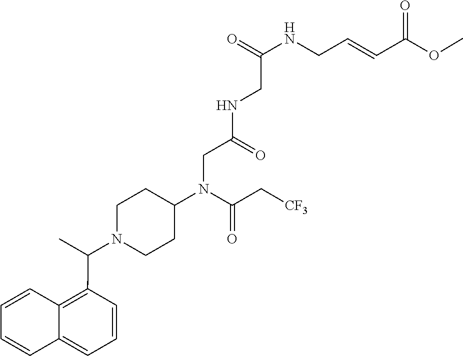 [SMILES: COC(=O)/C=C/CNC(=O)CNC(=O)CN(C(=O)CC(F)(F)F)C1CCN(C(C)c2cccc3ccccc23)CC1]

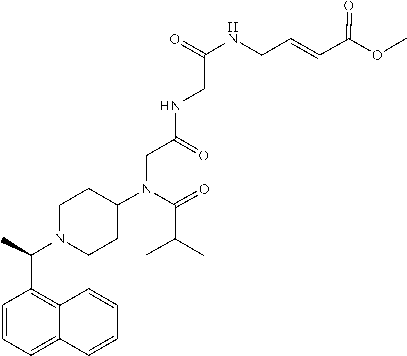 [SMILES: COC(=O)/C=C/CNC(=O)CNC(=O)CN(C(=O)C(C)C)C1CCN([C@H](C)c2cccc3ccccc23)CC1]

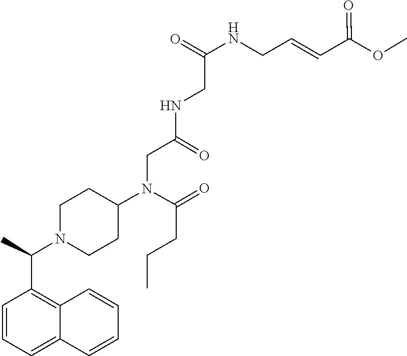 [SMILES: CCCC(=O)N(CC(=O)NCC(=O)NC/C=C/C(=O)OC)C1CCN([C@H](C)c2cccc3ccccc23)CC1]

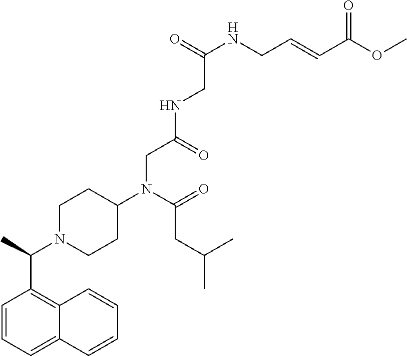 [SMILES: COC(=O)/C=C/CNC(=O)CNC(=O)CN(C(=O)CC(C)C)C1CCN([C@H](C)c2cccc3ccccc23)CC1]

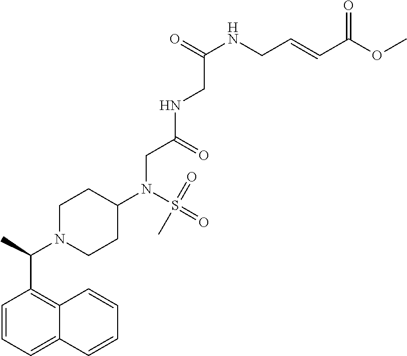 [SMILES: COC(=O)/C=C/CNC(=O)CNC(=O)CN(C1CCN([C@H](C)c2cccc3ccccc23)CC1)S(C)(=O)=O]

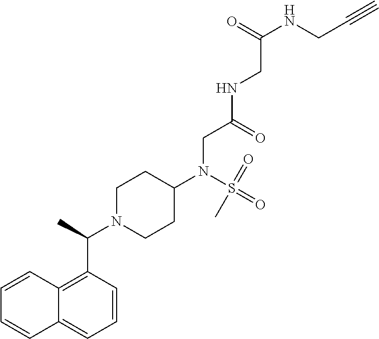 [SMILES: C#CCNC(=O)CNC(=O)CN(C1CCN([C@H](C)c2cccc3ccccc23)CC1)S(C)(=O)=O]

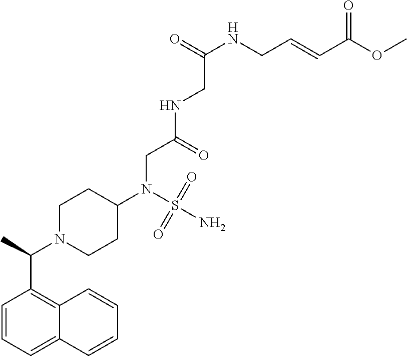 [SMILES: COC(=O)/C=C/CNC(=O)CNC(=O)CN(C1CCN([C@H](C)c2cccc3ccccc23)CC1)S(N)(=O)=O]

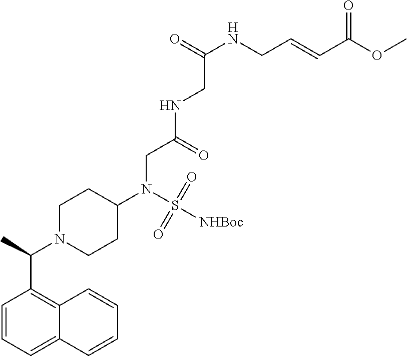 [SMILES: COC(=O)/C=C/CNC(=O)CNC(=O)CN(C1CCN([C@H](C)c2cccc3ccccc23)CC1)S(=O)(=O)NC(=O)OC(C)(C)C]

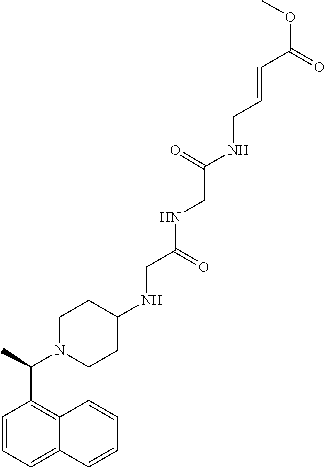 [SMILES: COC(=O)/C=C/CNC(=O)CNC(=O)CNC1CCN([C@H](C)c2cccc3ccccc23)CC1]

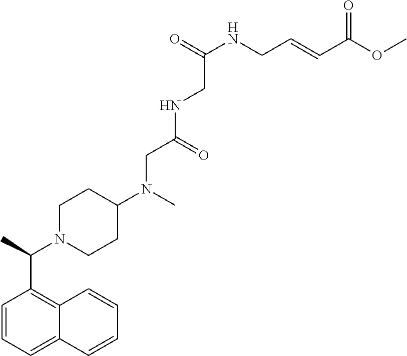 [SMILES: COC(=O)/C=C/CNC(=O)CNC(=O)CN(C)C1CCN([C@H](C)c2cccc3ccccc23)CC1]

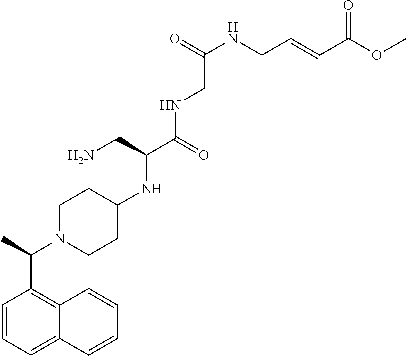 [SMILES: COC(=O)/C=C/CNC(=O)CNC(=O)[C@H](CN)NC1CCN([C@H](C)c2cccc3ccccc23)CC1]

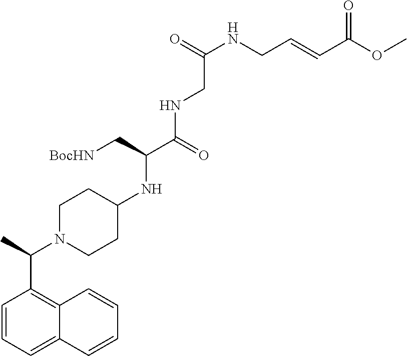 [SMILES: COC(=O)/C=C/CNC(=O)CNC(=O)[C@H](CNC(=O)OC(C)(C)C)NC1CCN([C@H](C)c2cccc3ccccc23)CC1]

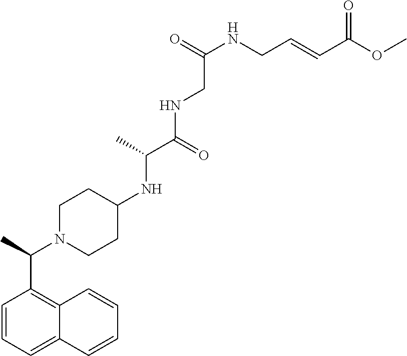 [SMILES: COC(=O)/C=C/CNC(=O)CNC(=O)[C@@H](C)NC1CCN([C@H](C)c2cccc3ccccc23)CC1]

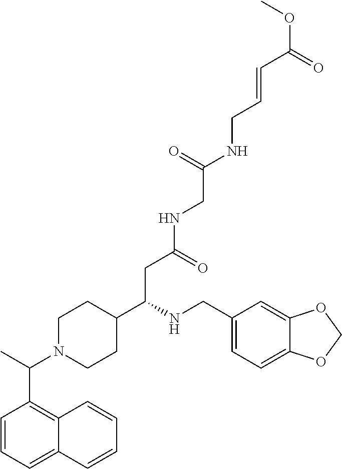 [SMILES: COC(=O)/C=C/CNC(=O)CNC(=O)C[C@H](NCc1ccc2c(c1)OCO2)C1CCN(C(C)c2cccc3ccccc23)CC1]

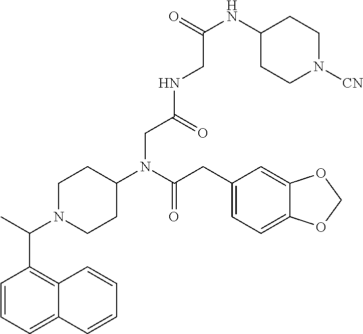 [SMILES: CC(c1cccc2ccccc12)N1CCC(N(CC(=O)NCC(=O)NC2CCN(C#N)CC2)C(=O)Cc2ccc3c(c2)OCO3)CC1]

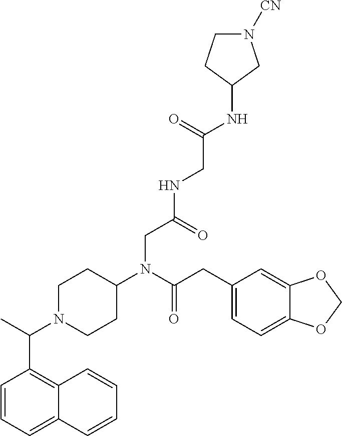 [SMILES: CC(c1cccc2ccccc12)N1CCC(N(CC(=O)NCC(=O)NC2CCN(C#N)C2)C(=O)Cc2ccc3c(c2)OCO3)CC1]

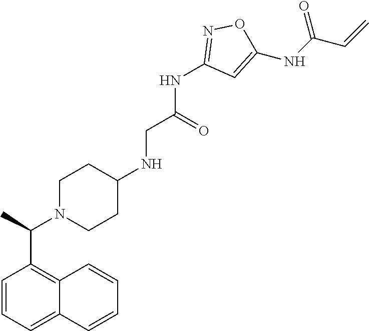 [SMILES: C=CC(=O)Nc1cc(NC(=O)CNC2CCN([C@H](C)c3cccc4ccccc34)CC2)no1]

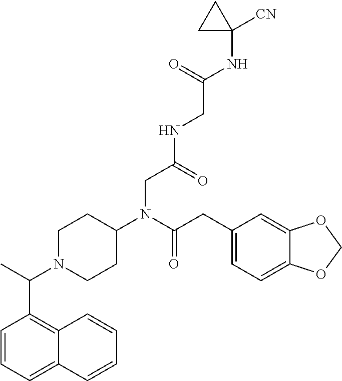 [SMILES: CC(c1cccc2ccccc12)N1CCC(N(CC(=O)NCC(=O)NC2(C#N)CC2)C(=O)Cc2ccc3c(c2)OCO3)CC1]

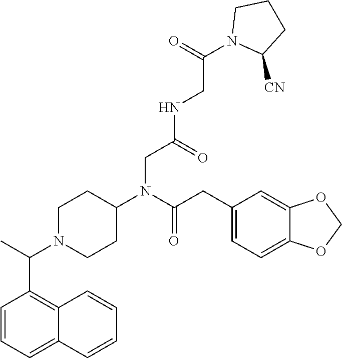 [SMILES: CC(c1cccc2ccccc12)N1CCC(N(CC(=O)NCC(=O)N2CCC[C@H]2C#N)C(=O)Cc2ccc3c(c2)OCO3)CC1]

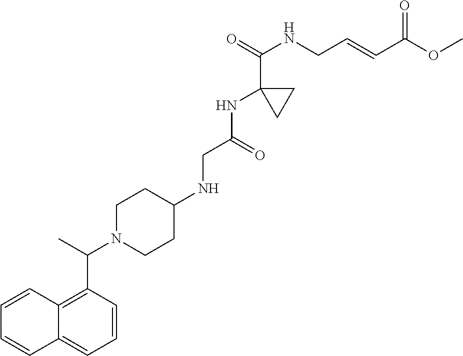 [SMILES: COC(=O)/C=C/CNC(=O)C1(NC(=O)CNC2CCN(C(C)c3cccc4ccccc34)CC2)CC1]

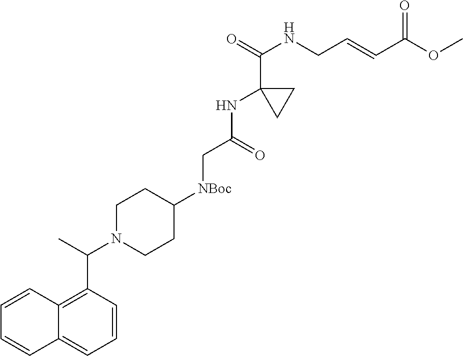 [SMILES: COC(=O)/C=C/CNC(=O)C1(NC(=O)CN(C(=O)OC(C)(C)C)C2CCN(C(C)c3cccc4ccccc34)CC2)CC1]

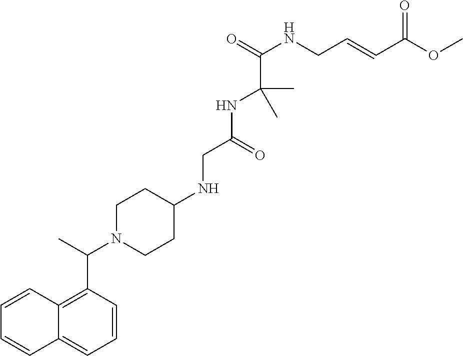 [SMILES: COC(=O)/C=C/CNC(=O)C(C)(C)NC(=O)CNC1CCN(C(C)c2cccc3ccccc23)CC1]

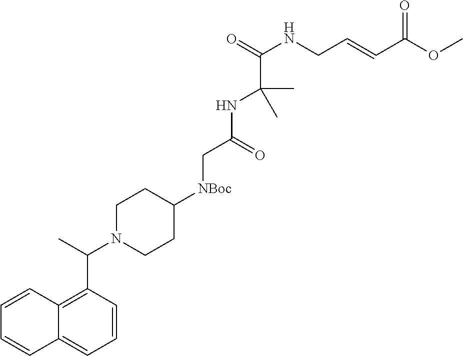 [SMILES: COC(=O)/C=C/CNC(=O)C(C)(C)NC(=O)CN(C(=O)OC(C)(C)C)C1CCN(C(C)c2cccc3ccccc23)CC1]

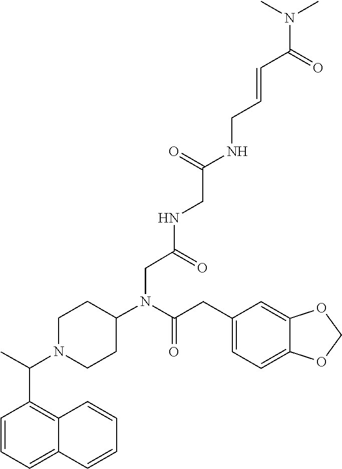 [SMILES: CC(c1cccc2ccccc12)N1CCC(N(CC(=O)NCC(=O)NC/C=C/C(=O)N(C)C)C(=O)Cc2ccc3c(c2)OCO3)CC1]

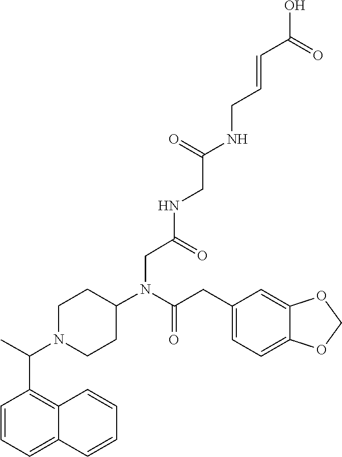 [SMILES: CC(c1cccc2ccccc12)N1CCC(N(CC(=O)NCC(=O)NC/C=C/C(=O)O)C(=O)Cc2ccc3c(c2)OCO3)CC1]

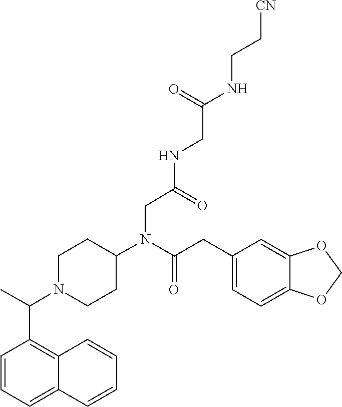 [SMILES: CC(c1cccc2ccccc12)N1CCC(N(CC(=O)NCC(=O)NCCC#N)C(=O)Cc2ccc3c(c2)OCO3)CC1]

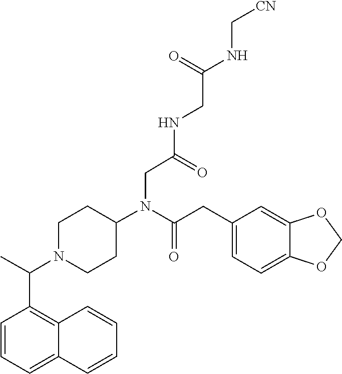 [SMILES: CC(c1cccc2ccccc12)N1CCC(N(CC(=O)NCC(=O)NCC#N)C(=O)Cc2ccc3c(c2)OCO3)CC1]

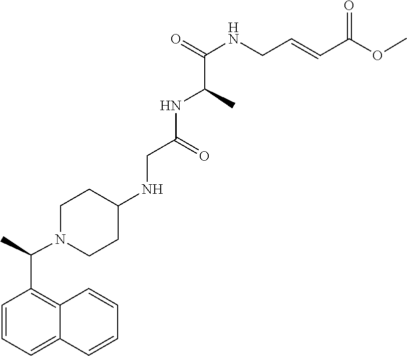 [SMILES: COC(=O)/C=C/CNC(=O)[C@@H](C)NC(=O)CNC1CCN([C@H](C)c2cccc3ccccc23)CC1]

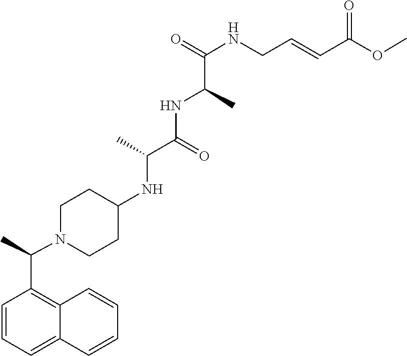 [SMILES: COC(=O)/C=C/CNC(=O)[C@@H](C)NC(=O)[C@@H](C)NC1CCN([C@H](C)c2cccc3ccccc23)CC1]

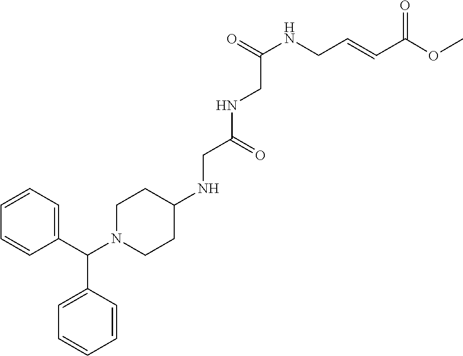 [SMILES: COC(=O)/C=C/CNC(=O)CNC(=O)CNC1CCN(C(c2ccccc2)c2ccccc2)CC1]

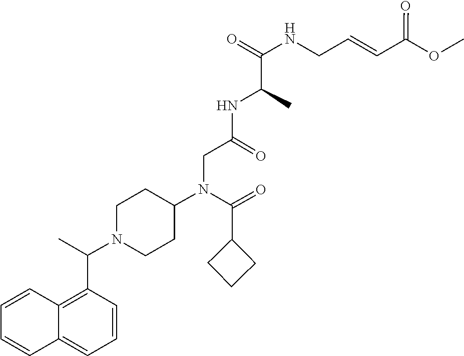 [SMILES: COC(=O)/C=C/CNC(=O)[C@@H](C)NC(=O)CN(C(=O)C1CCC1)C1CCN(C(C)c2cccc3ccccc23)CC1]

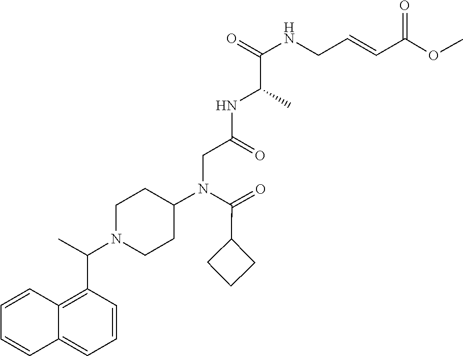 [SMILES: COC(=O)/C=C/CNC(=O)[C@H](C)NC(=O)CN(C(=O)C1CCC1)C1CCN(C(C)c2cccc3ccccc23)CC1]

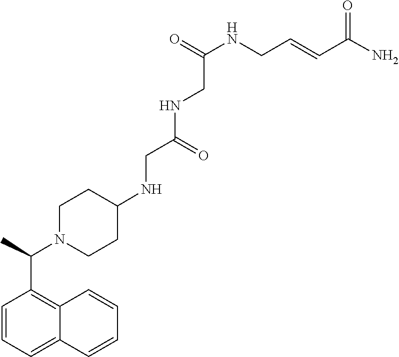 [SMILES: C[C@H](c1cccc2ccccc12)N1CCC(NCC(=O)NCC(=O)NC/C=C/C(N)=O)CC1]

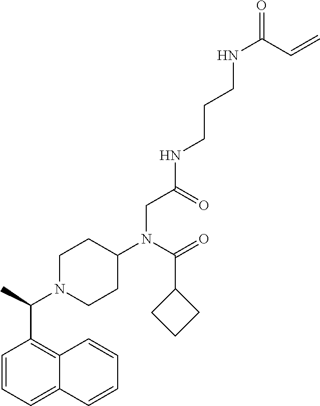 [SMILES: C=CC(=O)NCCCNC(=O)CN(C(=O)C1CCC1)C1CCN([C@H](C)c2cccc3ccccc23)CC1]

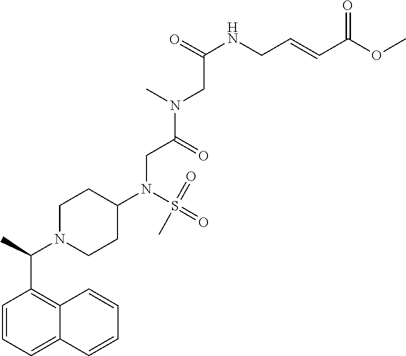 [SMILES: COC(=O)/C=C/CNC(=O)CN(C)C(=O)CN(C1CCN([C@H](C)c2cccc3ccccc23)CC1)S(C)(=O)=O]

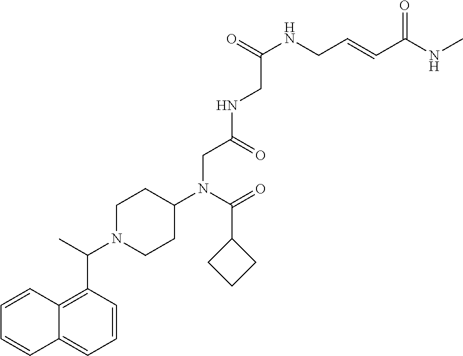 [SMILES: CNC(=O)/C=C/CNC(=O)CNC(=O)CN(C(=O)C1CCC1)C1CCN(C(C)c2cccc3ccccc23)CC1]

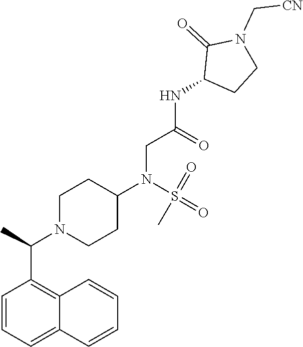 [SMILES: C[C@H](c1cccc2ccccc12)N1CCC(N(CC(=O)N[C@H]2CCN(CC#N)C2=O)S(C)(=O)=O)CC1]

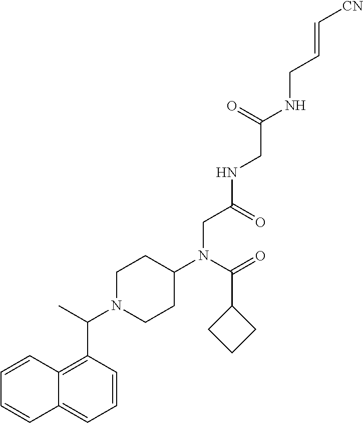 [SMILES: CC(c1cccc2ccccc12)N1CCC(N(CC(=O)NCC(=O)NC/C=C/C#N)C(=O)C2CCC2)CC1]

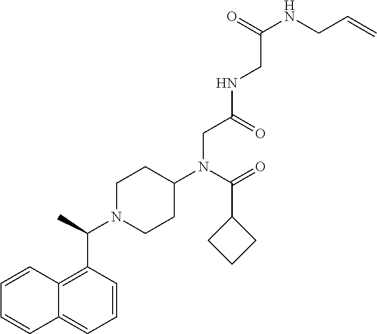 [SMILES: C=CCNC(=O)CNC(=O)CN(C(=O)C1CCC1)C1CCN([C@H](C)c2cccc3ccccc23)CC1]

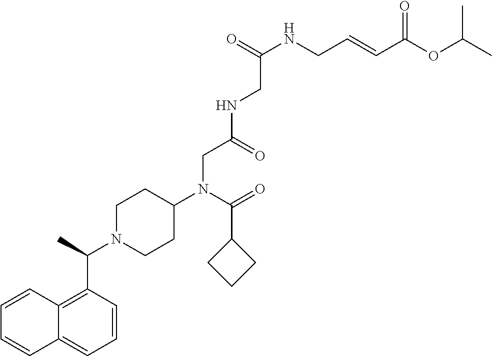 [SMILES: CC(C)OC(=O)/C=C/CNC(=O)CNC(=O)CN(C(=O)C1CCC1)C1CCN([C@H](C)c2cccc3ccccc23)CC1]

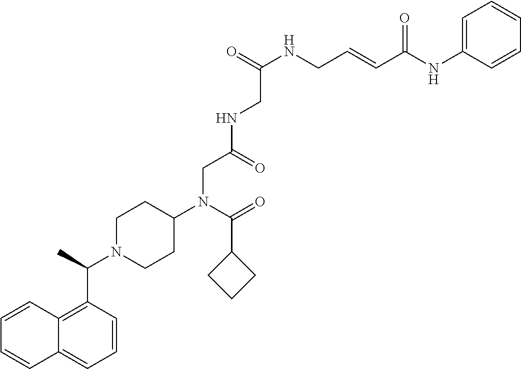 [SMILES: C[C@H](c1cccc2ccccc12)N1CCC(N(CC(=O)NCC(=O)NC/C=C/C(=O)Nc2ccccc2)C(=O)C2CCC2)CC1]

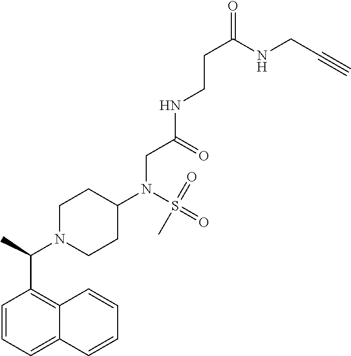 [SMILES: C#CCNC(=O)CCNC(=O)CN(C1CCN([C@H](C)c2cccc3ccccc23)CC1)S(C)(=O)=O]

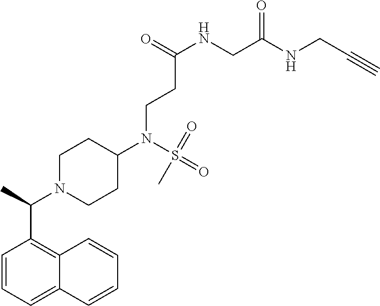 [SMILES: C#CCNC(=O)CNC(=O)CCN(C1CCN([C@H](C)c2cccc3ccccc23)CC1)S(C)(=O)=O]

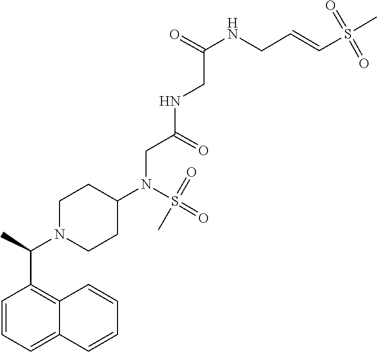 [SMILES: C[C@H](c1cccc2ccccc12)N1CCC(N(CC(=O)NCC(=O)NC/C=C/S(C)(=O)=O)S(C)(=O)=O)CC1]

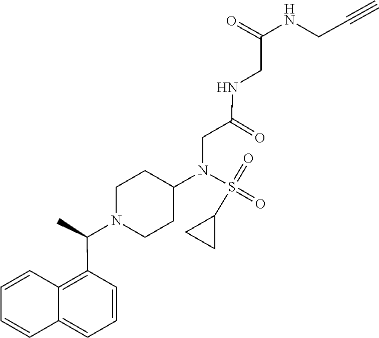 [SMILES: C#CCNC(=O)CNC(=O)CN(C1CCN([C@H](C)c2cccc3ccccc23)CC1)S(=O)(=O)C1CC1]

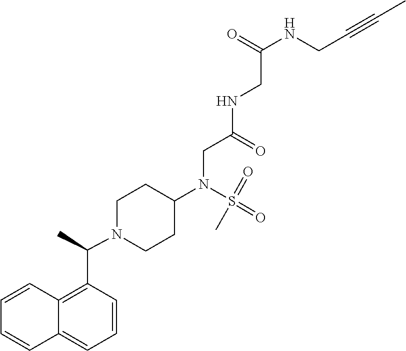 [SMILES: CC#CCNC(=O)CNC(=O)CN(C1CCN([C@H](C)c2cccc3ccccc23)CC1)S(C)(=O)=O]

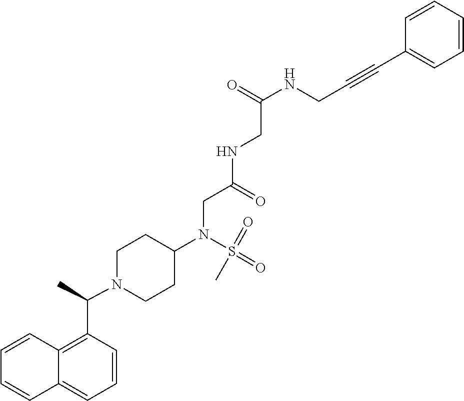 [SMILES: C[C@H](c1cccc2ccccc12)N1CCC(N(CC(=O)NCC(=O)NCC#Cc2ccccc2)S(C)(=O)=O)CC1]

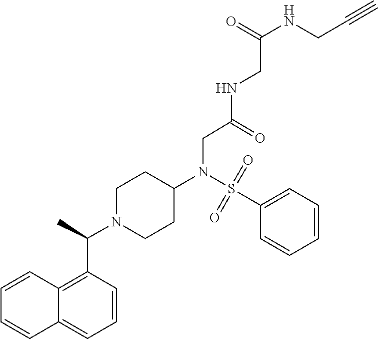 [SMILES: C#CCNC(=O)CNC(=O)CN(C1CCN([C@H](C)c2cccc3ccccc23)CC1)S(=O)(=O)c1ccccc1]

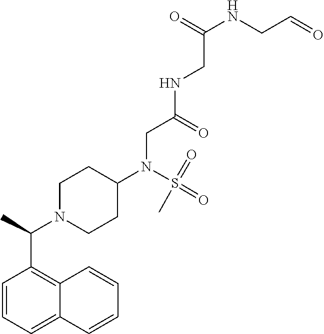 [SMILES: C[C@H](c1cccc2ccccc12)N1CCC(N(CC(=O)NCC(=O)NCC=O)S(C)(=O)=O)CC1]

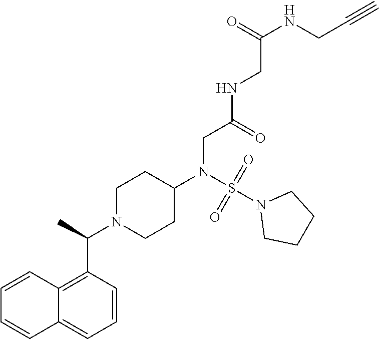 [SMILES: C#CCNC(=O)CNC(=O)CN(C1CCN([C@H](C)c2cccc3ccccc23)CC1)S(=O)(=O)N1CCCC1]

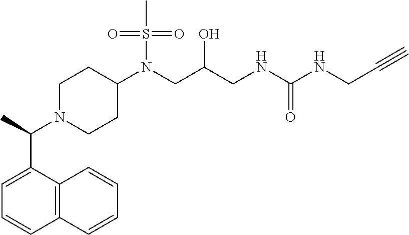 [SMILES: C#CCNC(=O)NCC(O)CN(C1CCN([C@H](C)c2cccc3ccccc23)CC1)S(C)(=O)=O]

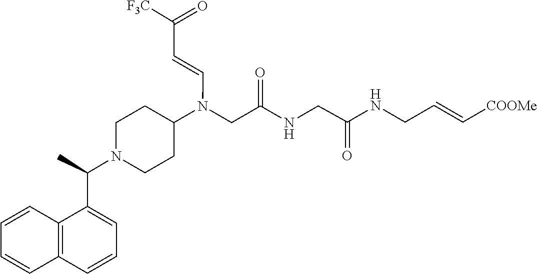 [SMILES: COC(=O)/C=C/CNC(=O)CNC(=O)CN(/C=C/C(=O)C(F)(F)F)C1CCN([C@H](C)c2cccc3ccccc23)CC1]

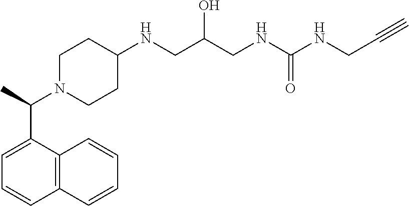 [SMILES: C#CCNC(=O)NCC(O)CNC1CCN([C@H](C)c2cccc3ccccc23)CC1]

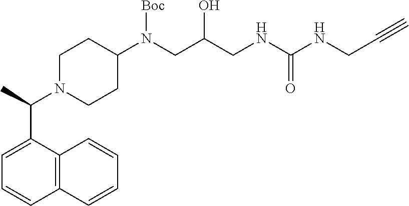 [SMILES: C#CCNC(=O)NCC(O)CN(C(=O)OC(C)(C)C)C1CCN([C@H](C)c2cccc3ccccc23)CC1]

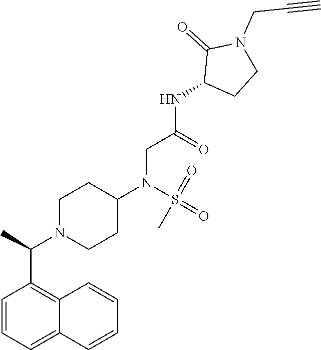 [SMILES: C#CCN1CC[C@H](NC(=O)CN(C2CCN([C@H](C)c3cccc4ccccc34)CC2)S(C)(=O)=O)C1=O]

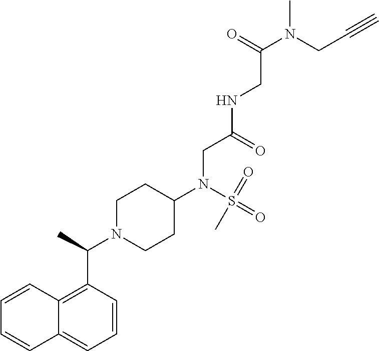 [SMILES: C#CCN(C)C(=O)CNC(=O)CN(C1CCN([C@H](C)c2cccc3ccccc23)CC1)S(C)(=O)=O]

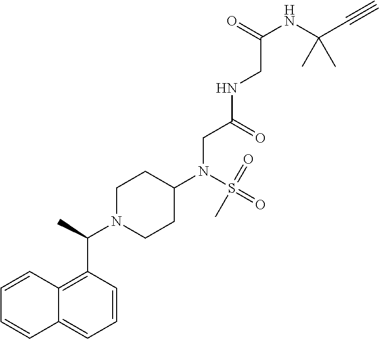 [SMILES: C#CC(C)(C)NC(=O)CNC(=O)CN(C1CCN([C@H](C)c2cccc3ccccc23)CC1)S(C)(=O)=O]

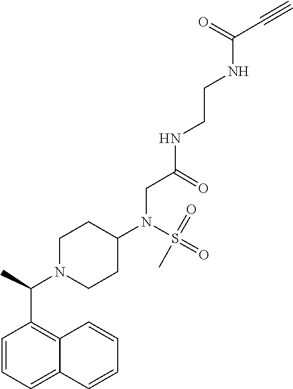 [SMILES: C#CC(=O)NCCNC(=O)CN(C1CCN([C@H](C)c2cccc3ccccc23)CC1)S(C)(=O)=O]

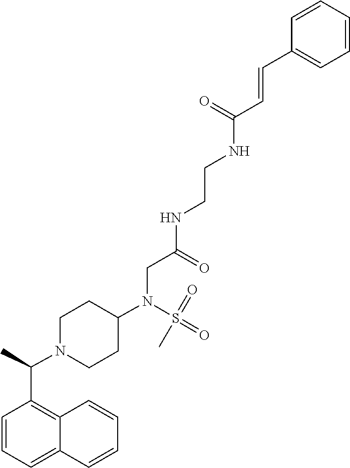 [SMILES: C[C@H](c1cccc2ccccc12)N1CCC(N(CC(=O)NCCNC(=O)/C=C/c2ccccc2)S(C)(=O)=O)CC1]

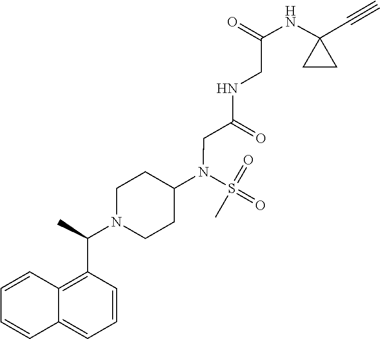 [SMILES: C#CC1(NC(=O)CNC(=O)CN(C2CCN([C@H](C)c3cccc4ccccc34)CC2)S(C)(=O)=O)CC1]

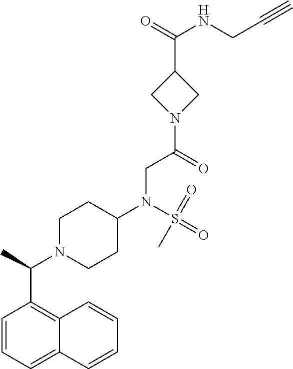 [SMILES: C#CCNC(=O)C1CN(C(=O)CN(C2CCN([C@H](C)c3cccc4ccccc34)CC2)S(C)(=O)=O)C1]

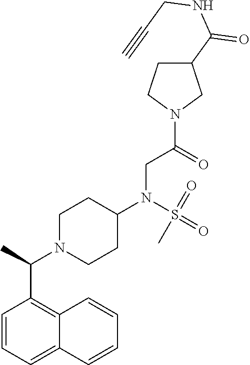 [SMILES: C#CCNC(=O)C1CCN(C(=O)CN(C2CCN([C@H](C)c3cccc4ccccc34)CC2)S(C)(=O)=O)C1]

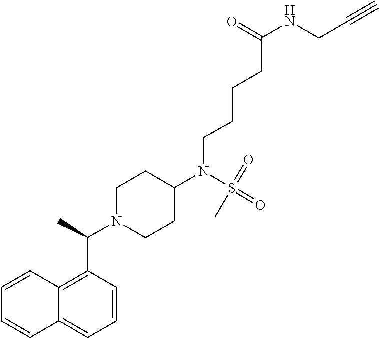 [SMILES: C#CCNC(=O)CCCCN(C1CCN([C@H](C)c2cccc3ccccc23)CC1)S(C)(=O)=O]

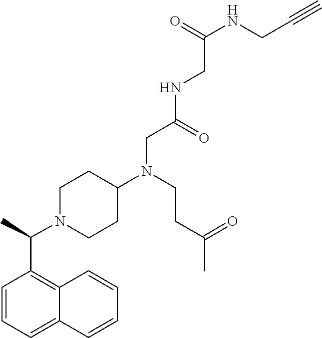 [SMILES: C#CCNC(=O)CNC(=O)CN(CCC(C)=O)C1CCN([C@H](C)c2cccc3ccccc23)CC1]

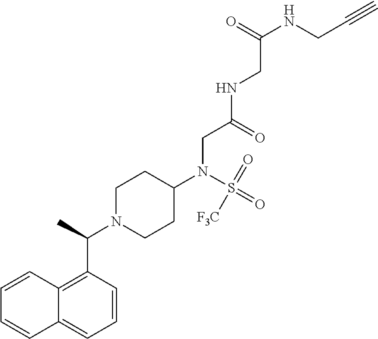 [SMILES: C#CCNC(=O)CNC(=O)CN(C1CCN([C@H](C)c2cccc3ccccc23)CC1)S(=O)(=O)C(F)(F)F]

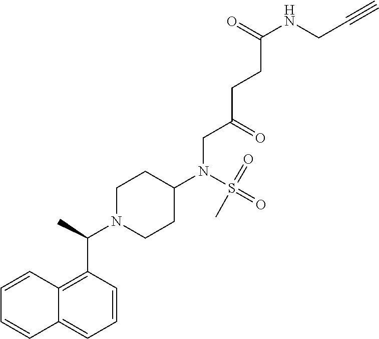 [SMILES: C#CCNC(=O)CCC(=O)CN(C1CCN([C@H](C)c2cccc3ccccc23)CC1)S(C)(=O)=O]

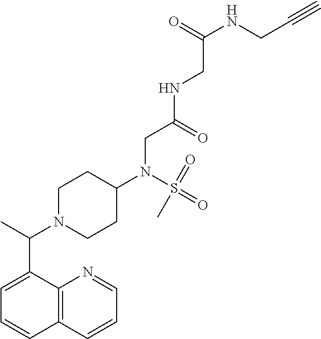 [SMILES: C#CCNC(=O)CNC(=O)CN(C1CCN(C(C)c2cccc3cccnc23)CC1)S(C)(=O)=O]

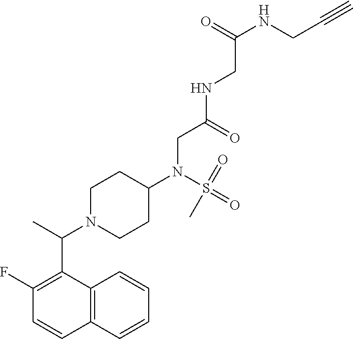 [SMILES: C#CCNC(=O)CNC(=O)CN(C1CCN(C(C)c2c(F)ccc3ccccc23)CC1)S(C)(=O)=O]

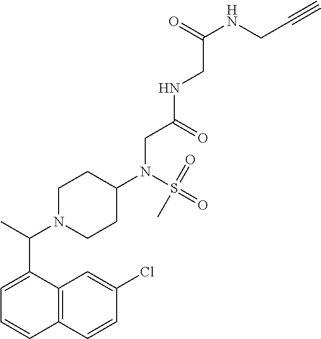 [SMILES: C#CCNC(=O)CNC(=O)CN(C1CCN(C(C)c2cccc3ccc(Cl)cc23)CC1)S(C)(=O)=O]

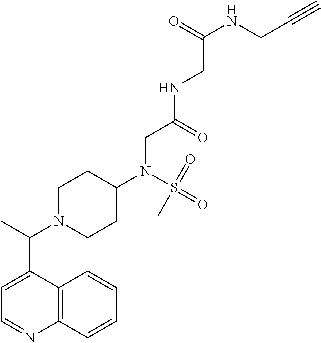 [SMILES: C#CCNC(=O)CNC(=O)CN(C1CCN(C(C)c2ccnc3ccccc23)CC1)S(C)(=O)=O]

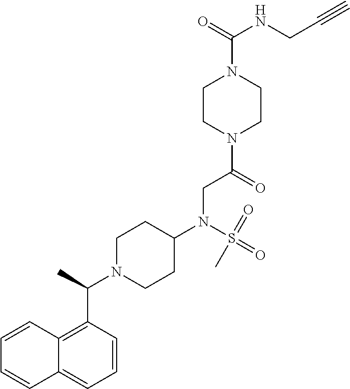 [SMILES: C#CCNC(=O)N1CCN(C(=O)CN(C2CCN([C@H](C)c3cccc4ccccc34)CC2)S(C)(=O)=O)CC1]

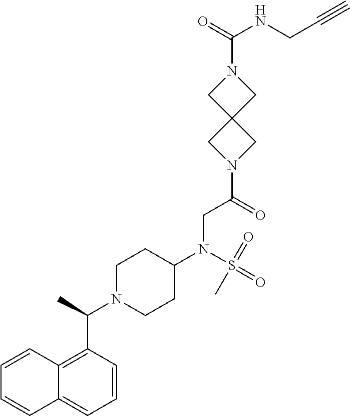 [SMILES: C#CCNC(=O)N1CC2(CN(C(=O)CN(C3CCN([C@H](C)c4cccc5ccccc45)CC3)S(C)(=O)=O)C2)C1]

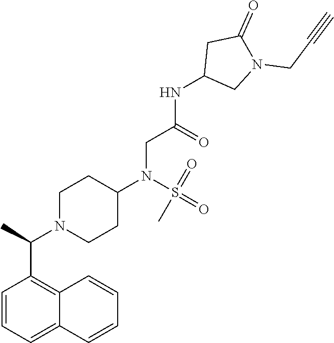 [SMILES: C#CCN1CC(NC(=O)CN(C2CCN([C@H](C)c3cccc4ccccc34)CC2)S(C)(=O)=O)CC1=O]

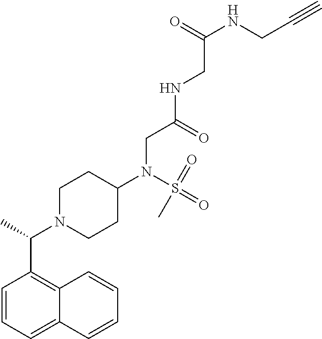 [SMILES: C#CCNC(=O)CNC(=O)CN(C1CCN([C@@H](C)c2cccc3ccccc23)CC1)S(C)(=O)=O]

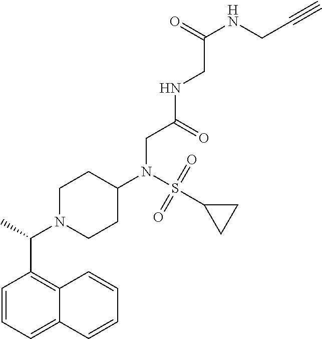 [SMILES: C#CCNC(=O)CNC(=O)CN(C1CCN([C@@H](C)c2cccc3ccccc23)CC1)S(=O)(=O)C1CC1]

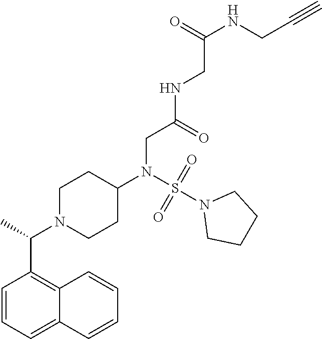 [SMILES: C#CCNC(=O)CNC(=O)CN(C1CCN([C@@H](C)c2cccc3ccccc23)CC1)S(=O)(=O)N1CCCC1]

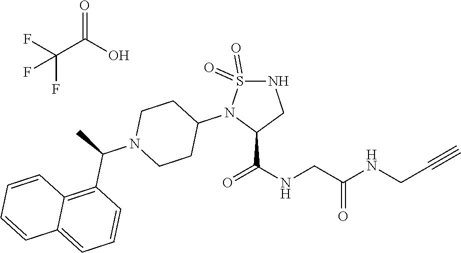 [SMILES: C#CCNC(=O)CNC(=O)[C@@H]1CNS(=O)(=O)N1C1CCN([C@H](C)c2cccc3ccccc23)CC1 ; O=C(O)C(F)(F)F]

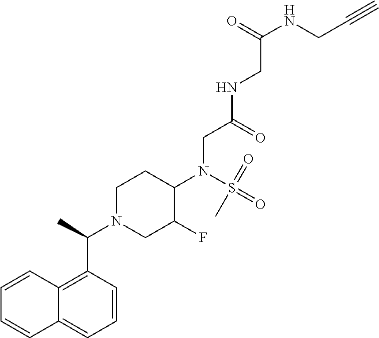 [SMILES: C#CCNC(=O)CNC(=O)CN(C1CCN([C@H](C)c2cccc3ccccc23)CC1F)S(C)(=O)=O]

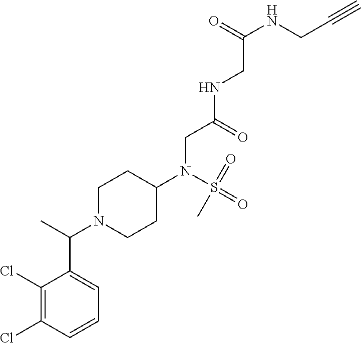 [SMILES: C#CCNC(=O)CNC(=O)CN(C1CCN(C(C)c2cccc(Cl)c2Cl)CC1)S(C)(=O)=O]

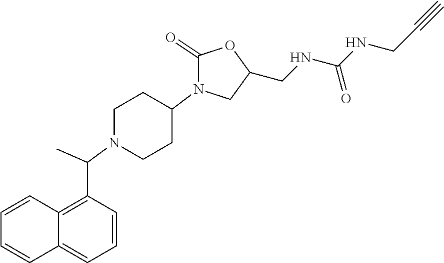 [SMILES: C#CCNC(=O)NCC1CN(C2CCN(C(C)c3cccc4ccccc34)CC2)C(=O)O1]

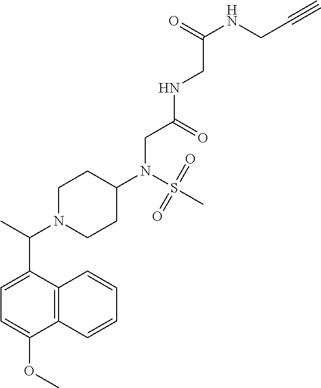 [SMILES: C#CCNC(=O)CNC(=O)CN(C1CCN(C(C)c2ccc(OC)c3ccccc23)CC1)S(C)(=O)=O]

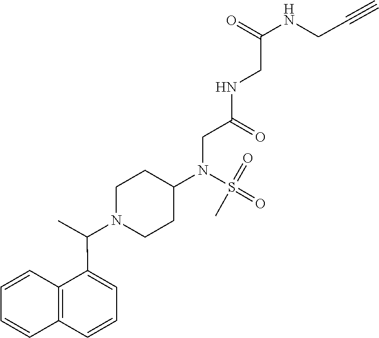 [SMILES: C#CCNC(=O)CNC(=O)CN(C1CCN(C(C)c2cccc3ccccc23)CC1)S(C)(=O)=O]

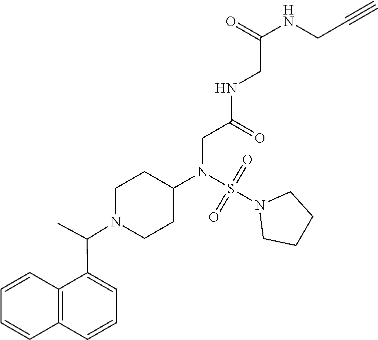 [SMILES: C#CCNC(=O)CNC(=O)CN(C1CCN(C(C)c2cccc3ccccc23)CC1)S(=O)(=O)N1CCCC1]

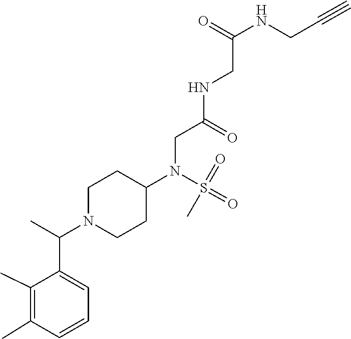 [SMILES: C#CCNC(=O)CNC(=O)CN(C1CCN(C(C)c2cccc(C)c2C)CC1)S(C)(=O)=O]

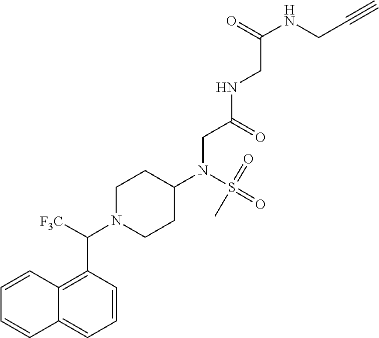 [SMILES: C#CCNC(=O)CNC(=O)CN(C1CCN(C(c2cccc3ccccc23)C(F)(F)F)CC1)S(C)(=O)=O]

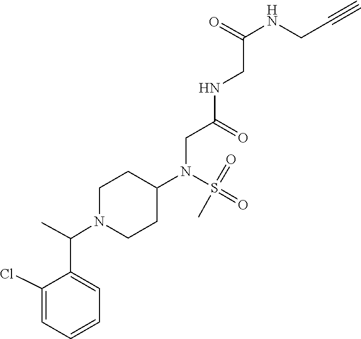 [SMILES: C#CCNC(=O)CNC(=O)CN(C1CCN(C(C)c2ccccc2Cl)CC1)S(C)(=O)=O]

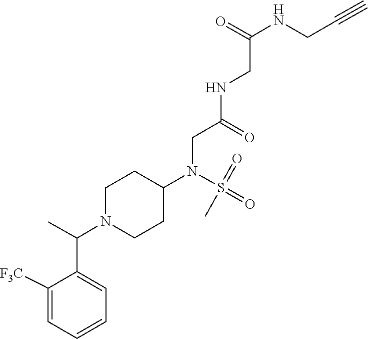 [SMILES: C#CCNC(=O)CNC(=O)CN(C1CCN(C(C)c2ccccc2C(F)(F)F)CC1)S(C)(=O)=O]

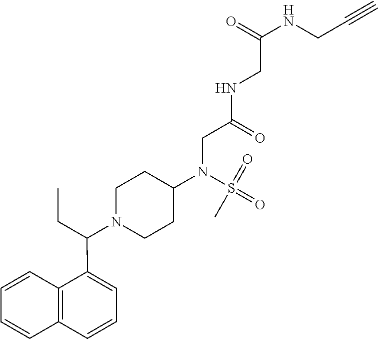 [SMILES: C#CCNC(=O)CNC(=O)CN(C1CCN(C(CC)c2cccc3ccccc23)CC1)S(C)(=O)=O]

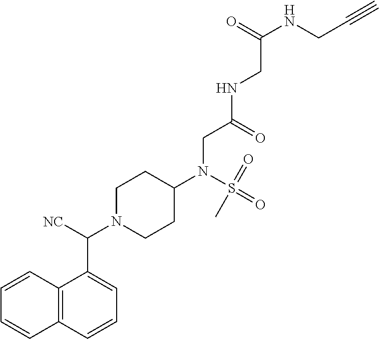 [SMILES: C#CCNC(=O)CNC(=O)CN(C1CCN(C(C#N)c2cccc3ccccc23)CC1)S(C)(=O)=O]

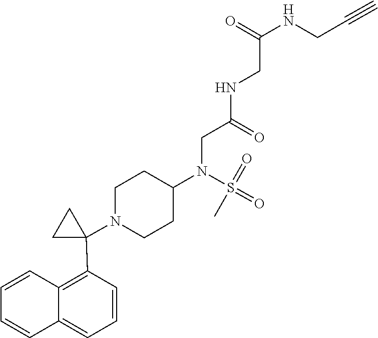 [SMILES: C#CCNC(=O)CNC(=O)CN(C1CCN(C2(c3cccc4ccccc34)CC2)CC1)S(C)(=O)=O]

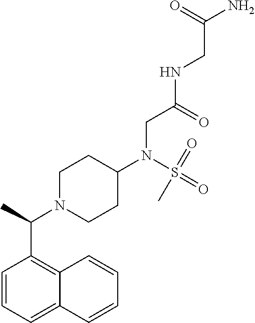 [SMILES: C[C@H](c1cccc2ccccc12)N1CCC(N(CC(=O)NCC(N)=O)S(C)(=O)=O)CC1]

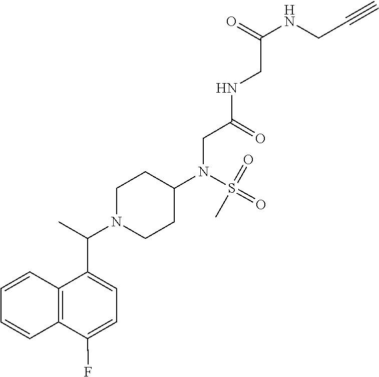 [SMILES: C#CCNC(=O)CNC(=O)CN(C1CCN(C(C)c2ccc(F)c3ccccc23)CC1)S(C)(=O)=O]

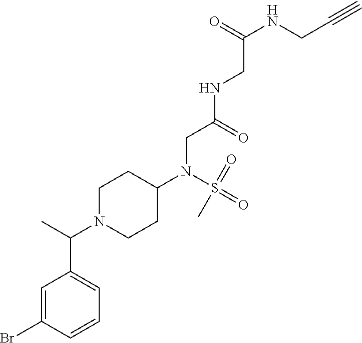 [SMILES: C#CCNC(=O)CNC(=O)CN(C1CCN(C(C)c2cccc(Br)c2)CC1)S(C)(=O)=O]

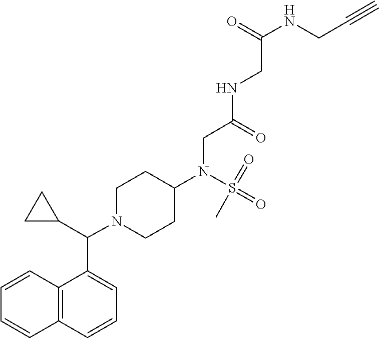 [SMILES: C#CCNC(=O)CNC(=O)CN(C1CCN(C(c2cccc3ccccc23)C2CC2)CC1)S(C)(=O)=O]

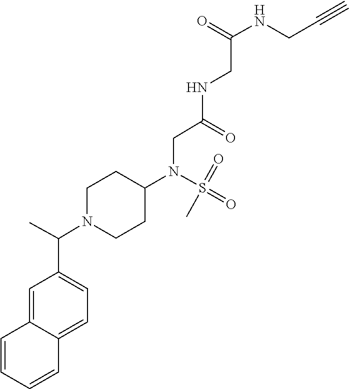 [SMILES: C#CCNC(=O)CNC(=O)CN(C1CCN(C(C)c2ccc3ccccc3c2)CC1)S(C)(=O)=O]

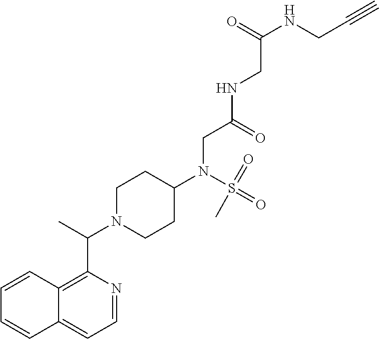 [SMILES: C#CCNC(=O)CNC(=O)CN(C1CCN(C(C)c2nccc3ccccc23)CC1)S(C)(=O)=O]

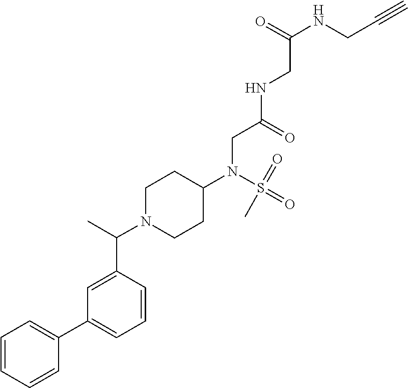 [SMILES: C#CCNC(=O)CNC(=O)CN(C1CCN(C(C)c2cccc(-c3ccccc3)c2)CC1)S(C)(=O)=O]

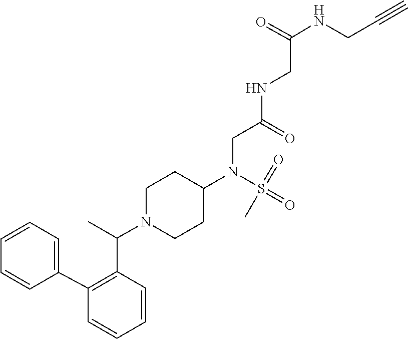 [SMILES: C#CCNC(=O)CNC(=O)CN(C1CCN(C(C)c2ccccc2-c2ccccc2)CC1)S(C)(=O)=O]

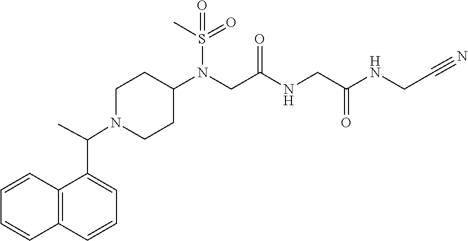 [SMILES: CC(c1cccc2ccccc12)N1CCC(N(CC(=O)NCC(=O)NCC#N)S(C)(=O)=O)CC1]

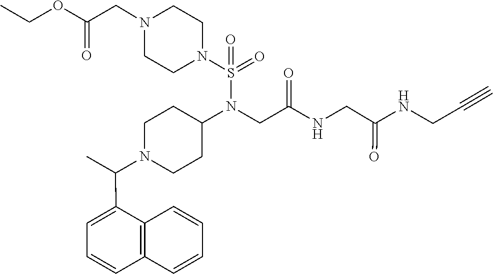 [SMILES: C#CCNC(=O)CNC(=O)CN(C1CCN(C(C)c2cccc3ccccc23)CC1)S(=O)(=O)N1CCN(CC(=O)OCC)CC1]

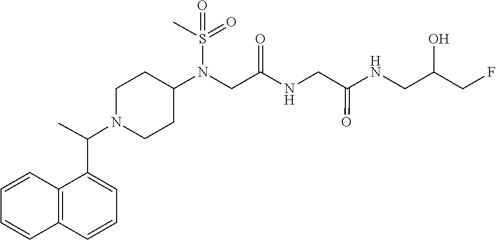 [SMILES: CC(c1cccc2ccccc12)N1CCC(N(CC(=O)NCC(=O)NCC(O)CF)S(C)(=O)=O)CC1]

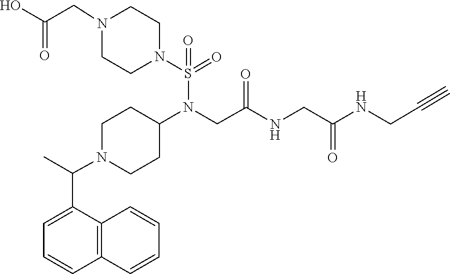 [SMILES: C#CCNC(=O)CNC(=O)CN(C1CCN(C(C)c2cccc3ccccc23)CC1)S(=O)(=O)N1CCN(CC(=O)O)CC1]

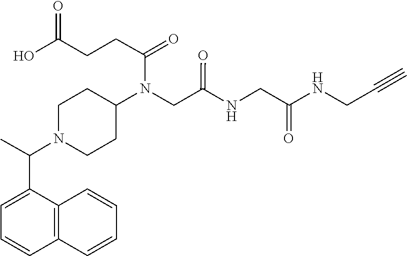 [SMILES: C#CCNC(=O)CNC(=O)CN(C(=O)CCC(=O)O)C1CCN(C(C)c2cccc3ccccc23)CC1]

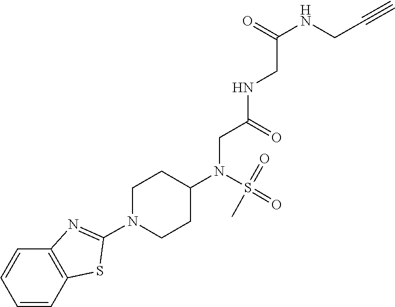 [SMILES: C#CCNC(=O)CNC(=O)CN(C1CCN(c2nc3ccccc3s2)CC1)S(C)(=O)=O]

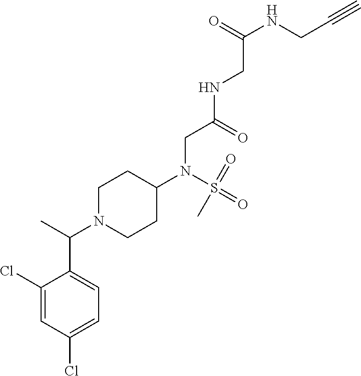 [SMILES: C#CCNC(=O)CNC(=O)CN(C1CCN(C(C)c2ccc(Cl)cc2Cl)CC1)S(C)(=O)=O]

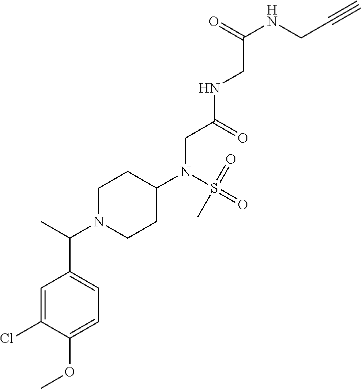 [SMILES: C#CCNC(=O)CNC(=O)CN(C1CCN(C(C)c2ccc(OC)c(Cl)c2)CC1)S(C)(=O)=O]

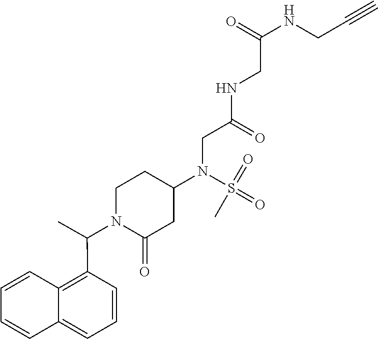 [SMILES: C#CCNC(=O)CNC(=O)CN(C1CCN(C(C)c2cccc3ccccc23)C(=O)C1)S(C)(=O)=O]

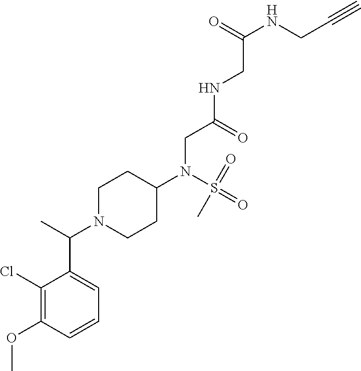 [SMILES: C#CCNC(=O)CNC(=O)CN(C1CCN(C(C)c2cccc(OC)c2Cl)CC1)S(C)(=O)=O]

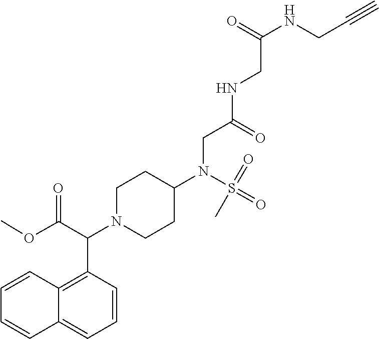 [SMILES: C#CCNC(=O)CNC(=O)CN(C1CCN(C(C(=O)OC)c2cccc3ccccc23)CC1)S(C)(=O)=O]

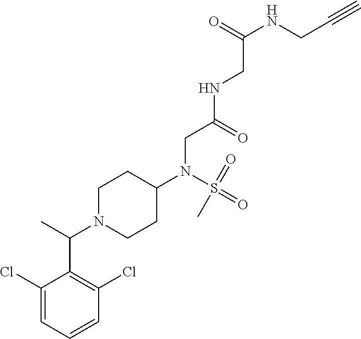 [SMILES: C#CCNC(=O)CNC(=O)CN(C1CCN(C(C)c2c(Cl)cccc2Cl)CC1)S(C)(=O)=O]

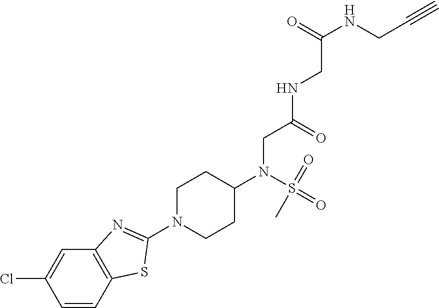 [SMILES: C#CCNC(=O)CNC(=O)CN(C1CCN(c2nc3cc(Cl)ccc3s2)CC1)S(C)(=O)=O]

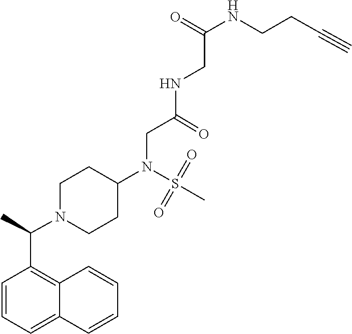 [SMILES: C#CCCNC(=O)CNC(=O)CN(C1CCN([C@H](C)c2cccc3ccccc23)CC1)S(C)(=O)=O]

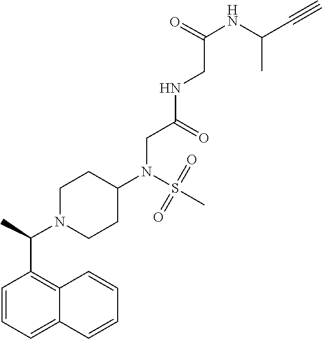 [SMILES: C#CC(C)NC(=O)CNC(=O)CN(C1CCN([C@H](C)c2cccc3ccccc23)CC1)S(C)(=O)=O]

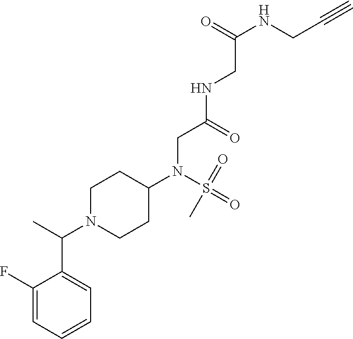 [SMILES: C#CCNC(=O)CNC(=O)CN(C1CCN(C(C)c2ccccc2F)CC1)S(C)(=O)=O]

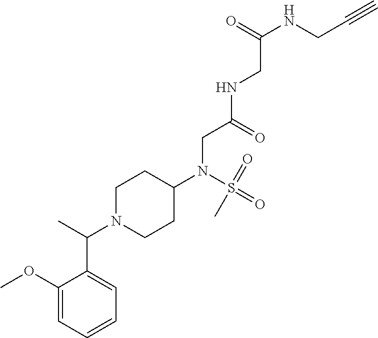 [SMILES: C#CCNC(=O)CNC(=O)CN(C1CCN(C(C)c2ccccc2OC)CC1)S(C)(=O)=O]

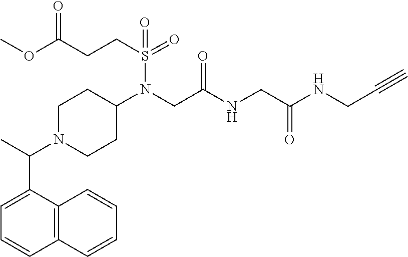 [SMILES: C#CCNC(=O)CNC(=O)CN(C1CCN(C(C)c2cccc3ccccc23)CC1)S(=O)(=O)CCC(=O)OC]

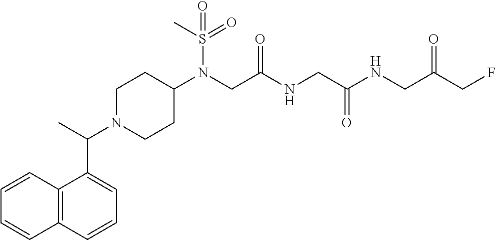 [SMILES: CC(c1cccc2ccccc12)N1CCC(N(CC(=O)NCC(=O)NCC(=O)CF)S(C)(=O)=O)CC1]

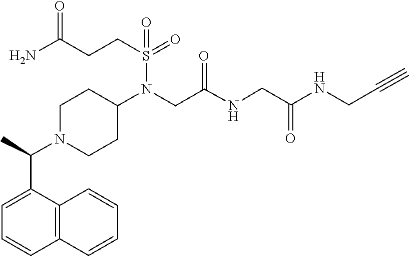 [SMILES: C#CCNC(=O)CNC(=O)CN(C1CCN([C@H](C)c2cccc3ccccc23)CC1)S(=O)(=O)CCC(N)=O]

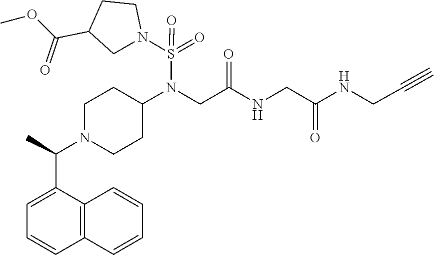 [SMILES: C#CCNC(=O)CNC(=O)CN(C1CCN([C@H](C)c2cccc3ccccc23)CC1)S(=O)(=O)N1CCC(C(=O)OC)C1]

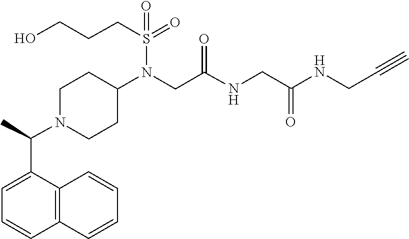 [SMILES: C#CCNC(=O)CNC(=O)CN(C1CCN([C@H](C)c2cccc3ccccc23)CC1)S(=O)(=O)CCCO]

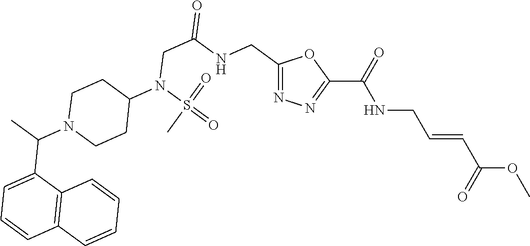 [SMILES: COC(=O)/C=C/CNC(=O)c1nnc(CNC(=O)CN(C2CCN(C(C)c3cccc4ccccc34)CC2)S(C)(=O)=O)o1]

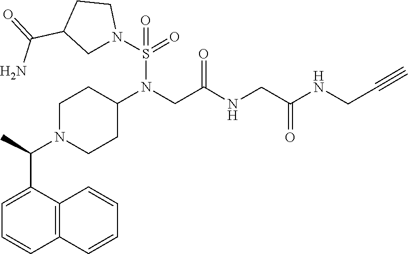 [SMILES: C#CCNC(=O)CNC(=O)CN(C1CCN([C@H](C)c2cccc3ccccc23)CC1)S(=O)(=O)N1CCC(C(N)=O)C1]

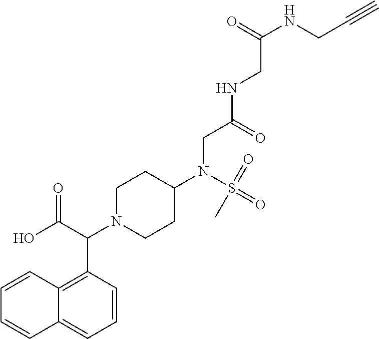 [SMILES: C#CCNC(=O)CNC(=O)CN(C1CCN(C(C(=O)O)c2cccc3ccccc23)CC1)S(C)(=O)=O]

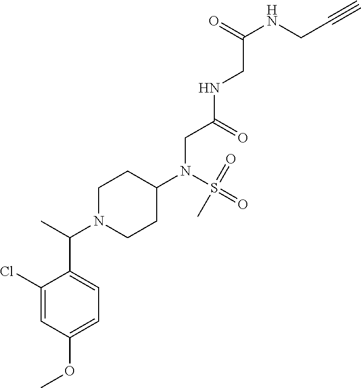 [SMILES: C#CCNC(=O)CNC(=O)CN(C1CCN(C(C)c2ccc(OC)cc2Cl)CC1)S(C)(=O)=O]

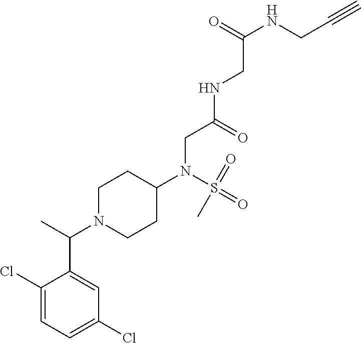 [SMILES: C#CCNC(=O)CNC(=O)CN(C1CCN(C(C)c2cc(Cl)ccc2Cl)CC1)S(C)(=O)=O]

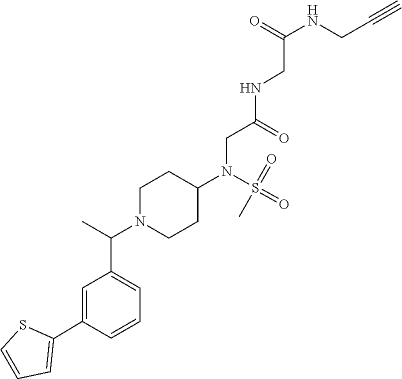 [SMILES: C#CCNC(=O)CNC(=O)CN(C1CCN(C(C)c2cccc(-c3cccs3)c2)CC1)S(C)(=O)=O]

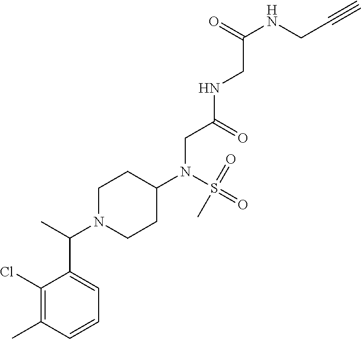 [SMILES: C#CCNC(=O)CNC(=O)CN(C1CCN(C(C)c2cccc(C)c2Cl)CC1)S(C)(=O)=O]

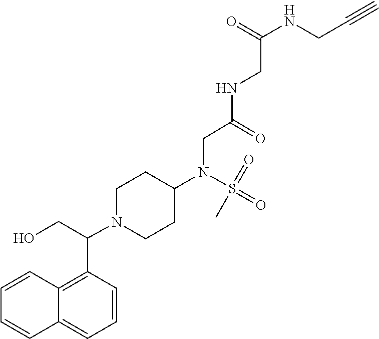 [SMILES: C#CCNC(=O)CNC(=O)CN(C1CCN(C(CO)c2cccc3ccccc23)CC1)S(C)(=O)=O]

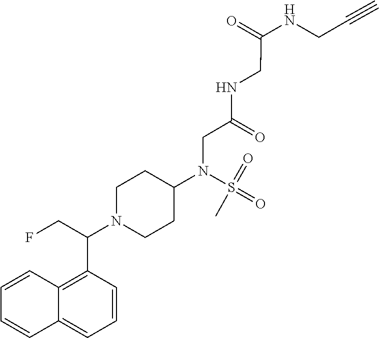 [SMILES: C#CCNC(=O)CNC(=O)CN(C1CCN(C(CF)c2cccc3ccccc23)CC1)S(C)(=O)=O]

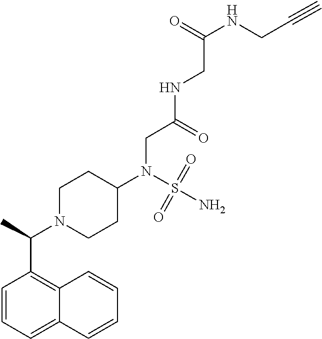 [SMILES: C#CCNC(=O)CNC(=O)CN(C1CCN([C@H](C)c2cccc3ccccc23)CC1)S(N)(=O)=O]

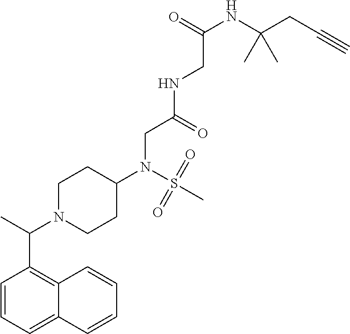 [SMILES: C#CCC(C)(C)NC(=O)CNC(=O)CN(C1CCN(C(C)c2cccc3ccccc23)CC1)S(C)(=O)=O]

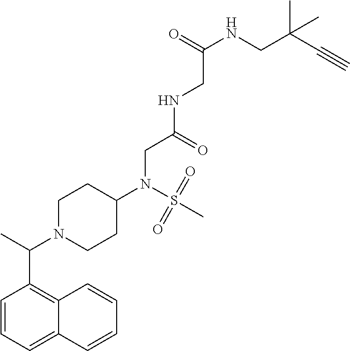 [SMILES: C#CC(C)(C)CNC(=O)CNC(=O)CN(C1CCN(C(C)c2cccc3ccccc23)CC1)S(C)(=O)=O]

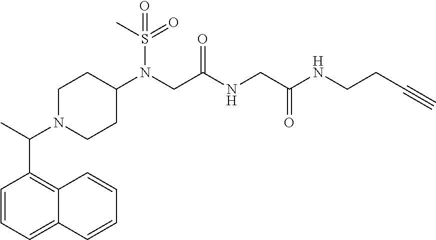 [SMILES: C#CCCNC(=O)CNC(=O)CN(C1CCN(C(C)c2cccc3ccccc23)CC1)S(C)(=O)=O]

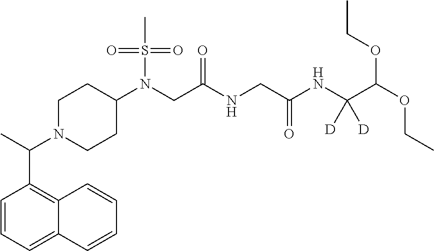 [SMILES: [2H]C([2H])(NC(=O)CNC(=O)CN(C1CCN(C(C)c2cccc3ccccc23)CC1)S(C)(=O)=O)C(OCC)OCC]

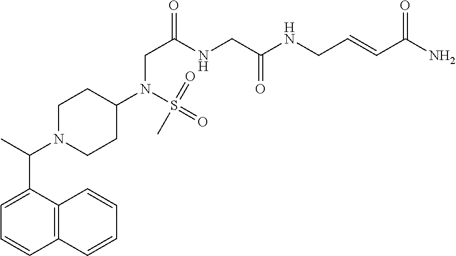 [SMILES: CC(c1cccc2ccccc12)N1CCC(N(CC(=O)NCC(=O)NC/C=C/C(N)=O)S(C)(=O)=O)CC1]

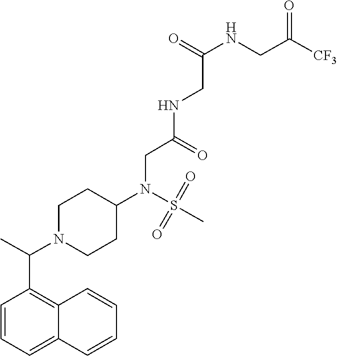 [SMILES: CC(c1cccc2ccccc12)N1CCC(N(CC(=O)NCC(=O)NCC(=O)C(F)(F)F)S(C)(=O)=O)CC1]

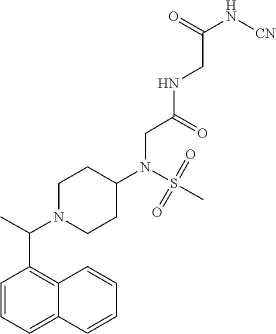 [SMILES: CC(c1cccc2ccccc12)N1CCC(N(CC(=O)NCC(=O)NC#N)S(C)(=O)=O)CC1]

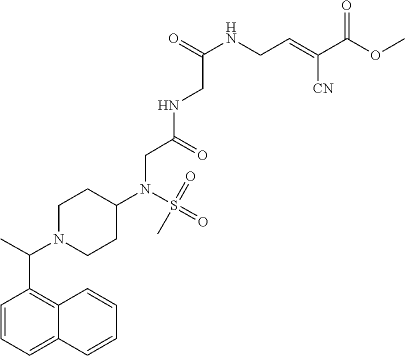 [SMILES: COC(=O)/C(C#N)=C/CNC(=O)CNC(=O)CN(C1CCN(C(C)c2cccc3ccccc23)CC1)S(C)(=O)=O]

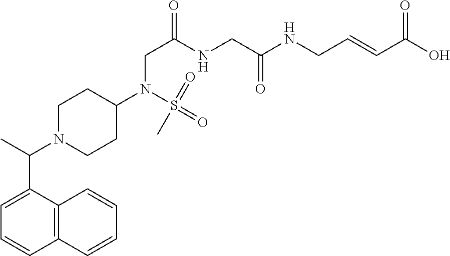 [SMILES: CC(c1cccc2ccccc12)N1CCC(N(CC(=O)NCC(=O)NC/C=C/C(=O)O)S(C)(=O)=O)CC1]

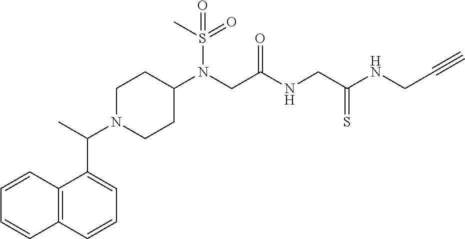 [SMILES: C#CCNC(=S)CNC(=O)CN(C1CCN(C(C)c2cccc3ccccc23)CC1)S(C)(=O)=O]

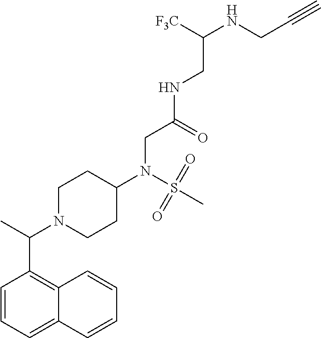 [SMILES: C#CCNC(CNC(=O)CN(C1CCN(C(C)c2cccc3ccccc23)CC1)S(C)(=O)=O)C(F)(F)F]

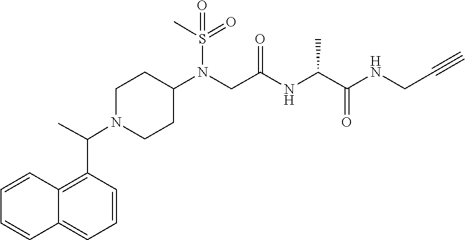 [SMILES: C#CCNC(=O)[C@@H](C)NC(=O)CN(C1CCN(C(C)c2cccc3ccccc23)CC1)S(C)(=O)=O]

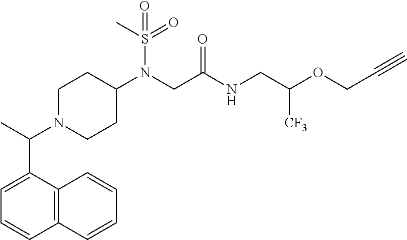 [SMILES: C#CCOC(CNC(=O)CN(C1CCN(C(C)c2cccc3ccccc23)CC1)S(C)(=O)=O)C(F)(F)F]

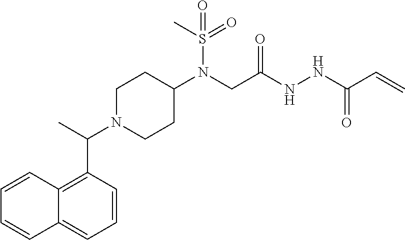 [SMILES: C=CC(=O)NNC(=O)CN(C1CCN(C(C)c2cccc3ccccc23)CC1)S(C)(=O)=O]

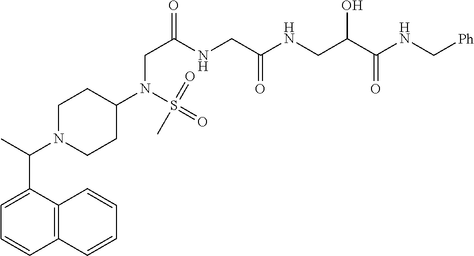 [SMILES: CC(c1cccc2ccccc12)N1CCC(N(CC(=O)NCC(=O)NCC(O)C(=O)NCc2ccccc2)S(C)(=O)=O)CC1]

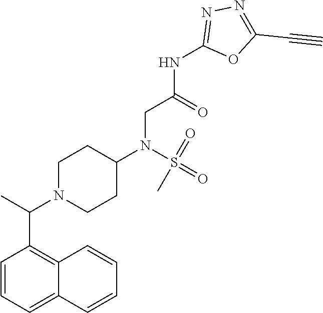 [SMILES: C#Cc1nnc(NC(=O)CN(C2CCN(C(C)c3cccc4ccccc34)CC2)S(C)(=O)=O)o1]

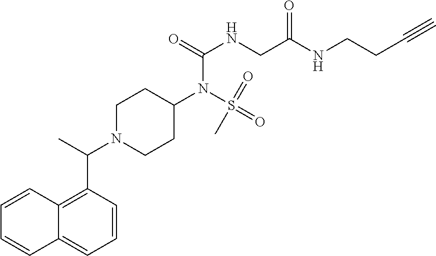 [SMILES: C#CCCNC(=O)CNC(=O)N(C1CCN(C(C)c2cccc3ccccc23)CC1)S(C)(=O)=O]

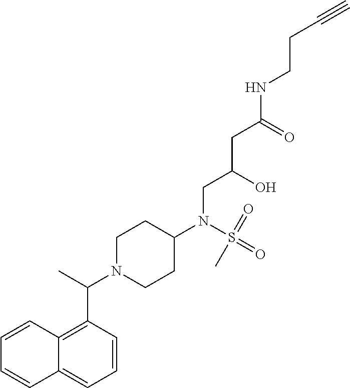 [SMILES: C#CCCNC(=O)CC(O)CN(C1CCN(C(C)c2cccc3ccccc23)CC1)S(C)(=O)=O]

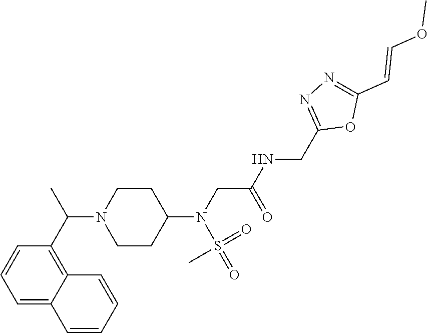 [SMILES: CO/C=C/c1nnc(CNC(=O)CN(C2CCN(C(C)c3cccc4ccccc34)CC2)S(C)(=O)=O)o1]

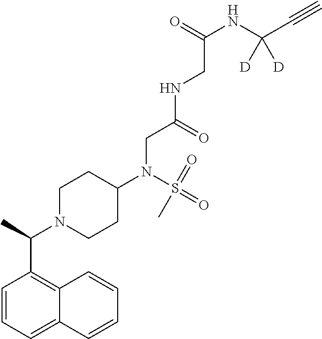 [SMILES: [2H]C([2H])(C#C)NC(=O)CNC(=O)CN(C1CCN([C@H](C)c2cccc3ccccc23)CC1)S(C)(=O)=O]

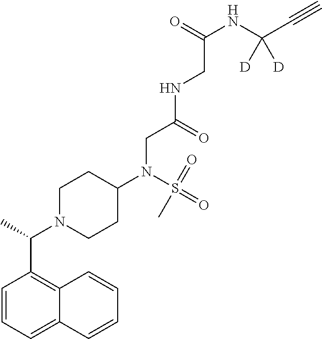 [SMILES: [2H]C([2H])(C#C)NC(=O)CNC(=O)CN(C1CCN([C@@H](C)c2cccc3ccccc23)CC1)S(C)(=O)=O]

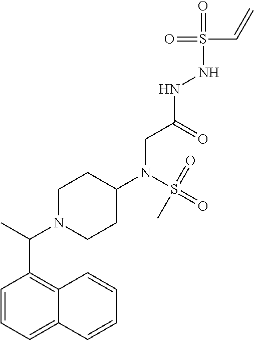 [SMILES: C=CS(=O)(=O)NNC(=O)CN(C1CCN(C(C)c2cccc3ccccc23)CC1)S(C)(=O)=O]

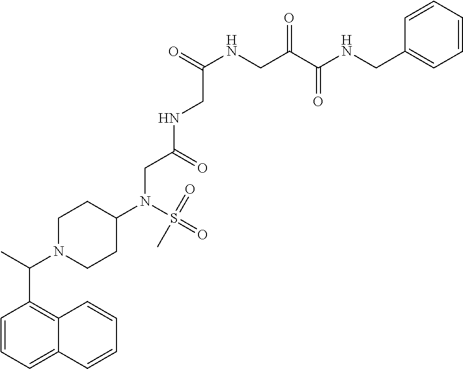 [SMILES: CC(c1cccc2ccccc12)N1CCC(N(CC(=O)NCC(=O)NCC(=O)C(=O)NCc2ccccc2)S(C)(=O)=O)CC1]

Abbreviations used herein have their meanings commonly understood in the art unless otherwise defined or contrary from context. For example, Ms is commonly understood in the art as mesyl or —SO₂Me, and Boc means tert-butoxycarbonyl.

The compounds of the present disclosure can be readily synthesized in view of the present disclosure. Exemplified syntheses are also shown in the Examples section. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Definition

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula X, Y, I, II, III, IV, or V or a subformula thereof, as applicable, are independently selected. Non-limiting useful groups for the variables in compounds of Formula X, Y, I, II, III, IV, or V, or a subformula thereof, as applicable, include any of the respective groups, individually or in any combination, as shown in the compounds of Table A herein or specific Compound Nos. 1-177.

The described embodiments of the present disclosure can be combined. Such combination is contemplated and within the scope of the present disclosure. For example, it is contemplated that the definition(s) of any one or more of X¹, X², R⁹, R¹⁰ , Y¹, Y², Y³, Y⁴, Y⁵, Y⁶, Y⁷, Y⁸, Y⁹, and W of Formula Y can be combined with the definition of any one or more of the other(s) of X¹, X², R⁸, R¹⁰, Y¹, Y², Y³, Y⁴, Y⁵, Y⁶, Y⁷, Y⁸, Y⁹, and W, as applicable, and the resulted compounds from the combination are within the scope of the present disclosure.

The symbol, [symbol] when displayed perpendicular to (or otherwise crossing) a bond, indicates the point at which the displayed moiety is attached to the remainder of the molecule. It should be noted that for a divalent structure (or multivalent structure), the immediately connected group or groups or appropriate variable(s) shown in a formula may be shown in the divalent structure (or multivalent structure) beyond the symbol, [symbol] to indicate direction of attachment. When the immediately connected group(s) is not shown for either of the two attaching points of a divalent structure, it should mean that either direction of attachment to the remainder of the molecule is allowed, unless otherwise specified or obviously contrary from context. Using a structure of “A-heteroarylene-B” as an example, if the heteroarylene is defined as

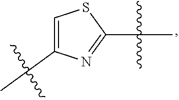 [SMILES: *c1csc(*)n1]

i.e., the immediately connected group(s) is not shown, then the structure can be either

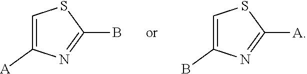

When the symbol, [symbol] is used as a bond in a structure, it should be understood that the stereochemistry of the structure can be any of the chemically possible stereoisomer.

For example, for the following moiety,

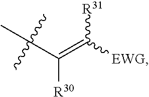

the [symbol] that crosses the upper left bond should be understood as meaning that the carbon atom bonded with R³⁰ is the attaching point of the moiety to the remainder of the molecule, as discussed above; whereas the [symbol] used as a bond on the right side indicates that the stereochemistry of the double bond can be either E or Z configuration.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s), for example, the compound can have an enantiomeric excess (ee) of greater than 60%, such as greater than 80%, greater than 90%, greater than 95%, greater than 98%, greater than 99%, etc. The presence and/or amounts of stereoisomers can be determined by those skilled in the art in view of the present disclosure, including through the use of a chiral HPLC or other methods.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, and/or possible pharmaceutically acceptable salts thereof (e.g., acid addition salt such as HCl, methane sulfonate salt or base addition salt such as Na salt). When a particular position(s) of a structure or partial structure herein, such as a compound or a substituent herein, is designated as holding deuterium (stated as “D” or “deuterium”), it is understood that the abundance of deuterium at that position(s) is greater than the natural abundance of deuterium, which is about 0.0156%, preferably, the abundance of deuterium at that position is significantly greater than its natural abundance, for example, at that position, there is at least 50.1%, at least 70%, at least 80%, at least 90%, at least 95%, at least 98%, or at least 99% incorporation of deuterium. Hydrates and solvates of the compounds of the present disclosure are considered compositions of the present disclosure, wherein the compound(s) is in association with water or solvent, respectively. In any of the embodiments described herein, unless otherwise specified or contrary from context, the compound or pharmaceutically acceptable salt thereof can be selected from those exemplified embodiments EM1 to EM22 described herein. In some preferred embodiments, the compound or pharmaceutically acceptable salt thereof for the pharmaceutical compositions or methods of treatment herein can have an IC50 of less than 100 nM when tested under the PLpro enzymatic inhibition assay herein.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the term “alkyl” refers to a saturated linear or branched-chain monovalent hydrocarbon radical. In some embodiments, the alkyl refers to linear or branched C1-C32 alkyl, C1-C24 alkyl, C1-C12 alkyl, C1-C8 alkyl, or C1-C6 alkyl. In some embodiments, an alkyl refers to linear or branched C1-C6 alkyl. Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C1-C3 alkyl group. In some embodiments, an alkyl group is a C1-C2 alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C1-C8 alkylene). In some embodiments, an alkylene group contains 1 to 6 carbon atoms (C1-C6 alkylene). In some embodiments, an alkylene group contains 1 to 5 carbon atoms (C1-C5 alkylene). In some embodiments, an alkylene group contains 1 to 4 carbon atoms (C1-C4 alkylene). In some embodiments, an alkylene contains 1 to three carbon atoms (C1-C3 alkylene). In some embodiments, an alkylene group contains 1 to two carbon atoms (C1-C2 alkylene). In some embodiments, an alkylene group contains one carbon atom (C1 alkylene).

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). In some embodiments, “carbocyclyl” is fully saturated. The term carbocyclyl includes mono-, bi-, tri-, fused, bridged, and spiro-ring systems, and combinations thereof. In one embodiment, carbocyclyl includes 3 to 15 carbon atoms (C3-C15). In one embodiment, carbocyclyl includes 3 to 12 carbon atoms (C3-C12). In another embodiment, carbocyclyl includes C3-C8, C3-C10 or C5-C10. In another embodiment, carbocyclyl, as a monocycle, includes C3-C8, C3-C6 or C5-C6. In some embodiments, carbocyclyl, as a bicycle, includes C7-C12. In another embodiment, carbocyclyl, as a spiro system, includes C5-C12. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

As used herein, the term “halogen” (or “halo” or “halide”) refers to fluorine, chlorine, bromine, or iodine.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and Spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternalized (e.g., [NR₄]⁺Br, [NR₄]⁺OH). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4, 5,6,7-tetrahydrobenzo[d]imidazolyl, 1,6-dihydroimidazol[4,5-d]pyrrolo[2,3-b]pyridinyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3. 1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl.

In some embodiments, the heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

As used herein, the term “heteroarylene” refers to a bivalent heteroaryl radical which may be optionally substituted. The term “heteroaryl” used alone or as part of a larger moiety (e.g., “heteroaryl alkyl” (also“heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 4-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, purinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group.

Typically, the salts of the present disclosure are pharmaceutically acceptable salts. As used herein, the term “pharmaceutically acceptable salts” refers to non-toxic salts of the PLpro compounds as described herein. Salts of the PLpro compounds as described herein may comprise acid addition salts. Representative salts include acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, magnesium, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this disclosure and these should be considered to form a further aspect of the disclosure.

As used herein, the term “prodrug” refers to a derivative of any bioactive compound having a reactive functional group (e.g., —COOH from naproxen, aspirin) that, when administered to a site of action, is cleaved by hydrolysis and/or enzymatic action in such a manner as to release the bioactive agent at its target site or sites of activity, with the remaining residues being non-toxic or metabolized to non-toxic compounds. Particularly favored prodrugs are those that increase the bioavailability of the compound of the present disclosure when such compounds are administered to a mammal, for example, by allowing an orally administered compound to be more readily absorbed into the blood, or which enhance delivery of the parent compound to a biological compartment, for example, the brain or lymphatic system, relative to the parent species.

As used herein, the term “solvate” refers to a complex of variable stoichiometry formed by a solute (in this disclosure, a compound of the present disclosure) and a solvent. Such solvents, for the purpose of the disclosure, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water. In some embodiments, the solvate of compounds of the present disclosure may include, but not limited to hemihydrate, monohydrate, or trihydrate etc.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocyclyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein for use in connection with the formulae described herein include acyclic and cyclic, branched and unbranched, carbocyclic and heterocyclic, aromatic and non-aromatic substituents of organic compounds. The permissible substituents for use in connection with the formulae described herein can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents suitable for use in connection with the formulae described herein include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, OH, hydroxyalkyl, haloalkyl, —O-alkyl, —O-haloalkyl, -alkylene-O-alkyl, O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents for use in connection with the formulae described herein include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to CF₃), O(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents suitable for use in connection with the formulae described herein include, but are not limited to, halogen, —CN, —N02, —N3, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —C₀₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂,—SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents suitable for use in connection with the formulae described herein include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents suitable for use in connection with the formulae described herein include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

As used herein, the term “subject” refers to a tissue, system, animal, mammal, or human that is being sought, for instance, by a researcher or clinician to elicit the biological or medical response thereof by a biologically active agent such as therapeutic agent. In some embodiments, the subject refers to a mammal. In some embodiments, the subject refers to a human.

As used herein, the terms “treat,” “treatment,” “treating,” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder, such as coronavirus infections. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein, the term “therapeutically effective amount” means that amount of a drug or pharmaceutical agent that will elicit the biological or medical response of a tissue, system, animal, or human that is being sought, for instance, by a researcher or clinician. The biological or medical response may be considered a prophylactic response or a treatment response. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. An effective amount as used herein would also include an amount sufficient to delay the development of a symptom of the disease, alter the course of a symptom of disease (for example but not limited to slow the progression of a symptom of the disease), or reverse a symptom of disease.

### Pharmaceutical Composition and Use Thereof as Therapy for Treating Coronavirus Infections

In some embodiments, the present disclosure also provides a pharmaceutical composition comprising a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) and optionally one or more pharmaceutically acceptable excipient. In any of the embodiments described herein, unless specified or otherwise contrary from context, the pharmaceutical composition can comprise a compound selected from those described in Table A herein, or a pharmaceutically acceptable salt thereof.

In one embodiment, the present disclosure further provides pharmaceutical compositions comprising an effective amount of a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) and one or more pharmaceutically acceptable carriers, diluents, or excipients. In some embodiments, the pharmaceutical composition herein comprises a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) in an amount effective for treating coronavirus infections and associated conditions in a subject in need of such treatment. In some embodiments, the coronavirus infections and associated conditions are caused by a coronavirus selected from the group of SARS-CoV, HCoV-229E, HCoV-OC43, HCoV-NL63, MERS-CoV, HCoV-HKU1 and SARS-CoV-2. In some embodiments, the coronavirus infections and associated conditions are caused by SARS-CoV-2. In some embodiments, the pharmaceutical composition herein comprises the compound of the present disclosure in an amount effective in reducing the activity of the viral PLpro, which can translate to inhibition of replication of a SARS-CoV-2 virus thereby treating infections and associated conditions caused by the SARS-CoV-2 virus.

Pharmaceutical formulations may be presented in unit dose forms containing a predetermined amount of active ingredient per unit dose. Such a unit may contain, as a non-limiting example, 0.5 mg to 1 g of one or more compounds of the present disclosure, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

In some embodiments, this disclosure provides pharmaceutical compositions suitable for injectable use comprising the one or more compounds of the present disclosure and a liquid carrier. In some embodiments, the injectable composition comprises sterile aqueous solutions (where water soluble) or dispersions, and sterile powders for the extemporaneous preparation of sterile injectable solutions or dispersion. In some embodiments, this disclosure provides pharmaceutical compositions suitable for intravenous injection administration comprising the one or more compounds of the present disclosure and a carrier selected from the group of physiological salines, sterile water, Cremophor® EL.TM. (BASF, Parsippany, N.J.), or phosphate buffered saline (PBS). In all cases, the injectable pharmaceutical compositions are sterile and of fluidity sufficient for administration by syringe. In all cases, the injectable pharmaceutical compositions are stable under the conditions of manufacture and storage and free from contamination by microorganisms such as bacteria and fungi.

In some embodiments, the carrier for the injectable pharmaceutical composition comprises a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Action of microorganisms can be prevented by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, aluminum monostearate and gelatin.

Sterile injectable solutions can be prepared by incorporating the compounds in the required amounts in an appropriate solvent with one or a combination of ingredients enumerated above, as required, followed by filtered sterilization.

Generally, dispersions are prepared by incorporating the one or more compounds of the present disclosure into a sterile vehicle containing a basic dispersion medium and required other ingredients as enumerated above. In the case of sterile powders for the preparation of sterile injectable solutions, the preferred methods of preparation are vacuum drying and freeze-drying which yields a powder of the one or more compounds of the present disclosure and any additional suitable formulation ingredients from a previously sterile-filtered solution thereof.

In some embodiments, this disclosure provides pharmaceutical compositions suitable for oral administration (oral pharmaceutical dosage) comprising the one or more compounds of the present disclosure and an inert diluent or an edible carrier. For oral administration, the one or more compounds of the present disclosure are admixed with excipients and formulated in the form of tablets, orally disintegrating tablets; capsules; oral liquids including solutions or suspensions, each with aqueous or non-aqueous liquids; soft gelatin capsules (softgels); effervescent tablets; chewable tablets; oral sprays; thin films; powders or granules; edible foams or whips; or oil-in-water liquid emulsions or water-in-oil liquid emulsions; or loosely compressed tablets. Pharmaceutically compatible binding agents and/or adjuvant materials may be optionally included as part of the oral pharmaceutical dosage. The tablets, pills, capsules, lozenges, and the like may comprise any of the following ingredients, or compounds of a similar nature: a binder such as microcrystalline cellulose or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. For instance, for oral administration in the form of a tablet or capsule, the active drug component may be combined with an oral, non-toxic pharmaceutically acceptable inert carrier such as ethanol, glycerol, water, and the like.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol may be added to the mixture before the encapsulation. A disintegrating or solubilizing agent such as agar-agar, calcium carbonate or sodium carbonate may also be added to improve the availability of the medicament when the capsule is ingested. Moreover, when desired or necessary, suitable binders, lubricants, disintegrating agents, and coloring agents may also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets may be formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, alginates, gelatins, or polyvinyl pyrrolidone, solution retardants such as paraffin, resorption accelerators such as a quaternary salt, and/or absorption agents such as bentonite, kaolin, or dicalcium phosphate. The powder mixture may be wet-granulated with a binder such as syrup, starch paste, acadia mucilage or solutions of cellulosic or polymeric materials, and forcing through a screen. As an alternative to granulating, the powder mixture may be run through the tablet machine and the result is imperfectly formed slugs broken into granules. The granules may be lubricated to prevent sticking to the tablet forming dies by means of the addition of stearic acid, a stearate salt, talc or mineral oil. The lubricated mixture is then compressed into tablets. The one or more compounds of the present disclosure may also be combined with a free-flowing inert carrier and compressed into tablets directly without going through the granulating or slugging steps. A clear or opaque protective coating consisting of a sealing coat of shellac, a coating of sugar or polymeric material, and a polish coating of wax may be provided. Dyestuffs may be added to these coatings to distinguish different unit dosages.

Oral fluids such as solutions, syrups, and elixirs may be prepared in dosage unit form so that a given quantity contains a predetermined amount of the compound. Syrups may be prepared, for example, by dissolving the compound in a suitably flavored aqueous solution, while elixirs may be prepared through the use of a non-toxic alcoholic vehicle. Suspensions may be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like may also be added.

Where appropriate, dosage unit formulations for oral administration may be microencapsulated. The formulation may also be prepared to prolong or sustain the release as for example by coating or embedding particulate material in polymers, wax or the like.

The one or more compounds of the present disclosure may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation or Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

In some embodiments, this disclosure provides pharmaceutical compositions suitable for administration by inhalation comprising the one or more compounds of the present disclosure (inhaled composition). In some embodiments, the inhaled compositions are formulated in the form of aerosol spray generated from a pressured container or dispenser that contains a suitable propellant, e.g., a gas such as carbon dioxide, C₃-C₅ alkane (propane, butane, or heptane), hydrogen-containing fluorocarbon propellant is selected from the group consisting of CHF₂CHF, CF₃CH₂F, CHF₂CH₃, CF₃CHFCF₃ and mixtures thereof, or a nebulizer.

In some embodiments, this disclosure provides pharmaceutical compositions suitable for topical, transmucosal or transdermal administration comprising the one or more compounds of the present disclosure and dermal penetration enhancing agent. Such dermal penetration enhancing agent are generally known in the art, for example, dimethyl sulfone (DMSO), emu oil, detergents, bile salts, and fusidic acid derivatives. Transmucosal administration can be accomplished using nasal sprays or suppositories. For transdermal administration, the compounds are formulated into ointments, salves, gels, hydrogels, or creams as generally known in the art. Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems. Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

In some embodiments, this disclosure provides pharmaceutically acceptable suppositories compositions (e.g., with conventional suppository bases such as cocoa butter and other glycerides), or retention enemas for rectal delivery comprising the one or more compounds of the present disclosure.

In some embodiments, the one or more compounds of the present disclosure may also be coupled with soluble polymers as targetable drug carriers. Such polymers may include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the one or more compounds of the present disclosure may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

In some embodiments, the pharmaceutical formulations are formulated for parenteral administration including aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents.

The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It is advantageous to formulate oral or parenteral compositions in unitary dosage form for ease of administration and uniformity of dosage. As used herein, the term “unitary dosage form” as used herein, refers to physically discrete units suited for the subject to be treated; each unit containing a predetermined quantity of active compound calculated to produce the desired therapeutic effect in association with the required pharmaceutical carrier.

A therapeutically effective amount of one or more compounds of the present disclosure will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. In some embodiments, the one or more compounds of the present disclosure may be effective over a wide range of doses.

In an embodiment, the present disclosure provides compounds of the present disclosure for use in medical therapy particularly for the treatment of viral infections such as coronavirus infection. The compounds of the present disclosure have been shown to be active against SARS-CoV-2 infections.

The compounds of the present disclosure are particularly suited to the treatment of SARS-CoV-2 infections and associated conditions. Reference herein to treatment extends to treatment of established infections, symptoms, and associated clinical conditions such as cytokine storm.

The compounds of the present disclosure may also be used in adjuvant therapy in the treatment of coronavirus infections or associated symptoms or effects, for example cytokine storm induced by the coronavirus [21].

The compounds of the present disclosure can be employed alone or in combination with other therapeutic agents. The compounds of the present disclosure and the other pharmaceutically active agent(s) may be administered together or separately and, when administered separately, administration may occur simultaneously or sequentially, in any order. The amounts of one or more compounds of the present disclosure and the other pharmaceutically active agent(s) and the relative timings of administration can be selected in order to achieve the desired combined therapeutic effect. The administration in combination of one or more compounds of the present disclosure with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The one or more compounds of the present disclosure may be used in combination with one or more agents useful in the prevention or treatment of coronavirus infections. Examples of such agents include, but are not limited to interferons, remdesivir, favipiravir, ribavirin or its analogs, protease inhibitors, 3CLpro inhibitor such as nirmatrelvir, alpha-glueosidase 1 inhibitors, hepatoprotectants, nucleoside or nucleotide inhibitors of SARS Coronavirus polymerase, non-nucleoside inhibitors of SARS coronavirus polymerase, SARS coronavirus helicase inhibitors, TLR-7 agonists, cyclophilin inhibitors, pharmacokinetic enhancers, and other drugs for treating SARS coronavirus, or mixtures thereof. Combinations of the one or more compounds of the present disclosure are typically selected based on the condition to be treated, cross-reactivities of ingredients and pharmaco-properties of the combination. For example, when treating an infection (e.g., SARS CoV-2), the one or more compounds of the present disclosure can be combined with other active therapeutic agents (such as those described herein).

Suitable active therapeutic agents or ingredients which can be combined with the one or more compounds of the present disclosure can include interferons, e.g., pegylated rIFN-alpha 2b, pegylated rIFN-alpha 2a, rIFN-alpha 2b, IFN alpha-2b XL, rIFN-alpha 2a, consensus IFN alpha, infer gen, rebif, locteron, AVI-005, PEG-infergen, pegylated IFN-beta, oral interferon alpha, feron, reaferon, intermax alpha, r-EFN-beta, infergen+actimmune, IFN-omega with DUROS, and albuferon; remdesivir, remdesivir analogs, favipiravir, favipiravir analogs, ribavirin, ribavirin analogs, e.g., rebetol, copegus, VX-497, and viramidine (taribavirin); hepatoprotectants, e.g., 1DN-6556, ME 3738, MitoQ, and LB-84451; non-nucleoside inhibitors of SARS Coronavirus.

In yet another embodiment, the present application provides pharmaceutical compositions comprising one or more compounds of the present disclosure, in combination with at least one additional therapeutic agent, and a pharmaceutically acceptable carrier or excipient.

In another embodiment, the present application provides pharmaceutical compositions comprising one or more compounds of the present disclosure, in combination with at least one additional therapeutic agent selected from the group consisting of biologics, interleukin-6 inhibitors, sarilumab, siltuximab, tocilizumab, TNF alpha inhibitors, adalimumab, Etanercept, Infliximab, Certolizumab, Interleukin-12/23 inhibitor, Ustekinumab, interleukin-17A inhibitor, secukinumab, brodalumab, Ixekizumab, interleukin-23 inhibitor, guselkumab, tildrakizumab, Risankizumab.

It is also possible to combine one or more compounds of the present disclosure with one or more other active therapeutic agents in a unitary dosage form for simultaneous or sequential administration to a subject. The combination therapy may be administered as a simultaneous or sequential regimen. When administered sequentially, the combination may be administered in two or more administrations.

Co-administration of one or more compounds of the present disclosure with one or more other active therapeutic agents generally refers to simultaneous or sequential administration of one or more compounds of the present disclosure and one or more other active therapeutic agents, such that therapeutically effective amounts of the compound of the disclosure and one or more other active therapeutic agents are both present in the body of the subject.

Co-administration includes administration of unit dosages of the one or more compounds of the present disclosure before or after administration of unit dosages of one or more other active therapeutic agents, for example, administration of the one or more compounds of the present disclosure within seconds, minutes, or hours of the administration of one or more other active therapeutic agents. For example, a unit dose of one or more compounds of the present disclosure can be administered first, followed within seconds or minutes by administration of a unit dose of one or more other active therapeutic agents. Alternatively, a unit dose of one or more other therapeutic agents can be administered first, followed by administration of a unit dose of one or more compounds of the present disclosure within seconds or minutes. In some cases, it may be desirable to administer a unit dose of one or more compounds of the present disclosure first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of one or more compounds of the present disclosure.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

In an embodiment, the present disclosure provides a process for the preparation of a pharmaceutical formulation including admixing one or more compounds of the present disclosure with one or more pharmaceutically acceptable carriers, diluents or excipients.

In an embodiment, the present disclosure provides a method of treating coronavirus infections and associated conditions in a subject in need thereof, for example, an infected animal or a mammal including a human (or a use of one or more compounds of the present disclosure in the manufacture of a medicament for use in the treatment of coronavirus infections and associated conditions) comprising: administering to the subject a therapeutically effective amount of the one or more compounds of the present disclosure. In some embodiment, the present application provides for methods of inhibiting SARS coronavirus proteases in a cell, comprising: contacting a cell infected with coronavirus with an effective amount of one or more compounds of the present disclosure, and at least one additional active therapeutic agent selected from the group consisting of interferons, remdesivir, flavipiravir, ribavirin or its analogs, protease inhibitors, alpha-glucosidase I inhibitors, hepatoprotectants, nucleoside or nucleotide inhibitors of coronavirus polymerase, non-nucleoside inhibitors of coronavirus polymerase, pharmacokinetic enhancers, and other drugs for treating SARS coronavirus, or mixtures thereof.

In some embodiments, the method comprising administering to the subject an effective amount of one or more compounds of the present disclosure, wherein the one or more compounds modulate the activity of the viral protein in the subject.

Without wishing to be bound by theories, it is believed that the one or more compounds may be used to modulate the activity of viral protein encoded by the coronavirus which is responsible for the infection, and when the one or more compounds modulate the activity of the viral protein in the subject, inflammatory and/or immunoregulatory activities induced by the coronavirus can be prevented and treated.

In some embodiments, the present disclosure provides a method of treating or preventing viral infection, the method comprising administering to a subject in need thereof an effective amount of a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) or a pharmaceutical composition comprising the compound of the present disclosure. In some embodiments, the method further comprises administering to the subject one or more other antiviral agents, such as those described herein. In some embodiments, the viral infection is caused by a virus selected from SARS-CoV, HCoV-229E, HCoV-OC43, HCoV-NL63, MERS-CoV, HCoV-HKU1, and SARS-CoV-2. In some preferred embodiments, the viral infection is SARS-CoV-2 infection.

In some embodiments, the present disclosure provides a method of inhibiting papain-like protease (PLpro), the method comprising administering to a subject in need thereof an effective amount of a compound of the present disclosure (e.g., a compound according to Formula X, Formula Y (e.g., Y-1, Y-1-A, Y-1-B, Y-1-C, Y-1-D, Y-2, Y-2-A, Y-2-B, Y-2-C, Y-2-D, Y-2-E, Y-2-F, Y-3, Y-3A, Y-3B, Y-3C, Y-4, Y-5, Y-6, or Y-7), Formula I, II, III, IV, or V, any of the compounds described in Table A herein, or any of Compound Nos. 1-177, or a pharmaceutically acceptable salt thereof) or a pharmaceutical composition comprising the compound of the present disclosure. In some embodiments, the subject suffers from viral infection caused by a virus selected from SARS-CoV, HCoV-229E, HCoV-OC43, HCoV-NL63, MERS-CoV, HCoV-HKU1, and SARS-CoV-2. In some preferred embodiments, the subject suffers from SARS-CoV-2 infection.

### Examples

The following definitions describe terms and abbreviations used herein:
A549:hACE2 A549 cells with over-expressed human ACE2 receptor
BSA bovine serum albumin
DAB 3,3′-Diaminobenzidine
DCM dichloromethane
DMEM Dulbecco's Modified Eagle Medium
DIPEA diisopropylethylamine
DMF N,N-dimethylformamide
DMSO dimethyl sulfoxide
EtOAc ethyl acetate
EtOH ethanol
H₂O Water
HATU (1-[Bis(dimethylamino)methylene]-1H-1,2,3-triazolo[4,5-b]pyridinium 3-oxide hexafluorophosphate)
Hex hexanes
HPLC high performance liquid chromatography
HRP horse radish peroxidase
IC50 half-maximal inhibitory concentration
K₂CO₃ potassium carbonate
LC-MS liquid chromatography-mass spectrometer
LiOH lithium hydroxide
MeOH methanol
MOI multiplicity of infection
MS mass
N₂ nitrogen
PBS phosphate buffered saline
PBST phosphate buffered saline/Tween
PLpro papain-like Protease
rt room temperature
rac racemate
RT retention time
THE tetrahydrofuran
TFA trifluoroacetic acid
FA formic acid
UV ultraviolet

### Chemistry Experimental

General. All reactions utilizing air- or moisture-sensitive reagents were performed in dried glassware under an atmosphere of dry N2. Solvents were purchased from Sigma-Aldrich or Oakwood chemicals and used without further purification. Reagents and other chemicals were purchased from commercial sources with purity >95% without further purification. Flash column chromatography was performed using a Combi-Flash NextGen 300+ and RediSep cartridges with EA/hexanes or MeOH/DCM as eluents. Purity was assessed by LC/MS/UV using a UHPLC-MS (Thermo Scientific Ultimate 3000 High Performance Liquid Chromatography (HPLC) system coupled to a Thermo Scientific mass spectrometer (LTQ XL MS) for qualitative analysis). Compounds were detected by fluorescence under 220 nm and 254 nm UV light. All compounds were confirmed to >95% purity prior to testing. The compounds with <95% purity are marked separately (*).

LC-MS conditions: Thermo Scientific Ultimate 3000 High Performance Liquid Chromatography (HPLC) system coupled to a Thermo Scientific mass spectrometer (LTQ XL MS); detection wavelength: 220 and 254 nM; Method 1: Column: Phenomenex Luna® (C18, 5 μm 50×2 mm); Mobile phase A: H₂O/0.11% FA; Mobile phase B: CH₃CN/0.1% FA; Flow rate: 0.400 ml/min; Gradient: 0 min 5% B, 6.0 min 95% B, 8.0 min 95% B, 8.1 min 5% B, Stop 10.0 min; Column temperature: 40° C. Method 2: Column: Waters ACQUITY UPLC® (C18, 1.7 pm 50×2.1 mm); Mobile phase A: H₂O/0.1% FA; Mobile phase B: CH₃CN/0.1% FA; Flow rate: 0.400 ml/min; Gradient: 0 min 5% B, 6.0 min 95% B, 8.0 min 95% B, 8.1 min 5% B, Stop 10.0 min; Column temperature: 40° C. Method 3: Column: Phenomenex Luna® (C18, 5 μm 50×2 mm); Mobile phase A: H₂O/0.1% FA; Mobile phase B: CH₃CN/0.1% FA; Flow rate: 1.000 ml/min; Gradient: 0 min 5% B, 4.0 min 95% B, 4.8 min 95% B, 4.9 min 5% B, Stop 6.0 min; Column temperature: 40° C.

Examples of compounds of the present disclosure include, but are not limited to, one or more of the compounds shown in TABLE 1 below.

TABLE 1

| | Inhibition of SARS-CoV-2 |
|---|---|
| Compound No. | PLpro IC₅₀ range^{a} |
| 1 | ++ |
| 2 | ++ |
| 3 | ++ |
| 4 | ++ |
| 5 | +++ |
| 6 | +++ |
| 7 | ++ |
| 8 | ++ |
| 9 | ++ |
| 10 | ++ |
| 11 | ++ |
| 12 | ++ |
| 13 | +++ |
| 14 | ++ |
| 15 | +++ |
| 16 | ++ |
| 17 | ++ |
| 18 | +++ |
| 19 | +++ |
| 20 | +++ |
| 21 | +++ |
| 22 | +++ |
| 23 | ++ |
| 24 | ++ |
| 25 | +++ |
| 26 | ++ |
| 27 | + |
| 28 | + |
| 29 | ++ |
| 57 | ++ |
| 59 | ++ |
| 60 | +++ |
| 63 | +++ |
| 64 | +++ |
| 65 | +++ |
| 67 | +++ |
| 79 | +++ |
| 80 | +++ |
| 81 | + |
| 82 | ++ |
| 83 | ++ |
| 84 | +++ |
| 85 | ++ |
| 89 | +++ |
| 90 | +++ |
| 91 | +++ |
| 92 | + |
| 93 | ++ |
| 94 | ++ |
| 96 | +++ |
| 97 | +++ |
| 98 | +++ |
| 99 | +++ |
| 100 | ++ |
| 101 | +++ |
| 102 | ++ |
| 103 | ++ |
| 105 | + |
| 107 | +++ |
| 108 | ++ |
| 110 | +++ |
| 111 | ++ |
| 112 | ++ |
| 113 | ++ |
| 114 | + |
| 115 | +++ |
| 117 | + |
| 118 | + |
| 119 | ++ |
| 120 | ++ |
| 121 | +++ |
| 123 | ++ |
| 125 | ++ |
| 126 | + |
| 127 | ++ |
| 128 | + |
| 129 | ++ |
| 130 | +++ |
| 131 | +++ |
| 132 | +++ |
| 133 | +++ |
| 134 | +++ |
| 135 | +++ |
| 136 | ++ |
| 137 | +++ |
| 139 | ++ |
| 140 | ++ |
| 141 | ++ |
| 142 | ++ |
| 143 | + |
| 144 | + |
| 145 | +++ |
| 147 | +++ |
| 148 | + |
| 151 | +++ |
| 152 | +++ |
| 153 | +++ |
| 154 | + |
| 156 | + |
| 157 | ++ |
| 159 | ++ |
| 162 | ++ |
| 164 | +++ |
| 168 | + |
| 170 | + |

^{a}PLpro enzymatic inhibition level is expressed by the IC₅₀ values. It is indicated as +++ (IC₅₀: less than 100 nM), ++ (IC₅₀: 0.1 μM to less than 1 μM), or + (IC₅₀: 1 μM to 10 μM).

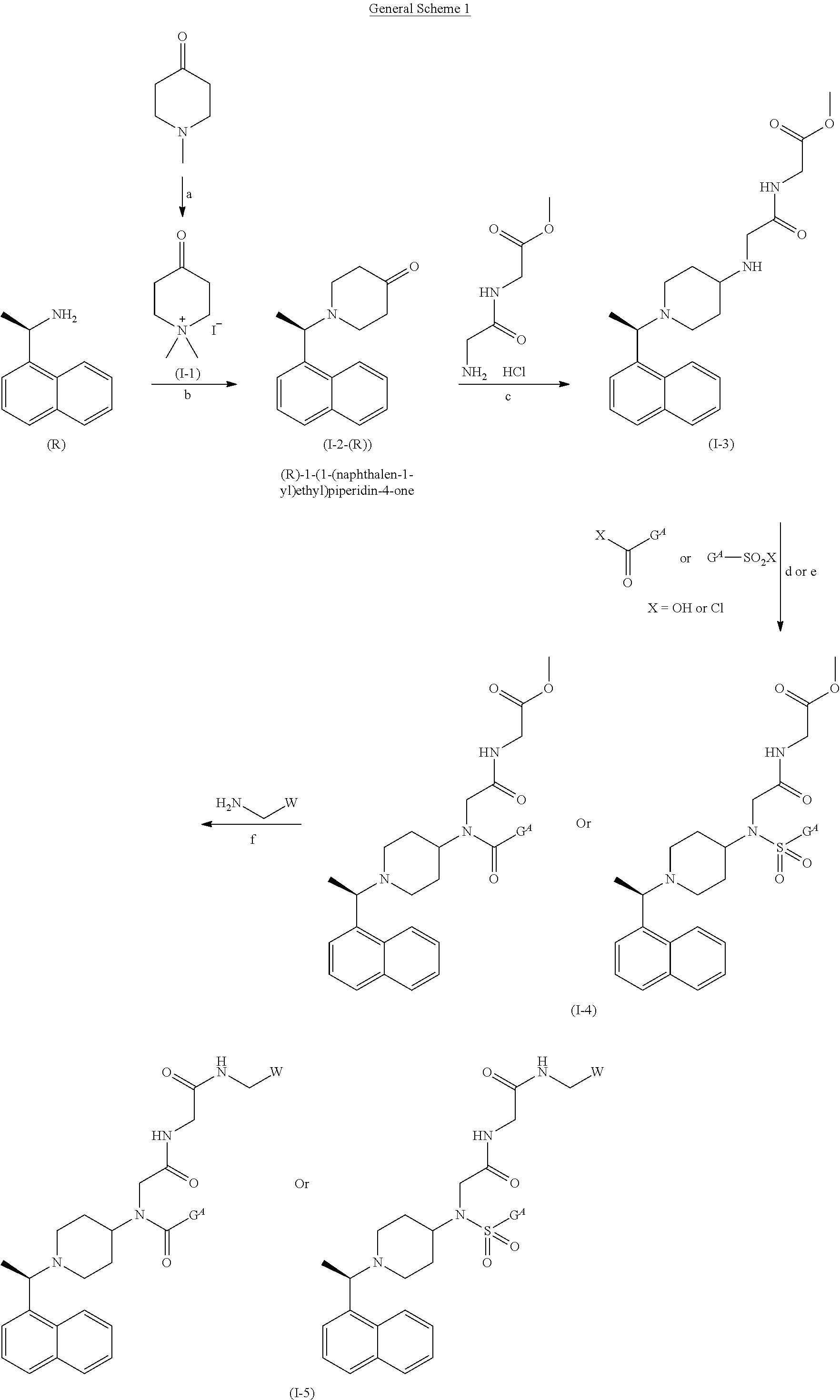

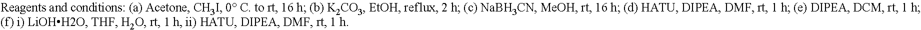

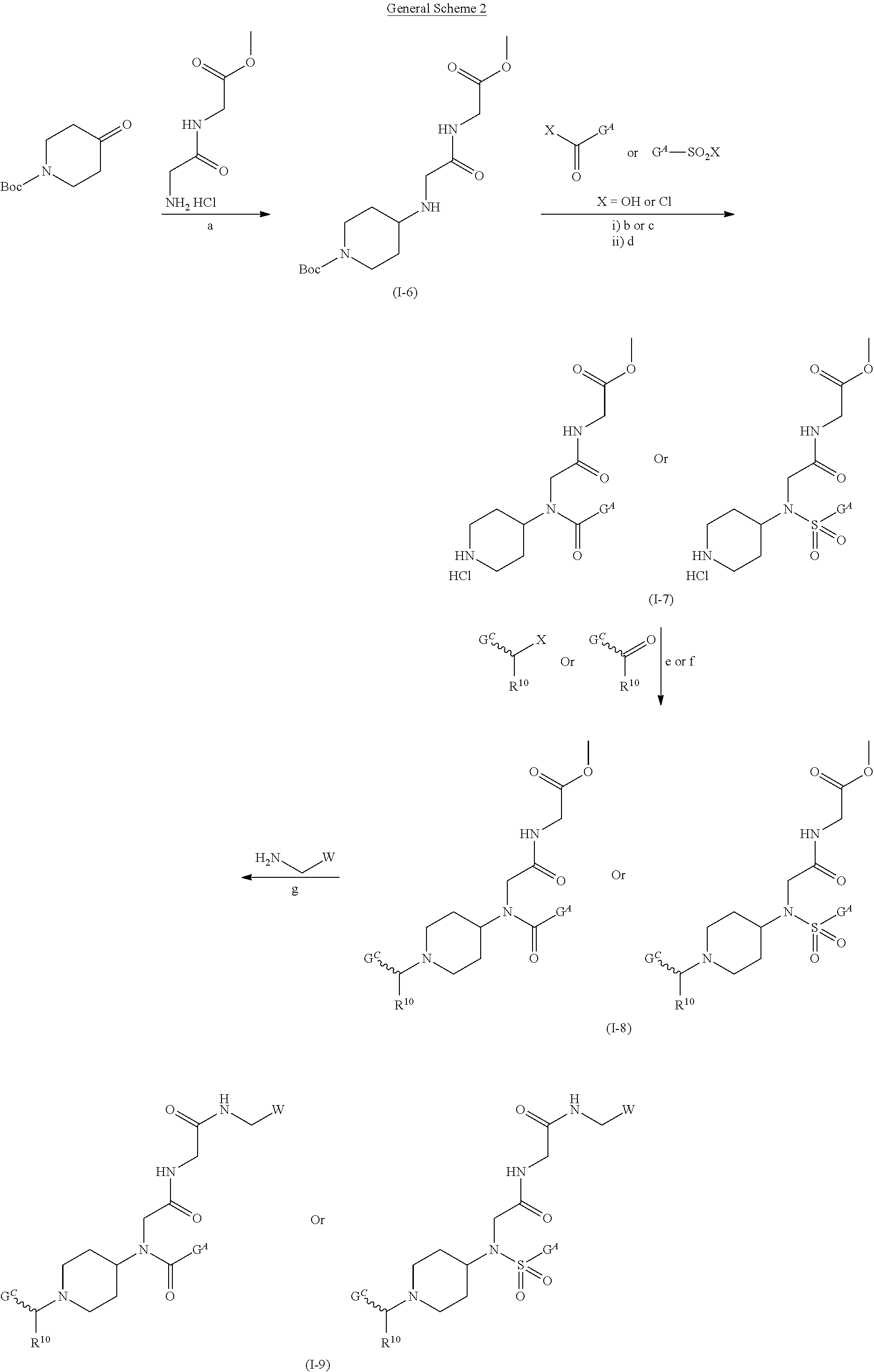

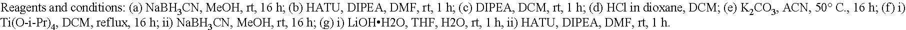

The following examples describe in detail the specific synthetic methods shown in the Scheme, which were used for the synthesis of some preferable compounds of the present disclosure. However, it should be understood by those skilled in the art that the chemical reactions can be modified slightly for the synthesis of other compounds of the present disclosure. The variables G^{A}, R¹⁰ and W in the scheme include any of those respective definitions described herein. G^{C} can have any of the definition described herein for R¹⁴ or R¹⁵ as applicable, for example, G^{C} can be an optionally substituted C₁₋₆ alkyl or optionally substituted C₃₋₆ cycloalkyl. In some examples, R¹⁰ is an optionally substituted phenyl, optionally substituted naphthyl, an optionally substituted ring structure having 4-10 ring atoms with 1-3 ring heteroatoms.

These examples are only used for illustration but are not to limit the scope of the present disclosure in any way.

### General Procedure

### A. Alkylation to synthesize 1,1-dimethyl-4-oxopiperidin-1-ium iodide (I-1)

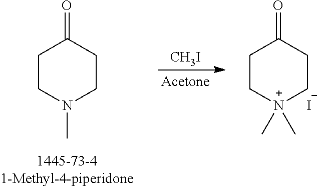

To a solution of 1-Methyl-4-piperidone (30 mmol, 1.0 eq) in acetone (15.0 mL), Iodomethane (33 mmol, 1.1 eq) was added at 0° C. and the mixture was stirred at room temperature overnight. The reaction mixture was filtered and the dried under the reduced pressure. The compound was used without any further purification.

### B. Substitution to synthesize (R)-1-(1-(naphthalen-1-yl)ethyl)piperidin-4-one (I-2)

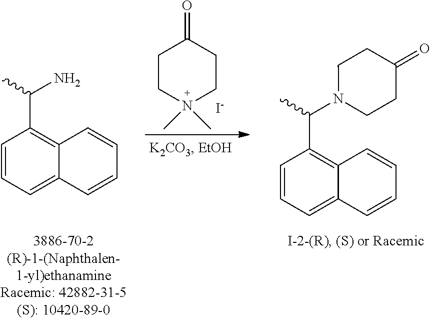

To a solution of (R)-1-(Naphthalen-1-yl)ethanamine (20 mmol, 1.0 eq) (or (S) or racemic-1-(Naphthalen-1-yl)ethanamine) in EtOH/Water (2:1) (100.0 mL), K₂CO₃ (50 mmol, 2.5 eq) and the compound I-1 (21 mmol, 1.05 eq) were added and the mixture was added and the mixture was stirred at 110° C. for 2 h. Then EtOH was removed by reduced pressure and the aqueous layer was extracted with EtOAc. The organic layer was dried over Na₂SO₄, filtered and collected. Then the crude product was purified by flash column chromatography.

HPLC (Method 1) RT=3.52 min, Purity >99.9%, MS (ESI+) m/z calcd for C₁₇H₂₀NO⁺[M+H]⁺ 254.4 found 254.2.

C. Reductive amination to synthesize methyl (R)-(R)-(1-(1-(naphthalen-1-yl)ethyl)piperidin-4-yl)glycylglycinate (I-3) or tert-butyl 4-((2-((2-methoxy-2-oxoethyl)amino)-2-oxoethyl)amino)piperidine-1-carboxylate (I-6) methyl ((aminoacetyl)amino)acetate hydrochloride

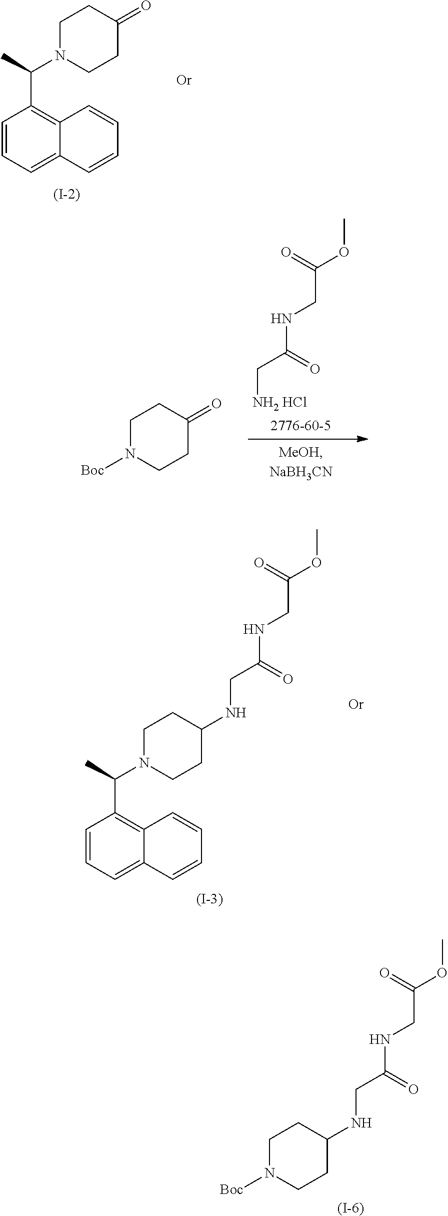

To a suspension of the compound I-2, 1-Boc-4-Piperidone or the corresponding ketone (2 mmol, 1.0 eq) in MeOH (4.0 mL), methyl ((aminoacetyl)amino)acetate hydrochloride or the corresponding amine (2 mmol, 1.0 eq) and Sodium cyanoborohydride (4 mmol, 2.0 eq) were added and the mixture was stirred at room temperature for 1 h. Then MeOH was removed by reduced pressure and the reaction mixture was quenched by sat. NaHCO₃ soln., extracted with EtOAc. The organic layer was dried over Na₂SO₄, filtered and concentrated. Then the crude material was purified by flash column chromatography.

HPLC (Method 1) RT=2.44 min, Purity >99.9%, MS (ESI+) m/z calcd for C₂₂H₃₀N₃O₃⁺ [M+H]⁺ 384.5, found 384.3.—Compound I-3

HPLC (Method 1) RT=3.51 min, Purity >99.9%, MS (ESI+) m/z calcd for C₁₅H₂₈N₃O₅⁺ [M+H]⁺ 330.2, found 330.2.—Compound I-6

### D. Amide or sulfonamide synthesis (I-4, I-7)

To a solution of the compound I-3, I-6 or the corresponding intermediate (0.1 mmol, 1.0 eq) in DMF (0.3 mL), the corresponding carboxylic acid or sulfonic acid (0.1 mmol, 1.0 eq) and DIPEA (0.2 mmol, 2.0 eq) were added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

When the corresponding reagent was a carbonyl chloride or sulfonyl chloride, the substitution procedure as described below was used. To a solution of the compound (I-3) (0.1 mmol, 1.0 eq) in DCM (0.3 mL), DIPEA (0.2 mmol, 2.0 eq) was added. The corresponding carbonyl chloride or sulfonyl chloride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

### E. Hydrolysis and amide coupling (1-5, 1-9)

To a solution of the compound I-4, I-8 or the corresponding ester (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and the corresponding amine or its salt form (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### F. Synthesis of I-8 through reductive amination or substitution

To a solution of the compound (I-7) (0.1 mmol, 1.0 eq) in ACN (0.3 mL), K₂CO₃ (0.2 mmol, 2.0 eq) and the corresponding halides, mesylates or tosylates (0.1 mmol, 1.0 eq) were added. The reaction mixture was stirred at room temperature for 1 h. Then the reaction mixture was heated up to 50° C. overnight. The reaction mixture was quenched by water, extracted with DCM. The organic layer was dried over Na₂SO₄, filtered and concentrated. Then the crude material was purified by flash column chromatography.

When the corresponding reagent was a ketone, the reductive amination procedure as described below was used. To a solution of the compound (I-7) (0.1 mmol, 1.0 eq) and the corresponding ketone (0.1 mmol, 1.0 eq) in DCM (0.3 mL), Ti(O-i-Pr)₄ (0.1 mmol, 1.0 eq) was added. The reaction mixture was refluxed overnight. Then the reaction mixture was concentrated under the reduced pressure. The mixture was diluted in MeOH (0.3 mL). To the mixture, NaBH3CN (0.3 mmol, 3.0 eq) was added and the mixture was refluxed for 4 h. The solvent was removed under the reduced pressure and the reaction mixture was quenched by water, extracted with DCM. The organic layer was dried over Na₂SO₄, filtered and concentrated. Then the crude material was purified by flash column chromatography.

### Synthesis of Compound I-3-Racemic

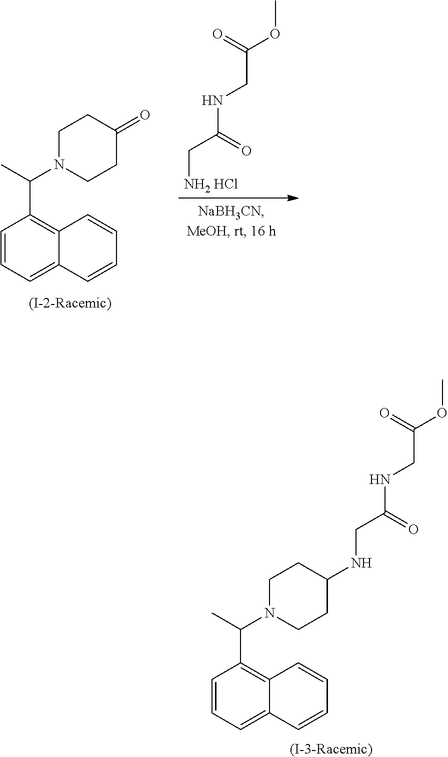

To a suspension of the compound I-2-Racemic (2 mmol, 1.0 eq) in MeOH (4.0 mL), methyl ((aminoacetyl)amino)acetate hydrochloride (2 mmol, 1.0 eq) and sodium cyanoborohydride (4 mmol, 2.0 eq) were added and the mixture was stirred at room temperature for 1 h. Then MeOH was removed under the reduced pressure and the reaction mixture was quenched by sat. NaHCO₃ soln., and extracted with EtOAc. The organic layer was dried over Na₂SO₄, filtered and concentrated. Then the crude material was purified by flash column chromatography.

HPLC (Method 1) RT=2.44 min, Purity >99.9%, MS (ESI+) m/z calcd for C₂₂H₃₀N₃O₃⁺ [M+H]⁺ 384.5, found 384.3.

### Synthesis of Compound I-10

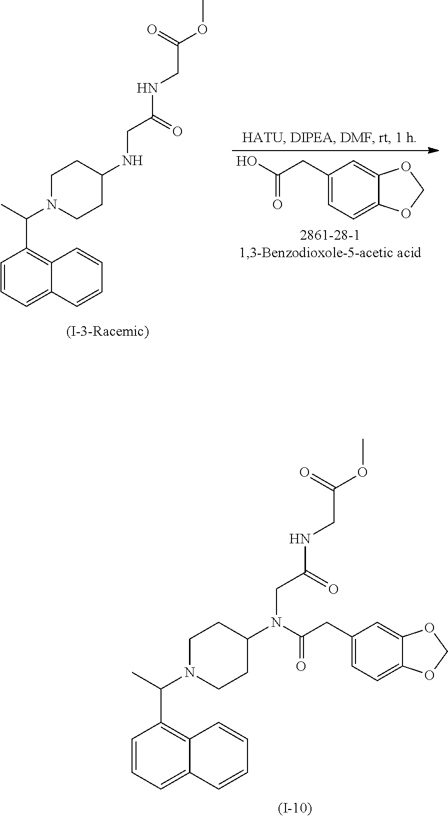

To a solution of the compound I-3-Racemic (0.1 mmol, 1.0 eq) in DMF (0.3 mL), 1,3-Benzodioxole-5-acetic acid (0.1 mmol, 1.0 eq) and DIPEA (0.2 mmol, 2.0 eq) were added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

HPLC (Method 1) RT=4.30 min, Purity >99.9%, MS (ESI+) m/z calcd for C₃₁H₃₆N₃O₆⁺ [M+H]⁺ 546.3, found 546.4.

Synthesis of Compound 1

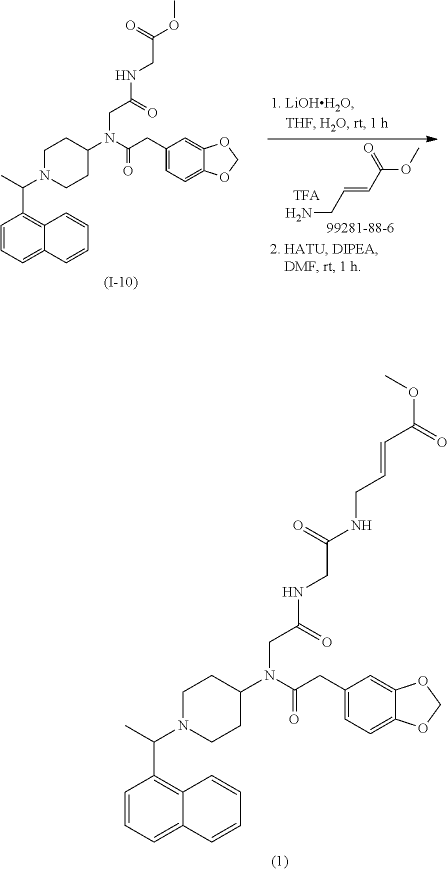

To a solution of the compound I-10 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and 2-Butenoic acid, 4-amino-, methyl ester, (E)-, trifluoroacetate (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 12

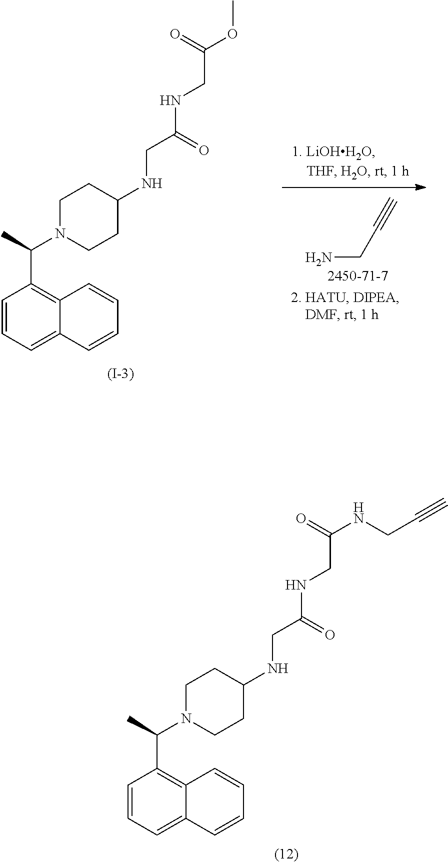

To a solution of the compound I-3 (0.1 mmol, 1.0 eq) in DMF (0.3 mL), propargylamine (0.1 mmol, 1.0 eq) and DIPEA (0.2 mmol, 2.0 eq) were added. Then HATU (0.1 mmol, 1.0 eq) was slowly added to the mixture at room temperature. The mixture was stirred for 1h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 63

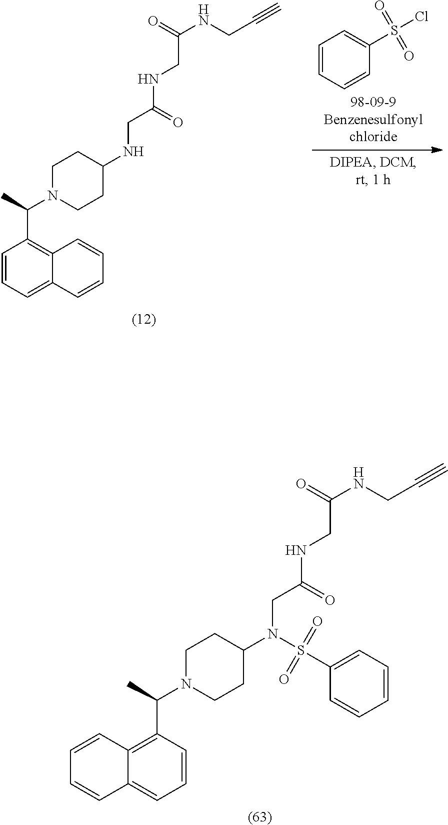 [SMILES: C#CCNC(=O)CNC(=O)CNC1CCN([C@H](C)c2cccc3ccccc23)CC1 ; O=S(=O)(Cl)c1ccccc1 ; "(12)" ; C#CCNC(=O)CNC(=O)CN(C1CCN([C@H](C)c2cccc3ccccc23)CC1)S(=O)(=O)c1ccccc1 ; "(63)"]

To a solution of the compound (12) (0.1 mmol, 1.0 eq) in DCM (0.3 mL), DIPEA (0.2 mmol, 2.0 eq) was added. Benzenesulfonyl chloride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

### Synthesis of Compound 60

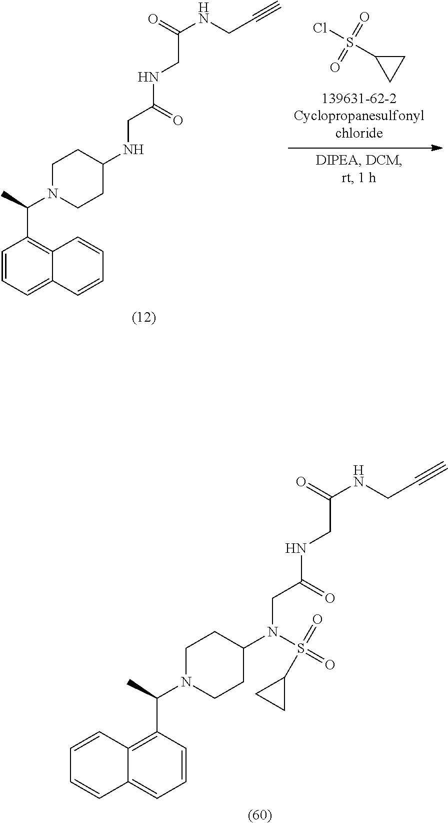 [SMILES: C#CCNC(=O)CNC(=O)CNC1CCN([C@H](C)c2cccc3ccccc23)CC1 ; O=S(=O)(Cl)C1CC1 ; "(12)" ; C#CCNC(=O)CNC(=O)CN(C1CCN([C@H](C)c2cccc3ccccc23)CC1)S(=O)(=O)C1CC1 ; "(60)"]

To a solution of the compound (12) (0.1 mmol, 1.0 eq) in DCM (0.3 mL), DIPEA (0.2 mmol, 2.0 eq) was added. Cyclopropanesulfonylchloride (0.1 mmol, 1.0 eq) was slowly added to the mixture at 0° C. The reaction mixture was warmed up to room temperature and stirred for 1 h. The reaction mixture was quenched by water, extracted with DCM. The organic layer was dried over Na₂SO₄, filtered and concentrated. Then the crude material was purified by flash column chromatography.

### Synthesis of Compound I-11

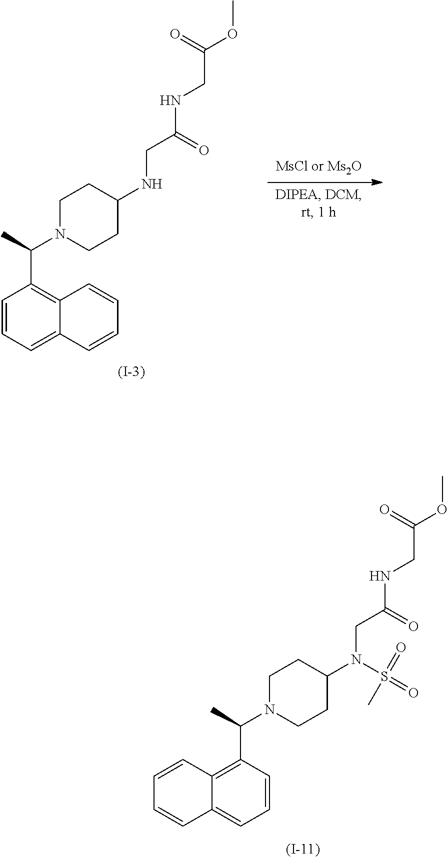

To a solution of the compound (I-3) (0.1 mmol, 1.0 eq) in DCM (0.3 mL), DIPEA (0.2 mmol, 2.0 eq) was added. Methanesulfonyl chloride or methanesulfonic anhydride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

HPLC (Method 1) RT=3.98 min, Purity >99.9%, MS (ESI+) m/z calcd for C₂₃H₃₂N₃O₅S⁺ [M+H]⁺ 462.2, found 462.3.

### Synthesis of Compound I-11-Racemic

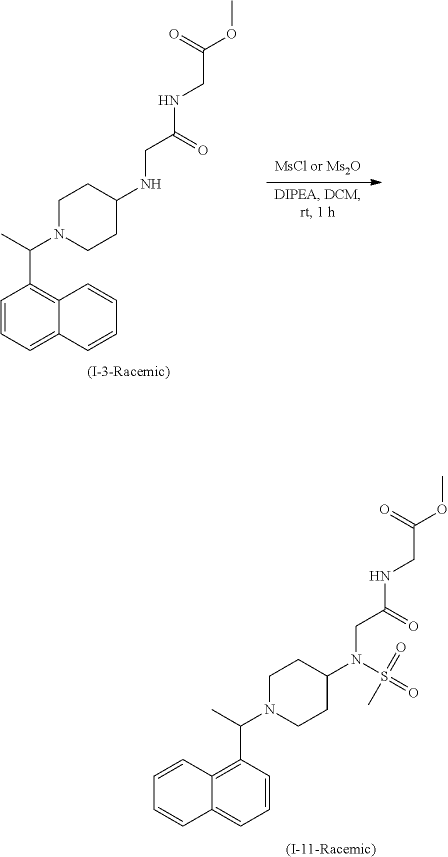

To a solution of the compound (I-3-Racemic) (0.1 mmol, 1.0 eq) in DCM (0.3 mL), DIPEA (0.2 mmol, 2.0 eq) was added. Methanesulfonyl chloride or methanesulfonic anhydride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

HPLC (Method 1) RT=3.98 min, Purity >99.9%, MS (ESI+) m/z calcd for C₂₃H₃₂N₃O₅S⁺ [M+H]⁺ 462.2, found 462.3.

### Synthesis of Compound 20

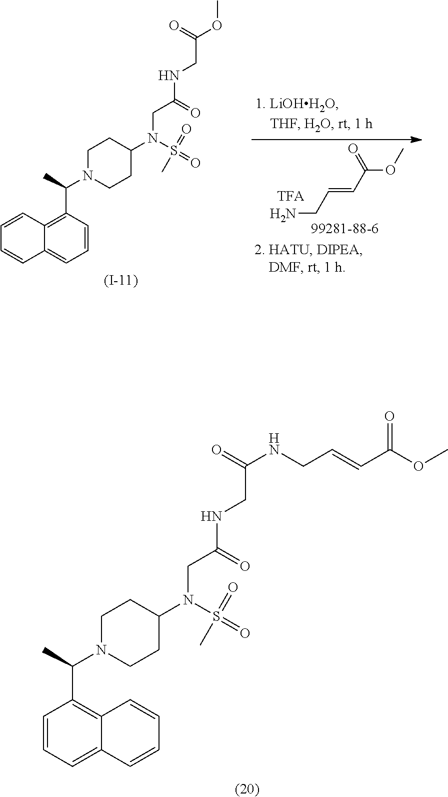

To a solution of the compound I-11 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and 2-Butenoic acid, 4-amino-, methyl ester, (E)-, trifluoroacetate (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 21

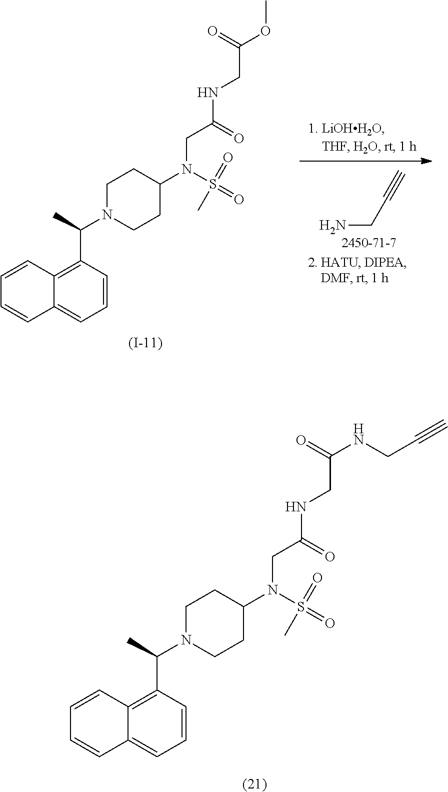

To a solution of the compound I-11 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and propargylamine (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 127

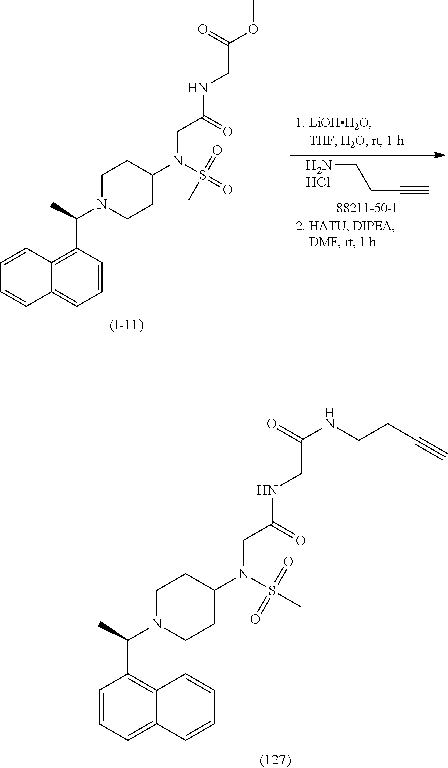

To a solution of the compound I-11 (0.1 mmol, 1.0 eq) in DMF (0.3 mL), But-3-yn-1-amine hydrochloride (0.1 mmol, 1.0 eq) and DIPEA (0.2 mmol, 2.0 eq) were added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-12

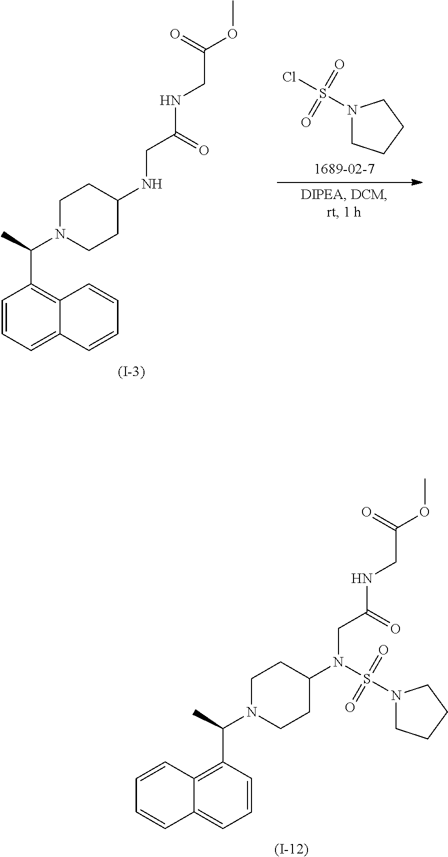

To a solution of the compound (I-3) (0.1 mmol, 1.0 eq) in DCM (0.3 mL), DIPEA (0.2 mmol, 2.0 eq) was added. 1-Pyrrolidinesulfonyl chloride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

HPLC (Method 1) RT=4.33 min, Purity >99.9%, MS (ESI+) m/z calcd for C₂₆H₃₇N₄O₅S⁺ [M+H]⁺ 517.2, found 517.4.

### Synthesis of Compound 65

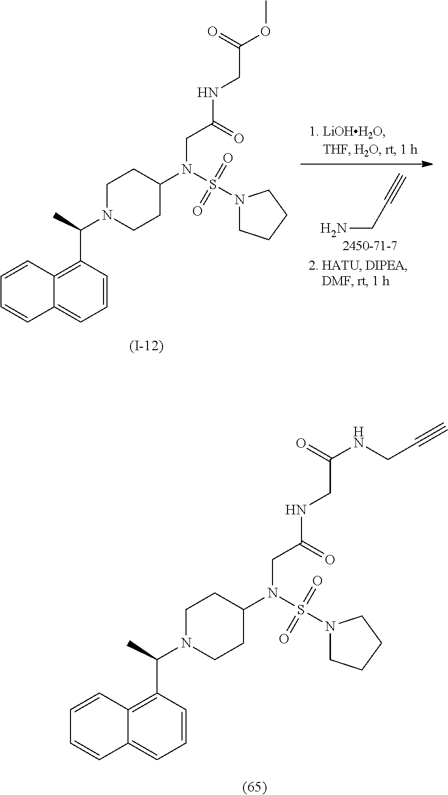

To a solution of the compound I-12 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and propargylamine (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-13

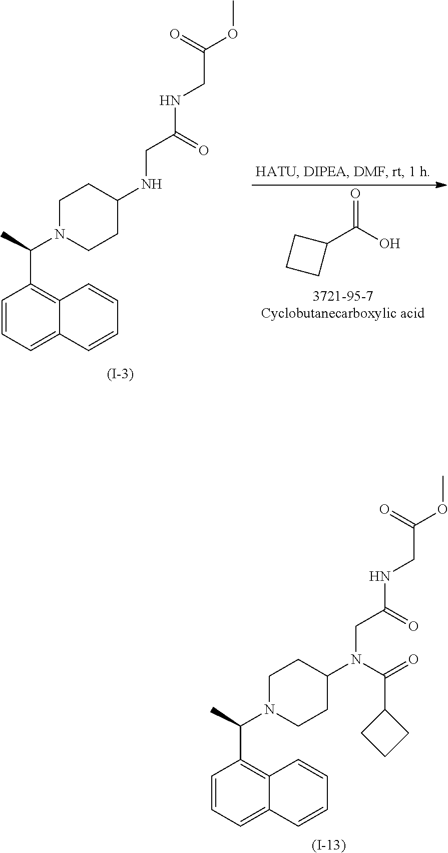

To a solution of the compound I-3 (0.1 mmol, 1.0 eq) in DMF (0.3 mL), cyclobutanecarboxylic acid (0.1 mmol, 1.0 eq) and DIPEA (0.2 mmol, 2.0 eq) were added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

HPLC (Method 1) RT=4.13 min, Purity >99.9%, MS (ESI+) m/z calcd for C₂₇H₃₆N₃O₄⁺ [M+H]⁺ 466.3, found 466.5.

### Synthesis of Compound 7

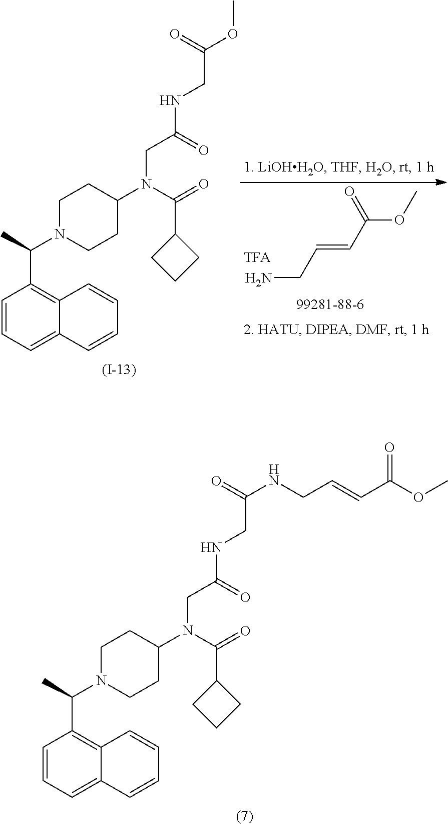

To a solution of the compound I-13 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and 2-Butenoic acid, 4-amino-, methyl ester, (E)-, trifluoroacetate (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-14

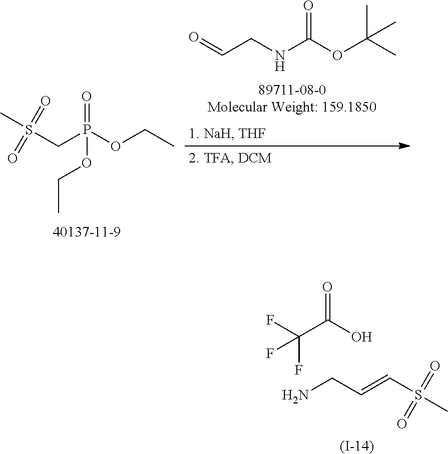

To a solution of diethyl(methylsulfonylmethyl)phosphonate (1 mmol, 1.0 eq) in THF (10 mL), 60% NaH (1.3 mmol, 1.3 eq) was slowly added at 0° C. The reaction mixture was stirred at 0° C. for 10 min. Then N-Boc-2-aminoacetaldehyde (190 mg, 1.2 mmol, 1.2 eq) in THE (1 mL) was slowly added to the mixture. The reaction was allowed to warm to room temperature and stirred for 1 h. Then the excessive solvents were dried off under the reduced pressure. The mixture was extracted with EtOAc, dried over MgSO₄, filtered and concentrated. The crude material was purified by flash column chromatography. To a solution of purified boc-protected amine (0.2 mmol) in DCM (0.3 mL) at 0° C., TFA (0.1 mL) was slowly added. The reaction mixture was stirred for 30 min at room temperature. Then the excessive solvent was dried off under the reduced pressure. The compound was used without further purification.

### Synthesis of Compound I-13-Racemic

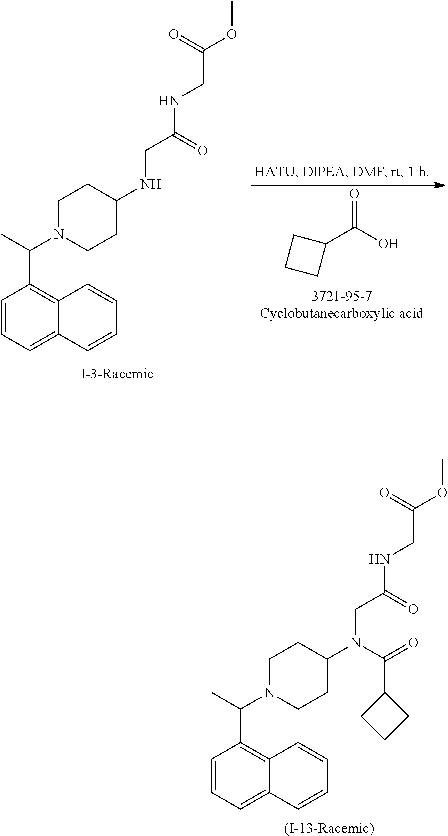

To a solution of the compound I-3-Racemic (0.1 mmol, 1.0 eq) in DMF (0.3 mL), cyclobutanecarboxylic acid (0.1 mmol, 1.0 eq) and DIPEA (0.2 mmol, 2.0 eq) were added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

HPLC (Method 1) RT=4.17 min, Purity=88.4%, MS (ESI+) m/z calcd for C₂₇H₃₆N₃O₄⁺ [M+H]⁺ 466.3, found 466.4.

### Synthesis of Compound 9

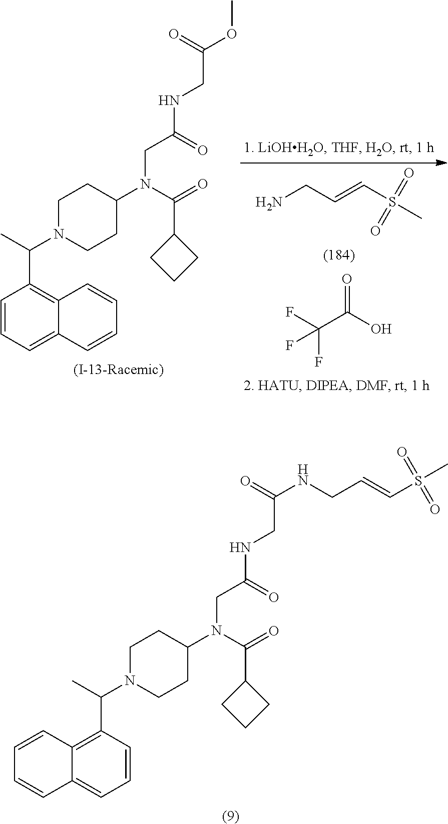

To a solution of the compound I-13-Racemic (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and the compound I-14 (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 59

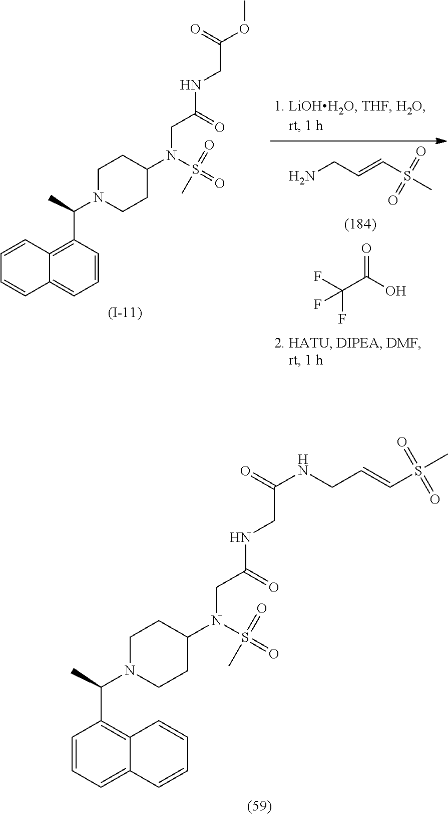

To a solution of the compound I-11 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and the compound I-14 (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-15

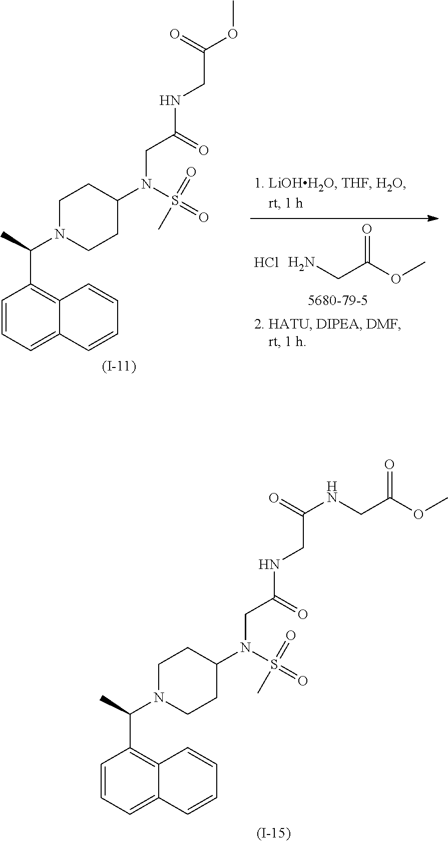

To a solution of the compound I-11 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and H-Gly-OMe HCl (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

HPLC (Method 1) RT=4.17 min, Purity=87.5%, MS (ESI+) m/z calcd for C₂₅H₃₅N₄O₆S⁺ [M+H]⁺ 519.2, found 519.4.

### Synthesis of Compound I-16

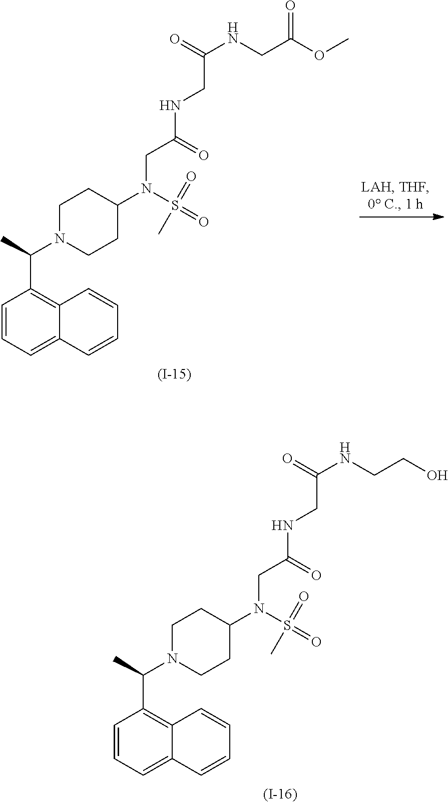 [SMILES: COC(=O)CNC(=O)CNC(=O)CN(C1CCN([C@H](C)c2cccc3ccccc23)CC1)S(C)(=O)=O ; "(I-15)" ; C[C@H](c1cccc2ccccc12)N1CCC(N(CC(=O)NCC(=O)NCCO)S(C)(=O)=O)CC1 ; "(I-16)"]

To a solution of the compound I-15 (0.1 mmol, 1.0 eq) in THF (0.1 mL), lithium aluminum hydride (0.3 mmol, 3.0 eq) was added at 0° C. and the reaction mixture was stirred for 1 h. The reaction mixture was quenched with 1N NaOH soln. (0.1 mL), filtered, extracted with EtOAc, washed with brine, dried over Na₂SO₄. Then the crude material was purified by flash column chromatography.

HPLC (Method 1) RT=3.73 min, Purity >99.9%, MS (ESI+) m/z calcd for C₂₄H₃₅N₄O₅S⁺ [M+H]⁺ 491.2, found 491.4.

### Synthesis of Compound 64

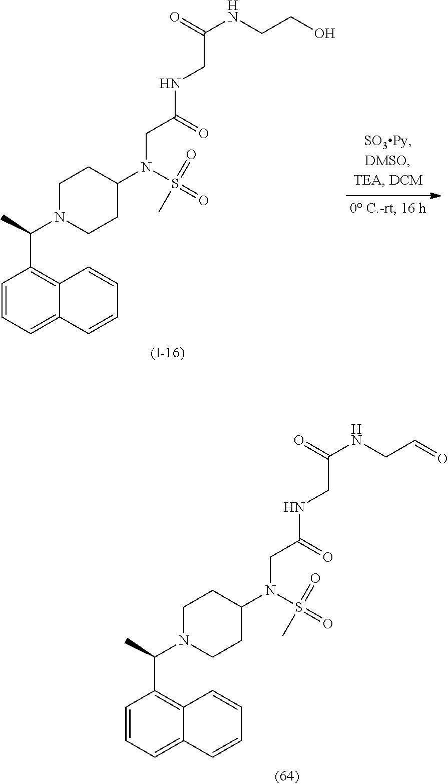 [SMILES: C[C@H](c1cccc2ccccc12)N1CCC(N(CC(=O)NCC(=O)NCCO)S(C)(=O)=O)CC1 ; "(I-16)" ; C[C@H](c1cccc2ccccc12)N1CCC(N(CC(=O)NCC(=O)NCC=O)S(C)(=O)=O)CC1 ; "(64)"]

To a solution of the compound I-16 (0.1 mmol, 1.0 eq) in DCM (0.4 mL) and DMSO (0.1 mL), SO3 Py complex (0.3 mmol, 3.0 eq) and TEA (0.6 mmol, 6.0 eq) were added at 0° C. and the reaction mixture was stirred for 16 h at room temperature. The reaction mixture was extracted with EtOAc, washed with brine, dried over Na₂SO₄. Then the crude material was purified by flash column chromatography. MS showed its hydroxy acetal from (MS=507) along with the protonated form (MS=489)

### Synthesis of Compound I-17

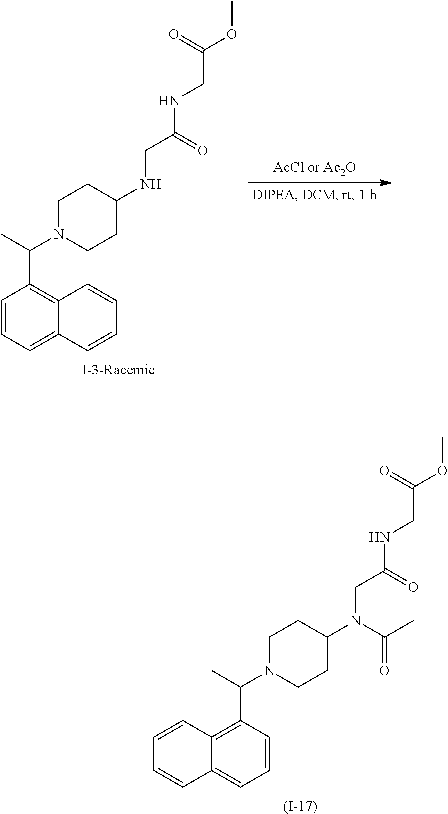

To a solution of the compound (I-3-Racemic) (0.1 mmol, 1.0 eq) in DCM (0.3 mL), DIPEA (0.2 mmol, 2.0 eq) was added. Acetyl chloride or acetic anhydride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

HPLC (Method 1) RT=3.75 min, Purity=88.4%, MS (ESI+) m/z calcd for C₂₄H₃₂N₃O₄⁺ [M+H]⁺ 426.2, found 426.3.

### Synthesis of Compound 13

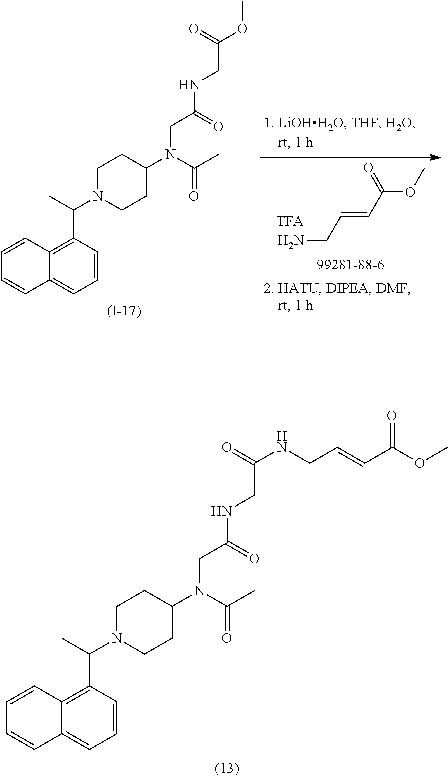

To a solution of the compound I-17 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and 2-Butenoic acid, 4-amino-, methyl ester, (E)-, trifluoroacetate (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-18

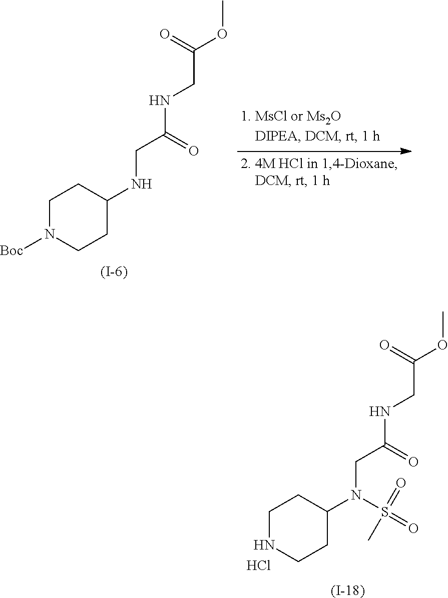

To a solution of the compound (I-6) (0.1 mmol, 1.0 eq) in DCM (0.3 mL), DIPEA (0.2 mmol, 2.0 eq) was added. Methanesulfonyl chloride or methanesulfonic anhydride (0.1 mmol, 1.0 eq) was added slowly to the mixture at 0° C. The reaction mixture was slowly warmed up to room temperature and stirred for 1 h. The reaction mixture was quenched by water, extracted with DCM. The organic layer was dried over Na₂SO₄, filtered and concentrated. Then the crude material was purified by flash column chromatography.

To a solution of methanesulfonylated intermediate (1 mmol, 1.0 eq) in DCM (3 mL), 4M HCl in 1,4-Dioxane (3 mmol, 3.0 eq) was added at 0° C. The reaction mixture was slowly warmed up to room temperature and stirred for 1 h. The mixture was diluted with DCM and the precipitated was filtered and collected. The crude product was used without further purification.

HPLC (Method 1) MS (ESI+) m/z calcd for C₁₁H₂₂N₃O₅S⁺ [M+H]⁺ 308.1, found 308.2.

### Synthesis of Compound I-19

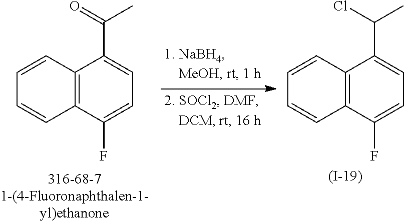

To a solution of 1-(4-Fluoronaphthalen-1-yl)ethanone (1 mmol, 1.0 eq) in MeOH (3 mL), NaBH4 (2 mmol, 2.0 eq) was added. The reaction mixture was stirred at room temperature for 1 h. The mixture was quenched by 1N HCl, concentrated and diluted and extracted with DCM. The organic layer was dried over Na₂SO₄, filtered and concentrated. Then the crude material was purified by flash column chromatography.

To a solution of hydroxyl intermediate (0.1 mmol) in DCM (0.3 mL), SOCl₂ (0.6 mmol, 6.0 eq) and DMF (0.01 mmol, 0.1 eq) were added. The reaction mixture was stirred at room temperature for 16 h. Then the mixture was concentrated and dried under the reduced pressure. The crude mixture was used without further purification.

### Synthesis of Compound 1-20

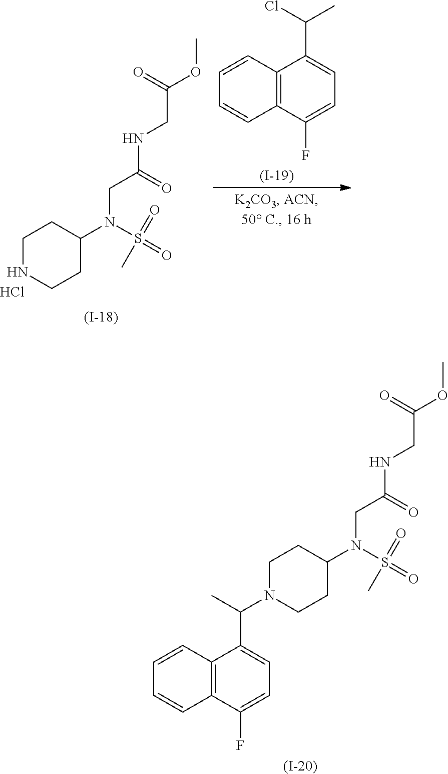

To a solution of the compound I-18 (0.1 mmol, 1.0 eq) in ACN (0.3 mL), K₂CO₃ (0.2 mmol, 2.0 eq) and the compound I-19 (0.1 mmol, 1.0 eq) were added. The reaction mixture was stirred at room temperature for 1 h. Then the reaction mixture was heated up to 50° C. and stirred overnight. The reaction mixture was quenched by water, extracted with DCM. The organic layer was dried over Na₂SO₄, filtered and concentrated. Then the crude material was purified by flash column chromatography.

HPLC (Method 1) RT=4.11 min, MS (ESI+) m/z calcd for C₂₃H₃₁FN₃O₅S⁺ [M+H]⁺ 480.2, found 480.2.

### Synthesis of Compound 107

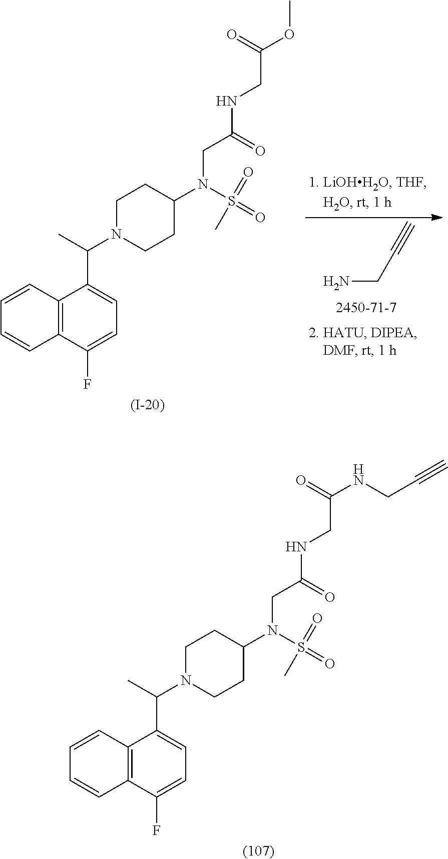

To a solution of the compound I-20 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and propargylamine (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-21

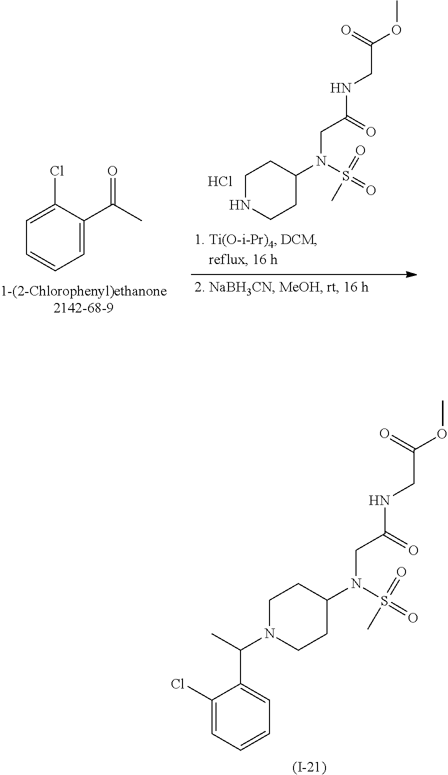

To a solution of 1-(2-chlorophenyl)ethanone (0.1 mmol, 1.0 eq) and the compound I-18 in DCM (0.3 mL), Ti(O-i-Pr)₄ (0.1 mmol, 1.0 eq) was added. The reaction mixture was refluxed overnight. Then the reaction mixture was concentrated under the reduced pressure. The mixture was diluted in MeOH (0.3 mL). To the mixture, NaBH3CN (0.3 mmol, 3.0 eq) was added and the mixture was stirred for 16 h. The solvent was removed under the reduced pressure and the reaction mixture was quenched by water, extracted with DCM. The organic layer was dried over Na₂SO₄, filtered and concentrated. Then the crude material was purified by flash column chromatography.

HPLC (Method 1) RT=3.59 min, Purity >99.9%, MS (ESI+) m/z calcd for C₁₉H₂₉ClN₃O₅S⁺ [M+H]⁺ 446.2, found 446.1.

### Synthesis of Compound 101

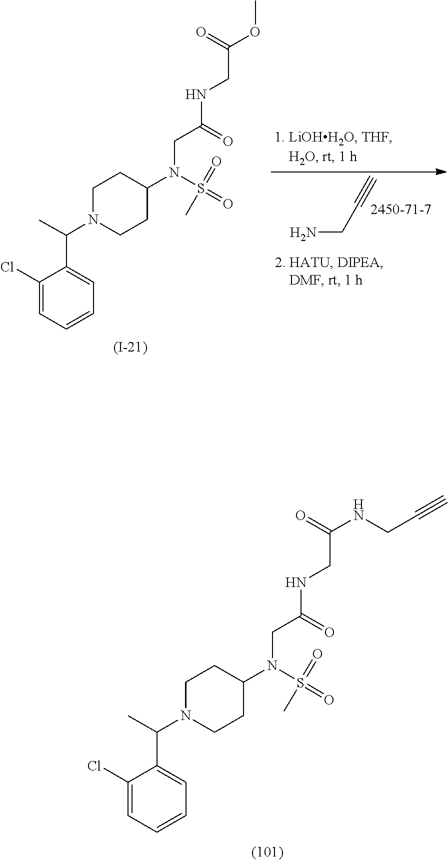

To a solution of the compound I-21 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and propargylamine (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 1-22

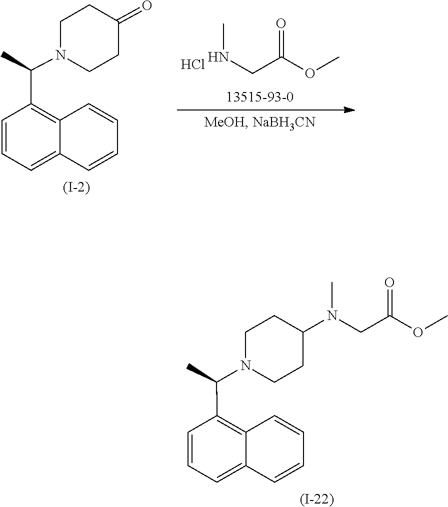

To a suspension of the compound I-2 (2 mmol, 1.0 eq) in MeOH (4.0 mL), methyl N-methylglycinate hydrochloride (2 mmol, 1.0 eq) and sodium cyanoborohydride (4 mmol, 2.0 eq) were added and the mixture was stirred at room temperature for 1 h. Then MeOH was removed by reduced pressure and the reaction mixture was quenched by sat. NaHCO₃ soln., extracted with EtOAc. The organic layer was dried over Na₂SO₄, filtered and concentrated. Then the crude material was purified by flash column chromatography.

HPLC (Method 1) RT=3.02 min, Purity=92.6%, MS (ESI+) m/z calcd for C₂₁H₂₉N₂O₂⁺ [M+H]⁺ 341.2, found 341.2.

### Synthesis of Compound I-23

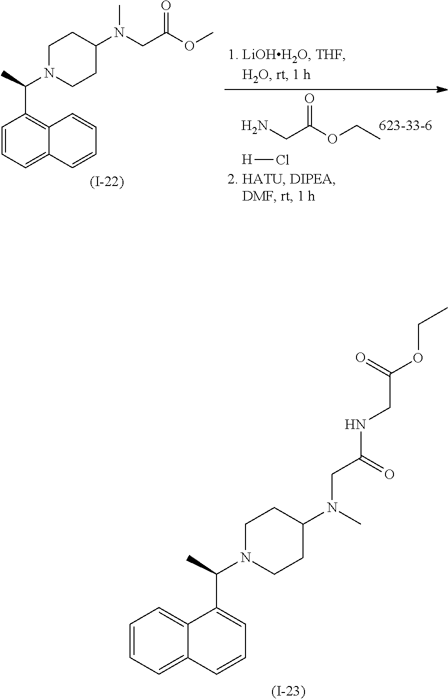

To a solution of the compound I-22 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and glycine ethyl ester hydrochloride (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

HPLC (Method 1) RT=3.34 min, Purity=92.9%, MS (ESI+) m/z calcd for C₂₄H₃₄N₃O₃⁺ [M+H]⁺ 412.3, found 412.4.

### Synthesis of Compound 25

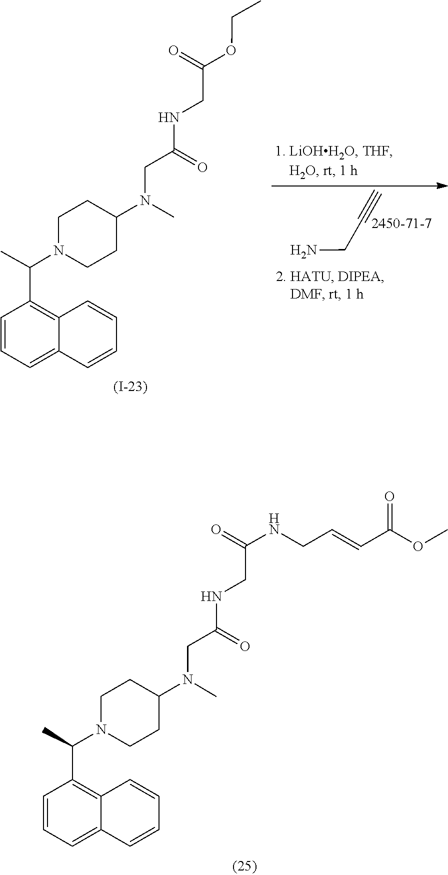

To a solution of the compound I-23 (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and glycine ethyl ester hydrochloride (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound I-24

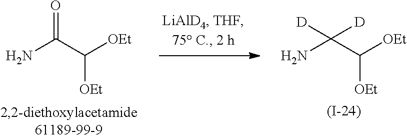

To a suspension of LiAlD₄ (12 mmol, 1.7 eq) in dry THF (5 ml) was added dropwise a solution of 2,2-diethoxylacetamide (6.8 mmol, 1.0 eq) in dry THF (0.60 ml) at 0° C. The resulting mixture was stirred at 25° C. for 30 min, then heated at 75° C. for 2 h. The mixture was cooled and quenched with H₂O (1 ml) and 2M NaOH (1 ml). After stirring for 30 min, the mixture was filtered through a peddle of silica gel and washed with small amount of ethyl ether to remove the white precipitate. The mother liquor was evaporated, residue was dissolved in 2 ml of DCM and dried over Na₂SO₄, filtered and concentrated.

### Synthesis of Compound 158

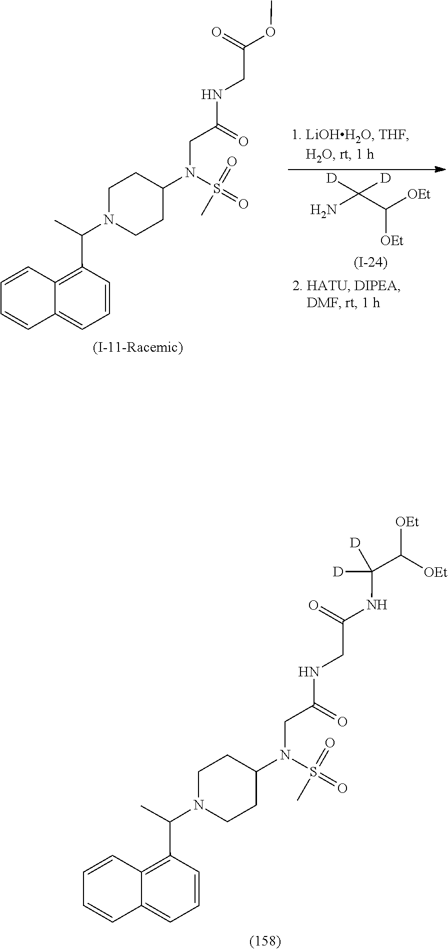

To a solution of the compound I-11-Racemic (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and the compound I-24 (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 153

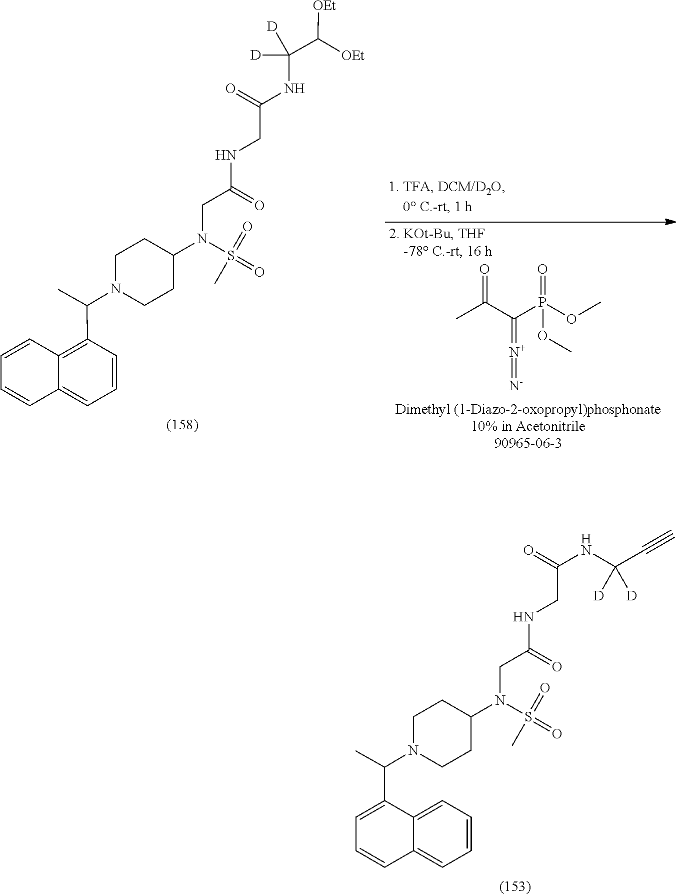 [SMILES: [2H]C([2H])(NC(=O)CNC(=O)CN(C1CCN(C(C)c2cccc3ccccc23)CC1)S(C)(=O)=O)C(OCC)OCC ; COP(=O)(OC)C(=[N+]=[N-])C(C)=O ; "(158)" ; [2H]C([2H])(C#C)NC(=O)CNC(=O)CN(C1CCN(C(C)c2cccc3ccccc23)CC1)S(C)(=O)=O ; "(153)"]

To a solution of the compound 158 (0.1 mmol, 1.0 eq) in DCM (0.8 mL) and D20 (0.2 mL), TFA (0.5 mmol, 5.0 eq) was added at 0° C., and the reaction mixture was stirred at room temperature for 1 h. The mixture was diluted in DCM and cooled to 0° C., neutralized by NaHCO₃/D20 soln. (10%, 3 ml), extracted with DCM, dried over Na₂SO₄, filtered and concentrated. Crude product was dried under the reduced pressure and used to the next step without further purification.

To a solution of KOt-Bu (0.2 mmol, 2.0 eq) in THE (3.0 mL), the bestmann reagent (0.2 mmol, 2.0 eq) was added at −78° C. The reaction mixture was stirred for 5 minutes at the same temperature. To the mixture, the aldehyde from the previous step in THF (1.5 mL) was added dropwise. The reaction mixture was allowed to be warmed up to room temperature slowly. The reaction was diluted by DCM and quenched by water at 0° C. The reaction mixture extracted by DCM and washed by water several times. The organic layer was dried over Na₂SO₄, filtered and concentrated. Then the crude material was purified by flash column chromatography.

### Synthesis of Compound I-25

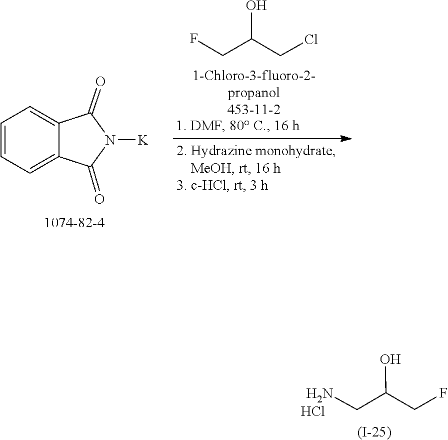

To a solution of potassium phthalimide (1.0 mmol, 1.0 eq) in DMF (1.0 mL), 1-chloro-3-fluoro-2-propanol (1.0 mmol, 1.0 eq) was added. The reaction mixture was stirred at 80° C. for 16 h. The reaction mixture extracted by EtOAc and washed by water several times. The organic layer was dried over Na₂SO₄, filtered and concentrated. Then the crude material was purified by flash column chromatography.

To a solution of the phthalimide protected compound from the previous step (1.0 mmol, 1.0 eq) in MeOH (2.0 mL), hydrazine monohydrate (1.0 mmol, 1.0 eq) was added. The reaction mixture was stirred at room temperature for 16 h. To the mixture, conc. aq. HCl (2 mL) was added, and the reaction mixture was stirred for 3 h. The precipitated was then collected by filtration and dried under the reduced pressure.

### Synthesis of Compound 116

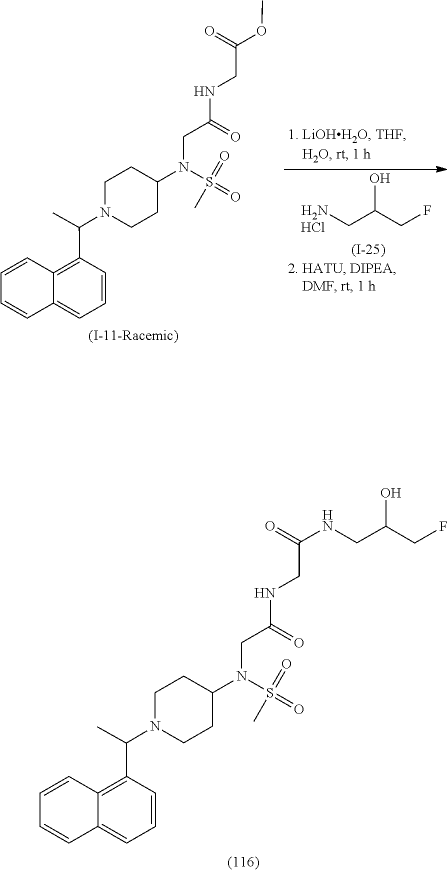

To a solution of the compound I-11-Racemic (0.1 mmol, 1.0 eq) in THF/Water (4:1, 0.5 mL), lithium hydroxide monohydrate (0.1 mmol, 1.0 eq) was added and the reaction mixture was stirred at room temperature for 1 h. The mixture was filtered through the thin layer of silica pad and washed with methanol. The filtrate was concentrated and thoroughly dried under the reduced pressure. The hydrolyzed carboxylic acid was used without further purification.

The carboxylic acid (0.1 mmol, 1.0 eq) was redissolved in DMF (0.3 mL), DIPEA (0.2 mmol, 2.0 eq) and the compound I-25 (0.1 mmol, 1.0 eq) was added. Then HATU (0.1 mmol, 1.0 eq) was slowly added to the mixture at room temperature. The mixture was stirred for 1 h. The reaction mixture was quenched by water, extracted with EtOAc and washed by brine several times. The organic layer was dried over Na₂SO₄, filtered and concentrated. Then the crude material was purified by flash column chromatography.

### Synthesis of Compound 132

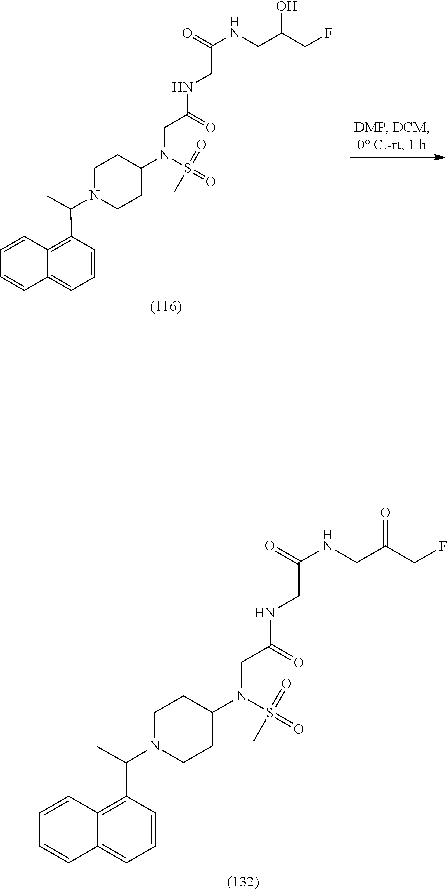 [SMILES: CC(c1cccc2ccccc12)N1CCC(N(CC(=O)NCC(=O)NCC(O)CF)S(C)(=O)=O)CC1 ; "(116)" ; CC(c1cccc2ccccc12)N1CCC(N(CC(=O)NCC(=O)NCC(=O)CF)S(C)(=O)=O)CC1 ; "(132)"]

To a solution of the compound 116 (0.1 mmol, 1.0 eq) in DCM (0.3 mL), DMP (0.2 mmol, 2.0 eq) was added at 0° C. and the reaction mixture was stirred for 1 h. Then the reaction mixture was quenched by sat. NaHCO₃ soln., extracted by DCM and washed by water several times. The organic layer was dried over Na₂SO₄, filtered and concentrated. Then the crude material was purified by flash column chromatography.

### PLpro Inhibitors—Examples

Examples of compounds according to Formula I include, but are not limited to, one or more of the compounds shown in ‘PLpro Inhibitors Examples’ as indicated below. Some compounds are synthesized following the general scheme and procedures with corresponding chemical reagents as described earlier.

### Compound 1, methyl (E)-4-(2-(2-(2-(benzo[d][1,3]dioxol-5-yl)-N-(1-(1-(naphthalen-1-yl)ethyl)piperidin-4-yl)acetamido)acetamido)acetamido)but-2-enoate

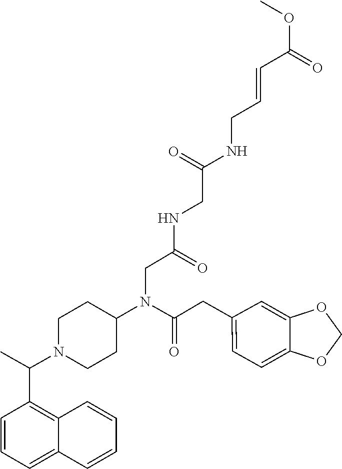 [SMILES: COC(=O)/C=C/CNC(=O)CNC(=O)CN(C(=O)Cc1ccc2c(c1)OCO2)C1CCN(C(C)c2cccc3ccccc23)CC1]

HPLC (Method 2) RT=4.78 min, Purity 95.0%, MS (ESI+) m/z calcd for C₃₅H₄₁N₄O₇⁺ [M+H]⁺ 629.7, found 629.4.

### Compound 2, methyl (R,E)-4-(2-(2-(2-(benzo[d][1,3]dioxol-5-yl)-N-(1-(1-(naphthalen-1-yl)ethyl)piperidin-4-yl)acetamido)acetamido)acetamido)but-2-enoate

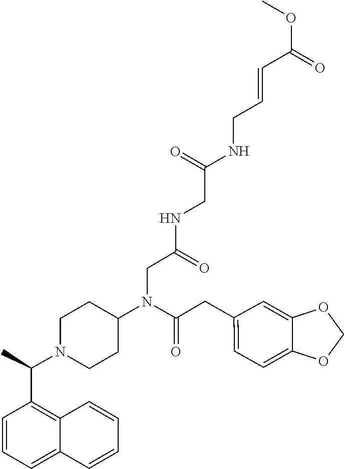 [SMILES: COC(=O)/C=C/CNC(=O)CNC(=O)CN(C(=O)Cc1ccc2c(c1)OCO2)C1CCN([C@H](C)c2cccc3ccccc23)CC1]

HPLC (Method 1) RT=4.42 min, Purity 95.0% MS (ESI+) m/z calcd for C₃₅H₄₁N₄O₇⁺ [M+H]⁺ 629.7, found 629.6.

### Compound 3, methyl (R,E)-4-(2-(2-(2-(3-fluorophenyl)-N-(1-(1-(naphthalen-1-yl)ethyl)piperidin-4-yl)acetamido)acetamido)acetamido)but-2-enoate

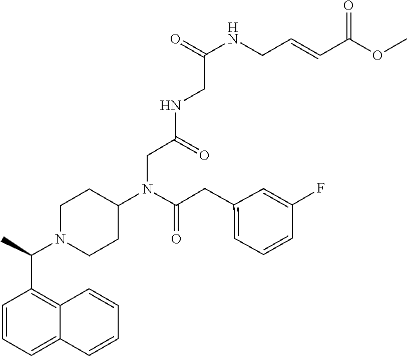 [SMILES: COC(=O)/C=C/CNC(=O)CNC(=O)CN(C(=O)Cc1cccc(F)c1)C1CCN([C@H](C)c2cccc3ccccc23)CC1]

HPLC (Method 1) RT=4.51 min, Purity >99.9%, MS (ESI+) m/z calcd for C₃₄H₄₀FN₄₀S⁺ [M+H]⁺ 603.7, found 603.5.

### Compound 4, methyl (E)-4-(2-(2-(N-(1-(1-(naphthalen-1-yl)ethyl)piperidin-4-yl)-2-phenylacetamido)acetamido)acetamido)but-2-enoate

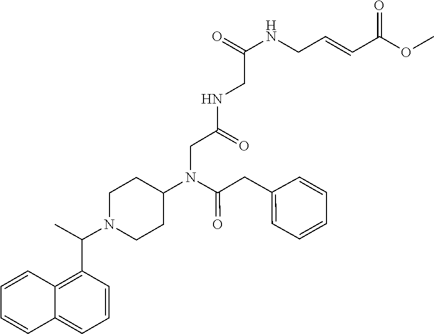 [SMILES: COC(=O)/C=C/CNC(=O)CNC(=O)CN(C(=O)Cc1ccccc1)C1CCN(C(C)c2cccc3ccccc23)CC1]

HPLC (Method 1) RT=4.44 min, Purity >99.9%, MS (ESI+) m/z calcd for C₃₄H₄₀N₄O₅⁺ [M+H]⁺ 585.7, found 585.5.

### Compound 5, methyl (R,E)-4-(2-(2-(N-(1-(1-(naphthalen-1-yl)ethyl)piperidin-4-yl)cyclopropanecarboxamido)acetamido)acetamido)but-2-enoate

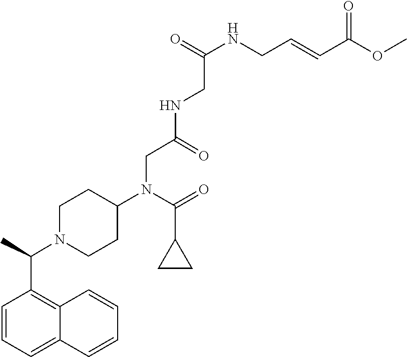 [SMILES: COC(=O)/C=C/CNC(=O)CNC(=O)CN(C(=O)C1CC1)C1CCN([C@H](C)c2cccc3ccccc23)CC1]

HPLC (Method 1) RT=4.11 min, Purity >99.9%, MS (ESI+) m/z calcd for C₃₀H₃₉N₄O₅⁺ [M+H]⁺ 535.7, found 535.5.

### Compound 6, methyl (E)-4-(2-(2-(N-(1-(1-(naphthalen-1-yl)ethyl)piperidin-4-yl)cyclobutanecarboxamido)acetamido)acetamido)but-2-enoate

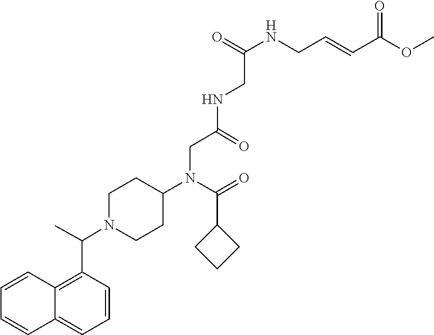 [SMILES: COC(=O)/C=C/CNC(=O)CNC(=O)CN(C(=O)C1CCC1)C1CCN(C(C)c2cccc3ccccc23)CC1]

HPLC (Method 1) RT=4.26 min, Purity >99.9%, MS (ESI+) m/z calcd for C₃₁H₄₁N₄O₅⁺ [M+H]⁺ 549.7, found 549.5.

### Compound 7, methyl (R,E)-4-(2-(2-(N-(1-(1-(naphthalen-1-yl)ethyl)piperidin-4-yl)cyclobutanecarboxamido)acetamido)acetamido)but-2-enoate

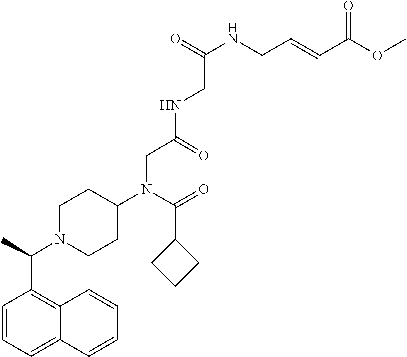 [SMILES: COC(=O)/C=C/CNC(=O)CNC(=O)CN(C(=O)C1CCC1)C1CCN([C@H](C)c2cccc3ccccc23)CC1]

HPLC (Method 1) RT=4.28 min, Purity >99.9%, MS (ESI+) m/z calcd for C₃₁H₄₁N₄O₅⁺ [M+H]⁺ 549.7, found 549.5.

### Compound 8, (E)-N-(2-((2-((4-amino-4-oxobut-2-en-1-yl)amino)-2-oxoethyl)amino)-2-oxoethyl)-N-(1-(1-(naphthalen-1-yl)ethyl)piperidin-4-yl)cyclobutanecarboxamide

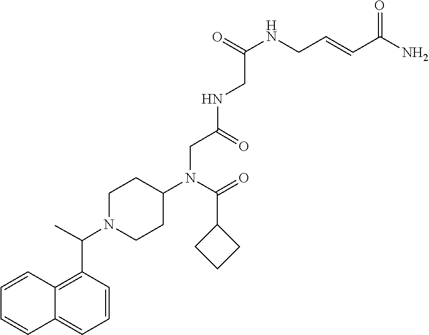 [SMILES: CC(c1cccc2ccccc12)N1CCC(N(CC(=O)NCC(=O)NC/C=C/C(N)=O)C(=O)C2CCC2)CC1]

HPLC (Method 1) RT=3.93 min, Purity 96.6% MS (ESI+) m/z calcd for C₃₀H₄₀NSO₄⁺ [M+H]⁺ 534.7, found 534.5.

### Compound 9, (E)-N-(2-((2-((3-(methylsulfonyl)allyl)amino)-2-oxoethyl)amino)-2-oxoethyl)-N-(1-(1-(naphthalen-1-yl)ethyl)piperidin-4-yl)cyclobutanecarboxamide

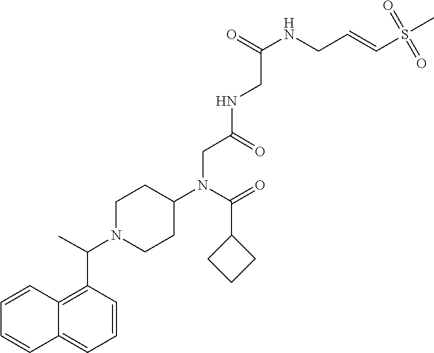 [SMILES: CC(c1cccc2ccccc12)N1CCC(N(CC(=O)NCC(=O)NC/C=C/S(C)(=O)=O)C(=O)C2CCC2)CC1]

HPLC (Method 1) RT=4.23 min, Purity 97.7%, MS (ESI+) m/z calcd for C₃₀H₄₁N₄O₅S⁺ [M+H]⁺ 569.7, found 569.5.

### Compound 10, methyl (R,E)-4-(2-(2-(N-(1-(1-(naphthalen-1-yl)ethyl)piperidin-4-yl)cyclopentanecarboxamido)acetamido)acetamido)but-2-enoate

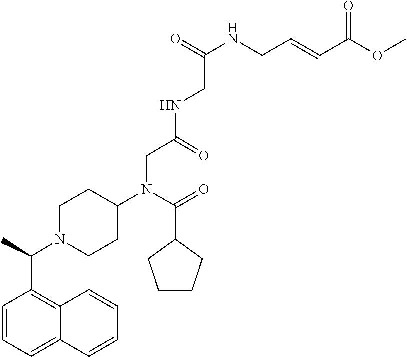 [SMILES: COC(=O)/C=C/CNC(=O)CNC(=O)CN(C(=O)C1CCCC1)C1CCN([C@H](C)c2cccc3ccccc23)CC1]

HPLC (Method 1) RT=4.41 min, Purity >99.9%, MS (ESI+) m/z calcd for C₃₂H₄₃N₄O₅⁺ [M+H]⁺ 563.7, found 563.6.

### Compound 11, methyl (R,E)-4-(2-(2-(N-(1-(1-(naphthalen-1-yl)ethyl)piperidin-4-yl)cyclohexanecarboxamido)acetamido)acetamido)but-2-enoate

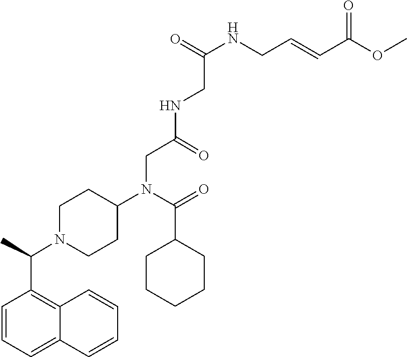 [SMILES: COC(=O)/C=C/CNC(=O)CNC(=O)CN(C(=O)C1CCCCC1)C1CCN([C@H](C)c2cccc3ccccc23)CC1]

HPLC (Method 1) RT=4.53 min, Purity >99.9%, MS (ESI+) m/z calcd for C₃₃H₄₅N₄O₅⁺ [M+H]⁺ 577.7, found 577.6.

### Compound 12, (R)-2-((1-(1-(naphthalen-1-yl)ethyl)piperidin-4-yl)amino)-N-(2-oxo-2-(prop-2-yn-1-ylamino)ethyl)acetamide

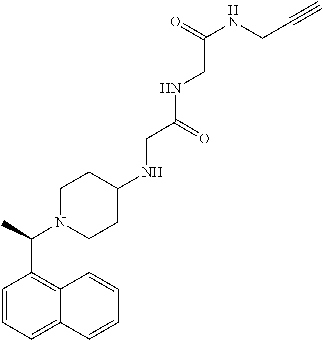 [SMILES: C#CCNC(=O)CNC(=O)CNC1CCN([C@H](C)c2cccc3ccccc23)CC1]

HPLC (Method 1) RT=2.81 min, Purity >99.9%, MS (ESI+) m/z calcd for C₂₄H₃₁N₄O₂⁺ [M+H]⁺ 407.4, found 407.5.

### Compound 13, methyl (E)-4-(2-(2-(N-(1-(1-(naphthalen-1-yl)ethyl)piperidin-4-yl)acetamido)acetamido)acetamido)but-2-enoate

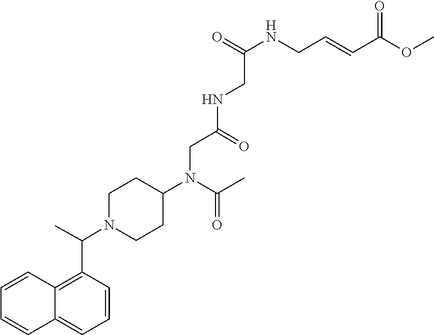 [SMILES: COC(=O)/C=C/CNC(=O)CNC(=O)CN(C(C)=O)C1CCN(C(C)c2cccc3ccccc23)CC1]

HPLC (Method 1) RT=3.94 min, Purity >99.9%, MS (ESI+) m/z calcd for C₂₉H₃₉N₄O₅⁺ [M+H]⁺ 509.3, found 509.3.

### Compound 14, methyl (R,E)-4-(2-(2-(2,2,2-trifluoro-N-(1-(1-(naphthalen-1-yl)ethyl)piperidin-4-yl)acetamido)acetamido)acetamido)but-2-enoate

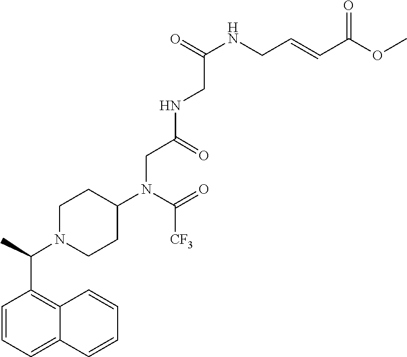 [SMILES: COC(=O)/C=C/CNC(=O)CNC(=O)CN(C(=O)C(F)(F)F)C1CCN([C@H](C)c2cccc3ccccc23)CC1]

HPLC (Method 1) RT=4.30 min, Purity >99.9%, MS (ESI+) m/z calcd for C₂₈H₃₄F3N₄O₅⁺ [M+H]⁺ 563.6, found 563.4.

### Compound 15, methyl (E)-4-(2-(2-(N-(1-(1-(naphthalen-1-yl)ethyl)piperidin-4-yl)propionamido)acetamido)acetamido)but-2-enoate

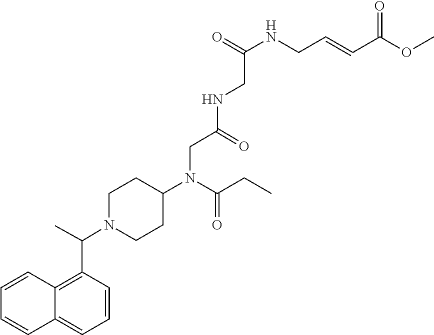 [SMILES: CCC(=O)N(CC(=O)NCC(=O)NC/C=C/C(=O)OC)C1CCN(C(C)c2cccc3ccccc23)CC1]

HPLC (Method 1) RT=4.05 min, Purity >99.9%, MS (ESI+) m/z calcd for C₂₉H₃₉N₄O₅⁺ [M+H]⁺ 523.3, found 523.5.

### Compound 16, methyl (E)-4-(2-(2-(3,3,3-trifluoro-N-(1-(1-(naphthalen-1-yl)ethyl)piperidin-4-yl)propanamido)acetamido)acetamido)but-2-enoate

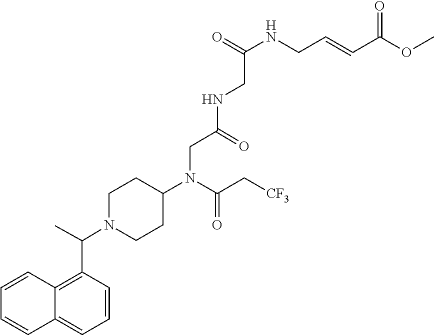 [SMILES: COC(=O)/C=C/CNC(=O)CNC(=O)CN(C(=O)CC(F)(F)F)C1CCN(C(C)c2cccc3ccccc23)CC1]

HPLC (Method 1) RT=4.28 min, *Purity 91.09% MS (ESI+) m/z calcd for C₂₉H₃₆F₃N₄O₅⁺ [M+H]⁺ 577.6, found 577.5.

### Compound 17, methyl (R,E)-4-(2-(2-(N-(1-(1-(naphthalen-1-yl)ethyl)piperidin-4-yl)isobutyramido)acetamido)acetamido)but-2-enoate

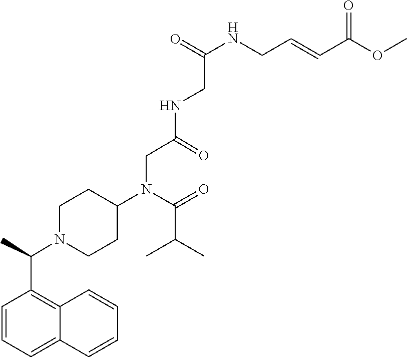 [SMILES: COC(=O)/C=C/CNC(=O)CNC(=O)CN(C(=O)C(C)C)C1CCN([C@H](C)c2cccc3ccccc23)CC1]

HPLC (Method 1) RT=4.18 min, Purity >99.9%, MS (ESI+) m/z calcd for C₃₀H₄₁N₄O₅⁺ [M+H]⁺ 537.7, found 537.4.

### Compound 18, methyl (R,E)-4-(2-(2-(N-(1-(1-(naphthalen-1-yl)ethyl)piperidin-4-yl)butyramido)acetamido)acetamido)but-2-enoate

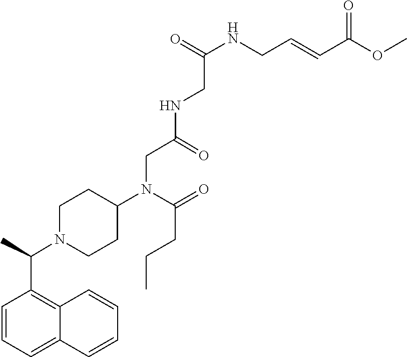 [SMILES: CCCC(=O)N(CC(=O)NCC(=O)NC/C=C/C(=O)OC)C1CCN([C@H](C)c2cccc3ccccc23)CC1]

HPLC (Method 1) RT=4.21 min, Purity >99.9%, MS (ESI+) m/z calcd for C₃₀H₄₁N₄₀S⁺ [M+H]⁺ 537.7, found 537.4.

### Compound 19, methyl (R,E)-4-(2-(2-(3-methyl-N-(1-(1-(naphthalen-1-yl)ethyl)piperidin-4-yl)butanamido)acetamido)acetamido)but-2-enoate

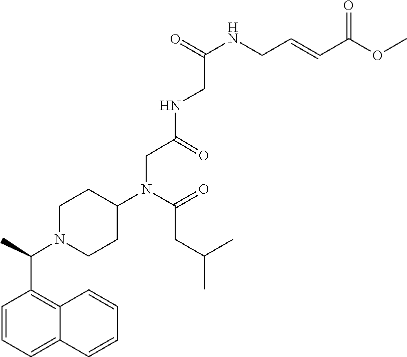 [SMILES: COC(=O)/C=C/CNC(=O)CNC(=O)CN(C(=O)CC(C)C)C1CCN([C@H](C)c2cccc3ccccc23)CC1]

HPLC (Method 1) RT=4.37 min, Purity >99.9%, MS (ESI+) m/z calcd for C₃₁H₄₃N₄O₅⁺ [M+H]⁺ 551.7, found 551.5.

### Compound 20, methyl (R,E)-4-(2-(2-(N-(1-(1-(naphthalen-1-yl)ethyl)piperidin-4-yl)methylsulfonamido)acetamido)acetamido)but-2-enoate

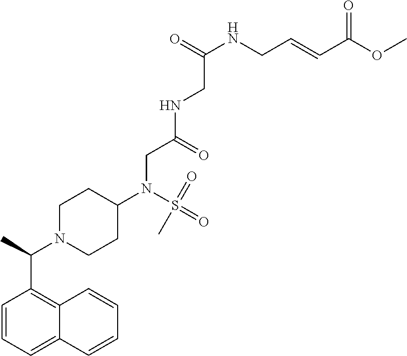 [SMILES: COC(=O)/C=C/CNC(=O)CNC(=O)CN(C1CCN([C@H](C)c2cccc3ccccc23)CC1)S(C)(=O)=O]

HPLC (Method 1) RT=4.08 min, Purity >99.9%, MS (ESI+) m/z calcd for C₂₇H₃₇N₄O₆S⁺ [M+H]⁺ 545.7, found 545.4.

### Compound 21, (R)-2-(N-(1-(1-(naphthalen-1-yl)ethyl)piperidin-4-yl)methylsulfonamido)-N-(2-oxo-2-(prop-2-yn-1-ylamino)ethyl)acetamide

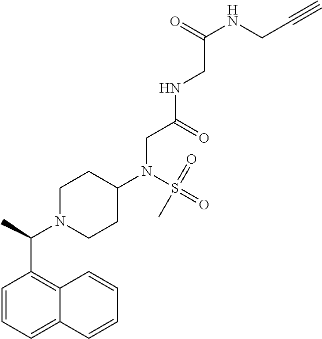 [SMILES: C#CCNC(=O)CNC(=O)CN(C1CCN([C@H](C)c2cccc3ccccc23)CC1)S(C)(=O)=O]

HPLC (Method 1) RT=3.96 min, Purity >99.9%, MS (ESI+) m/z calcd for C₂₅H₃₃N₄O₄S⁺ [M+H]⁺ 485.6, found 485.4.

### Compound 22, methyl (R,E)-4-(2-(2-((1-(1-(naphthalen-1-yl)ethyl)piperidin-4-yl)(sulfamoyl)amino)acetamido)acetamido)but-2-enoate

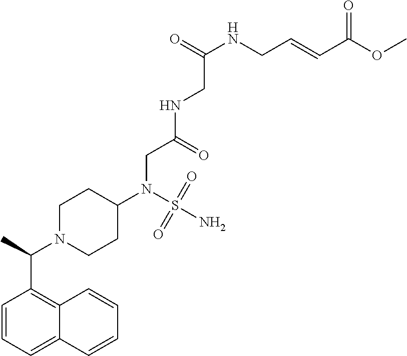 [SMILES: COC(=O)/C=C/CNC(=O)CNC(=O)CN(C1CCN([C@H](C)c2cccc3ccccc23)CC1)S(N)(=O)=O]

HPLC (Method 1) RT=3.97 min, Purity >99.9%, MS (ESI+) m/z calcd for C₂₆H₃₆N₅O₆S⁺ [M+H]⁺ 546.7, found 546.3.

### Compound 23, methyl (R,E)-4-(2-(2-((N-(tert-butoxycarbonyl)sulfamoyl)(1-(1-(naphthalen-1-yl)ethyl)piperidin-4-yl)amino)acetamido)acetamido)but-2-enoate

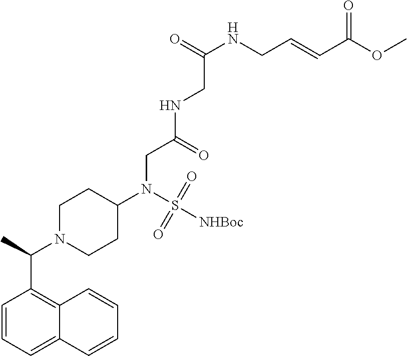 [SMILES: COC(=O)/C=C/CNC(=O)CNC(=O)CN(C1CCN([C@H](C)c2cccc3ccccc23)CC1)S(=O)(=O)NC(=O)OC(C)(C)C]

HPLC (Method 1) RT=4.53 min, Purity >99.9%, MS (ESI+) m/z calcd for C₃₁H₄₄N₅O₈S⁺ [M+H]⁺ 646.8, found 646.4.

### Compound 24, methyl (R,E)-4-(2-(2-((1-(1-(naphthalen-1-yl)ethyl)piperidin-4-yl)amino)acetamido)acetamido)but-2-enoate

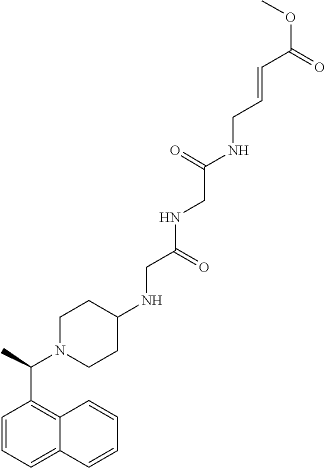 [SMILES: COC(=O)/C=C/CNC(=O)CNC(=O)CNC1CCN([C@H](C)c2cccc3ccccc23)CC1]

HPLC (Method 1) RT=3.21 min, Purity >99.9%, MS (ESI+) m/z calcd for C₂₆H₃₅N₄O₄⁺ [M+H]⁺ 467.6, found 467.5.

### Compound 25, methyl (R,E)-4-(2-(2-(methyl(1-(1-(naphthalen-1-yl)ethyl)piperidin-4-yl)amino)acetamido)acetamido)but-2-enoate

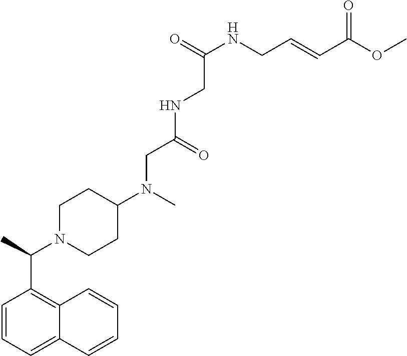 [SMILES: COC(=O)/C=C/CNC(=O)CNC(=O)CN(C)C1CCN([C@H](C)c2cccc3ccccc23)CC1]

HPLC (Method 1) RT=3.38 min, Purity >99.9%, MS (ESI+) m/z calcd for C₂₇H₃₇N₄O₄⁺ [M+H]⁺ 481.7, found 481.4.

### Compound 26, methyl (E)-4-(2-((S)-3-amino-2-((1-((R)-1-(naphthalen-1-yl)ethyl)piperidin-4-yl)amino)propanamido)acetamido)but-2-enoate

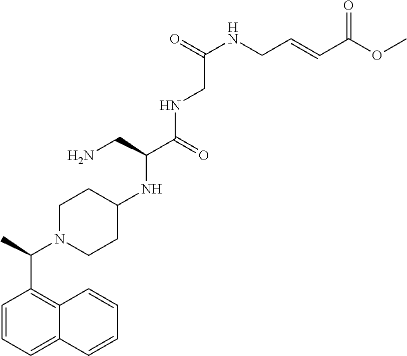 [SMILES: COC(=O)/C=C/CNC(=O)CNC(=O)[C@H](CN)NC1CCN([C@H](C)c2cccc3ccccc23)CC1]

HPLC (Method 1) RT=3.42 min, Purity 97.1%, MS (ESI+) m/z calcd for C₂₇H₃₈N₅O₄⁺ [M+H]⁺ 496.6, found 496.5.

### Compound 27, methyl (S,E)-2,2-dimethyl-7-((1-((R)-1-(naphthalen-1-yl)ethyl)piperidin-4-yl)amino)-4,8,11-trioxo-3-oxa-5,9,12-triazahexadec-14-en-16-oate

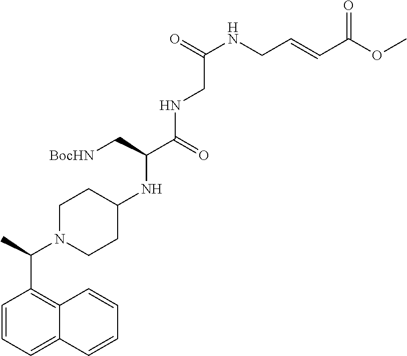 [SMILES: COC(=O)/C=C/CNC(=O)CNC(=O)[C@H](CNC(=O)OC(C)(C)C)NC1CCN([C@H](C)c2cccc3ccccc23)CC1]

HPLC (Method 1) RT=4.07 min, Purity >99.9%, MS (ESI+) m/z calcd for C₃₂H₄₆N₅O₄⁺ [M+H]⁺ 596.7, found 596.6.

### Compound 28, methyl (E)-4-(2-((R)-2-((1-((R)-1-(naphthalen-1-yl)ethyl)piperidin-4-yl)amino)propanamido)acetamido)but-2-enoate

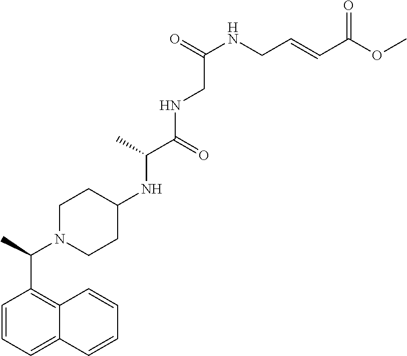 [SMILES: COC(=O)/C=C/CNC(=O)CNC(=O)[C@@H](C)NC1CCN([C@H](C)c2cccc3ccccc23)CC1]

HPLC (Method 1) RT=3.38 min, Purity >99.9%, MS (ESI+) m/z calcd for C₂₇H₃₇N₄O₄⁺ [M+H]⁺ 481.6, found 481.3.

### Compound 29, methyl (E)-4-(2-((3S)-3-((benzo[d][1,3]dioxol-5-ylmethyl)amino)-3-(1-(1-(naphthalen-1-yl)ethyl)piperidin-4-yl)propanamido)acetamido)but-2-enoate

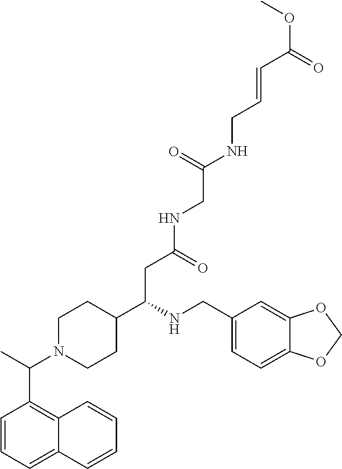 [SMILES: COC(=O)/C=C/CNC(=O)CNC(=O)C[C@H](NCc1ccc2c(c1)OCO2)C1CCN(C(C)c2cccc3ccccc23)CC1]

HPLC (Method 1) RT=3.70 min, Purity 95.0%, MS (ESI+) m/z calcd for C₃₅H₄₃N₄O₆⁺ [M+H]⁺ 615.7, found 615.5.

### Compound 30, 2-(benzo[d][1,3]dioxol-5-yl)-N-(2-((2-((1-cyanopiperidin-4-yl)amino)-2-oxoethyl)amino)-2-oxoethyl)-N-(1-(1-(naphthalen-1-yl)ethyl)piperidin-4-yl)acetamide

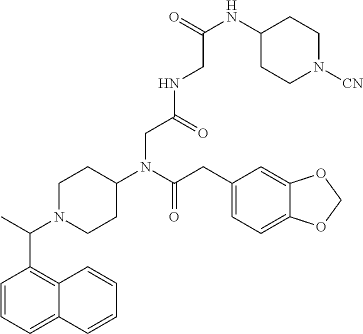 [SMILES: CC(c1cccc2ccccc12)N1CCC(N(CC(=O)NCC(=O)NC2CCN(C#N)CC2)C(=O)Cc2ccc3c(c2)OCO3)CC1]

HPLC (Method 2) RT=4.70 min, Purity >99.9%, MS (ESI+) m/z calcd for C₃₆H₄₃N₆O₅⁺ [M+H]⁺ 639.8, found 639.4.

### Compound 31, 2-(benzo[d][1,3]dioxol-5-yl)-N-(2-((2-((1-cyanopyrrolidin-3-yl)amino)-2-oxoethyl)amino)-2-oxoethyl)-N-(1-(1-(naphthalen-1-yl)ethyl)piperidin-4-yl)acetamide

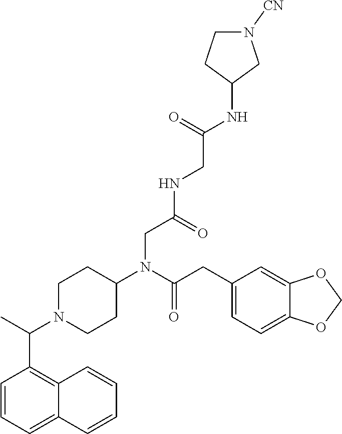 [SMILES: CC(c1cccc2ccccc12)N1CCC(N(CC(=O)NCC(=O)NC2CCN(C#N)C2)C(=O)Cc2ccc3c(c2)OCO3)CC1]

HPLC (Method 1) RT=4.35 min, Purity >99.9%, MS (ESI+) m/z calcd for C₃₅H₄₁N₆O₄⁺ [M+H]⁺ 625.7, found 625.5.

### Compound 32, (R)—N-(3-(2-((1-(1-(naphthalen-1-yl)ethyl)piperidin-4-yl)amino)acetamido)isoxazol-5-yl)acrylamide

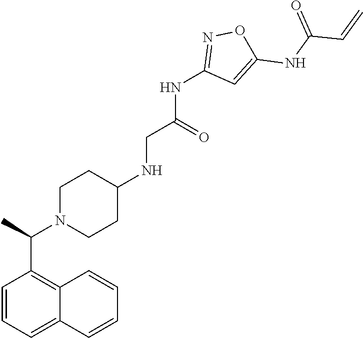 [SMILES: C=CC(=O)Nc1cc(NC(=O)CNC2CCN([C@H](C)c3cccc4ccccc34)CC2)no1]

HPLC (Method 1) RT=3.42 min, Purity 95.0% MS (ESI+) m/z calcd for C₂₅H₃₀N₅O₃⁺ [M+H]⁺ 448.5, found 448.3.

### Compound 33, 2-(benzo[d][1,3]dioxol-5-yl)-N-(2-((2-((1-cyanocyclopropyl)amino)-2-oxoethyl)amino)-2-oxoethyl)-N-(1-(1-(naphthalen-1-yl)ethyl)piperidin-4-yl)acetamide

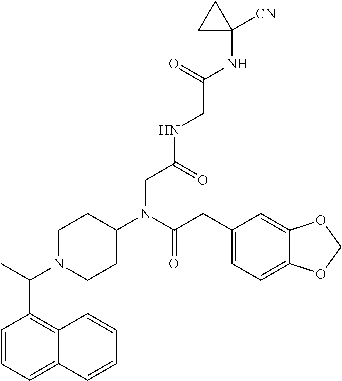 [SMILES: CC(c1cccc2ccccc12)N1CCC(N(CC(=O)NCC(=O)NC2(C#N)CC2)C(=O)Cc2ccc3c(c2)OCO3)CC1]

HPLC RT=4.76 min, Purity >99.9%, MS (ESI+) m/z calcd for C₃₄H₃₈N₅O⁵[M+H]⁺ 596.7, found 596.4.

### Compound 34, 2-(benzo[d][1,3]dioxol-5-yl)-N-(2-((2-((S)-2-cyanopyrrolidin-1-yl)-2-oxoethyl)amino)-2-oxoethyl)-N-(1-(1-(naphthalen-1-yl)ethyl)piperidin-4-yl)acetamide

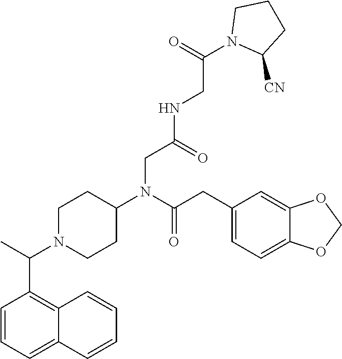 [SMILES: CC(c1cccc2ccccc12)N1CCC(N(CC(=O)NCC(=O)N2CCC[C@H]2C#N)C(=O)Cc2ccc3c(c2)OCO3)CC1]

HPLC (Method 1) RT=4.81 min, Purity 95.0%, MS (ESI+) m/z calcd for C₃₅H₄₀N₅O₅⁺ [M+H]⁺ 610.7, found 610.5.

### Compound 35, methyl (E)-4-(1-(2-((1-(1-(naphthalen-1-yl)ethyl)piperidin-4-yl)amino)acetamido cyclopropane-1-carboxamido)but-2-enoate

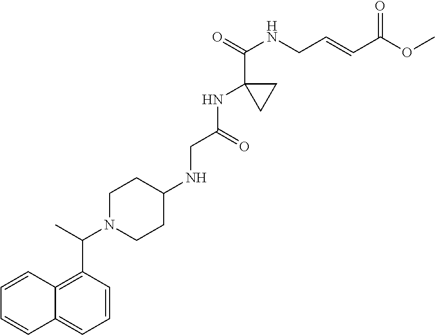 [SMILES: COC(=O)/C=C/CNC(=O)C1(NC(=O)CNC2CCN(C(C)c3cccc4ccccc34)CC2)CC1]

HPLC (Method 1) RT=3.25 min, Purity >99.9%, MS (ESI+) m/z calcd for C₂₈H₃₇N₄O₄⁺ [M+H]⁺ 493.6, found 493.5.

### Compound 36, methyl (E)-4-(1-(2-((tert-butoxycarbonyl)(1-(1-(naphthalen-1-yl)ethyl)piperidin-4-yl)amino)acetamido)cyclopropane-1-carboxamido)but-2-enoate

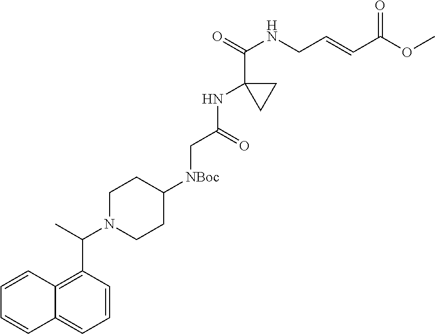 [SMILES: COC(=O)/C=C/CNC(=O)C1(NC(=O)CN(C(=O)OC(C)(C)C)C2CCN(C(C)c3cccc4ccccc34)CC2)CC1]

HPLC (Method 1) RT=4.50 min, Purity >99.9%, MS (ESI+) m/z calcd for C₃₃H₄₅N₄O₆⁺ [M+H]⁺ 593.7, found 593.5.

### Compound 37, methyl (E)-4-(2-methyl-2-(2-((1-(1-(naphthalen-1-yl)ethyl)piperidin-4-yl)amino)acetamido)propanamido)but-2-enoate

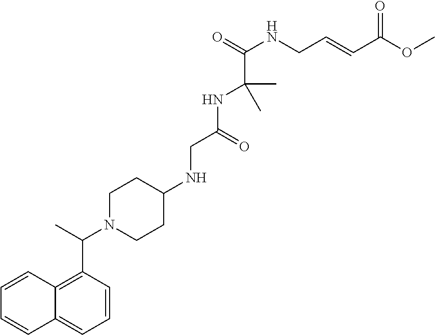 [SMILES: COC(=O)/C=C/CNC(=O)C(C)(C)NC(=O)CNC1CCN(C(C)c2cccc3ccccc23)CC1]

HPLC (Method 1) RT=3.34 min, Purity >99.9%, MS (ESI+) m/z calcd for C₂₈H₃₉N₄O₄⁺ [M+H]⁺ 495.6, found 495.4.

### Compound 38, methyl (E)-2,2,9,9-tetramethyl-5-(1-(1-(naphthalen-1-yl)ethyl)piperidin-4-yl)-4,7, 10-trioxo-3-oxa-5,8,1,1-triazapentadec-13-en-15-oate

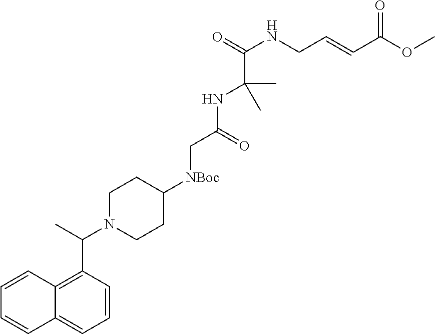 [SMILES: COC(=O)/C=C/CNC(=O)C(C)(C)NC(=O)CN(C(=O)OC(C)(C)C)C1CCN(C(C)c2cccc3ccccc23)CC1]

HPLC (Method 1) RT=4.50 min, Purity >99.9%, MS (ESI+) m/z calcd for C₃₃H₄₇N₄O₆⁺ [M+H]⁺ 595.8, found 595.5.

### Compound 39, (E)-4-(2-(2-(2-(benzo[d][1,3]dioxol-5-yl)-N-(1-(1-(naphthalen-1-yl)ethyl)piperidin-4-yl)acetamido)acetamido)acetamido)-N,N-dimethylbut-2-enamide

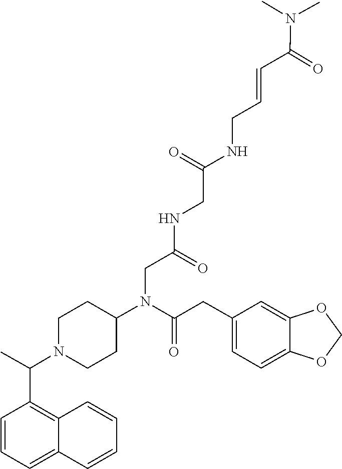 [SMILES: CC(c1cccc2ccccc12)N1CCC(N(CC(=O)NCC(=O)NC/C=C/C(=O)N(C)C)C(=O)Cc2ccc3c(c2)OCO3)CC1]

HPLC (Method 1) RT=4.34 min, Purity 95.0%, MS (ESI+) m/z calcd for C₃₆H₄₄N₅O₆⁺ [M+H]⁺ 642.8, found 642.6.

### Compound 40, (E)-4-(2-(2-(2-(benzo[d][1,3]dioxol-5-yl)-N-(1-(1-(naphthalen-1-yl)ethyl)piperidin-4-yl)acetamido)acetamido)acetamido)but-2-enoic acid

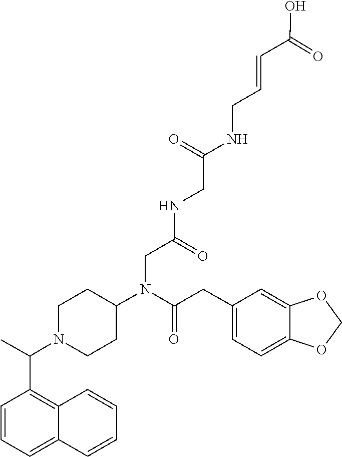 [SMILES: CC(c1cccc2ccccc12)N1CCC(N(CC(=O)NCC(=O)NC/C=C/C(=O)O)C(=O)Cc2ccc3c(c2)OCO3)CC1]

HPLC (Method 1) RT=4.22 min, Purity >99.9%, MS (ESI+) m/z calcd for C₃₄H₃₉N₄O₇⁺ [M+H]⁺ 615.7, found 615.5.

### Compound 41, 2-(benzo[d][1,3]dioxol-5-yl)-N-(2-((2-((2-cyanoethyl)amino)-2-oxoethyl)amino)-2-oxoethyl)-N-(1-(1-(naphthalen-1-yl)ethyl)piperidin-4-yl)acetamide

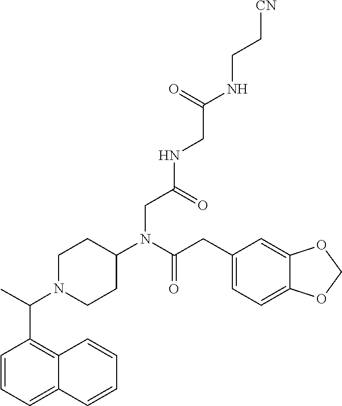 [SMILES: CC(c1cccc2ccccc12)N1CCC(N(CC(=O)NCC(=O)NCCC#N)C(=O)Cc2ccc3c(c2)OCO3)CC1]

HPLC (Method 1) RT=4.26 min, Purity 95.0%, MS (ESI+) m/z calcd for C₃₃H₃₈N₅O₅⁺ [M+H]⁺ 584.7, found 584.5.

### Compound 42, 2-(benzo[d][1,3]dioxol-5-yl)-N-(2-((2-((cyanomethyl)amino)-2-oxoethyl)amino)-2-oxoethyl)-N-(1-(1-(naphthalen-1-yl)ethyl)piperidin-4-yl)acetamide

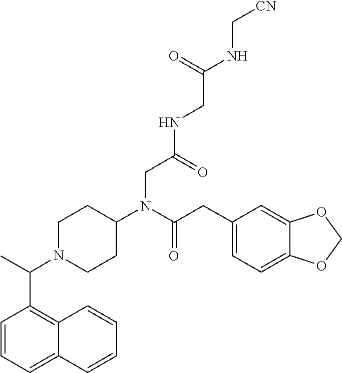 [SMILES: CC(c1cccc2ccccc12)N1CCC(N(CC(=O)NCC(=O)NCC#N)C(=O)Cc2ccc3c(c2)OCO3)CC1]

HPLC (Method 1) RT=4.28 min, Purity 95.0% MS (ESI+) m/z calcd for C₃₂H₃₆N₅O₅⁺ [M+H]⁺ 570.7, found 570.4.

### Compound 43, methyl (E)-4-((R)-2-(2-((1-((R)-1-(naphthalen-1-yl)ethyl)piperidin-4-yl)amino)acetamido)propanamido)but-2-enoate

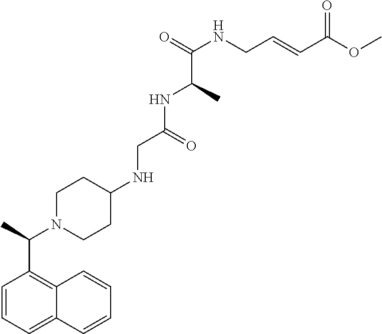 [SMILES: COC(=O)/C=C/CNC(=O)[C@@H](C)NC(=O)CNC1CCN([C@H](C)c2cccc3ccccc23)CC1]

HPLC (Method 1) RT=3.36 min, Purity 97.24% MS (ESI+) m/z calcd for C₂₇H₃₇N₄O₄⁺ [M+H]⁺ 481.6, found 481.4.

### Compound 44, methyl (E)-4-((R)-2-((R)-2-((1-((R)-1-(naphthalen-1-yl)ethyl)piperidin-4-yl)amino)propanamido)propanamido)but-2-enoate

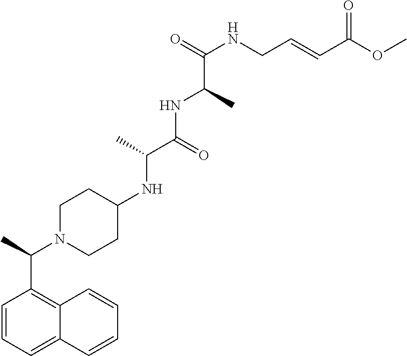 [SMILES: COC(=O)/C=C/CNC(=O)[C@@H](C)NC(=O)[C@@H](C)NC1CCN([C@H](C)c2cccc3ccccc23)CC1]

HPLC (Method 1) RT=3.38 min, Purity >99.9%, MS (ESI+) m/z calcd for C₂₈H₃₉N₄O₄⁺ [M+H]⁺ 495.6, found 495.4.

### Compound 45, methyl (E)-4-(2-(2-((1-benzhydrylpiperidin-4-yl)amino)acetamido)acetamido)but-2-enoate

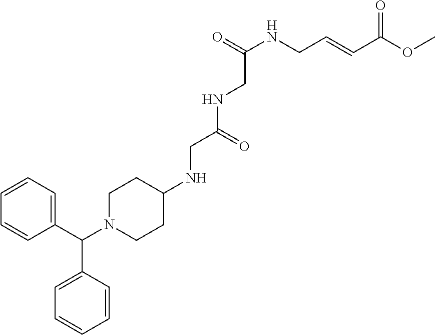 [SMILES: COC(=O)/C=C/CNC(=O)CNC(=O)CNC1CCN(C(c2ccccc2)c2ccccc2)CC1]

HPLC (Method 1) RT=3.49 min, Purity >99.9%, MS (ESI+) m/z calcd for C₂₇H₃₅N₄O₄⁺ [M+H]⁺ 479.6, found 479.4.

### Compound 46, methyl (E)-4-((2R)-2-(2-(N-(1-(1-(naphthalen-1-yl)ethyl)piperidin-4-yl)cyclobutanecarboxamido)acetamido)propanamido)but-2-enoate

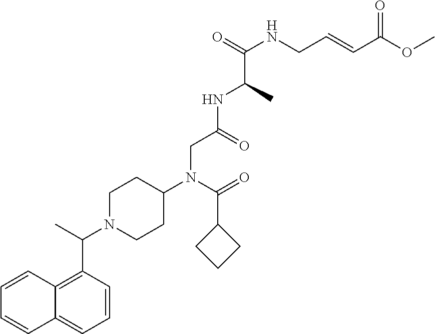 [SMILES: COC(=O)/C=C/CNC(=O)[C@@H](C)NC(=O)CN(C(=O)C1CCC1)C1CCN(C(C)c2cccc3ccccc23)CC1]

HPLC (Method 1) RT=4.22 min, Purity 99.8%, MS (ESI+) m/z calcd for C₃₂H₄₃N₄O₅⁺ [M+H]⁺ 563.7, found 563.5.

### Compound 47, methyl (E)-4-((2S)-2-(2-(N-(1-(1-(naphthalen-1-yl)ethyl)piperidin-4-yl)cyclobutanecarboxamido)acetamido)propanamido)but-2-enoate

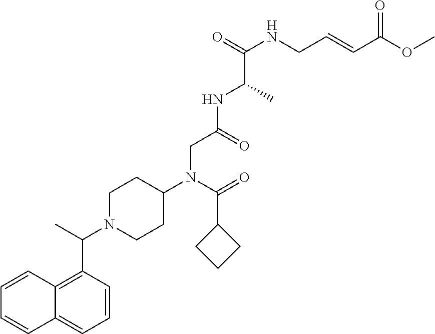 [SMILES: COC(=O)/C=C/CNC(=O)[C@H](C)NC(=O)CN(C(=O)C1CCC1)C1CCN(C(C)c2cccc3ccccc23)CC1]

HPLC (Method 1) RT=4.29 min, Purity 95.7%, MS (ESI+) m/z calcd for C₃₂H₄₃N₄O₅⁺ [M+H]⁺ 563.7, found 563.5.

### Compound 48, (R,E)-4-(2-(2-((1-(1-(naphthalen-1-yl)ethyl)piperidin-4-yl)amino)acetamido)acetamido)but-2-enamide

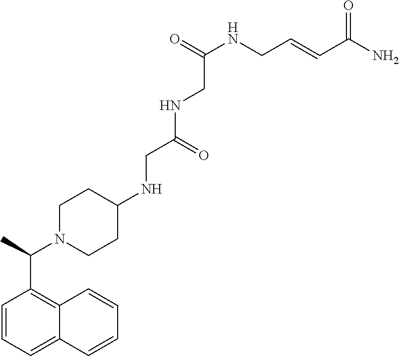 [SMILES: C[C@H](c1cccc2ccccc12)N1CCC(NCC(=O)NCC(=O)NC/C=C/C(N)=O)CC1]

HPLC (Method 1) RT=2.44 min, Purity >99.9%, MS (ESI+) m/z calcd for C₂₅H₃₄N₅O₃⁺ [M+H]⁺ 452.6, found 452.4.

### Compound 49, (R)—N-(2-((3-acrylamidopropyl)amino)-2-oxoethyl)-N-(1-(1-(naphthalen-1-yl)ethyl)piperidin-4-yl)cyclobutanecarboxamide

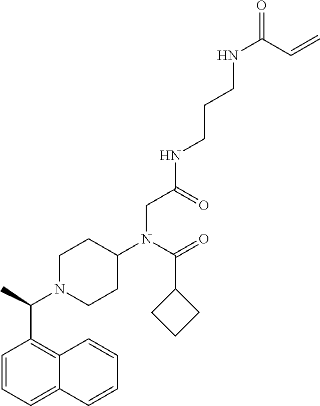 [SMILES: C=CC(=O)NCCCNC(=O)CN(C(=O)C1CCC1)C1CCN([C@H](C)c2cccc3ccccc23)CC1]

HPLC (Method 1) RT=4.12 min, Purity >99.9%, MS (ESI+) m/z calcd for C₃₀H₄₁N₄O₃⁺ [M+H]⁺ 505.7, found 505.5.

### Compound 50, methyl (R,E)-4-(2-(N-methyl-2-(N-(1-(1-(naphthalen-1-yl)ethyl)piperidin-4-yl)methylsulfonamido)acetamido)acetamido)but-2-enoate

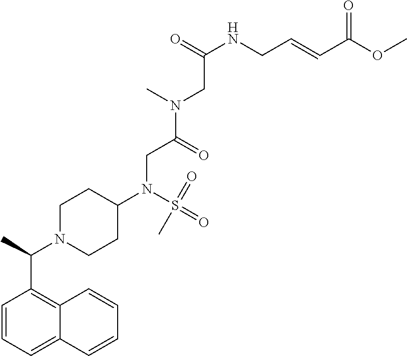 [SMILES: COC(=O)/C=C/CNC(=O)CN(C)C(=O)CN(C1CCN([C@H](C)c2cccc3ccccc23)CC1)S(C)(=O)=O]

HPLC (Method 1) RT=4.14 min, Purity >99.9%, MS (ESI+) m/z calcd for C₂₈H₃₉N₄O₆S⁺ [M+H]⁺ 559.7, found 559.4.

### Compound 51, (E)-N-(2-((2-((4-(methylamino)-4-oxobut-2-en-1-yl)amino)-2-oxoethyl)amino)-2-oxoethyl)-N-(1-(1-(naphthalen-1-yl)ethyl)piperidin-4-yl)cyclobutanecarboxamide

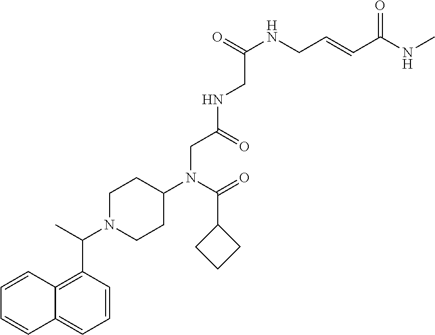 [SMILES: CNC(=O)/C=C/CNC(=O)CNC(=O)CN(C(=O)C1CCC1)C1CCN(C(C)c2cccc3ccccc23)CC1]

HPLC (Method 1) RT=3.99 min, Purity 97.7% MS (ESI+) m/z calcd for C₃₁H₄₂N₅O₄⁺ [M+H]⁺ 548.7, found 548.5.

### Compound 52, N—((S)-1-(cyanomethyl)-2-oxopyrrolidin-3-yl)-2-(N-(1-((R)-1-(naphthalen-1-yl)et yl)piperidin-4-yl)methylsulfonamido)acetamide

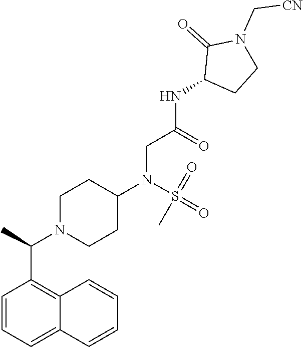 [SMILES: C[C@H](c1cccc2ccccc12)N1CCC(N(CC(=O)N[C@H]2CCN(CC#N)C2=O)S(C)(=O)=O)CC1]

HPLC (Method 1) RT=3.96 min, Purity 96.1%, MS (ESI+) m/z calcd for C₂₆H₃₄N₅O₄S⁺ [M+H]⁺ 512.6, found 512.4.

### Compound 53, (E)-N-(2-((2-((3-cyanoallyl)amino)-2-oxoethyl)amino)-2-oxoethyl)-N-(1-(1-(naphthalen-1-yl)ethyl)piperidin-4-yl)cyclobutanecarboxamide

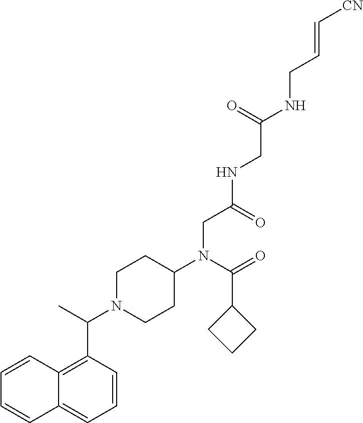 [SMILES: CC(c1cccc2ccccc12)N1CCC(N(CC(=O)NCC(=O)NC/C=C/C#N)C(=O)C2CCC2)CC1]

HPLC (Method 1) RT=4.31 min, Purity >99.9%, MS (ESI+) m/z calcd for C₃₀H₃₈N₅O₃⁺ [M+H]⁺ 516.6, found 516.4.

### Compound 54, (R)—N-(2-((2-(allylamino)-2-oxoethyl)amino)-2-oxoethyl)-N-(1-(1-(naphthalen-1-yl)ethyl)piperidin-4-yl)cyclobutanecarboxamide

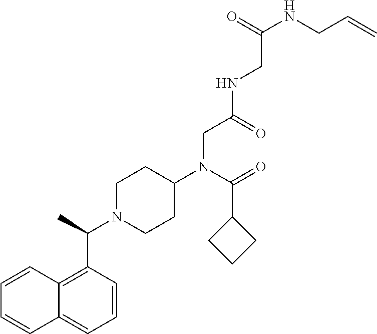 [SMILES: C=CCNC(=O)CNC(=O)CN(C(=O)C1CCC1)C1CCN([C@H](C)c2cccc3ccccc23)CC1]

HPLC (Method 1) RT=4.58 min, Purity 98.2% MS (ESI+) m/z calcd for C₂₉H₃₉N₄O₃⁺ [M+H]⁺ 491.6, found 491.5.

### Compound 55, isopropyl (R,E)-4-(2-(2-(N-(1-(1-(naphthalen-1-yl)ethyl)piperidin-4-yl)cyclobutanecarboxamido)acetamido)acetamido)but-2-enoate

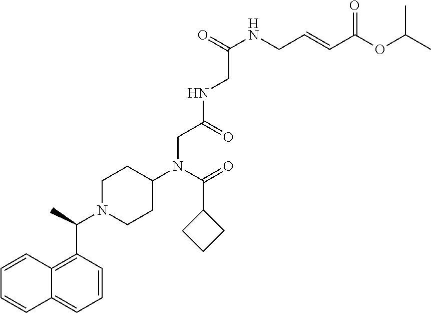 [SMILES: CC(C)OC(=O)/C=C/CNC(=O)CNC(=O)CN(C(=O)C1CCC1)C1CCN([C@H](C)c2cccc3ccccc23)CC1]

HPLC (Method 1) RT=4.68 min, Purity 98.8%, MS (ESI+) m/z calcd for C₃₃H₄₅N₄O₅⁺ [M+H]⁺ 577.7, found 577.5.

### Compound 56, (R,E)-N-(1-(1-(naphthalen-1-yl)ethyl)piperidin-4-yl)-N-(2-oxo-2-((2-oxo-2-((4-oxo-4-(phenylamino)but-2-en-1-yl)amino)ethyl)amino)ethyl)cyclobutanecarboxamide

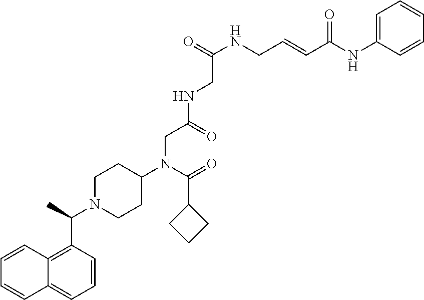 [SMILES: C[C@H](c1cccc2ccccc12)N1CCC(N(CC(=O)NCC(=O)NC/C=C/C(=O)Nc2ccccc2)C(=O)C2CCC2)CC1]

HPLC (Method 1) RT=4.62 min, Purity 95.1%, MS (ESI+) m/z calcd for C₃₆H₄₄N₅O₄⁺ [M+H]⁺ 610.8, found 610.6.

### Compound 57, (R)-3-(2-(N-(1-(1-(naphthalen-1-yl)ethyl)piperidin-4-yl)methylsulfonamido)acetamido)-N-(prop-2-yn-1-yl)propanamide

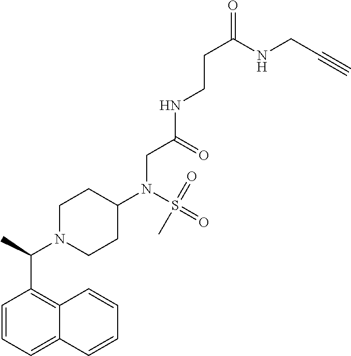 [SMILES: C#CCNC(=O)CCNC(=O)CN(C1CCN([C@H](C)c2cccc3ccccc23)CC1)S(C)(=O)=O]

HPLC (Method 1) RT=3.95 min, Purity 98.6% MS (ESI+) m/z calcd for C₂₆H₃₅N₄O₄S⁺ [M+H]⁺ 499.6, found 499.6.

### Compound 58, (R)-3-(N-(1-(1-(naphthalen-1-yl)ethyl)piperidin-4-yl)methylsulfonamido)-N-(2-oxo-2-(prop-2-yn-1-ylamino)ethyl)propanamide

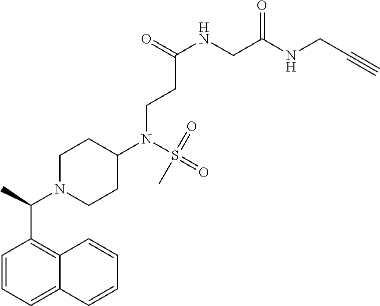 [SMILES: C#CCNC(=O)CNC(=O)CCN(C1CCN([C@H](C)c2cccc3ccccc23)CC1)S(C)(=O)=O]

HPLC (Method 1) RT=3.97 min, Purity 97.6%, MS (ESI+) m/z calcd for C₂₆H₃₅N₄O₄S⁺ [M+H]⁺ 499.6, found 499.4.

### Compound 59, (R,E)-N-(3-(methylsulfonyl)allyl)-2-(2-(N-(1-(1-(naphthalen-1-yl)ethyl)piperidin-4-yl)methylsulfonamido)acetamido)acetamide

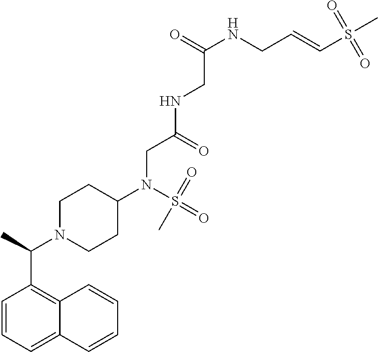 [SMILES: C[C@H](c1cccc2ccccc12)N1CCC(N(CC(=O)NCC(=O)NC/C=C/S(C)(=O)=O)S(C)(=O)=O)CC1]

HPLC (Method 1) RT=3.90 min, Purity >99.9%, MS (ESI+) m/z calcd for C₂₆H₃₇N₄O₆S₂⁺ [M+H]⁺ 565.7, found 565.3.

### Compound 60, (R)-2-(N-(1-(1-(naphthalen-1-yl)ethyl)piperidin-4-yl)cyclopropanesulfonamido)-N-(2-oxo-2-(prop-2-yn-1-ylamino)ethyl)acetamide

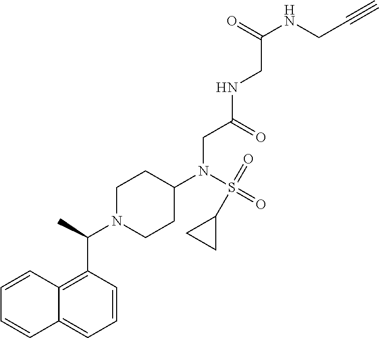 [SMILES: C#CCNC(=O)CNC(=O)CN(C1CCN([C@H](C)c2cccc3ccccc23)CC1)S(=O)(=O)C1CC1]

HPLC (Method 1) RT=4.16 min, Purity >99.9%, MS (ESI+) m/z calcd for C₂₇H₃₅N₄O₄S⁺ [M+H]⁺ 511.7, found 511.5.

### Compound 61, (R)—N-(but-2-yn-1-yl)-2-(2-(N-(1-(1-(naphthalen-1-yl)ethyl)piperidin-4-yl)methylsulfonamido)acetamido)acetamide

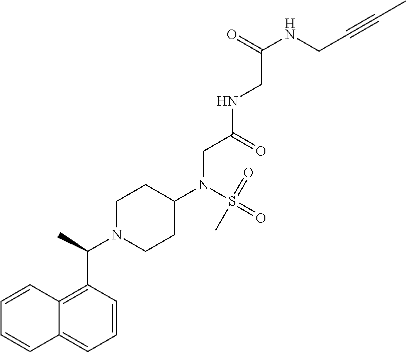 [SMILES: CC#CCNC(=O)CNC(=O)CN(C1CCN([C@H](C)c2cccc3ccccc23)CC1)S(C)(=O)=O]

HPLC (Method 1) RT=4.09 min, Purity >99.9%, MS (ESI+) m/z calcd for C₂₆H₃₅N₄O₄S⁺ [M+H]⁺ 499.6, found 499.4.

### Compound 62, (R)-2-(N-(1-(1-(naphthalen-1-yl)ethyl)piperidin-4-yl)methylsulfonamido)-N-(2-oxo-2-((3-phenylprop-2-yn-1-yl)amino)ethyl)acetamide

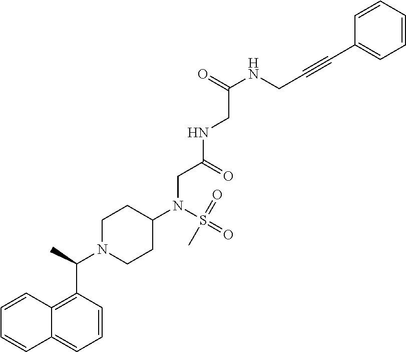 [SMILES: C[C@H](c1cccc2ccccc12)N1CCC(N(CC(=O)NCC(=O)NCC#Cc2ccccc2)S(C)(=O)=O)CC1]

HPLC (Method 1) RT=4.63 min, Purity >99.9%, MS (ESI+) m/z calcd for C₃₁H₃₇N₄O₄S⁺ [M+H]⁺ 561.7, found 561.3.

### Compound 63, (R)-2-(N-(1-(1-(naphthalen-1-yl)ethyl)piperidin-4-yl)phenylsulfonamido)-N-(2-oxo-2-(prop-2-yn-1-ylamino)ethyl)acetamide

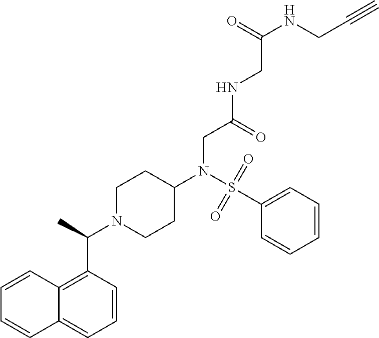 [SMILES: C#CCNC(=O)CNC(=O)CN(C1CCN([C@H](C)c2cccc3ccccc23)CC1)S(=O)(=O)c1ccccc1]

HPLC (Method 1) RT=4.43 min, Purity >99.9%, MS (ESI+) m/z calcd for C₃₀H₃₅N₄O₄S⁺ [M+H]⁺ 546.7, found 547.4.

### Compound 64, (R)-2-(N-(1-(1-(naphthalen-1-yl)ethyl)piperidin-4-yl)methylsulfonamido)-N-(2-oxo-2-((2-oxoethyl)amino)ethyl)acetamide

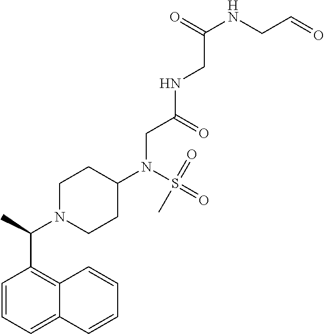 [SMILES: C[C@H](c1cccc2ccccc12)N1CCC(N(CC(=O)NCC(=O)NCC=O)S(C)(=O)=O)CC1]

HPLC (Method 1) RT=3.67 min, Purity >99.9%, MS (ESI+) m/z calcd for C₂₄H₃₃N₄O₅S⁺ [M+H]⁺ 489.6, found 489.4.

### Compound 65, (R)-2-(N-(1-(1-(naphthalen-1-yl)ethyl)piperidin-4-yl)pyrrolidine-1-sulfonamido)-N-(2-oxo-2-(prop-2-yn-1-ylamino)ethyl)acetamide

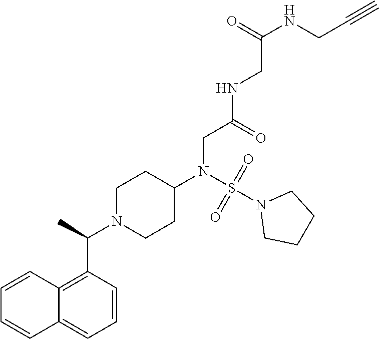 [SMILES: C#CCNC(=O)CNC(=O)CN(C1CCN([C@H](C)c2cccc3ccccc23)CC1)S(=O)(=O)N1CCCC1]

HPLC (Method 1) RT=4.36 min, Purity >99.9%, MS (ESI+) m/z calcd for C₂₈H₃₈N₅O₄S⁺ [M+H]⁺ 540.7, found 540.5.

### Compound 66, N-(2-hydroxy-3-(3-(prop-2-yn-1-yl)ureido)propyl)-N-(1-((R)-1-(naphthalen-1-yl)ethyl)piperidin-4-yl)methanesulfonamide

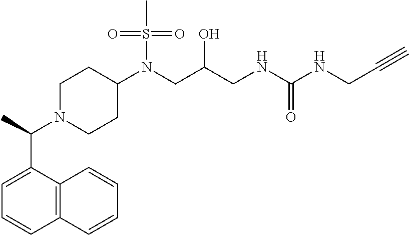 [SMILES: C#CCNC(=O)NCC(O)CN(C1CCN([C@H](C)c2cccc3ccccc23)CC1)S(C)(=O)=O]

HPLC (Method 1) RT=3.96 min, Purity >99.9%, MS (ESI+) m/z calcd for C₂₅H₃₅N₄O₄S⁺ [M+H]⁺ 487.6, found 487.4.

### Compound 67, methyl (E)-4-(2-(2-((1-((R)-1-(naphthalen-1-yl)ethyl)piperidin-4-yl)((E)-4,4,4-trifluoro-3-oxobut-1-en-1-yl)amino)acetamido)acetamido)but-2-enoate

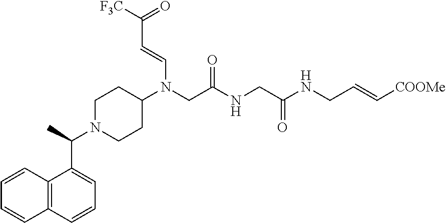 [SMILES: COC(=O)/C=C/CNC(=O)CNC(=O)CN(/C=C/C(=O)C(F)(F)F)C1CCN([C@H](C)c2cccc3ccccc23)CC1]

HPLC (Method 1) RT=4.33 min, Purity=98.5%, MS (ESI+) m/z calcd for C₃₀H₃₆F₃N₄O₅⁺ [M+H]⁺ 589.3, found 589.5.

### Compound 68, 1-(2-hydroxy-3-((1-((R)-1-(naphthalen-1-yl)ethyl)piperidin-4-yl)amino)propyl)-3-(prop-2-yn-1-yl)urea

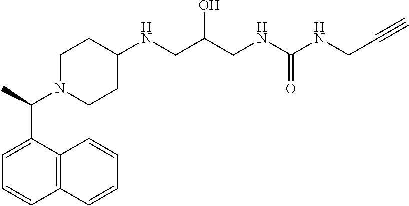 [SMILES: C#CCNC(=O)NCC(O)CNC1CCN([C@H](C)c2cccc3ccccc23)CC1]

HPLC (Method 1) RT=2.89 min, Purity=99.4%, MS (ESI+) m/z calcd for C₂₄H₃₃N₄O₂⁺ [M+H]⁺ 409.3, found 409.4.

### Compound 69, tert-butyl (2-hydroxy-3-(3-(prop-2-yn-1-yl)ureido)propyl)(1-((R)-1-(naphthalen-1-yl)ethyl)piperidin-4-yl)carbamate

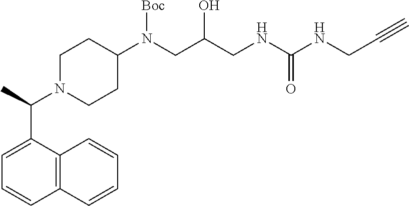 [SMILES: C#CCNC(=O)NCC(O)CN(C(=O)OC(C)(C)C)C1CCN([C@H](C)c2cccc3ccccc23)CC1]

HPLC (Method 1) RT=4.46 min, Purity=96.4%, MS (ESI+) m/z calcd for C₂₉H₄₁N₄O₄⁺ [M+H]⁺ 509.3, found 509.5.

### Compound 70, 2-(N-(1-((R)-1-(naphthalen-1-yl)ethyl)piperidin-4-yl)methylsulfonamido)-N—((S)-2-oxo-1-(prop-2-yn-1-yl)pyrrolidin-3-yl)acetamide

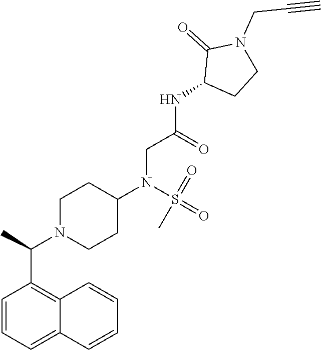 [SMILES: C#CCN1CC[C@H](NC(=O)CN(C2CCN([C@H](C)c3cccc4ccccc34)CC2)S(C)(=O)=O)C1=O]

HPLC (Method 1) RT=4.12 min, Purity >99.9%, MS (ESI+) m/z calcd for C₂₇H₃₅N₄O₄S⁺ [M+H]⁺ 511.2, found 511.4.

### Compound 71, (R)—N-methyl-2-(2-(N-(1-(1-(naphthalen-1-yl)ethyl)piperidin-4-yl)methylsulfonamido)acetamido)-N-(prop-2-yn-1-yl)acetamide

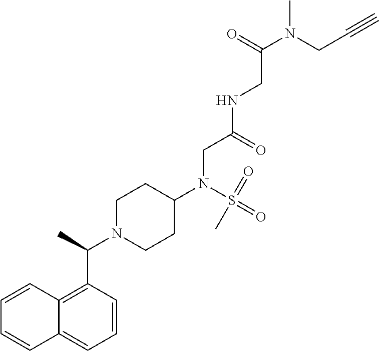 [SMILES: C#CCN(C)C(=O)CNC(=O)CN(C1CCN([C@H](C)c2cccc3ccccc23)CC1)S(C)(=O)=O]

HPLC (Method 1) RT=4.11 min, Purity >99.9%, MS (ESI+) m/z calcd for C₂₆H₃₅N₄O₄S⁺ [M+H]⁺ 499.2, found 499.4.

### Compound 72, (R)—N-(2-methylbut-3-yn-2-yl)-2-(2-(N-(1-(1-(naphthalen-1-yl)ethyl)piperidin-4-yl)methylsulfonamido)acetamido)acetamide

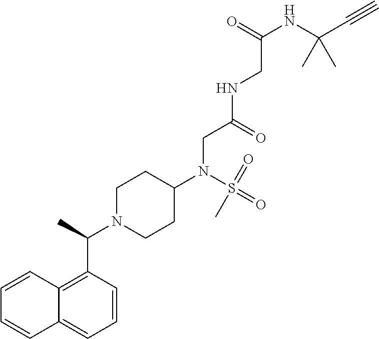 [SMILES: C#CC(C)(C)NC(=O)CNC(=O)CN(C1CCN([C@H](C)c2cccc3ccccc23)CC1)S(C)(=O)=O]

HPLC (Method 1) RT=4.24 min, Purity >99.9%, MS (ESI+) m/z calcd for C₂₇H₃₇N₄O₄S⁺ [M+H]⁺ 513.3, found 513.4.

### Compound 73, (R)—N-(2-(2-(N-(1-(1-(naphthalen-1-yl)ethyl)piperidin-4-yl)methylsulfonamido)acetamido)ethyl)propiolamide

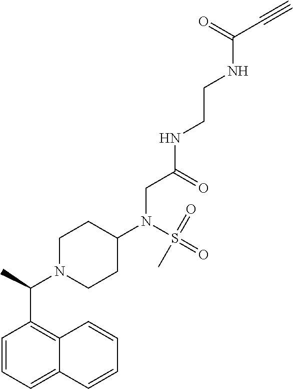 [SMILES: C#CC(=O)NCCNC(=O)CN(C1CCN([C@H](C)c2cccc3ccccc23)CC1)S(C)(=O)=O]

HPLC (Method 1) RT=3.94 min, Purity >99.9%, MS (ESI+) m/z calcd for C₂₅H₃₃N₄O₄S⁺ [M+H]⁺ 485.2, found 485.4.

### Compound 74, (R)—N-(2-(2-(N-(1-(1-(naphthalen-1-yl)ethyl)piperidin-4-yl)methylsulfonamido)acetamido)ethyl)cinnamamide

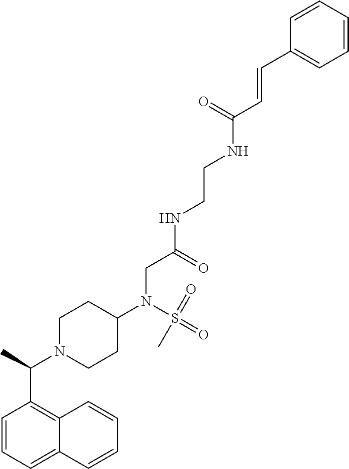 [SMILES: C[C@H](c1cccc2ccccc12)N1CCC(N(CC(=O)NCCNC(=O)/C=C/c2ccccc2)S(C)(=O)=O)CC1]

HPLC (Method 1) RT=4.59 min, Purity >99.9%, MS (ESI+) m/z calcd for C₃₁H₃₉N₄O₄S⁺ [M+H]⁺ 563.3, found 563.4.

### Compound 75, (R)—N-(1-ethynylcyclopropyl)-2-(2-(N-(1-(1-(naphthalen-1-yl)ethyl)piperidin-4-y)methylsulfonamido)acetamido)acetamide

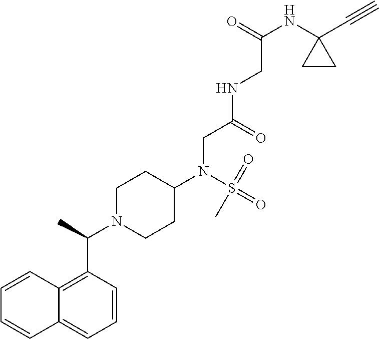 [SMILES: C#CC1(NC(=O)CNC(=O)CN(C2CCN([C@H](C)c3cccc4ccccc34)CC2)S(C)(=O)=O)CC1]

HPLC (Method 1) RT=4.12 min, Purity >99.9%, MS (ESI+) m/z calcd for C₂₇H₃₅N₄O₄S⁺ [M+H]⁺ 511.2, found 511.4.

### Compound 76, (R)-1-((R)—N-(methylsulfonyl)-N-(1-(1-(naphthalen-1-yl)ethyl)piperidin-4-yl)glycyl)-N-(prop-2-yn-1-yl)azetidine-3-carboxamide

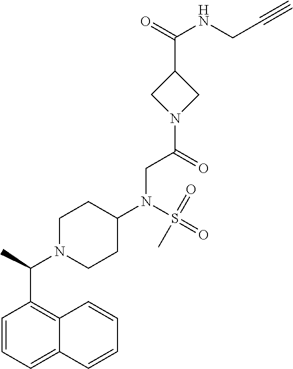 [SMILES: C#CCNC(=O)C1CN(C(=O)CN(C2CCN([C@H](C)c3cccc4ccccc34)CC2)S(C)(=O)=O)C1]

HPLC (Method 1) RT=4.03 min, Purity=99.6%, MS (ESI+) m/z calcd for C₂₇H₃₅N₄O₄S⁺ [M+H]⁺ 511.2, found 511.5.

### Compound 77, 1-(N-(methylsulfonyl)-N-(1-((R)-1-(naphthalen-1-yl)ethyl)piperidin-4-yl)glycyl)-N-(prop-2-yn-1-yl)pyrrolidine-3-carboxamide

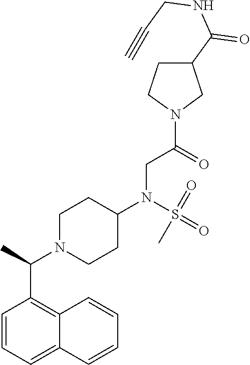 [SMILES: C#CCNC(=O)C1CCN(C(=O)CN(C2CCN([C@H](C)c3cccc4ccccc34)CC2)S(C)(=O)=O)C1]

HPLC (Method 1) RT=4.02 min, Purity >98.8%, MS (ESI+) m/z calcd for C₂₈H₃₇N₄O₄S⁺ [M+H]⁺ 525.2, found 525.4.

### Compound 78, (R)-5-(N-(1-(1-(naphthalen-1-yl)ethyl)piperidin-4-yl)methylsulfonamido)-N-(prop-2-yn-1-yl)pentanamide

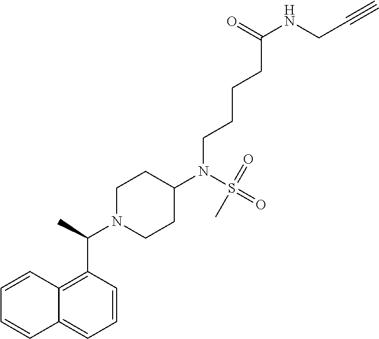 [SMILES: C#CCNC(=O)CCCCN(C1CCN([C@H](C)c2cccc3ccccc23)CC1)S(C)(=O)=O]

HPLC (Method 1) RT=4.15 min, Purity=98.2% MS (ESI+) m/z calcd for C₂₆H₃₆N₃O₃S⁺ [M+H]⁺ 470.2, found 470.4.

### Compound 79, (R)-2-((1-(1-(naphthalen-1-yl)ethyl)piperidin-4-yl)(3-oxobutyl)amino)-N-(2-oxo-2-(prop-2-yn-1-ylamino)ethyl)acetamide

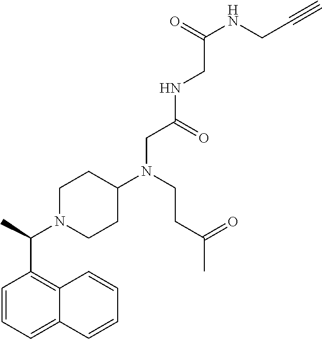 [SMILES: C#CCNC(=O)CNC(=O)CN(CCC(C)=O)C1CCN([C@H](C)c2cccc3ccccc23)CC1]

HPLC (Method 1) RT=3.83 min, Purity >99.9%, MS (ESI+) m/z calcd for C₂₈H₃₇N₄O₃⁺ [M+H]⁺ 477.3, found 477.5.

### Compound 80, (R)—N-(2-oxo-2-(prop-2-yn-1-ylamino)ethyl)-2-((1,1,1-trifluoro-N-(1-(1-(naphthalen-1-yl)ethyl)piperidin-4-yl)methyl)sulfonamido)acetamide

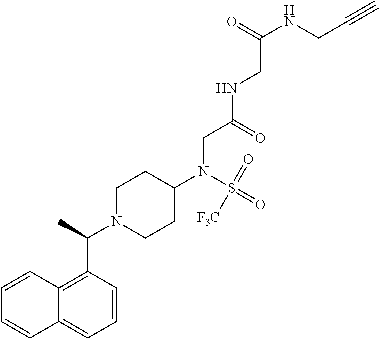 [SMILES: C#CCNC(=O)CNC(=O)CN(C1CCN([C@H](C)c2cccc3ccccc23)CC1)S(=O)(=O)C(F)(F)F]

HPLC (Method 1) RT=4.54 min, Purity >99.9%, MS (ESI+) m/z calcd for C₂₅H₃₀F₃N₄O₄S⁺ [M+H]⁺ 539.2, found 539.3.

### Compound 81, (R)-5-(N-(1-(1-(naphthalen-1-yl)ethyl)piperidin-4-yl)methylsulfonamido)-4-oxo-N-(prop-2-yn-1-yl)pentanamide

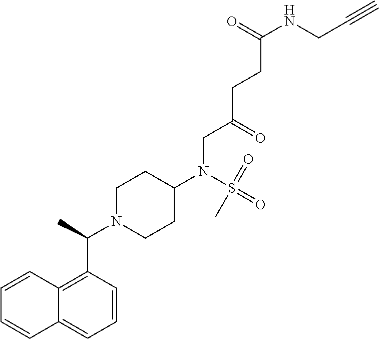 [SMILES: C#CCNC(=O)CCC(=O)CN(C1CCN([C@H](C)c2cccc3ccccc23)CC1)S(C)(=O)=O]

HPLC (Method 1) RT=4.20 min, Purity >99.9%, MS (ESI+) m/z calcd for C₂₆H₃₄N₃O₄S⁺ [M+H]⁺ 484.2, found 484.4.

### Compound 82, N-(2-oxo-2-(prop-2-yn-1-ylamino)ethyl)-2-(N-(1-(1-(quinolin-8-yl)ethyl)piperidin-4-yl)methylsulfonamido)acetamide

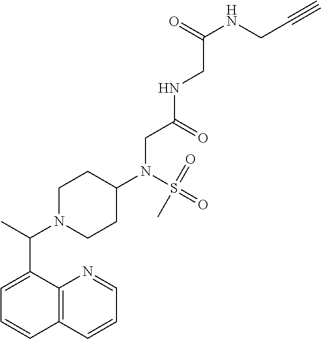 [SMILES: C#CCNC(=O)CNC(=O)CN(C1CCN(C(C)c2cccc3cccnc23)CC1)S(C)(=O)=O]

HPLC (Method 1) RT=3.59 min, Purity=97.6%, MS (ESI+) m/z calcd for C₂₄H₃₂N₅O₄S⁺ [M+H]⁺ 486.2, found 486.4.

### Compound 83, 2-(N-(1-(1-(2-fluoronaphthalen-1-yl)ethyl)piperidin-4-yl)methylsulfonamido)-N-(2-oxo-2-(prop-2-yn-1-ylamino)ethyl)acetamide

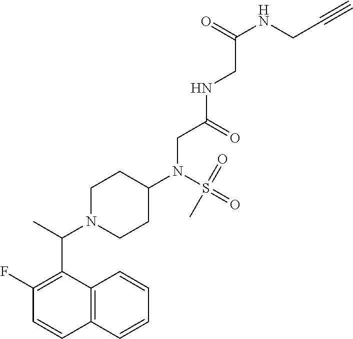 [SMILES: C#CCNC(=O)CNC(=O)CN(C1CCN(C(C)c2c(F)ccc3ccccc23)CC1)S(C)(=O)=O]

HPLC (Method 1) RT=4.09 min, Purity >99.9%, MS (ESI+) m/z calcd for C₂₅H₃₂FN₄O₄S⁺ [M+H]⁺ 503.2, found 503.3.

### Compound 84, 2-(N-(1-(1-(7-chloronaphthalen-1-yl)ethyl)piperidin-4-yl)methylsulfonamido)-N-(2-oxo-2-(prop-2-yn-1-ylamino)ethyl)acetamide

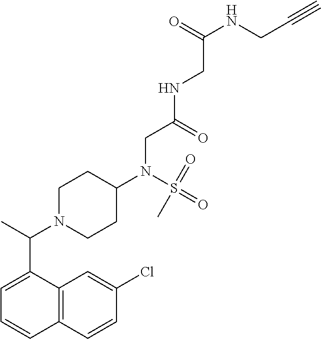 [SMILES: C#CCNC(=O)CNC(=O)CN(C1CCN(C(C)c2cccc3ccc(Cl)cc23)CC1)S(C)(=O)=O]

HPLC (Method 1) RT=4.22 min, Purity >99.9%, MS (ESI+) m/z calcd for C₂₅H₃₂ClN₄O₄S⁺ [M+H]⁺ 519.2, found 519.4.

### Compound 85, N-(2-oxo-2-(prop-2-yn-1-ylamino)ethyl)-2-(N-(1-(1-(quinolin-4-yl)ethyl)piperidin-4-yl)methylsulfonamido)acetamide

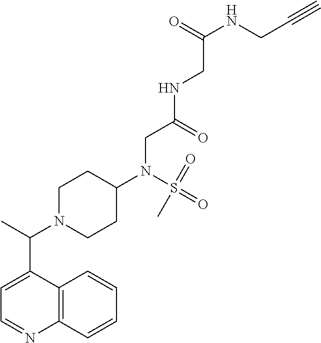 [SMILES: C#CCNC(=O)CNC(=O)CN(C1CCN(C(C)c2ccnc3ccccc23)CC1)S(C)(=O)=O]

HPLC (Method 1) RT=2.93 min, Purity >99.9%, MS (ESI+) m/z calcd for C₂₄H₃₂N₅O₄S⁺ [M+H]⁺ 486.2, found 486.4.

### Compound 86, (R)-4-((R)—N-(methylsulfonyl)-N-(1-(1-(naphthalen-1-yl)ethyl)piperidin-4-yl)glycyl)-N-(prop-2-yn-1-yl)piperazine-1-carboxamide

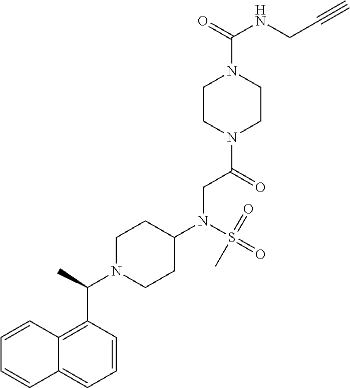 [SMILES: C#CCNC(=O)N1CCN(C(=O)CN(C2CCN([C@H](C)c3cccc4ccccc34)CC2)S(C)(=O)=O)CC1]

HPLC (Method 1) RT=4.03 min, Purity >99.9%, MS (ESI+) m/z calcd for C₂₈H₃₈N₅O₄S⁺ [M+H]⁺ 540.3, found 540.4.

### Compound 87, (R)-6-((R)—N-(methylsulfonyl)-N-(1-(1-(naphthalen-1-yl)ethyl)piperidin-4-yl)glycyl)-N-(prop-2-yn-1-yl)-2,6-diazaspiro[3.3]heptane-2-carboxamide

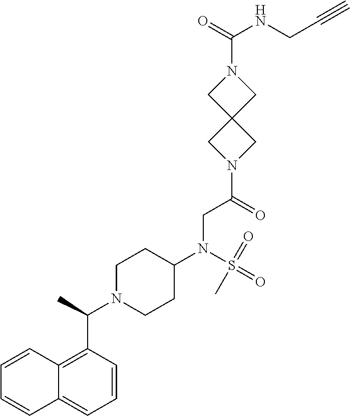 [SMILES: C#CCNC(=O)N1CC2(CN(C(=O)CN(C3CCN([C@H](C)c4cccc5ccccc45)CC3)S(C)(=O)=O)C2)C1]

HPLC (Method 1) RT=4.00 min, Purity >99.9%, MS (ESI+) m/z calcd for C₂₉H₃₈N₅O₄S⁺ [M+H]⁺ 552.3, found 552.4.

### Compound 88, 2-(N-(1-((R)-1-(naphthalen-1-yl)ethyl)piperidin-4-yl)methylsulfonamido)-N-(5-oxo-1-(prop-2-yn-1-yl)pyrrolidin-3-yl)acetamide

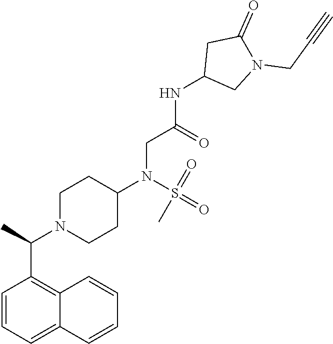 [SMILES: C#CCN1CC(NC(=O)CN(C2CCN([C@H](C)c3cccc4ccccc34)CC2)S(C)(=O)=O)CC1=O]

HPLC (Method 1) RT 3.99 min, Purity >99.9%, MS (ESI+) m/z calcd for C₂₇H₃₅N₄O₄S⁺ [M+H]⁺ 511.2, found 511.3.

### Compound 89, (S)-2-(N-(1-(1-(naphthalen-1-yl)ethyl)piperidin-4-yl)methylsulfonamido)-N-(2-oxo-2-(prop-2-yn-1-ylamino)ethyl)acetamide

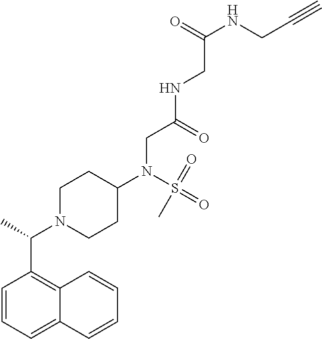 [SMILES: C#CCNC(=O)CNC(=O)CN(C1CCN([C@@H](C)c2cccc3ccccc23)CC1)S(C)(=O)=O]

HPLC (Method 1) RT=3.95 min, Purity=99.0% MS (ESI+) m/z calcd for C₂₅H₃₃N₄O₄S⁺ [M+H]⁺ 485.2, found 485.4.

### Compound 90, (S)-2-(N-(1-(1-(naphthalen-1-yl)ethyl)piperidin-4-yl)cyclopropanesulfonamido)-N-(2-oxo-2-(prop-2-yn-1-ylamino)ethyl)acetamide

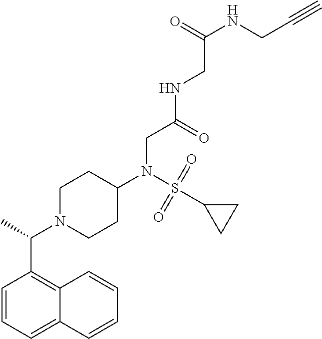 [SMILES: C#CCNC(=O)CNC(=O)CN(C1CCN([C@@H](C)c2cccc3ccccc23)CC1)S(=O)(=O)C1CC1]

HPLC (Method 1) RT=4.13 min, Purity=98.3%, MS (ESI+) m/z calcd for C₂₇H₃₅N₄O₄S⁺ [M+H]⁺ 511.2, found 511.4.

### Compound 91, (S)-2-(N-(1-(1-(naphthalen-1-yl)ethyl)piperidin-4-yl)pyrrolidine-1-sulfonamido)-N-(2-oxo-2-(prop-2-yn-1-ylamino)ethyl)acetamide

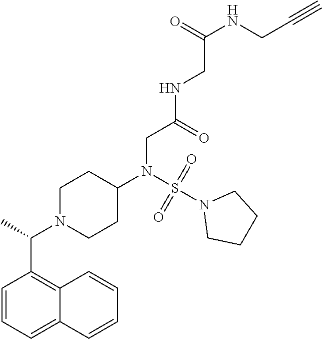 [SMILES: C#CCNC(=O)CNC(=O)CN(C1CCN([C@@H](C)c2cccc3ccccc23)CC1)S(=O)(=O)N1CCCC1]

HPLC (Method 1) RT=4.28 min, Purity=92.3%, MS (ESI+) m/z calcd for C₂₈H₃₈N₅O₄S⁺ [M+H]⁺ 540.3, found 540.5.

### Compound 92, (S)-2-(1-((R)-1-(naphthalen-1-yl)ethyl)piperidin-4-yl)-N-(2-oxo-2-(prop-2-yn-1-ylamino)ethyl)-1,2,5-thiadiazolidine-3-carboxamide 1,1-dioxide 2,2,2-trifluoroacetate

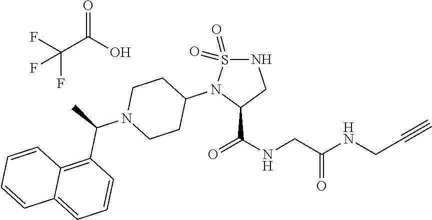

HPLC (Method 1) RT=4.02 min, Purity=97.2%, MS (ESI+) m/z calcd for C₂₅H₃₂N₅O₄S⁺ [M+H]⁺ 498.2, found 498.2.

### Compound 93, 2-(N-(3-fluoro-1-((R)-1-(naphthalen-1-yl)ethyl)piperidin-4-yl)methylsulfonamido)-N-(2-oxo-2-(prop-2-yn-1-ylamino)ethyl)acetamide

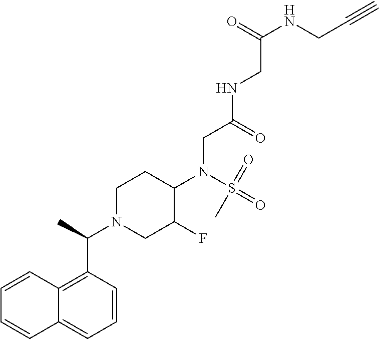 [SMILES: C#CCNC(=O)CNC(=O)CN(C1CCN([C@H](C)c2cccc3ccccc23)CC1F)S(C)(=O)=O]

HPLC (Method 1) RT=4.45 min, Purity=95.9%, MS (ESI+) m/z calcd for C₂₅H₃₂FN₄O₄S⁺ [M+H]⁺ 503.2, found 503.2.

### Compound 94, 2-(N-(1-(1-(2,3-dichlorophenyl)ethyl)piperidin-4-yl)methylsulfonamido)-N-(2-oxo-2-(prop-2-yn-1-ylamino)ethyl)acetamide

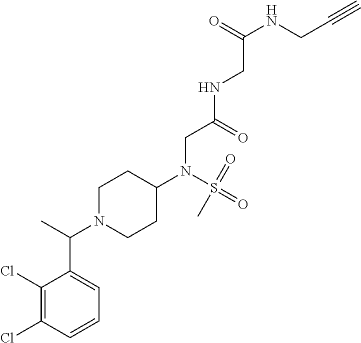 [SMILES: C#CCNC(=O)CNC(=O)CN(C1CCN(C(C)c2cccc(Cl)c2Cl)CC1)S(C)(=O)=O]

HPLC (Method 1) RT=3.92 min, Purity >99.9%, MS (ESI+) m/z calcd for C₂₁H₂₉Cl₂N₄O₄S⁺ [M+H]⁺ 503.1, found 503.1.

### Compound 95, 1-((3-(1-(1-(naphthalen-1-yl)ethyl)piperidin-4-yl)-2-oxooxazolidin-5-yl)methyl)-3-(prop-2-yn-1-yl)urea

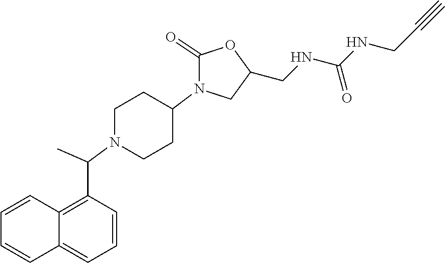 [SMILES: C#CCNC(=O)NCC1CN(C2CCN(C(C)c3cccc4ccccc34)CC2)C(=O)O1]

HPLC (Method 1) RT=3.88 min, Purity >99.9%, MS (ESI+) m/z calcd for C₂₅H₃₁N₄O₃⁺ [M+H]⁺ 435.2, found 435.2.

### Compound 96, 2-(N-(1-(1-(4-methoxynaphthalen-1-yl)ethyl)piperidin-4-yl)methylsulfonamido)-N-(2-oxo-2-(prop-2-yn-1-ylamino)ethyl)acetamide

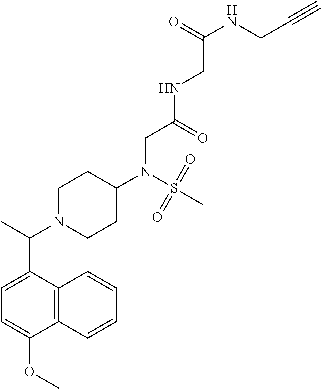 [SMILES: C#CCNC(=O)CNC(=O)CN(C1CCN(C(C)c2ccc(OC)c3ccccc23)CC1)S(C)(=O)=O]

HPLC (Method 1) RT=4.18 min, Purity=97.0% MS (ESI+) m/z calcd for C₂₆H₃₅N₄O₅S⁺ [M+H]⁺ 515.2, found 515.3.

### Compound 97, 2-(N-(1-(1-(naphthalen-1-yl)ethyl)piperidin-4-yl)methylsulfonamido)-N-(2-oxo-2-(prop-2-yn-1-ylamino)ethyl)acetamide

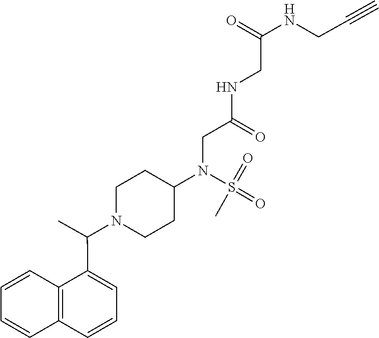 [SMILES: C#CCNC(=O)CNC(=O)CN(C1CCN(C(C)c2cccc3ccccc23)CC1)S(C)(=O)=O]

¹H NMR (400 MHz, CDCl3) δ 1.46 (d, J=8 Hz, 3H), 1.67-1.73 (m, 3H), 1.83-1.86 (m, 1H), 2.05-2.18 (m, 3H), 2.90-2.92 (m, 1H), 2.98 (s, 3H), 3.05-3.15 (m, 1H), 3.20-3.30 (m, 1H), 3.60-3.70 (m, 1H), 3.70-3.80 (m, 1H), 3.85-3.99 (m, 4H), 4.15(q, J=8 Hz, 1H), 6.83 (s, 1H), 7.06-7.07 (m, 1H), 7.43-7.55 (m, 4H), 7.74 (d, J=8 Hz, 1H), 7.85 (d, J=8 Hz, 1H), 8.35 (d, J=8 Hz, 1H).

HPLC (Method 1) RT=3.94 min, Purity=99.3%, MS (ESI+) m/z calcd for C₂₅H₃₃N₄O₄S⁺ [M+H]⁺ 485.2, found 485.2.

### Compound 98, 2-(N-(1-(1-(naphthalen-1-yl)ethyl)piperidin-4-yl)pyrrolidine-1-sulfonamido)-N-(2-oxo-2-(prop-2-yn-1-ylamino)ethyl)acetamide

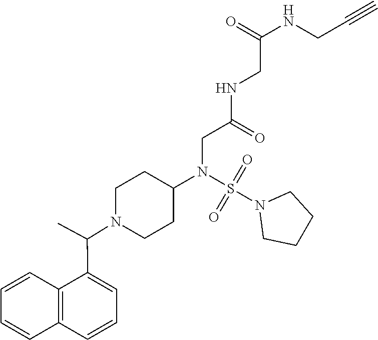 [SMILES: C#CCNC(=O)CNC(=O)CN(C1CCN(C(C)c2cccc3ccccc23)CC1)S(=O)(=O)N1CCCC1]

HPLC (Method 1) RT=4.27 min, Purity=98.8% MS (ESI+) m/z calcd for C₂₈H₃₈N₅O₄S⁺ [M+H]⁺ 540.3, found 540.2.

### Compound 99, 2-(N-(1-(1-(2,3-dimethylphenyl)ethyl)piperidin-4-yl)methylsulfonamido)-N-(2-oxo-2-(prop-2-yn-1-ylamino)ethyl)acetamide

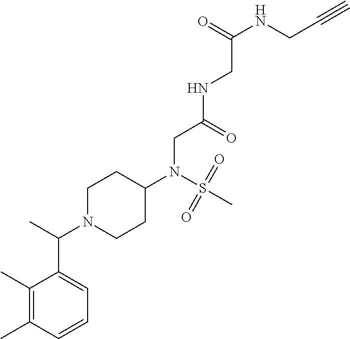 [SMILES: C#CCNC(=O)CNC(=O)CN(C1CCN(C(C)c2cccc(C)c2C)CC1)S(C)(=O)=O]

HPLC (Method 1) RT=3.81 min, Purity >99.9%, MS (ESI+) m/z calcd for C₂₃H₃₅N₄O₄S⁺ [M+H]⁺ 463.2, found 463.2.

### Compound 100, N-(2-oxo-2-(prop-2-yn-1-ylamino)ethyl)-2-(N-(1-(2,2,2-trifluoro-1-(naphthalen-1-yl)ethyl)piperidin-4-yl)methylsulfonamido)acetamide

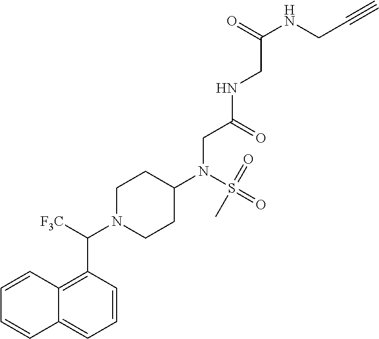 [SMILES: C#CCNC(=O)CNC(=O)CN(C1CCN(C(c2cccc3ccccc23)C(F)(F)F)CC1)S(C)(=O)=O]

HPLC (Method 1) RT=6.20 min, Purity >99.9%, MS (ESI+) m/z calcd for C₂₅H₃₀F₃N₄O₄S⁺ [M+H]⁺ 539.2, found 539.20.

### Compound 101, 2-(N-(1-(1-(2-chlorophenyl)ethyl)piperidin-4-yl)methylsulfonamido)-N-(2-oxo-2-(prop-2-yn-1-ylamino)ethyl)acetamide

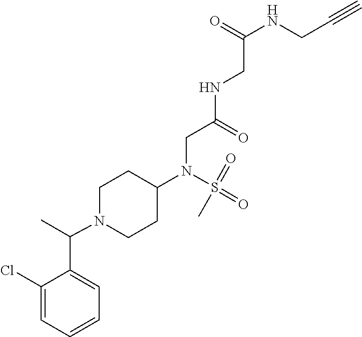 [SMILES: C#CCNC(=O)CNC(=O)CN(C1CCN(C(C)c2ccccc2Cl)CC1)S(C)(=O)=O]

HPLC (Method 1) RT=3.60 min, Purity >99.9%, MS (ESI+) m/z calcd for C₂₁H₃₀ClN₄O₄S⁺ [M+H]⁺ 469.2, found 469.3.

### Compound 102, N-(2-oxo-2-(prop-2-yn-1-ylamino)ethyl)-2-(N-(1-(1-(2-(trifluoromethyl)phenyl)ethyl)piperidin-4-yl)methylsulfonamido)acetamide

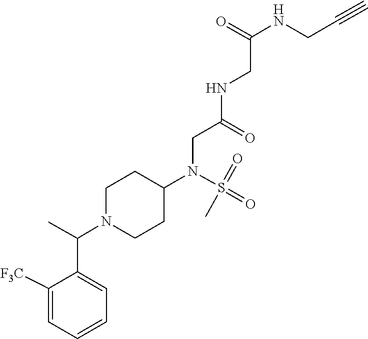 [SMILES: C#CCNC(=O)CNC(=O)CN(C1CCN(C(C)c2ccccc2C(F)(F)F)CC1)S(C)(=O)=O]

HPLC (Method 1) RT=3.88 min, Purity >99.9%, MS (ESI+) m/z calcd for C₂₂H₃₀F₃N₄O₄S⁺ [M+H]⁺ 503.2, found 503.2.

### Compound 103, 2-(N-(1-(1-(naphthalen-1-yl)propyl)piperidin-4-yl)methylsulfonamido)-N-(2-oxo-2-(prop-2-yn-1-ylamino)ethyl)acetamide

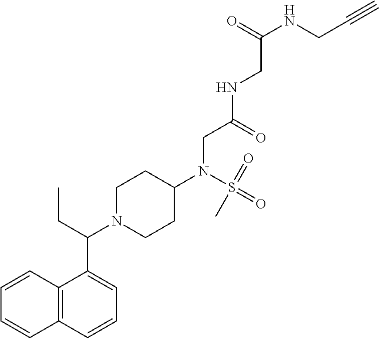 [SMILES: C#CCNC(=O)CNC(=O)CN(C1CCN(C(CC)c2cccc3ccccc23)CC1)S(C)(=O)=O]

HPLC (Method 1) RT=4.10 min, Purity >99.9%, MS (ESI+) m/z calcd for C₂₆H₃₅N₄O₄S⁺ [M+H]⁺ 499.2, found 499.2.

### Compound 104, 2-(N-(1-(cyano(naphthalen-1-yl)methyl)piperidin-4-yl)methylsulfonamido)-N-(2-oxo-2-(prop-2-yn-1-ylamino)ethyl)acetamide

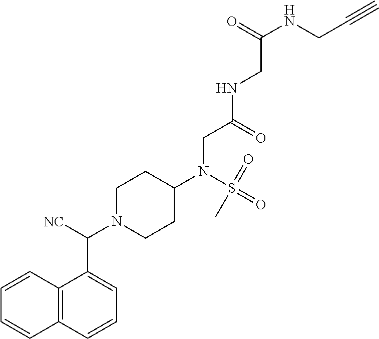 [SMILES: C#CCNC(=O)CNC(=O)CN(C1CCN(C(C#N)c2cccc3ccccc23)CC1)S(C)(=O)=O]

HPLC (Method 1) RT=5.69 min, Purity >99.9%, MS (ESI+) m/z calcd for C₂₅H₃₀NSO₄S⁺ [M+H]⁺ 496.2, found 496.0.

### Compound 105, 2-(N-(1-(1-(naphthalen-1-yl)cyclopropyl)piperidin-4-yl)methylsulfonamido)-N-(2-oxo-2-(prop-2-yn-1-ylamino)ethyl)acetamide

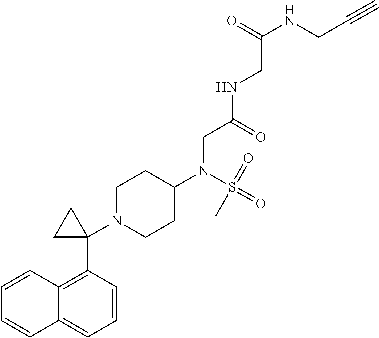 [SMILES: C#CCNC(=O)CNC(=O)CN(C1CCN(C2(c3cccc4ccccc34)CC2)CC1)S(C)(=O)=O]

HPLC (Method 1) RT=4.13 min, Purity >99.9%, MS (ESI+) m/z calcd for C₂₆H₃₃N₄O₄S⁺ [M+H]⁺ 497.2, found 497.2.

### Compound 106, (R)—N-(2-amino-2-oxoethyl)-2-(N-(1-(1-(naphthalen-1-yl)ethyl)piperidin-4-yl)methylsulfonamido)acetamide

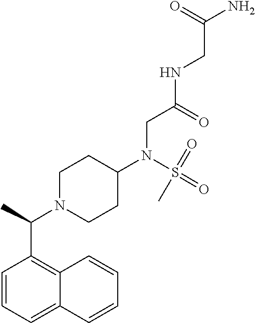 [SMILES: C[C@H](c1cccc2ccccc12)N1CCC(N(CC(=O)NCC(N)=O)S(C)(=O)=O)CC1]

HPLC (Method 1) RT=3.67 min, Purity >99.9%, MS (ESI+) m/z calcd for C₂₂H₃₁N₄O₄S⁺ [M+H]⁺ 447.2, found 447.2.

### Compound 107, 2-(N-(1-(1-(4-fluoronaphthalen-1-yl)ethyl)piperidin-4-yl)methylsulfonamido-N-(2-oxo-2-(prop-2-yn-1-ylamino)ethyl)acetamide

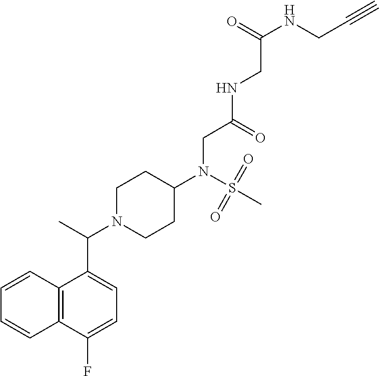 [SMILES: C#CCNC(=O)CNC(=O)CN(C1CCN(C(C)c2ccc(F)c3ccccc23)CC1)S(C)(=O)=O]

HPLC (Method 1) RT=4.09 min, Purity >99.9%, MS (ESI+) m/z calcd for C₂₅H₃₂FN₄O₄S⁺ [M+H]⁺ 503.2, found 503.2.

### Compound 108, 2-(N-(1-(1-(3-bromophenyl)ethyl)piperidin-4-yl)methylsulfonamido)-N-(2-oxo-2-(prop-2-yn-1-ylamino)ethyl)acetamide

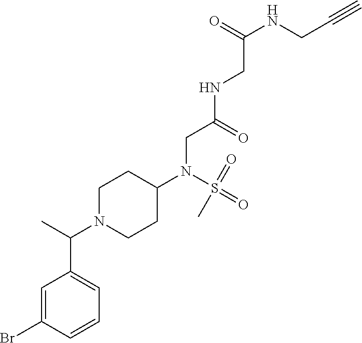 [SMILES: C#CCNC(=O)CNC(=O)CN(C1CCN(C(C)c2cccc(Br)c2)CC1)S(C)(=O)=O]

HPLC (Method 1) RT=3.78 min, Purity >99.9%, MS (ESI+) m/z calcd for C₂₁H₃₀BrN₄O₄S⁺ [M+H]⁺ 513.1, found 513.1.

### Compound 109, 2-(N-(1-(cyclopropyl(naphthalen-1-yl)methyl)piperidin-4-yl)methylsulfonamido)-N-(2-oxo-2-(prop-2-yn-1-ylamino)ethyl)acetamide

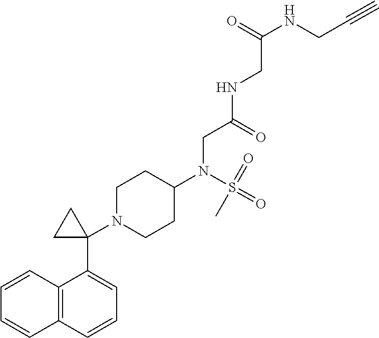 [SMILES: C#CCNC(=O)CNC(=O)CN(C1CCN(C2(c3cccc4ccccc34)CC2)CC1)S(C)(=O)=O]

HPLC (Method 1) RT=4.39 min, Purity >99.9%, MS (ESI+) m/z calcd for C₂₇H₃₅N₄O₄S⁺ [M+H]⁺ 511.2, found 511.3.

### Compound 110, 2-(N-(1-(1-(naphthalen-2-yl)ethyl)piperidin-4-yl)methylsulfonamido)-N-(2-oxo-2-(prop-2-yn-1-ylamino)ethyl)acetamide

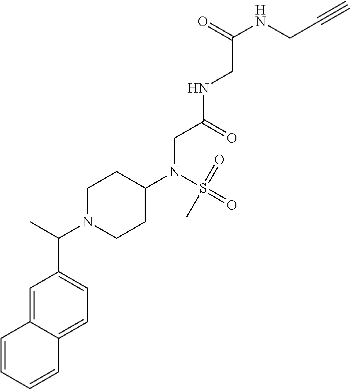 [SMILES: C#CCNC(=O)CNC(=O)CN(C1CCN(C(C)c2ccc3ccccc3c2)CC1)S(C)(=O)=O]

HPLC (Method 1) RT=4.03 min, Purity >99.9%, MS (ESI+) m/z calcd for C₂₅H₃₃N₄O₄S⁺ [M+H]⁺ 485.2, found 485.2.

### Compound 111, 2-(N-(1-(1-(isoquinolin-1-yl)ethyl)piperidin-4-yl)methylsulfonamido)-N-(2-oxo-2-(prop-2-yn-1-ylamino)ethyl)acetamide

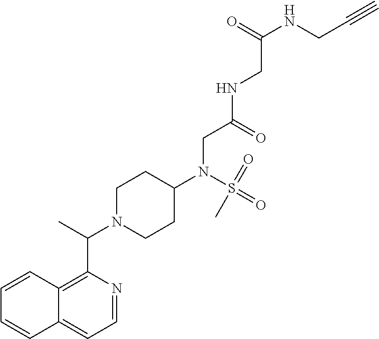 [SMILES: C#CCNC(=O)CNC(=O)CN(C1CCN(C(C)c2nccc3ccccc23)CC1)S(C)(=O)=O]

HPLC (Method 1) RT=3.71 min, Purity >99.9%, MS (ESI+) m/z calcd for C₂₄H₃₂N₅O₄S⁺ [M+H]⁺ 486.2, found 486.3.

### Compound 112, 2-(N-(1-(1-([1,1′-biphenyl]-3-yl)ethyl)piperidin-4-yl)methylsulfonamido)-N-(2-oxo-2-(prop-2-yn-1-ylamino)ethyl)acetamide

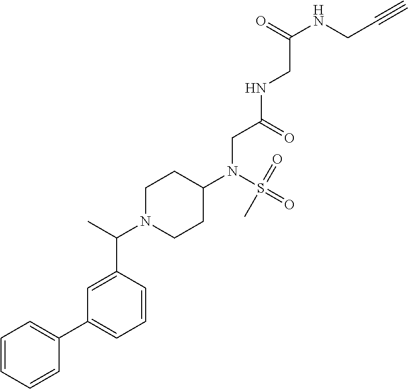 [SMILES: C#CCNC(=O)CNC(=O)CN(C1CCN(C(C)c2cccc(-c3ccccc3)c2)CC1)S(C)(=O)=O]

HPLC (Method 1) RT=4.33 min, Purity >99.9%, MS (ESI+) m/z calcd for C₂₇H₃₅N₄O₄S⁺ [M+H]⁺ 511.2, found 511.3.

### Compound 113, 2-(N-(1-(1-([1,1′-biphenyl]-2-yl)ethyl)piperidin-4-yl)methylsulfonamido)-N-(2-oxo-2-(prop-2-yn-1-ylamino)ethyl)acetamide

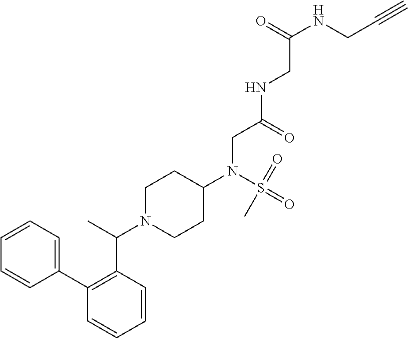 [SMILES: C#CCNC(=O)CNC(=O)CN(C1CCN(C(C)c2ccccc2-c2ccccc2)CC1)S(C)(=O)=O]

HPLC (Method 1) RT=4.23 min, Purity >99.9%, MS (ESI+) m/z calcd for C₂₇H₃₅N₄O₄S⁺ [M+H]⁺ 511.2, found 511.3.

### Compound 114, N-(cyanomethyl)-2-(2-(N-(1-(1-(naphthalen-1-yl)ethyl)piperidin-4-yl)methylsulfonamido)acetamido)acetamide

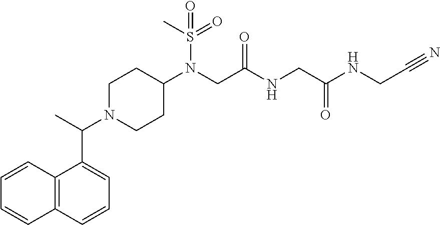 [SMILES: CC(c1cccc2ccccc12)N1CCC(N(CC(=O)NCC(=O)NCC#N)S(C)(=O)=O)CC1]

HPLC (Method 1) RT=3.88 min, Purity=95.1%, MS (ESI+) m/z calcd for C₂₄H₃₂N₅O₄S⁺ [M+H]⁺ 486.2, found 486.3.

### Compound 115, ethyl 2-(4-(N-(1-(1-(naphthalen-1-yl)ethyl)piperidin-4-yl)-N-(2-oxo-2-((2-oxo-2-(prop-2-yn-1-ylamino)ethyl)amino)ethyl)sulfamoyl)piperazin-1-yl)acetate

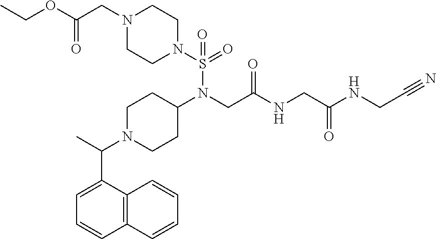 [SMILES: CCOC(=O)CN1CCN(S(=O)(=O)N(CC(=O)NCC(=O)NCC#N)C2CCN(C(C)c3cccc4ccccc34)CC2)CC1]

HPLC (Method 1) RT=3.93 min, Purity=97.1%, MS (ESI+) m/z calcd for C₃₂H₄₅N₆O₆S⁺ [M+H]⁺ 641.3, found 641.3.

### Compound 116, N-(3-fluoro-2-hydroxypropyl)-2-(2-(N-(1-(1-(naphthalen-1-yl)ethyl)piperidin-4-yl)methylsulfonamido)acetamido)acetamide

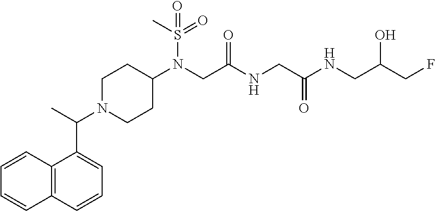 [SMILES: CC(c1cccc2ccccc12)N1CCC(N(CC(=O)NCC(=O)NCC(O)CF)S(C)(=O)=O)CC1]

HPLC (Method 1) RT=3.83 min, Purity >99.9%, MS (ESI+) m/z calcd for C₂₅H₃₆FN₄O₅S⁺ [M+H]⁺ 523.2, found 523.2.

### Compound 117, 2-(4-(N-(1-(1-(naphthalen-1-yl)ethyl)piperidin-4-yl)-N-(2-oxo-2-((2-oxo-2-(prop-2-yn-1-ylamino)ethyl)amino)ethyl)sulfamoyl)piperazin-1-yl acetic acid

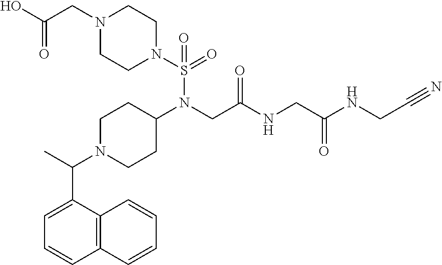 [SMILES: CC(c1cccc2ccccc12)N1CCC(N(CC(=O)NCC(=O)NCC#N)S(=O)(=O)N2CCN(CC(=O)O)CC2)CC1]

HPLC (Method 1) RT=3.68 min, Purity=96.2% MS (ESI+) m/z calcd for C₃₀H₄₁N₆O₆S⁺ [M+H]⁺ 613.3, found 613.4.

### Compound 118, 4-((1-(1-(naphthalen-1-yl)ethyl)piperidin-4-yl)(2-oxo-2-((2-oxo-2-(prop-2-yn-1-ylamino)ethyl)amino)ethyl)amino)-4-oxobutanoic acid

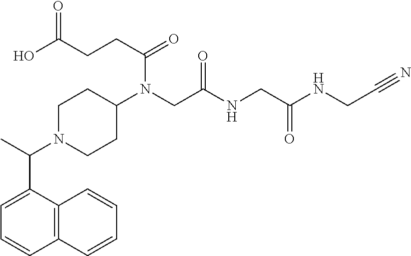 [SMILES: CC(c1cccc2ccccc12)N1CCC(N(CC(=O)NCC(=O)NCC#N)C(=O)CCC(=O)O)CC1]

HPLC (Method 1) RT=3.86 min, Purity=98.8%, MS (ESI+) m/z calcd for C₂₈H₃₅N₄O₅⁺ [M+H]⁺ 507.3, found 507.2.

### Compound 119, 2-(N-(1-(benzo[d]thiazol-2-yl)piperidin-4-yl)methylsulfonamido)-N-(2-oxo-2-(prop-2-yn-1-ylamino)ethyl)acetamide

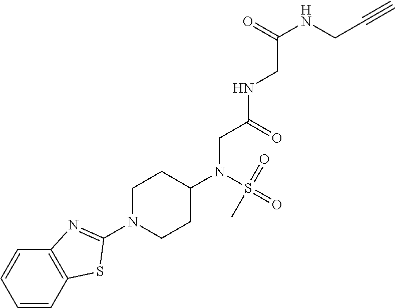 [SMILES: C#CCNC(=O)CNC(=O)CN(C1CCN(c2nc3ccccc3s2)CC1)S(C)(=O)=O]

HPLC (Method 1) RT=4.77 min, Purity >99.9%, MS (ESI+) m/z calcd for C₂₀H₂₆N₅O₄S₂⁺ [M+H]⁺ 464.1, found 464.1.

### Compound 120, 2-(N-(1-(1-(2,4-dichlorophenyl)ethyl)piperidin-4-yl)methylsulfonamido)-N-(2-oxo-2-(prop-2-yn-1-ylamino)ethyl)acetamide

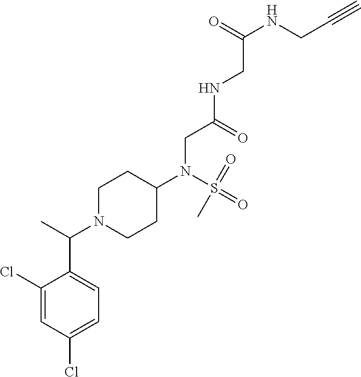 [SMILES: C#CCNC(=O)CNC(=O)CN(C1CCN(C(C)c2ccc(Cl)cc2Cl)CC1)S(C)(=O)=O]

HPLC (Method 1) RT=3.98 min, Purity >99.9%, MS (ESI+) m/z calcd for C₂₁H₂₉C₁₂N₄O₄S⁺ [M+H]⁺ 503.1, found 503.2.

### Compound 121, 2-(N-(1-(1-(3-chloro-4-methoxyphenyl)ethyl)piperidin-4-yl)methylsulfonamido)-N-(2-oxo-2-(prop-2-yn-1-ylamino)ethyl)acetamide

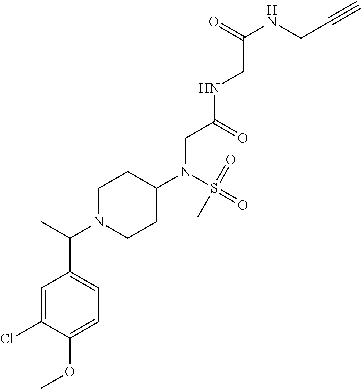 [SMILES: C#CCNC(=O)CNC(=O)CN(C1CCN(C(C)c2ccc(OC)c(Cl)c2)CC1)S(C)(=O)=O]

HPLC (Method 1) RT=3.77 min, Purity >99.9%, MS (ESI+) m/z calcd for C₂₂H₃₂ClN₄O₅S⁺ [M+H]⁺ 499.2, found 499.2.

### Compound 122, 2-(N-(1-(1-(naphthalen-1-yl)ethyl)-2-oxopiperidin-4-yl)methylsulfonamido)-N-(2-oxo-2-(prop-2-yn-1-ylamino)ethyl)acetamide

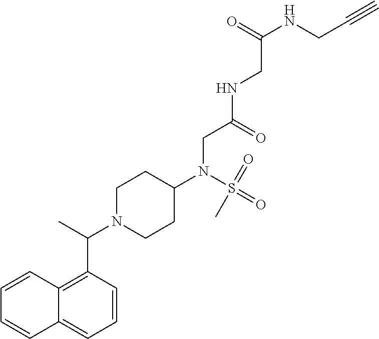 [SMILES: C#CCNC(=O)CNC(=O)CN(C1CCN(C(C)c2cccc3ccccc23)CC1)S(C)(=O)=O]

HPLC (Method 1) RT=5.26 min, Purity >99.9%, MS (ESI+) m/z calcd for C₂₅H₃₁N₄O₅⁺ [M+H]⁺ 499.2, found 499.2.

### Compound 123, 2-(N-(1-(1-(2-chloro-3-methoxyphenyl)ethyl)piperidin-4-yl)methylsulfonamido)-N-(2-oxo-2-(prop-2-yn-1-ylamino)ethyl)acetamide

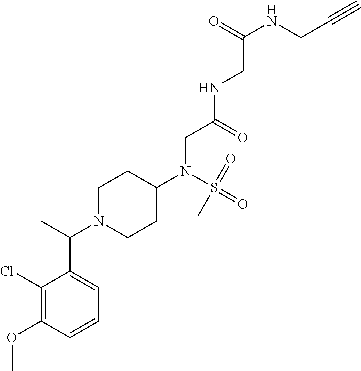 [SMILES: C#CCNC(=O)CNC(=O)CN(C1CCN(C(C)c2cccc(OC)c2Cl)CC1)S(C)(=O)=O]

HPLC (Method 1) RT=3.66 min, Purity >99.9%, MS (ESI+) m/z calcd for C₂₂H₃₂ClN₄O₅S⁺ [M+H]⁺ 499.2, found 499.2.

### Compound 124, methyl 2-(naphthalen-1-yl)-2-(4-(N-(2-oxo-2-((2-oxo-2-(prop-2-yn-1-ylamino)ethyl)amino)ethyl)methylsulfonamido)piperidin-1-yl)acetate

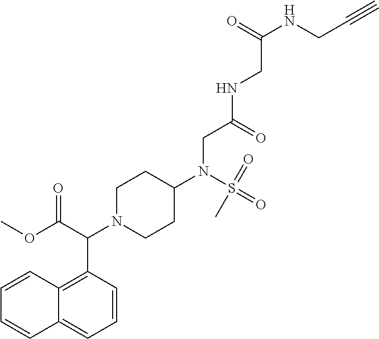 [SMILES: C#CCNC(=O)CNC(=O)CN(C1CCN(C(C(=O)OC)c2cccc3ccccc23)CC1)S(C)(=O)=O]

HPLC (Method 1) RT=4.30 min, Purity >99.9%, MS (ESI+) m/z calcd for C₂₆H₃₃N₄O₆S⁺ [M+H]⁺ 529.2, found 529.3.

### Compound 125, 2-(N-(1-(1-(2,6-dichlorophenyl)ethyl)piperidin-4-yl)methylsulfonamido)-N-(2-oxo-2-(prop-2-yn-1-ylamino)ethyl)acetamide

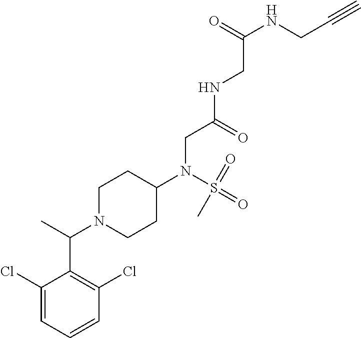 [SMILES: C#CCNC(=O)CNC(=O)CN(C1CCN(C(C)c2c(Cl)cccc2Cl)CC1)S(C)(=O)=O]

HPLC (Method 1) RT=3.58 min, Purity >99.9%, MS (ESI+) m/z calcd for C₂₁H₂₉Cl₂N₄O₄S⁺ [M+H]⁺ 503.1, found 503.1.

### Compound 126, 2-(N-(1-(5-chlorobenzo[d]thiazol-2-yl)piperidin-4-yl)methylsulfonamido)-N-(2-oxo-2-(prop-2-yn-1-ylamino)ethyl)acetamide

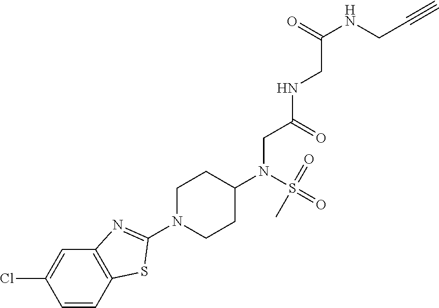 [SMILES: C#CCNC(=O)CNC(=O)CN(C1CCN(c2nc3cc(Cl)ccc3s2)CC1)S(C)(=O)=O]

HPLC (Method 1) RT=5.56 min, Purity >99.9%, MS (ESI+) m/z calcd for C₂₀H₂₅ClN₅O₄S₂⁺ [M+H]⁺ 498.1, found 498.2.

### Compound 127, (R)—N-(but-3-yn-1-yl)-2-(2-(N-(1-(1-(naphthalen-1-yl)ethyl)piperidin-4-yl)methylsulfonamido)acetamido)acetamide

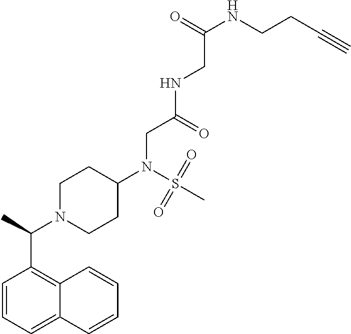 [SMILES: C#CCCNC(=O)CNC(=O)CN(C1CCN([C@H](C)c2cccc3ccccc23)CC1)S(C)(=O)=O]

HPLC (Method 1) RT=4.02 min, Purity=98.6%, MS (ESI+) m/z calcd for C₂₆H₃₅N₄O₄S⁺ [M+H]⁺ 499.2, found 499.3.

### Compound 128, N-(but-3-yn-2-yl)-2-(2-(N-(1-((R)-1-(naphthalen-1-yl)ethyl)piperidin-4-yl)methylsulfonamido)acetamido)acetamide

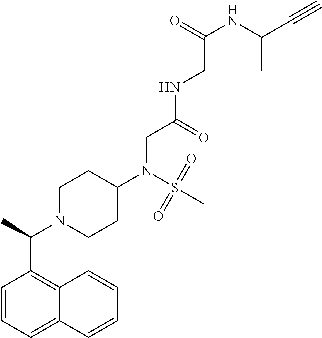 [SMILES: C#CC(C)NC(=O)CNC(=O)CN(C1CCN([C@H](C)c2cccc3ccccc23)CC1)S(C)(=O)=O]

HPLC (Method 1) RT=4.07 min, Purity >99.9%, MS (ESI+) m/z calcd for C₂₆H₃₅N₄O₄S⁺ [M+H]⁺ 499.2, found 499.3.

### Compound 129, 2-(N-(1-(1-(2-fluorophenyl)ethyl)piperidin-4-yl)methylsulfonamido)-N-(2-oxo-2-(prop-2-yn-1-ylamino)ethyl)acetamide

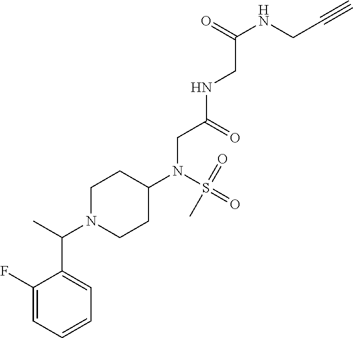 [SMILES: C#CCNC(=O)CNC(=O)CN(C1CCN(C(C)c2ccccc2F)CC1)S(C)(=O)=O]

HPLC (Method 1) RT=3.22 min, Purity >99.9%, MS (ESI+) m/z calcd for C₂₁H₃₀FN₄O₄S⁺ [M+H]⁺ 453.2, found 453.3.

### Compound 130, 2-(N-(1-(1-(2-methoxyphenyl)ethyl)piperidin-4-yl)methylsulfonamido)-N-(2-oxo-2-(prop-2-yn-1-ylamino)ethyl)acetamide

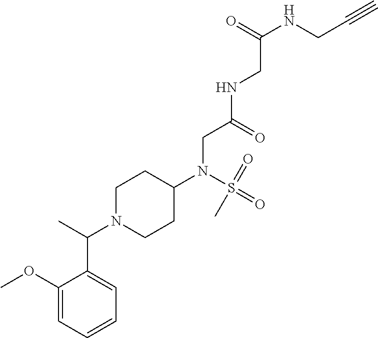 [SMILES: C#CCNC(=O)CNC(=O)CN(C1CCN(C(C)c2ccccc2OC)CC1)S(C)(=O)=O]

HPLC (Method 1) RT=3.53 min, Purity >99.9%, MS (ESI+) m/z calcd for C₂₂H₃₃N₄O₅S⁺ [M+H]⁺ 465.2, found 465.2.

### Compound 131, methyl 3-(N-(1-(1-(naphthalen-1-yl)ethyl)piperidin-4-yl)-N-(2-oxo-2-((2-oxo-2-(prop-2-yn-1-ylamino)ethyl)amino)ethyl)sulfamoyl)propanoate

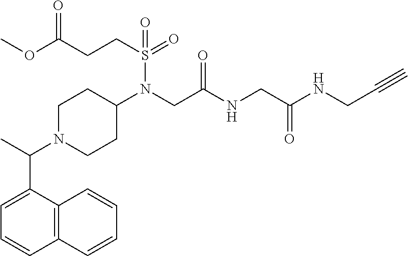 [SMILES: C#CCNC(=O)CNC(=O)CN(C1CCN(C(C)c2cccc3ccccc23)CC1)S(=O)(=O)CCC(=O)OC]

HPLC (Method 1) RT=4.21 min, Purity=97.6%, MS (ESI+) m/z calcd for C₂₈H₃₇N₄O₆S⁺ [M+H]⁺ 557.2, found 55.3.

### Compound 132, N-(3-fluoro-2-oxopropyl)-2-(2-(N-(1-(1-(naphthalen-1-yl)ethyl)piperidin-4-yl)methylsulfonamido)acetamido)acetamide

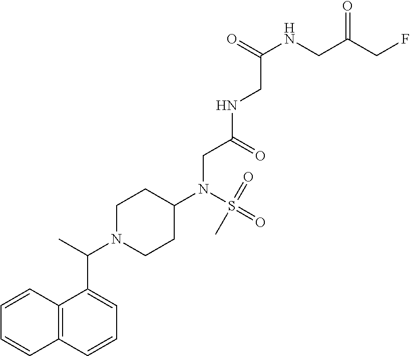 [SMILES: CC(c1cccc2ccccc12)N1CCC(N(CC(=O)NCC(=O)NCC(=O)CF)S(C)(=O)=O)CC1]

HPLC (Method 1) RT=3.87 min, Purity=96.8% MS (ESI+) m/z calcd for C₂₅H₃₄FN₄O₅S⁺ [M+H]⁺ 521.2, found 521.2.

### Compound 133, JL-02-140, (R)-3-(N-(1-(1-(naphthalen-1-yl)ethyl)piperidin-4-yl)-N-(2-oxo-2-((2-oxo-2-(prop-2-yn-1-ylamino)ethyl)amino)ethyl)sulfamoyl)propanamide

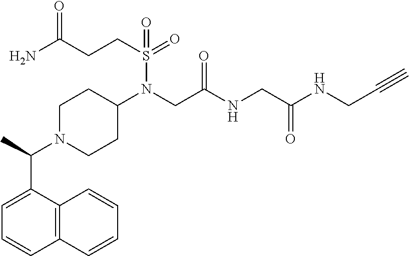 [SMILES: C#CCNC(=O)CNC(=O)CN(C1CCN([C@H](C)c2cccc3ccccc23)CC1)S(=O)(=O)CCC(N)=O]

HPLC (Method 1) RT=3.85 min, Purity=96.4%, MS (ESI+) m/z calcd for C₂₇H₃₆N₅O₅S⁺ [M+H]⁺ 542.2, found 542.3.

### Compound 134, methyl 1-(N-(1-((R)-1-(naphthalen-1-yl)ethyl)piperidin-4-yl)-N-(2-oxo-2-((2-oxo-2-(prop-2-yn-1-ylamino)ethyl)amino)ethyl)sulfamoyl)pyrrolidine-3-carboxylate

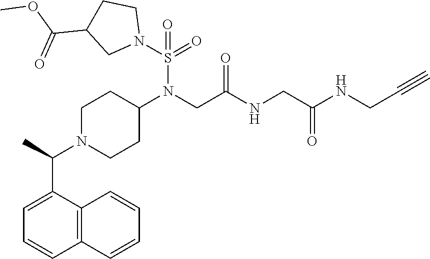 [SMILES: C#CCNC(=O)CNC(=O)CN(C1CCN([C@H](C)c2cccc3ccccc23)CC1)S(=O)(=O)N1CCC(C(=O)OC)C1]

HPLC (Method 1) RT=4.03 min, Purity >99.9%, MS (ESI+) m/z calcd for C₃₀H₄₀NSO₆S⁺ [M+H]⁺ 598.3, found 598.3.

### Compound 135, (R)-2-((3-hydroxy-N-(1-(1-(naphthalen-1-yl)ethyl)piperidin-4-yl)propyl)sulfonamido)-N-(2-oxo-2-(prop-2-yn-1-ylamino)ethyl)acetamide

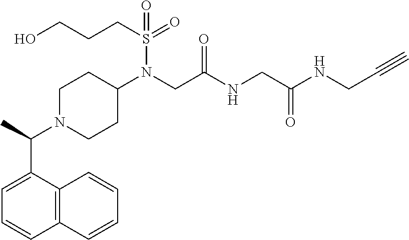 [SMILES: C#CCNC(=O)CNC(=O)CN(C1CCN([C@H](C)c2cccc3ccccc23)CC1)S(=O)(=O)CCCO]

HPLC (Method 1) RT=3.91 min, Purity >99.9%, MS (ESI+) m/z calcd for C₂₇H₃₇N₄O₅S⁺ [M+H]⁺ 529.2, found 529.3.

### Compound 136, methyl (E)-4-(5-((2-(N-(1-(1-(naphthalen-1-yl)ethyl)piperidin-4-yl)methylsulfonamido)acetamido)methyl)-1,3,4-oxadiazole-2-carboxamido)but-2-enoate

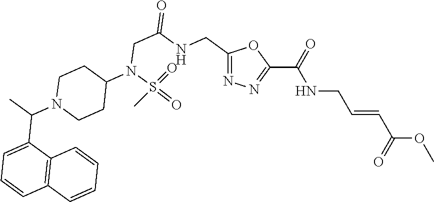 [SMILES: COC(=O)/C=C/CNC(=O)c1nnc(CNC(=O)CN(C2CCN(C(C)c3cccc4ccccc34)CC2)S(C)(=O)=O)o1]

HPLC (Method 1) RT=4.14 min, Purity=99.2%, MS (ESI+) m/z calcd for C₂₉H₃₇N₆O₇S⁺ [M+H]⁺ 613.2, found 613.3.

### Compound 137, 1-(N-(1-((R)-1-(naphthalen-1-yl)ethyl)piperidin-4-yl)-N-(2-oxo-2-((2-oxo-2-(prop-2-yn-1-ylamino)ethyl)amino)ethyl)sulfamoyl)pyrrolidine-3-carboxamide

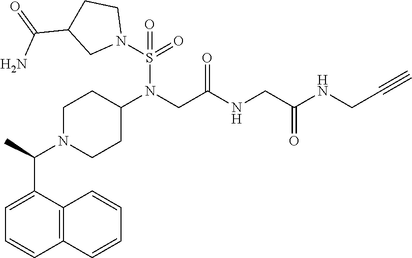 [SMILES: C#CCNC(=O)CNC(=O)CN(C1CCN([C@H](C)c2cccc3ccccc23)CC1)S(=O)(=O)N1CCC(C(N)=O)C1]

HPLC (Method 1) RT=3.95 min, Purity >99.9%, MS (ESI+) m/z calcd for C₂₉H₃₉N₆O₅S⁺ [M+H]⁺ 583.3, found 583.4.

### Compound 138, 2-(naphthalen-1-yl)-2-(4-(N-(2-oxo-2-((2-oxo-2-(prop-2-yn-1-ylamino)ethyl)amino)ethyl)methylsulfonamido)piperidin-1-yl)acetic acid

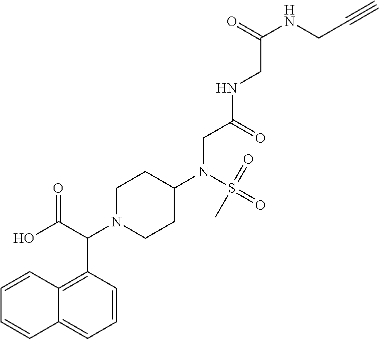 [SMILES: C#CCNC(=O)CNC(=O)CN(C1CCN(C(C(=O)O)c2cccc3ccccc23)CC1)S(C)(=O)=O]

HPLC (Method 1) RT=3.97 min, Purity >99.9%, MS (ESI+) m/z calcd for C₂₅H₃₁N₄O₆S⁺ [M+H]⁺ 515.2, found 515.3.

### Compound 139, 2-(N-(1-(1-(2-chloro-4-methoxyphenyl)ethyl)piperidin-4-yl)methylsulfonamido)-N-(2-oxo-2-(prop-2-yn-1-ylamino)ethyl)acetamide

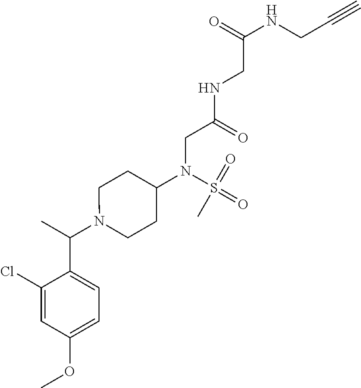 [SMILES: C#CCNC(=O)CNC(=O)CN(C1CCN(C(C)c2ccc(OC)cc2Cl)CC1)S(C)(=O)=O]

HPLC (Method 1) RT=3.79 min, Purity >99.9%, MS (ESI+) m/z calcd for C₂₂H₃₂ClN₄O₅S⁺ [M+H]⁺ 499.2, found 499.2.

### Compound 140, 2-(N-(1-(1-(2,5-dichlorophenyl)ethyl)piperidin-4-yl)methylsulfonamido)-N-(2-oxo-2-(prop-2-yn-1-ylamino)ethyl)acetamide

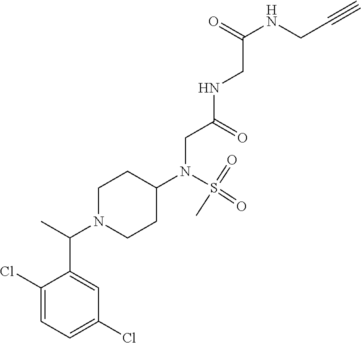 [SMILES: C#CCNC(=O)CNC(=O)CN(C1CCN(C(C)c2cc(Cl)ccc2Cl)CC1)S(C)(=O)=O]

HPLC (Method 1) RT=3.79 min, Purity >99.9%, MS (ESI+) m/z calcd for C₂₁H₂₉Cl₂N₄O₄S⁺ [M+H]⁺ 503.1, found 503.2.

### Compound 141, N-(2-oxo-2-(prop-2-yn-1-ylamino)ethyl)-2-(N-(1-(1-(3-(thiophen-2-yl)phenyl)ethyl)piperidin-4-yl)methylsulfonamido)acetamide

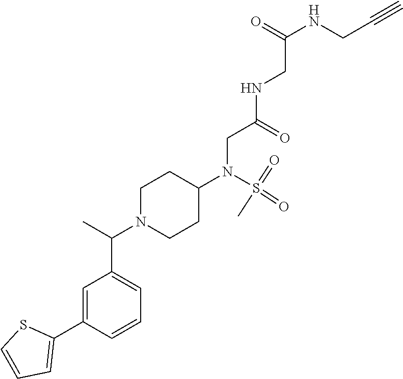 [SMILES: C#CCNC(=O)CNC(=O)CN(C1CCN(C(C)c2cccc(-c3cccs3)c2)CC1)S(C)(=O)=O]

HPLC (Method 1) RT=4.20 min, Purity >99.9%, MS (ESI+) m/z calcd for C₂₅H₃₃N₄O₄S₂⁺ [M+H]⁺ 517.2, found 517.3.

### Compound 142, 2-(N-(1-(1-(2-chloro-3-methylphenyl)ethyl)piperidin-4-yl)methylsulfonamido)-N-(2-oxo-2-(prop-2-yn-1-ylamino)ethyl)acetamide

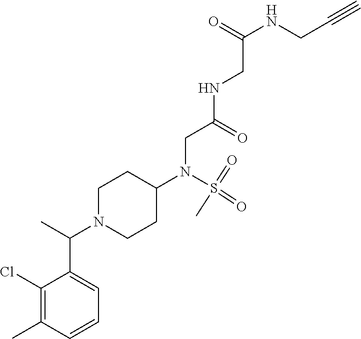 [SMILES: C#CCNC(=O)CNC(=O)CN(C1CCN(C(C)c2cccc(C)c2Cl)CC1)S(C)(=O)=O]

HPLC (Method 1) RT=3.84 min, Purity >99.9%, MS (ESI+) m/z calcd for C₂₂H₃₂ClN₄O₄S⁺ [M+H]⁺ 483.2, found 483.3.

### Compound 143, 2-(N-(1-(2-hydroxy-1-(naphthalen-1-yl)ethyl)piperidin-4-yl)methylsulfonamido)-N-(2-oxo-2-(prop-2-yn-1-ylamino)ethyl)acetamide

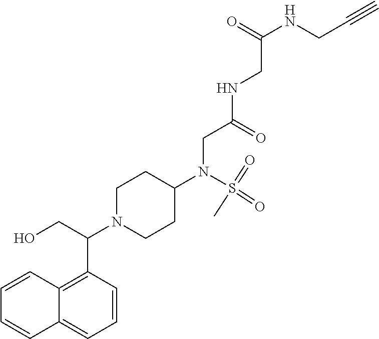 [SMILES: C#CCNC(=O)CNC(=O)CN(C1CCN(C(CO)c2cccc3ccccc23)CC1)S(C)(=O)=O]

HPLC (Method 1) RT=3.68 min, Purity >99.9%, MS (ESI+) m/z calcd for C₂₅H₃₃N₄O₅S⁺ [M+H]⁺ 501.2, found 501.2.

### Compound 144, KHS_NB6_066, 2-(N-(1-(2-fluoro-1-(naphthalen-1-yl)ethyl)piperidin-4-yl)methylsulfonamido)-N-(2-oxo-2-(prop-2-yn-1-ylamino)ethyl)acetamide

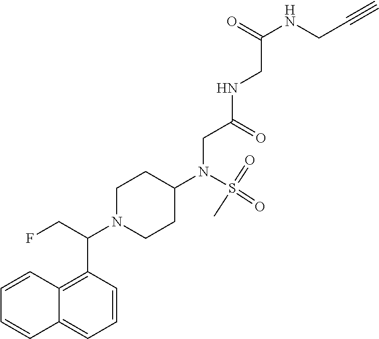 [SMILES: C#CCNC(=O)CNC(=O)CN(C1CCN(C(CF)c2cccc3ccccc23)CC1)S(C)(=O)=O]

HPLC (Method 1) RT=4.05 min, Purity >99.9%, MS (ESI+) m/z calcd for C₂₅H₃₂FN₄O₄S⁺ [M+H]⁺ 503.2, found 503.2.

### Compound 145, (R)-2-((1-(1-(naphthalen-1-yl)ethyl)piperidin-4-yl)(sulfamoyl)amino)-N-(2-oxo-2-(prop-2-yn-1-ylamino)ethyl)acetamide 2,2,2-trifluoroacetate

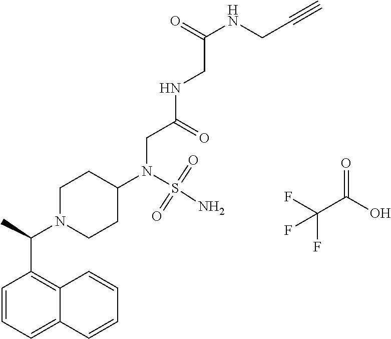 [SMILES: C#CCNC(=O)CNC(=O)CN(C1CCN([C@H](C)c2cccc3ccccc23)CC1)S(N)(=O)=O ; O=C(O)C(F)(F)F]

HPLC (Method 1) RT=3.84 min, Purity >99.9%, MS (ESI+) m/z calcd for C₂₄H₃₂N₅O₄S⁺ [M+H]⁺ 486.2, found 486.3.

### Compound 146, (R)—N-(3-cyano-2-oxopropyl)-2-(2-(N-(1-(1-(naphthalen-1-yl)ethyl)piperidin-4-yl)methylsulfonamido)acetamido)acetamide

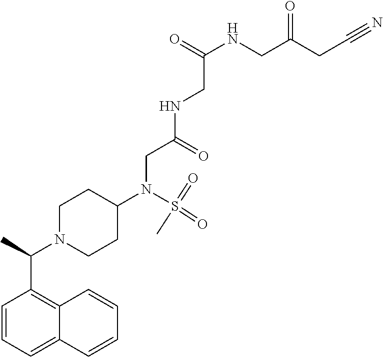 [SMILES: C[C@H](c1cccc2ccccc12)N1CCC(N(CC(=O)NCC(=O)NCC(=O)CC#N)S(C)(=O)=O)CC1]

HPLC (Method 1) RT=3.90 min, Purity >99.9%, MS (ESI+) m/z calcd for C₂₆H₃₄N₅O₅S⁺ [M+H]⁺ 528.2, found 528.3.

### Compound 147, (R)—N-(1-(1-(naphthalen-1-yl)ethyl)piperidin-4-yl)-N-(2-oxo-2-(2-propioloylhydrazineyl)ethyl)methanesulfonamide

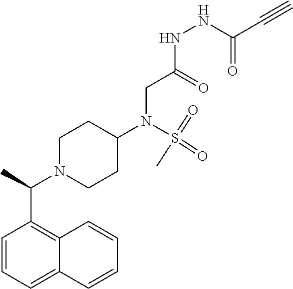 [SMILES: C#CC(=O)NNC(=O)CN(C1CCN([C@H](C)c2cccc3ccccc23)CC1)S(C)(=O)=O]

HPLC (Method 1) RT=3.84 min, Purity >99.9%, MS (ESI+) m/z calcd for C₂₃H₂₉N₄O₄S⁺ [M+H]⁺ 457.2, found 457.2.

### Compound 148, 2-(N-(1-(2,2-difluoro-1-(naphthalen-1-yl)ethyl)piperidin-4-yl)methylsulfonamido)-N-(2-oxo-2-(prop-2-yn-1-ylamino)ethyl)acetamide

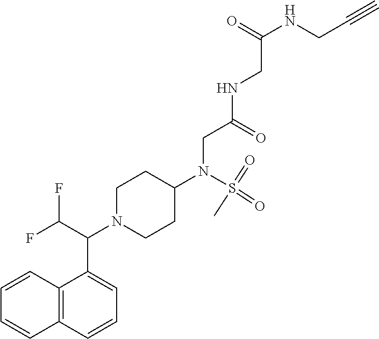 [SMILES: C#CCNC(=O)CNC(=O)CN(C1CCN(C(c2cccc3ccccc23)C(F)F)CC1)S(C)(=O)=O]

HPLC (Method 1) RT=5.00 min, Purity >99.9%, MS (ESI+) m/z calcd for C₂₅H₃₁F₂N₄O₄S⁺ [M+H]⁺ 521.2, found 521.3.

### Compound 149, 5-((2-(N-(1-(1-(naphthalen-1-yl)ethyl)piperidin-4-yl)methylsulfonamido)acetamido)methyl)-N-(prop-2-yn-1-yl)-1,3,4-oxadiazole-2-carboxamide

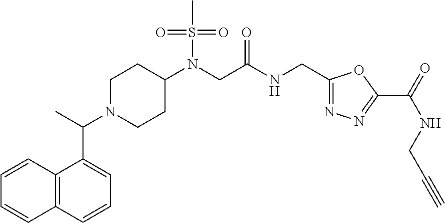 [SMILES: C#CCNC(=O)c1nnc(CNC(=O)CN(C2CCN(C(C)c3cccc4ccccc34)CC2)S(C)(=O)=O)o1]

HPLC (Method 1) RT=4.00 min, Purity=99.2%, MS (ESI+) m/z calcd for C₂₇H₃₃N₆O₅S⁺ [M+H]⁺ 553.2, found 553.3.

### Compound 150, N-((1,3,4-oxadiazol-2-yl)methyl)-2-(N-(1-(1-(naphthalen-1-yl)ethyl)piperidin-4-yl)methylsulfonamido)acetamide

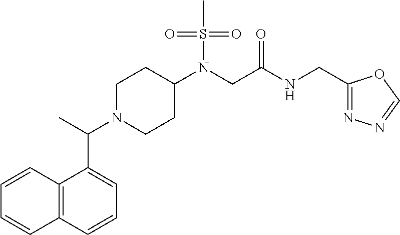 [SMILES: CC(c1cccc2ccccc12)N1CCC(N(CC(=O)NCc2nnco2)S(C)(=O)=O)CC1]

HPLC (Method 1) RT=3.79 min, Purity=99.2%, MS (ESI+) m/z calcd for C₂₃H₃₀N₅O₄S⁺ [M+H]⁺ 472.2, found 472.2.

### Compound 151, 2-(N-(1-(1-(naphthalen-1-yl)ethyl-2,2,2-d3)piperidin-4-yl)methylsulfonamido)-N-(2-oxo-2-(prop-2-yn-1-ylamino)ethyl)acetamide

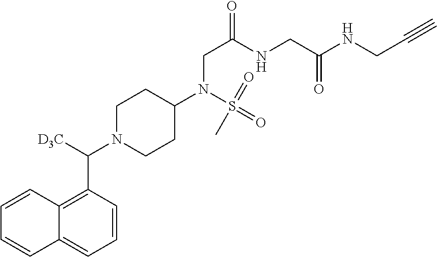 [SMILES: [2H]C([2H])([2H])C(c1cccc2ccccc12)N1CCC(N(CC(=O)NCC(=O)NCC#C)S(C)(=O)=O)CC1]

HPLC (Method 1) RT=3.95 min, Purity=97.0% MS (ESI+) m/z calcd for C₂₅H₃₀D₃N₄O₄S⁺ [M+H]⁺ 488.2, found 488.3.

### Compound 152, (R)-2-(N-(1-((R)-1-(naphthalen-1-yl)ethyl)piperidin-4-yl)methylsulfonamido)-N-(2-oxo-2-(prop-2-yn-1-ylamino)ethyl)propanamide

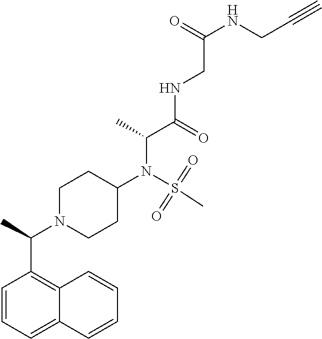 [SMILES: C#CCNC(=O)CNC(=O)[C@@H](C)N(C1CCN([C@H](C)c2cccc3ccccc23)CC1)S(C)(=O)=O]

HPLC (Method 1) RT=3.99 min, Purity=99.1%, MS (ESI+) m/z calcd for C₂₆H₃₅N₄O₄S⁺ [M+H]⁺ 499.2, found 499.3.

### Compound 153, 2-(N-(1-(1-(naphthalen-1-yl)ethyl)piperidin-4-yl)methylsulfonamido)-N-(2-oxo-2-((prop-2-yn-1-yl-1,l-d2)amino)ethyl)acetamide

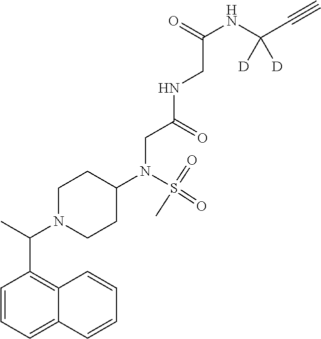 [SMILES: [2H]C([2H])(C#C)NC(=O)CNC(=O)CN(C1CCN(C(C)c2cccc3ccccc23)CC1)S(C)(=O)=O]

HPLC (Method 1) RT=3.95 min, Purity >99.9%, MS (ESI+) m/z calcd for C₂₅H₃₁D2N₄O₄S⁺ [M+H]⁺ 487.2, found 487.3.

### Compound 154, N-(2,2-dimethoxyethyl)-2-(2-(N-(1-(1-(naphthalen-1-yl)ethyl)piperidin-4-yl)methylsulfonamido)acetamido)acetamide

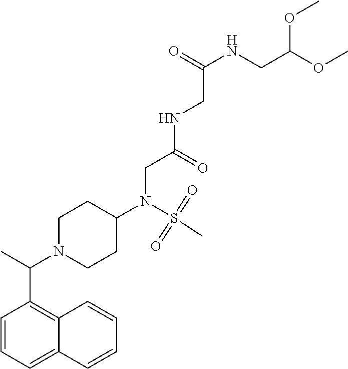 [SMILES: COC(CNC(=O)CNC(=O)CN(C1CCN(C(C)c2cccc3ccccc23)CC1)S(C)(=O)=O)OC]

HPLC (Method 1) RT=3.96 min, Purity >99.9%, MS (ESI+) m/z calcd for C₂₆H₃₉N₄O₆S⁺ [M+H]⁺ 535.3, found 535.3.

### Compound 155, N-(2-methylpent-4-yn-2-yl)-2-(2-(N-(1-(1-(naphthalen-1-yl)ethyl)piperidin-4-yl)methylsulfonamido)acetamido)acetamide

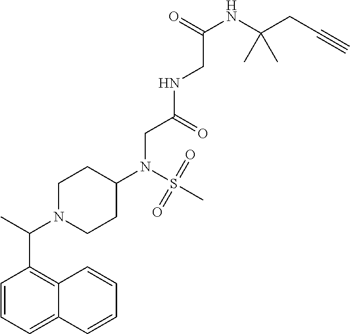 [SMILES: C#CCC(C)(C)NC(=O)CNC(=O)CN(C1CCN(C(C)c2cccc3ccccc23)CC1)S(C)(=O)=O]

HPLC (Method 1) RT=4.39 min, Purity >99.9%, MS (ESI+) m/z calcd for C₂₈H₃₉N₄O₄S⁺ [M+H]⁺ 527.3, found 527.3.

### Compound 156, N-(2,2-dimethylbut-3-yn-1-yl)-2-(2-(N-(1-(1-(naphthalen-1-yl)ethyl)piperidin-4-yl)methylsulfonamido)acetamido)acetamide

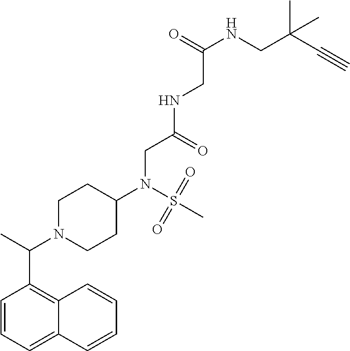 [SMILES: C#CC(C)(C)CNC(=O)CNC(=O)CN(C1CCN(C(C)c2cccc3ccccc23)CC1)S(C)(=O)=O]

HPLC (Method 1) RT=4.34 min, Purity >99.9%, MS (ESI+) m/z calcd for C₂₈H₃₉N₄O₄S⁺ [M+H]⁺ 527.3, found 527.3.

### Compound 157, N-(but-3-yn-1-yl)-2-(2-(N-(1-(1-(naphthalen-1-yl)ethyl)piperidin-4-yl)methylsulfonamido)acetamido)acetamide

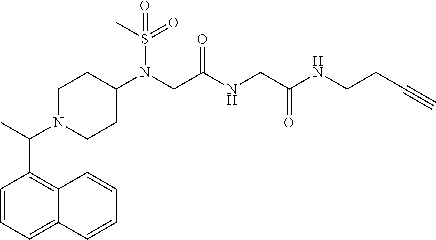 [SMILES: C#CCCNC(=O)CNC(=O)CN(C1CCN(C(C)c2cccc3ccccc23)CC1)S(C)(=O)=O]

HPLC (Method 1) RT=4.05 min, Purity=96.8% MS (ESI+) m/z calcd for C₂₆H₃₅N₄O₄S⁺ [M+H]⁺ 499.2, found 499.2.

### Compound 158, N-(2,2-diethoxyethyl-1,1-d2)-2-(2-(N-(1-(1-(naphthalen-1-yl)ethyl)piperidin-4-yl)methylsulfonamido)acetamido)acetamide

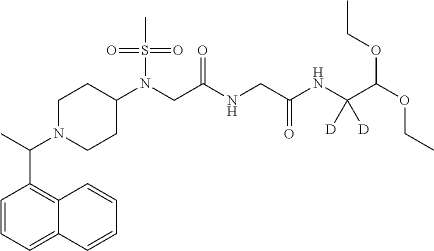 [SMILES: [2H]C([2H])(NC(=O)CNC(=O)CN(C1CCN(C(C)c2cccc3ccccc23)CC1)S(C)(=O)=O)C(OCC)OCC]

HPLC (Method 1) RT=4.21 min, Purity >99.9%, MS (ESI+) m/z calcd for C₂₈H₄₁D₂N₄O₆S⁺ [M+H]⁺ 565.3, found 565.2.

### Compound 159, (E)-4-(2-(2-(N-(1-(1-(naphthalen-1-yl)ethyl)piperidin-4-yl)methylsulfonamido)acetamido)acetamido)but-2-enamide

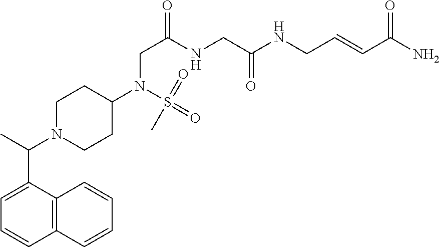 [SMILES: CC(c1cccc2ccccc12)N1CCC(N(CC(=O)NCC(=O)NC/C=C/C(N)=O)S(C)(=O)=O)CC1]

HPLC (Method 1) RT=3.69 min, Purity >99.9%, MS (ESI+) m/z calcd for C₂₆H₃₆N₅O₅S⁺ [M+H]⁺ 530.2, found 530.3.

### Compound 160, 2-(N-(1-(1-(naphthalen-1-yl)ethyl)piperidin-4-yl)methylsulfonamido)-N-(2-oxo-2-((3,3,3-trifluoro-2-oxopropyl)amino)ethyl)acetamide

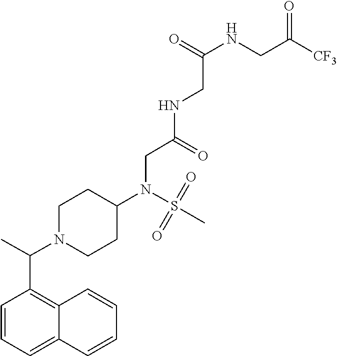 [SMILES: CC(c1cccc2ccccc12)N1CCC(N(CC(=O)NCC(=O)NCC(=O)C(F)(F)F)S(C)(=O)=O)CC1]

HPLC (Method 1) RT=5.58 min, Purity=99.0% MS (ESI+) m/z calcd for C₂₅H₃₂F₃N₄O₅S⁺ [M+H]⁺ 557.2, found 557.3.

### Compound 161, N-(2-cyanamido-2-oxoethyl)-2-(N-(1-(1-(naphthalen-1-yl)ethyl)piperidin-4-yl)methylsulfonamido)acetamide

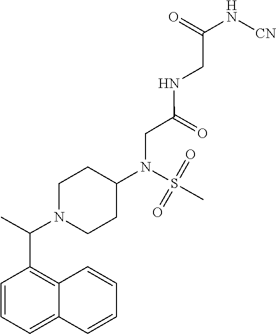 [SMILES: CC(c1cccc2ccccc12)N1CCC(N(CC(=O)NCC(=O)NC#N)S(C)(=O)=O)CC1]

HPLC (Method 1) RT=4.18 min, Purity=97.4%, MS (ESI+) m/z calcd for C₂₃H₃₀NSO₄S⁺ [M+H]⁺ 472.2, found 472.3.

### Compound 162, methyl (E)-2-cyano-4-(2-(2-(N-(1-(1-(naphthalen-1-yl)ethyl)piperidin-4-yl)methylsulfonamido)acetamido)acetamido)but-2-enoate

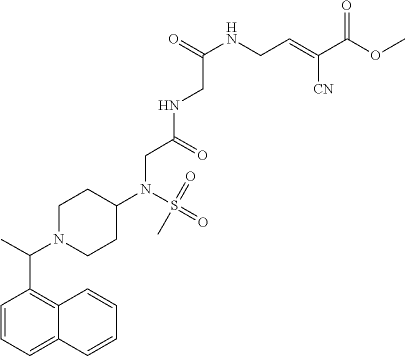 [SMILES: COC(=O)/C(C#N)=C/CNC(=O)CNC(=O)CN(C1CCN(C(C)c2cccc3ccccc23)CC1)S(C)(=O)=O]

HPLC (Method 1) RT=4.57 min, Purity=94.3%, MS (ESI+) m/z calcd for C₂₈H₃₆N₅O₆S⁺ [M+H]⁺ 570.2, found 570.2.

### Compound 163, methyl (E)-2-cyano-4-(2-(2-(N-(1-(1-(naphthalen-1-yl)ethyl)piperidin-4-yl)methylsulfonamido)acetamido)acetamido)but-2-enoate

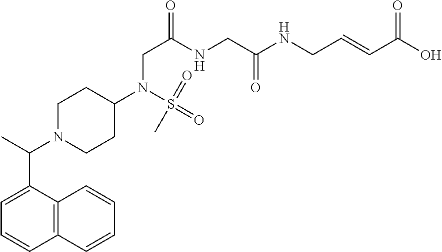 [SMILES: CC(c1cccc2ccccc12)N1CCC(N(CC(=O)NCC(=O)NC/C=C/C(=O)O)S(C)(=O)=O)CC1]

HPLC (Method 1) RT=3.79 min, Purity >99.9%, MS (ESI+) m/z calcd for C₂₆H₃₅N₄O₆S⁺ [M+H]⁺ 531.2, found 531.3.

### Compound 164, 2-(N-(1-(1-(naphthalen-1-yl)ethyl)piperidin-4-yl)methylsulfonamido)-N-(2-(prop-2-yn-1-ylamino)-2-thioxoethyl)acetamide

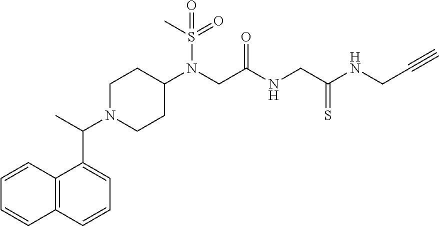 [SMILES: C#CCNC(=S)CNC(=O)CN(C1CCN(C(C)c2cccc3ccccc23)CC1)S(C)(=O)=O]

HPLC (Method 1) RT=4.37 min, Purity >99.9%, MS (ESI+) m/z calcd for C₂₅H₃₃N₄O₃S₂⁺ [M+H]⁺ 501.2, found 501.2.

### Compound 165, 2-(N-(1-(1-(naphthalen-1-yl)ethyl)piperidin-4-yl)methylsulfonamido)-N-(3,3,3-trifluoro-2-(prop-2-yn-1-ylamino)propyl)acetamide

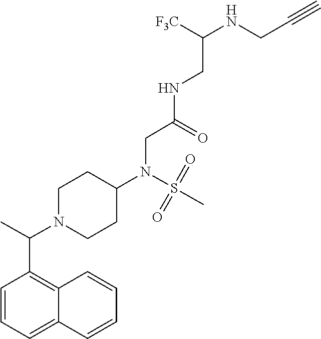 [SMILES: C#CCNC(CNC(=O)CN(C1CCN(C(C)c2cccc3ccccc23)CC1)S(C)(=O)=O)C(F)(F)F]

HPLC (Method 1) RT=4.33 min, Purity >99.9%, MS (ESI+) m/z calcd for C₂₆H₃₄F₃N₄O₃S⁺ [M+H]⁺ 539.2, found 539.3.

### Compound 166, (2R)-2-(2-(N-(1-(1-(naphthalen-1-yl)ethyl)piperidin-4-yl)methylsulfonamido)acetamido)-N-(prop-2-yn-1-yl)propanamide

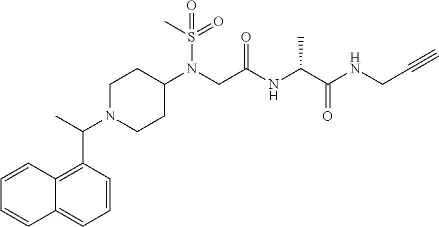 [SMILES: C#CCNC(=O)[C@@H](C)NC(=O)CN(C1CCN(C(C)c2cccc3ccccc23)CC1)S(C)(=O)=O]

HPLC (Method 1) RT=4.09 min, Purity >99.9%, MS (ESI+) m/z calcd for C₂₆H₃₅N₄O₄S⁺ [M+H]⁺ 499.2, found 499.2.

### Compound 167, 2-(N-(1-(1-(naphthalen-1-yl)ethyl)piperidin-4-yl)methylsulfonamido)-N-(3,3,3-trifluoro-2-(prop-2-yn-1-yloxy)propyl)acetamide

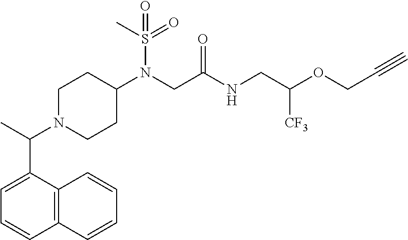 [SMILES: C#CCOC(CNC(=O)CN(C1CCN(C(C)c2cccc3ccccc23)CC1)S(C)(=O)=O)C(F)(F)F]

HPLC (Method 1) RT=4.63 min, Purity=96.3%, MS (ESI+) m/z calcd for C₂₆H₃₃F₃N₃O₄S⁺ [M+H]⁺ 540.2, found 540.3.

### Compound 168, N-(2-(2-acryloylhydrazineyl)-2-oxoethyl)-N-(1-(1-(naphthalen-1-yl)ethyl)piperidin-4-yl)methanesulfonamide

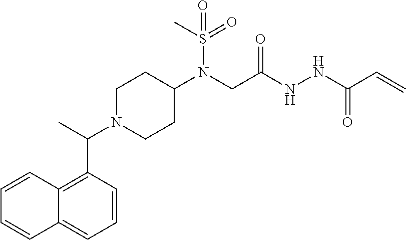 [SMILES: C=CC(=O)NNC(=O)CN(C1CCN(C(C)c2cccc3ccccc23)CC1)S(C)(=O)=O]

HPLC (Method 1) RT=3.90 min, Purity >99.9%, MS (ESI+) m/z calcd for C₂₃H₃₁N₄O₄S⁺ [M+H]⁺ 459.2, found 459.2.

### Compound 169, N-benzyl-2-hydroxy-3-(2-(2-(N-(1-(1-(naphthalen-1-yl)ethyl)piperidin-4-yl)methylsulfonamido)acetamido)acetamido)propanamide

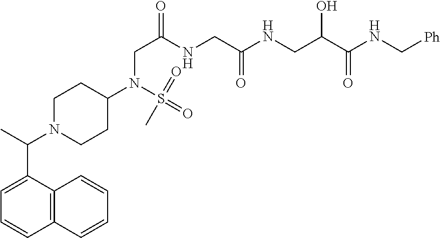 [SMILES: CC(c1cccc2ccccc12)N1CCC(N(CC(=O)NCC(=O)NCC(O)C(=O)NCc2ccccc2)S(C)(=O)=O)CC1]

HPLC (Method 1) RT=4.30 min, Purity=97.9%, MS (ESI+) m/z calcd for C₃₂H₄₂N₅O₆S⁺ [M+H]⁺ 624.3, found 624.4.

### Compound 170, N-(5-ethynyl-1,3,4-oxadiazol-2-yl)-2-(N-(1-(1-(naphthalen-1-yl)ethyl)piperidin-4-yl)methylsulfonamido)acetamide

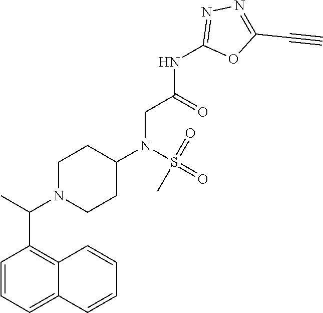 [SMILES: C#Cc1nnc(NC(=O)CN(C2CCN(C(C)c3cccc4ccccc34)CC2)S(C)(=O)=O)o1]

HPLC (Method 1) RT=4.22 min, Purity >99.9%, MS (ESI+) m/z calcd for C₂₄H₂₈N₅O₄S⁺ [M+H]⁺ 482.2, found 482.3.

### Compound 171, N-(but-3-yn-1-yl)-2-(3-(methylsulfonyl)-3-(1-(1-(naphthalen-1-yl)ethyl)piperidin-4-yl)ureido)acetamide

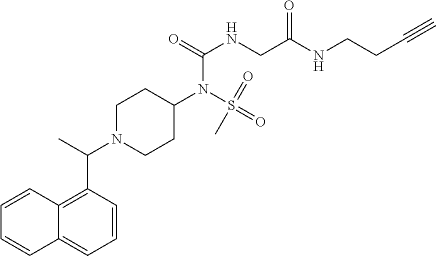 [SMILES: C#CCCNC(=O)CNC(=O)N(C1CCN(C(C)c2cccc3ccccc23)CC1)S(C)(=O)=O]

HPLC (Method 1) RT=4.22 min, Purity=98.2%, MS (ESI+) m/z calcd for C₂₅H₃₃N₄O₄S⁺ [M+H]⁺ 485.2, found 485.3.

### Compound 172, N-(but-3-yn-1-yl)-3-hydroxy-4-(N-(1-(1-(naphthalen-1-yl)ethyl)piperidin-4-yl)methylsulfonamido)butanamide

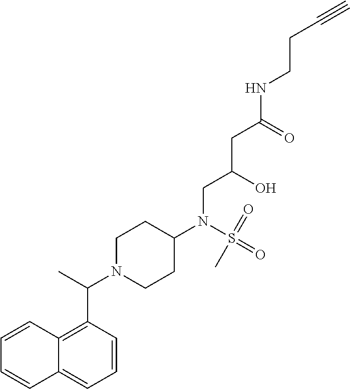 [SMILES: C#CCCNC(=O)CC(O)CN(C1CCN(C(C)c2cccc3ccccc23)CC1)S(C)(=O)=O]

HPLC (Method 1) RT=4.08 min, Purity=99.4%, MS (ESI+) m/z calcd for C₂₆H₃₆N₃O₄S⁺ [M+H]⁺ 486.2, found 486.3.

### Compound 173, (E)-N—((5-(2-methoxyvinyl)-1,3,4-oxadiazol-2-yl)methyl)-2-(N-(1-(1-(naphthalen-1-yl)ethyl)piperidin-4-yl)methylsulfonamido)acetamide

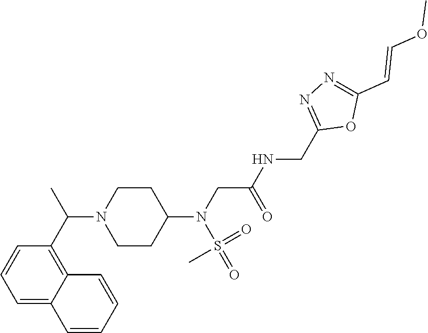 [SMILES: CO/C=C/c1nnc(CNC(=O)CN(C2CCN(C(C)c3cccc4ccccc34)CC2)S(C)(=O)=O)o1]

HPLC (Method 1) RT=4.06 min, Purity=82.1%, MS (ESI+) m/z calcd for C₂₆H₃₄N₅O₅S⁺ [M+H]⁺ 528.2, found 528.4.

### Compound 174, (R)-2-(N-(1-(1-(naphthalen-1-yl)ethyl)piperidin-4-yl)methylsulfonamido)-N-(2-oxo-2-((prop-2-yn-1-yl-1,1-d2)amino)ethyl)acetamide

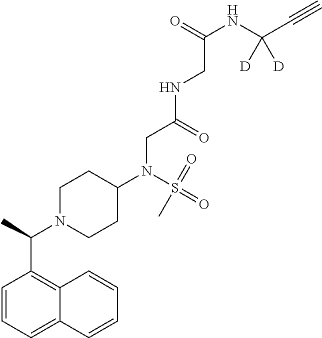 [SMILES: [2H]C([2H])(C#C)NC(=O)CNC(=O)CN(C1CCN([C@H](C)c2cccc3ccccc23)CC1)S(C)(=O)=O]

HPLC (Method 1) RT=4.01 min, Purity=99.7% MS (ESI+) m/z calcd for C₂₅H₃₁D₂N₄O₄S⁺ [M+H]⁺ 487.2, found 487.3.

### Compound 175, (S)-2-(N-(1-(1-(naphthalen-1-yl)ethyl)piperidin-4-yl)methylsulfonamido)-N-(2-oxo-2-((prop-2-yn-1-yl-1,1-d2)amino)ethyl)acetamide

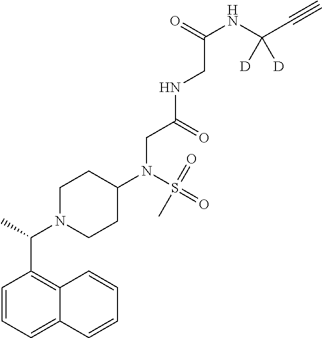 [SMILES: [2H]C([2H])(C#C)NC(=O)CNC(=O)CN(C1CCN([C@@H](C)c2cccc3ccccc23)CC1)S(C)(=O)=O]

HPLC (Method 1) RT=3.99 min, Purity >99.9%, MS (ESI+) m/z calcd for C₂₅H₃₁D₂N₄O₄S⁺ [M+H]⁺ 487.2, found 487.2.

### Compound 176, N-(1-(1-(naphthalen-1-yl)ethyl)piperidin-4-yl)-N-(2-oxo-2-(2-(vinylsulfonyl)hydrazineyl)ethyl)methanesulfonamide

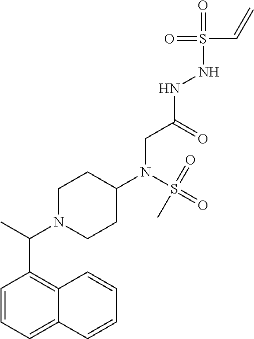 [SMILES: C=CS(=O)(=O)NNC(=O)CN(C1CCN(C(C)c2cccc3ccccc23)CC1)S(C)(=O)=O]

HPLC (Method 1) RT=4.02 min, Purity >99.9%, MS (ESI+) m/z calcd for C₂₂H₃₁N₄O₅S₂⁺ [M+H]⁺ 495.2, found 495.2.

### Compound 177, N-benzyl-3-(2-(2-(N-(1-(1-(naphthalen-1-yl)ethyl)piperidin-4-yl)methylsulfonamido acetamido)acetamido)-2-oxopropanamide

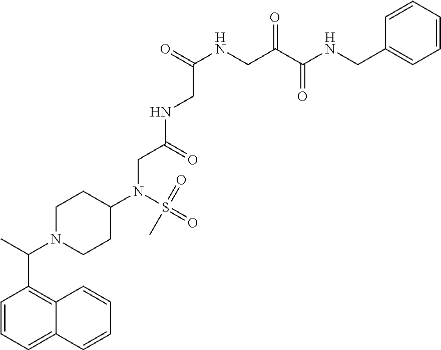 [SMILES: CC(c1cccc2ccccc12)N1CCC(N(CC(=O)NCC(=O)NCC(=O)C(=O)NCc2ccccc2)S(C)(=O)=O)CC1]

HPLC (Method 3) RT=2.24 min, MS (ESI+) m/z calcd for C₃₂H₄₀NSO₆S⁺ [M+H]⁺ 622.3 found 622.4.

### Biological Assays

### Biological Example 1. PLpro Preparation and Purification

The PLpro (pplab 1564-1878) was codon-optimized, synthesized, and cloned into pET11a vector with a TEV cleavable his6-tag at the N-terminus. The recombinant plasmid was transformed into BL21(DE3) expression cells and grown in Luria-Bertani (LB) media with carbenicillin (100 g/mL) at 37° C. while shaking at 220 rpm until the OD600 reached 0.6, when it was induced with 0.5 mM IPTG and incubated for an additional 16 h at 18° C. before harvesting. The cell pellet was resuspended and lysed by sonication in lysis buffer (50 mM Tris, pH 8.0, 500 mM NaCl, 20 mM imidazole, 5 mM 3-MCE, 1 mg/mL lysozyme, 1% Triton X-100 and 0.025 mg/mL DNase I). A HisTrap HP column was used to purify the histidine-tagged protein using a stepwise gradient of elution buffer (50 mM Tris, pH 8.0, 500 mM NaCl, 500 mM imidazole and 5 mM β-MCE) with an AKTA Pure FPLC system. The histidine-TEV tag was removed by incubating the eluted protein with 1 unit/100 g protein of TEV protease at 4° C. for 16 h. The digested protein was reloaded onto a HisTrap HP column equilibrated with 50 mM Tris, pH 8.0, 500 mM NaCl and 5 mM β-MCE, and the histidine-TEV tag cleaved PLpro was collected in the flowthrough and loaded onto a HiLoad 16/60 Superdex 75 PG gel filtration column that was equilibrated with 50 mM Tris, pH 8.0, 200 mM NaCl and 1 mM TCEP. Protein samples were analyzed by SDS-PAGE, and the final purity was above 95%.

### Biological Example 2. Compound Inhibition and IC50 Value Determination

The PLpro enzyme was purified as described above and prepared in assay buffer (50 mM HEPES, pH 7.5, 0.01% Triton X-100 (v/v), 0.1 mg mL-1 BSA, and 2 mM DTT). IC50 values were measured in triplicate. A series of increasing concentrations (0-100 μM final concentration at 3-fold serial dilution) in 100% DMSO were prepared in a 384-well plate. 7 μL of 225 nM (3X) enzyme solution was distributed into wells, and 7 μL of varying concentration of 3X compounds were added and incubated for 10 min and 60 min for non-covalent inhibitors and covalent inhibitors, respectively. The enzyme reaction was initiated by adding 7 μL of the 75 μM (3X) substrate, and its activity was continuously monitored for at least 10 min. The IC50 values were calculated by fitting with the Hill equation (1), with Sigmaplot v14, where y is percent inhibition, x is inhibitor concentration, n is the slope of the concentration-response curve (Hill slope), and Vmax is maximal inhibition from three independent assays.

$$y = {V}_{max} ( \frac{{x}^{n}}{{IC}_{50}^{n} + {x}^{n}} ) \qquad ( 1 )$$

The testing results for the compounds 1-177 in the enzymatic inhibition assay for SARS-CoV-2 PLpro are summarized in Table 1.

### Biological Example 3. Compound Binding Assay and Inactivation Rate Assay [22]

The purified SARS-CoV-2 PLpro enzyme was immobilized on flow channels 2 and 4 of a CM5 sensor chip using standard amine-coupling with running buffer HBS—P (10 mM HEPES, 150 mM NaCl, 0.05% surfactant P-20, pH 7.4) using a Biacore T200 instrument. Flow channels 1 and 3 were used as control surfaces. The PLpro enzyme was diluted in 10 mM sodium acetate (pH 5.0), and immobilized after sensor surface activation with 1-ethyl-3-(3-dimethylaminopropyl) carbodiimide hydrochloride (EDC)/N-hydroxy succinimide (NHS) mixture followed by ethanolamine (pH 8.5) blocking on unoccupied surface area. Selected compounds were initially prepared as 10 mM DMSO stock solutions, and compound solutions with a series of increasing concentrations (0-10 μM at 4-fold dilution) were applied to all four channels at a 30 μL/min flow rate at 25° C. The single-cycle kinetic method was run, and real-time response units were monitored. Sensorgrams were double referenced with the blank channel and zero concentration responses and fitted with 1 to 1 Langmuir kinetic equation embedded in the Biacore Insight software.

Surface Plasmon Resonance (SPR) binding analysis showed the direct binding response of selected PLpro inhibitors to the immobilized SARS-CoV-2 PLpro protein at a series of increasing concentration using single-cycle kinetics. Kd values were determined <100 nM for representative compounds.

The purified SARS-CoV2-PLpro protein was prepared in the assay buffer (50 mM HEPES, pH 7.5, 0.01% Triton X-100, 0.1 mg/mL BSA). The PLpro substrate, Z-RLRGG-AMC (Bachem Bioscience), is a small fluorogenic peptide. The cleavage of the substrate by PLpro releases the AMC (7-amido-4-methylcoumarin) which generates fluorescence signal. Assays were done using black low-volume 384-well plates (Greiner). All compounds were initially prepared as 1 mM stocks in 100% DMSO. A series of increasing concentrations were prepared in 100% DMSO through 2-fold serial dilutions. Final compound concentrations (1×) tested ranged from 1.5-1500 nM. 7 μL of the compound (3×) solutions and 7 μL of the substrate (3×) solution were added to the wells. The reaction was initiated by adding and mixing 7 μL of the SARS-CoV2-PLpro (3×) solution. The final concentration (1×) of the SARS-CoV2-PLpro was 20 nM and the final concentration (lx) of the substrate was 10 uM. The fluorescence intensity at 450 nm (excitation at 360 nm) was continuously monitored for 1.5 hr at 30° C. using the BMG LabTech PolarStar Optima Plate Reader. Using DynaFit, the kinetic curves were fit to the equation: F=F₀+r_{P}[S]₀{1−exp[−P(1−exp(−αt))]} to derive the values for α parameters. K_{inact}/Kᵢ was derived by fitting a hyperbolic equation, α=K_{inact}([I]₀/([I]₀+Kᵢ)), to the plot of α versus compound concentration.

### Biological Example 4. Covalent Mode of Inhibition Determined by MALDI

Covalent binding and selectivity for the catalytic cysteine was determined by comparing MALDI mass spectrometric analyses of wild-type SARS-CoV-2 PLpro incubated with inhibitor with that of C₁₁₁S—CoV-2 PLpro, in which the catalytic cysteine was mutated to serine. Treatment of PLpro with representative compounds results in a covalent irreversible adduct formation in the wild type enzyme but not in the active site cysteine mutant enzyme. The addition of a mass corresponding to one molecule of PLpro inhibitor to wild-type PLpro indicates selective covalent reaction with the catalytic cysteine, C111 of PLpro.

### Biological Example 5. SARS-CoV2 Antiviral Activity Assay

PLpro inhibitor antiviral activities were tested in a cell culture assay with A549:hACE2 cells (Invivogen) by the following protocol: seed 10K cells (A549:hACE2) per well the day before testing. Then make a serial dilution of the drug in 2% DMEM media to add to the cells. Cells are left to incubate with drugs for 2 h prior to infection. Then cells are infected with 0.5 MOI (multiplicity of infection) and 48 hours later they are fixed with 10% Formalin for 15-30 min. The cells then undergo immunohistochemistry antibody staining protocol. Cells are blocked with 1% BSA+0.025% Saponin for 1h, washed with 3% hydrogen peroxide for 5 min, washed with PBS and PBST, then primary antibody [mouse anti-Spike antibody (GTX632604, GeneTex)] in blocking buffer overnight. This was followed the next day by additional PBS/PBST washing, secondary HRP antibody for 1 hour then DAB stain for 15 minutes. The percentage of cells positive for Spike protein is then assessed under the microscope. The EC50 is determined from the following equation (2), where [I] is the inhibitor concentration and n is the Hill coefficient, using software such as Prism (Graphpad Software, San Diego) or the AAT Bioquest EC50 calculator (https://www.aatbio.com/tools/ee50-calculator).

$$y = Min + ( Max - Min ) / ( 1 + {( [ I ] / EC 50 )}^{⋀} n ) \qquad ( 2 )$$

The cellular EC50 results for the selected PLpro compounds in the SARS-CoV2 antiviral cellular activity assay are summarized in Table 2.

TABLE 2

| | Antiviral cellular EC₅₀ range |
|---|---|
| Compound No. | (Omicron)^{a} |
| 20 | +++ |
| 21 | +++ |
| 60 | +++ |
| 63 | +++ |
| 65 | +++ |
| 79 | ++ |
| 80 | ++ |
| 107 | ++ |

^{a}SARS-CoV2 antiviral cellular activity is expressed by the EC₅₀ range. It is indicated as +++ (EC₅₀: less than 100 nM) or ++ (EC₅₀: 0.1 μM to less than 1 μM).

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the ordinary skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the ordinarily skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

### REFERENCES

1. Lindner, H. A., et al., Selectivity in ISG15 and ubiquitin recognition by the SARS coronavirus papain-like protease. Arch Biochem Biophys, 2007. 466(1): p. 8-14.
2. Liu, G., et al., ISG15-dependent activation of the sensor MDA5 is antagonized by the SARS-CoV-2 papain-like protease to evade host innate immunity. Nat Microbiol, 2021. 6(4): p. 467-478.
3. Freitas, B. T., et al., Characterization and Noncovalent Inhibition of the Deubiquitinase and deISGylase Activity of SARS-CoV-2 Papain-Like Protease. ACS Infect Dis, 2020. 6(8): p. 2099-2109.
4. Shin, D., et al., Papain-like protease regulates SARS-CoV-2 viral spread and innate immunity. Nature, 2020. 587(7835): p. 657-662.
5. Macchiagodena, M., M. Pagliai, and P. Procacci, Identification of Potential Binders of the Main Protease 3CL(pro) of the COVID-19 via Structure-Based Ligand Design and Molecular Modeling. Chem Phys Lett, 2020: p. 137489.
6. Ul Qamar, M. T., et al., Structural basis of SARS-CoV-2 3CL(pro) and anti-COVID-19 drug discovery from medicinal plants. J Pharm Anal, 2020.
7. Sisay, M., 3CL(pro) inhibitors as a potential therapeutic option for COVID-19: Available evidence and ongoing clinical trials. Pharmacol Res, 2020. 156: p. 104779.
8. Chen, X., et al., SARS coronavirus papain-like protease inhibits the type I interferon signaling pathway through interaction with the STING-TRAF3-TBK1 complex. Protein Cell, 2014. 5(5): p. 369-81.
9. Fung, T. S. and D. X. Liu, Human Coronavirus: Host-Pathogen Interaction. Annu Rev Microbiol, 2019. 73: p. 529-557.
10. Ghosh, A. K., et al., Structure-based design, synthesis, and biological evaluation of a series of novel and reversible inhibitors for the severe acute respiratory syndrome-coronavirus papain-like protease. J Med Chem, 2009, 52(16): p. 5228-5240.
11. Baez-Santos, Y. M., et al., X-ray structural and biological evaluation of a series of potent and highly selective inhibitors of human coronavirus papain-like proteases. J Med Chem, 2014, 57(6): p. 2393-2412.
12. Nicola, M., et al., The Socio-Economic Implications of the Coronavirus and COVID-19 Pandemic: A Review. Int J Surg, 2020.
13. Di Gennaro, F., et al., Coronavirus Diseases (COVID-19) Current Status and Future Perspectives: A Narrative Review. Int J Environ Res Public Health, 2020. 17(8).
14. Snijder, E. J., et al., Unique and conserved features of genome and proteome of SARS-coronavirus, an early split-off from the coronavirus group 2 lineage. J Mol Biol, 2003. 331(5): p. 991-1004.
15. Rabaan, A. A., et al., SARS-CoV-2, SARS-CoV, and MERS-COV: A comparative overview. Infez Med, 2020. 28(2): p. 174-184.
16. Mielech, A. M., et al., MERS-CoV papain-like protease has deISGylating and deubiquitinating activities. Virology, 2014. 450-451: p. 64-70.
17. Baez-Santos, Y. M., S. E. St John, and A. D. Mesecar, The SARS-coronavirus papain-like protease: structure, function and inhibition by designed antiviral compounds. Antiviral Res, 2015. 115: p. 21-38.
18. Devaraj, S. G., et al., Regulation of IRF-3-dependent innate immunity by the papain-like protease domain of the severe acute respiratory syndrome coronavirus. J Biol Chem, 2007. 282(44): p. 32208-21.
19. Clementz, M. A., et al., Deubiquitinating and interferon antagonism activities of coronavirus papain-like proteases. J Virol, 2010. 84(9): p. 4619-29.
20. Frieman, M., et al., Severe acute respiratory syndrome coronavirus papain-like protease ubiquitin-like domain and catalytic domain regulate antagonism of IRF3 and NF-kappaB signaling. J Virol, 2009. 83(13): p. 6689-705.
21. Cron, R. Q., et al., Calming the cytokine storm in COVID-19. Nat Med, 2021. 27: p. 1674-75.
22. Kuzmic, P., et al., AN algebraic model for the kinetics of covalent enzyme inhibition at low substrate concentrations. Anal Biochem, 2015, 484, p. 82-90.

What is claimed is:

1. A compound of Formula X, or a pharmaceutically acceptable salt thereof,
A-L-W Formula X,
wherein:
A represents a residue of a papain-like protease (PLpro) inhibitor,
W represents a group capable of forming a covalent bond with PLpro, preferably, capable of forming a covalent bond with an SH group of a cysteine residue of PLpro, and
L represents a linker that covalently links A and W.

2. The compound of claim 1, or a pharmaceutically acceptable salt thereof, wherein A is characterized as having a structure according to Formula A-1: 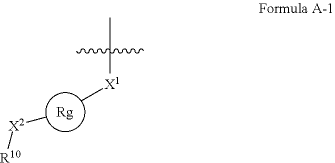
wherein:
X¹ is absent, CR¹¹R¹², NR¹³C(O), or —C(O)N(R¹³)—,
X² is absent, optionally substituted C₁₋₄ alkylene, or optionally substituted C₁₋₄ heteroalkylene,
Rg is an optionally substituted ring structure having 4-10 ring atoms, preferably, an optionally substituted 4-10 membered heterocyclic ring, and
wherein:
R¹⁰ is an optionally substituted aryl or an optionally substituted heteroaryl,
R¹¹ and R¹² are each independently hydrogen, halogen, OH, NH₂, COOH, CONH₂, SO₂NH₂, G^{A}, OG^{A}, NH(G^{A}), N(G^{A})(G^{A}), COOG^{A}, CONH(G^{A}), CON(G^{A})(G^{A}), SO₂NH(G^{A}), SO₂N(G^{A})(G^{A}), NHCOG^{A}, N(G^{A})COG^{A}, NHSO₂(G^{A}), N(G^{A})SO₂(G^{A}), COG^{A}, or SO₂(G^{A}), or R¹¹ and R¹² are joined to form a 3-8 membered ring structure;
R¹³ is hydrogen, G^{A}, COOG^{A}, CONH₂, CONH(G^{A}), CON(G^{A})(G^{A}), SO₂NI-2, SO₂NH(G^{A}), SO₂N(G^{A})(G^{A}), COG^{A}, or SO₂(G^{A}),
wherein G^{A} at each occurrence is independently an optionally substituted C₁₋₆ alkyl, optionally substituted C₂₋₆ alkenyl, optionally substituted C₂₋₆ alkynyl, an optionally substituted C₁₋₆ heteroalkyl, or an optionally substituted ring structure having 4-10 ring atoms.

3. The compound of claim 2, or a pharmaceutically acceptable salt thereof, wherein in the moiety of Formula A-1, Rg is an optionally substituted 4-7 membered monocyclic heterocyclic ring having one or two ring heteroatoms, each independently O, S, or N.

4. The compound of claim 2 or 3, or a pharmaceutically acceptable salt thereof, wherein the moiety of Formula A-1 is characterized as having a structure according to Formula A-2: 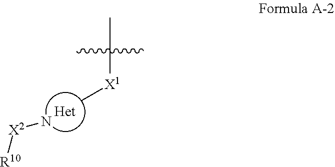
wherein Het represents an optionally substituted 4-10 membered heterocyclic ring, which is bonded with X² through the ring nitrogen atom as drawn, and is bonded with X¹ through another ring atom, wherein the 4-10 membered heterocyclic ring has 0-3 ring heteroatoms each independently O, S, or N, in addition to the as drawn ring nitrogen atom.

5. The compound of claim 2 or 3, or a pharmaceutically acceptable salt thereof, wherein the moiety of Formula A-1 is characterized as having a structure according to Formula A-3:

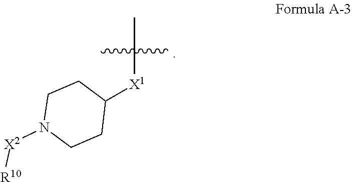

6. The compound of any of claims 2-5, or a pharmaceutically acceptable salt thereof, wherein X² is an optionally substituted C₁₋₄ alkylene, such as a linear or branched unsubstituted C₁₋₄ alkylene (e.g., CH₂, CH(CH₃), CH(C₂HS), etc.), or X² is absent.

7. The compound of any of claims 2-5, or a pharmaceutically acceptable salt thereof, wherein X² is represented by CR¹⁴R¹⁵, wherein (i) R¹⁴ and R¹⁵ are each independently hydrogen, an optionally substituted C₁₋₄ alkyl, optionally substituted C₂₋₄ alkenyl, optionally substituted C₂₋₄ alkynyl, or an optionally substituted ring structure having 4-10 ring atoms; (ii) one of R¹⁴ and R¹⁵ is COOH, or an ester or amide thereof, and the other of R¹⁴ and R¹⁵ is defined in (i); or (iii) R¹⁴ and R¹⁵ together with the carbon they are both attached to form a 3-6 membered ring which is optionally substituted.

8. The compound of claim 7, or a pharmaceutically acceptable salt thereof, wherein one of R¹⁴ and R¹⁵ is hydrogen, and the other of R¹⁴ and R¹⁵ is hydrogen, C₁₋₄ alkyl optionally substituted with 1-3 G^{S1}, C₃₋₆ cycloalkyl optionally substituted with 1-3 G^{S1}, or phenyl optionally substituted with 1-3 G^{S1}, wherein G^{S1} at each occurrence is independently halogen (preferably F), NH₂, OH, C₁₋₄ alkyl, or C₁₋₄ alkoxy.

9. The compound of claim 7, or a pharmaceutically acceptable salt thereof, wherein (i) one of R¹⁴ and R¹⁵ is hydrogen, and the other of R¹⁴ and R¹⁵ is hydrogen, methyl, or phenyl, (ii) one of R¹⁴ and R¹⁵ is hydrogen, and the other of R¹⁴ and R¹⁵ is CH₂F, CH₂OH, CHF₂, CD₃, CF₃, ethyl, CN, cyclopropyl, COOH, or COOCH₃; or (iii) R¹⁴ and R¹⁵ together with the carbon they are both attached to form a cyclopropyl.

10. The compound of any one of claims 2-9, or a pharmaceutically acceptable salt thereof, wherein R¹⁰ is an optionally substituted phenyl or optionally substituted naphthyl.

11. The compound of any one of claims 2-9, or a pharmaceutically acceptable salt thereof, wherein (i) R¹⁰ is 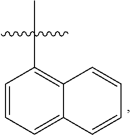 [SMILES: *c1cccc2ccccc12]
which is optionally substituted, such as with 1-3 G^{S1} wherein G^{S1} at each occurrence is independently halogen (preferably F or Cl), OH, C₁₋₄ alkyl (e.g., methyl), or C₁₋₄ alkoxy (e.g., methoxy); (ii) 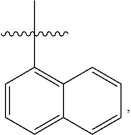 [SMILES: *c1cccc2ccccc12]
which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring; (iii) 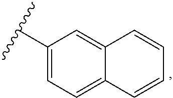 [SMILES: *c1ccc2ccccc2c1]
which is optionally substituted, such as with 1-3 G^{S1A}, wherein G^{S1} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring; (iv) 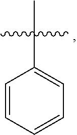 [SMILES: *c1ccccc1]
which is optionally substituted, such as with 1-3 G^{S1A}, wherein G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring; or (v) 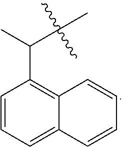 [SMILES: *C(C)c1cccc2ccccc12]
which is optionally substituted, such as with 1-3 G^{S1B}, wherein G^{S1B} at each occurrence is independently halogen (e.g., F, Cl, or Br), OH, C₁₋₄ alkyl optionally substituted with 1-3 F (e.g., methyl, CF₃), C₁₋₄ alkoxy optionally substituted with 1-3 F (e.g., methoxy), or an optionally substituted 3-7 membered ring (e.g., phenyl, thienyl, etc.).

12. The compound of any one of claims 2-11, or a pharmaceutically acceptable salt thereof, wherein X²—R¹⁰ is

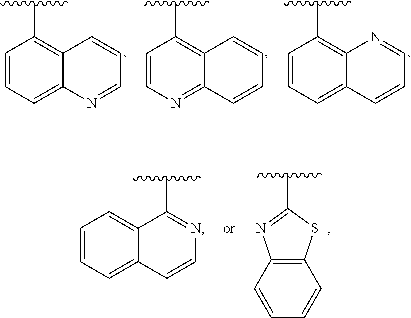

13. The compound of any one of claims 2-11, or a pharmaceutically acceptable salt thereof, wherein X²—R¹⁰ is

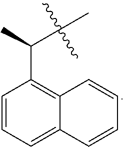 [SMILES: *[C@H](C)c1cccc2ccccc12]

14. The compound of any one of claims 2-13, or a pharmaceutically acceptable salt thereof, wherein X¹ is NR¹³.

15. The compound of claim 14, or a pharmaceutically acceptable salt thereof, wherein (i) R¹³ is hydrogen, optionally substituted C₁₋₆ alkyl, COG^{A} or SO₂G^{A}; or (ii) R¹³ is an optionally substituted C₁₋₆ alkyl, optionally substituted C₂₋₆ alkenyl or optionally substituted C₂₋₆ alkynyl, such as

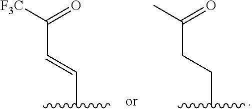

16. The compound of claim 14, or a pharmaceutically acceptable salt thereof, wherein R¹³ is COG^{A1}, wherein G^{A1} is an optionally substituted C₁₋₆ alkyl, optionally substituted C₃₋₆ cycloalkyl, optionally substituted phenyl, optionally substituted naphthyl, or an optionally substituted ring structure having 4-10 ring atoms with 1-3 ring heteroatoms.

17. The compound of claim 14, or a pharmaceutically acceptable salt thereof, wherein R¹³ is COG^{A2}, wherein (i) G^{A2} is a C₁₋₆ alkyl, which is optionally substituted with 1-3 G^{S2}, wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3}, or (ii) G^{A2} is a C₁₋₆ alkyl, optionally substituted with 1-3 G^{S2A}, wherein G^{S2A} at each occurrence is independently halogen (preferably F), OH, COOH, CONH₂, G^{S2B}, CONHG^{S2B}, CONG^{S2B} G^{S2B} , SO₂NH₂, SO₂NHG^{S2B} , SO₂NG^{S2B}G^{S2B}, COG^{S2B}, CO₂G^{S2B}, or SO₂G^{S2B}, wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3},
wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure.

18. The compound of claim 14, or a pharmaceutically acceptable salt thereof, wherein R¹³ is COG^{A3}, wherein G^{A3} is a C₃₋₆ cycloalkyl, which is optionally substituted with 1-5 G^{S3},
wherein
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure; and
G^{S1} at each occurrence is independently halogen (preferably F), OH, C₁₋₄ alkyl, or C₁₋₄ alkoxy.

19. The compound of claim 14, or a pharmaceutically acceptable salt thereof, wherein R¹³ is selected from:

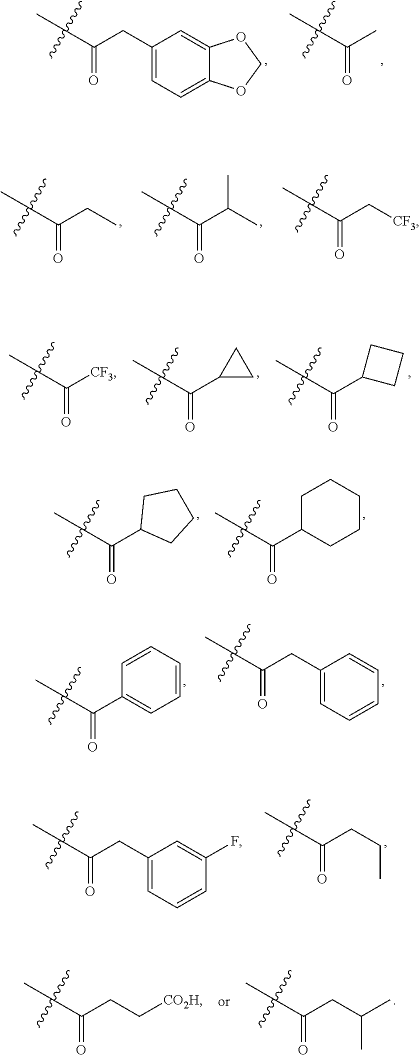

20. The compound of claim 14, or a pharmaceutically acceptable salt thereof, wherein R¹³ is SO₂G^{A4}, wherein (i) G^{A4} is a C₁₋₆ alkyl, which is optionally substituted with 1-3 G^{S2} wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3} phenyl optionally substituted with 1-5 G^{S3}, or 4-8 membered ring having 1-3 ring heteroatoms independently O, S, or N which is optionally substituted with 1-5 G^{S3}, or (ii) G^{A4} is a C₁₋₆ alkyl, optionally substituted with 1-3 G^{S2A}, wherein G^{S2}A at each occurrence is independently halogen (preferably F), OH, COOH, CONH₂, G^{S2B}, CONHG^{S2B}, CONG^{S2B} G^{S2B} , SO₂NH₂, SO₂NHG^{S2B} , SO₂NG^{S2B}G^{S2B}, COG^{S2B}, CO₂G^{S2B}, or SO₂G^{S2B}, wherein G^{S2B} at each occurrence is independently C₁₋₄ alkyl optionally substituted with 1-3 G^{S1}, C₁₋₄ alkoxy optionally substituted with 1-3 G^{S1}, C₃₋₆ cycloalkyl optionally substituted with 1-5 G^{S3}, phenyl optionally substituted with 1-5 G^{S3}, or 4-8 membered ring having 1-3 ring heteroatoms independently O, S, or N which is optionally substituted with 1-5 G^{S3},
wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure.

21. The compound of claim 14, or a pharmaceutically acceptable salt thereof, wherein R¹³ is SO₂G^{A5}, wherein (i) G^{A5} is a C₃₋₆ cycloalkyl or phenyl, which is optionally substituted with 1-5 G^{S3}, or (ii) G^{A5} is a 4-7 membered heterocyclic ring having 1 or 2 ring heteroatoms, which are independently N, O, or S, wherein the 4-7 membered heterocyclic ring is optionally substituted with 1-3 substituents independently selected from G^{S3A} (C₁ 4 alkylene)-G^{S3A}, OG^{S3A} NHG^{S3A}, NG^{S3A}G^{S3A}, or (C₁₋₄ heteroalkylene)-G^{S3A}, wherein
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be join to form an optionally substituted 3-6 membered ring structure; and
G^{S1} at each occurrence is independently halogen (preferably F), OH, C₁₋₄ alkyl, or C₁₋₄ alkoxy, and
G^{S3A} at each occurrence is independently halogen (preferably F), OH, COOH, CONH₂, G^{S2}, CONHG^{S2B} , CONG^{S2B} G^{S2B}, SO₂NH₂, SO₂NHG^{S2B} , SO₂NG^{S2B}G^{S2B}, COG^{S2B}, CO₂G^{S2B}, or SO₂G^{S2B}, wherein G^{S2B} at each occurrence is independently as defined in claim 20.

22. The compound of claim 14, or a pharmaceutically acceptable salt thereof, wherein R¹³ is selected from:

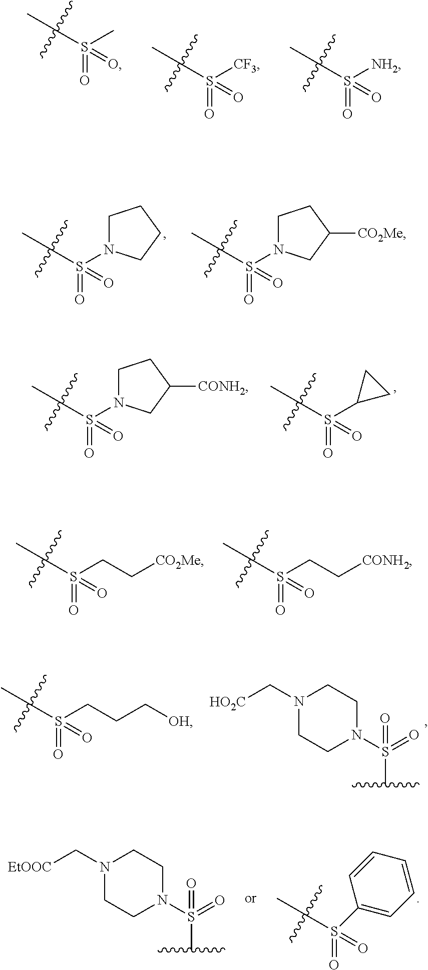

23. The compound of any one of claims 2-13, or a pharmaceutically acceptable salt thereof, wherein X¹ is CR¹¹R¹² .

24. The compound of claim 23, or a pharmaceutically acceptable salt thereof, wherein one of R¹¹ and R¹² is hydrogen, and the other of R¹¹ and R¹² is hydrogen, NH₂, NH(G^{A}), N(G^{A})(G^{A}), NHCO(G^{A}), or N(G)CO(G^{A}).

25. The compound of claim 23 or 24, or a pharmaceutically acceptable salt thereof, wherein one of R¹¹ and R¹² is hydrogen, and the other of R¹¹ and R¹² is hydrogen, NH₂, NH(G^{A6}), or N(G^{A6})(G^{A6}), wherein G^{A6} at each occurrence is independently a C₁₋₆ alkyl, which is optionally substituted with 1-3 G^{S2},
wherein G^{S2} at each occurrence is independently halogen (preferably F), OH, C₁₋₄ alkoxy optionally substituted with 1-3 G^{S1}, C₃₋₆ cycloalkyl optionally substituted with 1-5 G^{S3} phenyl optionally substituted with 1-5 G^{S3}, or 4-8 membered ring having 1-3 ring heteroatoms independently O, S, or N which is optionally substituted with 1-5 G^{S3} ,
wherein
G^{S1} at each occurrence is independently halogen (preferably F), OH, C₁₋₄ alkyl, or C₁₋₄ alkoxy and
G^{S3} at each occurrence is independently halogen (preferably F), OH, C₁₋₄ alkyl optionally substituted with 1-3 G^{S1}, C₁₋₄ alkoxy optionally substituted with 1-3 G^{S1}, or two instances of G^{S3} can be joined to form an optionally substituted 3-6 membered ring structure.

26. The compound of any one of claims 1-25, or a pharmaceutically acceptable salt thereof, wherein L has a structure according to Formula L-1: 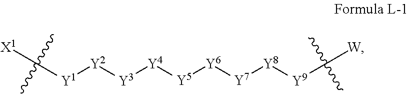
wherein:
Y¹ and Y⁹ are each independently absent, an optionally substituted C₁₋₄ alkylene, optionally substituted C₁₋₄ heteroalkylene, or optionally substituted 3-8 membered ring structure,
Y², Y³, Y⁴, Y⁵, Y⁶, Y⁷, and Y⁸ are each independently absent, CR²⁰R²¹, C(O), C(S), O, NR²², S, SO, or SO₂, wherein up to 4 of Y², Y³, Y⁴, Y⁵, Y⁶, Y⁷, and Y⁸ can be absent,
wherein
(i) two adjacent CR²⁰R²¹ units can optionally form a double bond to form —C(R²⁰)═C(R²¹)— or —C≡C—;
(ii) two adjacent CR²⁰R²¹ and NR²² units can optionally form a double bond to form —C(R²⁰)═N—; or
(iii) two adjacent NR²² units can optionally form a double bond to form —N═N—;
or three or four consecutive Y², Y³, Y⁴, Y⁵, Y⁶, Y⁷, and Y⁸ together (e.g., Y⁴, Y⁵, and Y⁶;
or Y⁵, Y⁶, and Y⁷, etc.) represent a 3-10 membered ring structure, and the remaining of Y², Y³, Y⁴, Y⁵, Y⁶, Y⁷, and Y⁸ are as defined above,
wherein:
R²⁰ and R²¹ at each occurrence are each independently hydrogen, halogen, OH, NH₂, COOH, CONH₂, SO₂NH₂, G^{A}, OG^{A}, NH(G^{A}), N(G^{A})(G^{A}), COOG^{A}, CONH(G^{A}), CON(G^{A})(G^{A}), SO₂NH(G^{A}), SO₂N(G^{A})(G^{A}), NHCOG^{A}, N(G^{A})COG^{A}, NHSO₂(G), N(G^{A})SO₂(G^{A}), COG^{A}, or SO₂(G^{A}),
R²² at each occurrence is independently hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G^{A}),
wherein G^{A} at each occurrence is independently an optionally substituted C₁₋₆ alkyl, optionally substituted C₂₋₆ alkenyl, optionally substituted C₂₋₆ alkynyl, an optionally substituted C₁₋₆ heteroalkyl, or an optionally substituted ring structure having 4-10 ring atoms;
or two or more instances of R²⁰, R²¹ and R²² (e.g., R²⁰ and R²¹ on the same carbon, R²⁰ and R²⁰ on two different carbons, R²⁰ and R²², etc.), can be joined to form a 3-8 membered ring structure.

27. The compound of claim 26, or a pharmaceutically acceptable salt thereof, wherein (i) Y¹ is absent, (ii) Y¹ is an optionally substituted C₁₋₄ alkylene, such as CH₂ or CD₂, or (iii) Y¹ is an optionally substituted 4-8 membered heterocyclic ring having 1-3 ring heteroatoms independently O, S, or N, such as

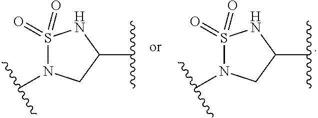 [SMILES: *C1CN(*)S(=O)(=O)N1 ; *C1CN(*)S(=O)(=O)N1]

28. The compound of claim 26 or 27, or a pharmaceutically acceptable salt thereof, wherein (i) Y⁹ is absent, an optionally substituted C₁₋₄ alkylene, such as CH₂, CD₂ or CF₂, or an optionally substituted 4-8 membered heterocyclic ring having 1 or 2 ring heteroatoms independently O, S, or N; (ii) Y⁹ is a C₃₋₆ membered carbocyclic ring, such as 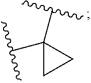 [SMILES: *C1(*)CC1]
or (iii) Y⁹ is a C₁₋₄ heteroalkylene (e.g., —NH—CH₂—, —CH₂—NH—CH₂—, etc.), which is optionally substituted, such as with oxo, F, CF₃, etc.

29. The compound of any one of claims 26-28, or a pharmaceutically acceptable salt thereof, wherein:
(i) Y² is CH₂ or CH(C₁₋₄ alkyl);
(ii) Y³ is C(O);
(iii) Y⁴ is NH, CH₂ or CH(C₁₋₄ alkyl);
(iv) Y⁵ is NH, CH₂, CH(C₁₋₄ alkyl), C(C₁₋₄ alkyl)(C₁₋₄ alkyl), or 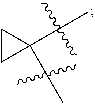 [SMILES: *C1(*)CC1]
(v) Y⁶ is C(O), CH₂ or CH(C₁₋₄ alkyl);
(vi) Y⁷ is absent, C(O), NH, CH₂ or CH(C₁₋₄ alkyl); and/or
(vii) Y⁸ is NH, CH₂ or CH(C₁₋₄ alkyl), or absent.

30. The compound of any one of claims 26-29, or a pharmaceutically acceptable salt thereof, wherein two of Y², Y³, Y⁴, Y⁵, Y⁶, Y⁷, and Y⁸ are C(O).

31. The compound of any one of claims 26-29, or a pharmaceutically acceptable salt thereof, wherein one of Y², Y³, Y⁴, Y⁵, Y⁶, Y⁷, and Y⁸ are C(O).

32. The compound of any one of claims 26-31, or a pharmaceutically acceptable salt thereof, wherein one of Y², Y³, Y⁴, Y⁵, Y⁶, Y⁷, and Y⁸ is SO₂.

33. The compound of any one of claims 26-32, or a pharmaceutically acceptable salt thereof, wherein up to four of Y², Y³, Y⁴, Y⁵, Y⁶, Y⁷, and Y⁸ are NR²².

34. The compound of any one of claims 26-33, or a pharmaceutically acceptable salt thereof, wherein Y³ is C(O) and Y⁴ is NR²².

35. The compound of any one of claims 26-34, or a pharmaceutically acceptable salt thereof, wherein Y⁴, Y⁵ and Y⁶ are joined to form a 3-10 membered ring, such as a 4, 5, or 6-membered heterocyclic ring having 1-3 ring heteroatoms independently selected from N, O, and S, such as 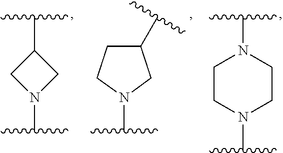
etc., or a spiro bicyclic ring, such as

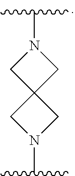 [SMILES: *N1CC2(C1)CN(*)C2]

36. The compound of any one of claims 26-35, or a pharmaceutically acceptable salt thereof, wherein Y⁵, Y⁶ and Y⁷ are joined to form a 3-6 membered ring, such as a 5-membered heteroaryl ring having 1-3 ring heteroatoms independently selected from N, O, and S, such as 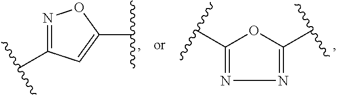 [SMILES: *c1cc(*)on1 ; *c1nnc(*)o1]
or a 4 or 5-membered heterocyclic ring having 1-3 ring heteroatoms independently selected from N, O, and S, such as 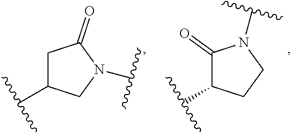 [SMILES: *C1CC(=O)N(*)C1 ; *[C@H]1CCN(*)C1=O]
etc.

37. The compound of any one of claims 26-36, or a pharmaceutically acceptable salt thereof, wherein Y⁶, Y⁷ and Y⁸ are joined to form a 3-6 membered ring, such as a 4 or 5-membered heterocyclic ring having 1-3 ring heteroatoms independently selected from N, O, and S.

38. The compound of any one of claims 26-37, or a pharmaceutically acceptable salt thereof, wherein Y⁶ is C(O) and Y⁷ is NR²².

39. The compound of any one of claims 26-37, or a pharmaceutically acceptable salt thereof, wherein Y⁷ is C(O) and Y^{s} is NR²².

40. The compound of any one of claims 26-38, or a pharmaceutically acceptable salt thereof, wherein Y⁸ is absent, CH₂ or CH(C₁₋₄ alkyl).

41. The compound of any one of claims 26-40, or a pharmaceutically acceptable salt thereof, wherein as applicable, Formula L-1 is characterized as having Formula L-2, L-2A, L-3, L-3A, L-4, L-4A, L-5 or L-6: 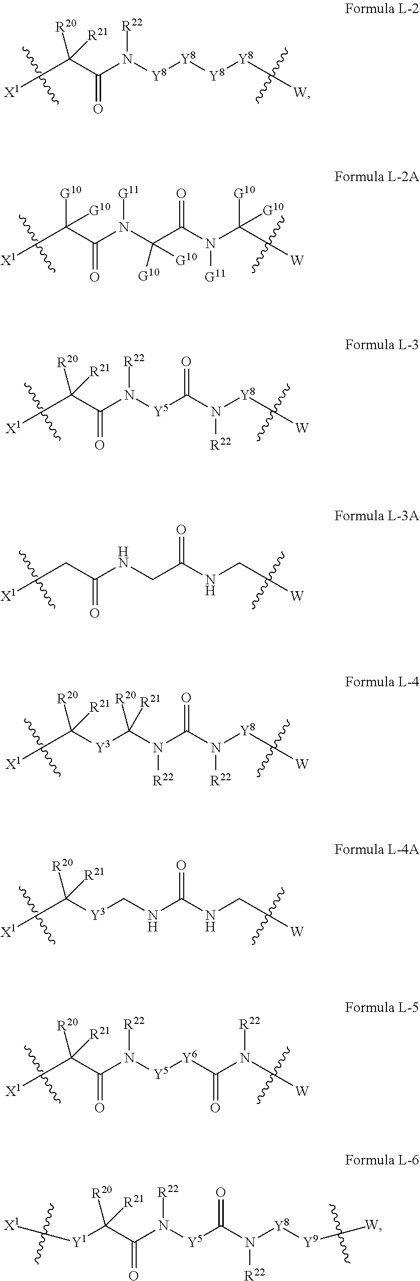
wherein in Formula L-2A, each G¹⁰ is independently hydrogen, deuterium, methyl, or two G¹⁰ together with the carbon they are both attached to form a cyclopropylene, 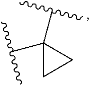 [SMILES: *C1(*)CC1]
and each G¹¹ is hydrogen or methyl, provided that out of all instances of G¹⁰ and G¹¹ combined, at most four instances are not hydrogen or deuterium, preferably, at most three instances are not hydrogen or deuterium, more preferably, only one or two instances are not hydrogen or deuterium,
wherein the remaining variables are as defined for the respective variable in any of claims 26-40.

42. The compound of any one of claims 1-41, or a pharmaceutically acceptable salt thereof, wherein W contains one or more of the following moiety: 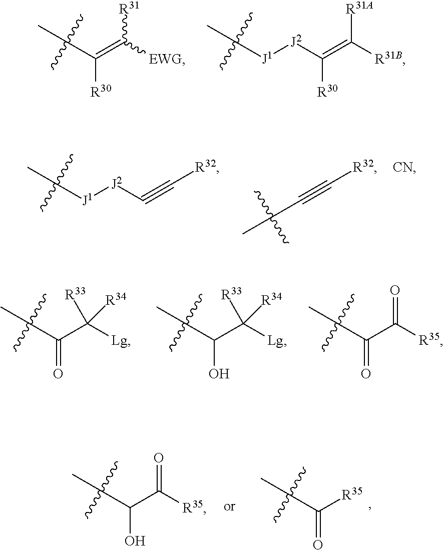
or a protected or masked derivative thereof,
wherein:
EWG is an electron withdrawing group, e.g., an aldehyde, ester, amide, sulfone, sulfonamide, CN, etc.,
Lg is OH, CN, or a leaving group, e.g., F, Cl, etc.
R³⁰ and R³¹ are each independently G^{B} or an electron withdrawing group, or R³⁰ and R³¹ or EWG are joined to form a non-aromatic ring structure, or R³¹ and EWG are joined to form a non-aromatic ring structure;
R^{31A} and R^{31B} are each independently G^{B} or an electron withdrawing group, or R³⁰ and R^{31B} are joined to form a non-aromatic ring structure, or R^{31A} and R^{31B} are joined to form a non-aromatic ring structure;
J¹ is O, NR⁶, C₁₋₄ alkylene, C₁₋₄ heteroalkylene, or absent;
J² is absent, C(O), SO, or SO₂;
R³² is G^{B} or an electron withdrawing group,
R³³ and R³⁴ are independently hydrogen or optionally substituted C₁₋₄ alkyl;
R³⁵ is G^{B}, OG^{B}, NH(G^{B}), N(G^{B})(G^{B}), and
R³⁶ is hydrogen or optionally substituted C₁₋₄ alkyl or an electron withdrawing group,
wherein G^{B} at each occurrence is independently hydrogen, an optionally substituted C₁₋₆ alkyl, optionally substituted C₂₋₆ alkenyl, optionally substituted C₂₋₆ alkynyl, or an optionally substituted ring structure having 4-10 ring atoms.

43. The compound of claim 42, or a pharmaceutically acceptable salt thereof, wherein W is selected from the following: 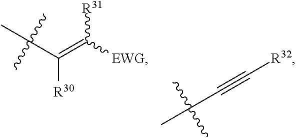
wherein:
EWG is selected from COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G^{A}),
wherein G^{A} at each occurrence is independently an optionally substituted C₁₋₆ alkyl, optionally substituted C₂₋₆ alkenyl, optionally substituted C₂₋₆ alkynyl, an optionally substituted C₁₋₆ heteroalkyl, or an optionally substituted ring structure having 4-10 ring atoms.

44. The compound of claim 43, or a pharmaceutically acceptable salt thereof, wherein:
R³⁰ and R³¹ are both hydrogen; and
R³² is hydrogen or a C₁₋₄ alkyl.

45. The compound of any one of claims 1-41, or a pharmaceutically acceptable salt thereof, wherein (i) W is 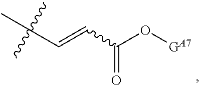
C(O)R³⁵ or 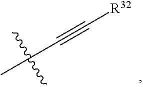
wherein G^{A} is an optionally substituted C₁₋₆ alkyl, R³⁵ is hydrogen or a C₁₋₄ alkyl, and R³² is hydrogen or a C₁₋₄ alkyl; (ii) W is as defined in any of claims 67-70 below; or (iii) W is any of those shown in Table A herein.

46. A compound of Formula Y, or a pharmaceutically acceptable salt thereof, 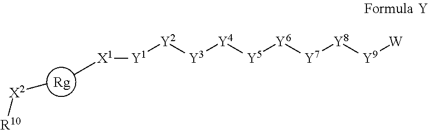
wherein:
X¹ is absent, CR¹¹R¹², NR¹³C(O), or —C(O)N(R¹³)—,
X² is absent, optionally substituted C₁₋₄ alkylene, or optionally substituted C₁₋₄ heteroalkylene,
Rg is an optionally substituted ring structure having 4-10 ring atoms, preferably, an optionally substituted 4-10 membered heterocyclic ring,
Y¹ and Y⁹ are each independently absent, an optionally substituted C₁₋₄ alkylene,
optionally substituted C₁₋₄ heteroalkylene, or optionally substituted 3-8 membered ring structure,
Y², Y³, Y⁴, Y⁵, Y⁶, Y⁷, and Y⁸ are each independently absent, CR²⁰R²¹, C(O), O, NR²², S, SO, or SO₂, wherein up to 4 of Y², Y³, Y⁴, Y⁵, Y⁶, Y⁷, and Y⁸ can be absent, wherein
(i) two adjacent CR²⁰R²¹ units can optionally form a double bond to form —C(R²⁰)═C(R²¹)— or —C≡C—;
(ii) two adjacent CR²⁰R²¹ and NR²² units can optionally form a double bond to form —C(R²⁰)═N—; or
(iii) two adjacent NR²² units can optionally form a double bond to form —N═N—;
or three or four consecutive Y², Y³, Y⁴, Y⁵, Y⁶, Y⁷, and Y⁸ together (e.g., Y⁴, Y⁵, and Y⁶;
or Y⁵, Y⁶, and Y⁷, etc.) represent a 3-10 membered ring structure, and the remaining of Y², Y³, Y⁴, Y⁵, Y⁶, Y⁷, and Y⁸ are as defined above,
(preferred definitions of Y¹, Y², Y³, Y⁴, Y⁵, Y⁶, Y⁷, Y, and Y⁹ include any of those defined in claims 27-40);
W is 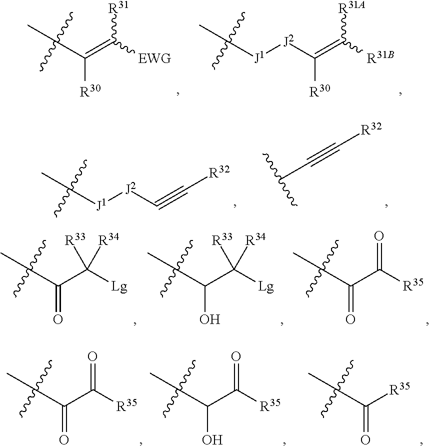
or a protected or masked derivative thereof,
wherein:
R¹⁰ is an optionally substituted aryl or an optionally substituted heteroaryl,
R¹¹ and R¹² are each independently hydrogen, halogen, OH, NH₂, COOH, CONH₂, SO₂NH₂, G^{A}, OG^{A}, NH(G^{A}), N(G^{A})(G^{A}), COOG^{A}, CONH(G^{A}), CON(G^{A})(G^{A}), SO₂NH(G^{A}), SO₂N(G^{A})(G^{A}), NHCOG^{A}, N(G^{A})COG^{A}, NHSO₂(G^{A}), N(G^{A})SO₂(G^{A}), COG^{A}, or SO₂(G^{A}), or R¹¹ and R¹² are joined to form a 3-8 membered ring structure;
R¹³ is hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G),
R²⁰ and R²¹ at each occurrence are each independently hydrogen, halogen, OH, NH₂, COOH, CONH₂, G^{A}, OG^{A}, NH(G^{A}), N(G^{A})(G^{A}), COOG^{A}, CONH₂, CONH(G^{A}), CON(G^{A})(G^{A}), SO₂NH₂, SO₂NH(G^{A}), SO₂N(G^{A})(G^{A}), NHCOG^{A}, N(G^{A})COG^{A}, NHSO₂(G^{A}), N(G^{A})SO₂(G^{A}), COG^{A}, or SO₂(G),
R²² at each occurrence is independently hydrogen, G^{A}, COOG^{A}, CONH₂, CONH(G^{A}), CON(G^{A})(G^{A}), SO₂NH₂, SO₂NH(G^{A}), SO₂N(G^{A})(G^{A}), COG^{A}, or SO₂(G),
or two or more instances of R²⁰, R²¹ and R²² (e.g., R²⁰ and R²¹ on the same carbon, R²⁰ a nd R²⁰ on two different carbons, R²⁰ and R²², etc.), can be joined to form a 3-8 membered ring structure;
EWG is an electron withdrawing group, e.g., an aldehyde, ester, amide, sulfone, sulfonamide, CN, etc.,
R³⁰ and R³¹ are each independently hydrogen, G^{A}, or an electron withdrawing group, or
R³⁰ and R³¹ or EWG are joined to form a non-aromatic ring structure, or R³¹ and EWG are joined to form a non-aromatic ring structure;
R^{31A} and R^{31B} are each independently hydrogen, G^{A}, or an electron withdrawing group, or
R³⁰ and R^{31B} are joined to form a non-aromatic ring structure, or R^{31A} and R^{31B} are joined to form a non-aromatic ring structure;
J¹ is O, NR³⁶, C₁₋₄ alkylene, C₁₋₄ heteroalkylene, or absent, wherein R³⁶ is hydrogen, optionally substituted C₁₋₄ alkyl, or an electron withdrawing group;
J² is absent, C(O), SO, or SO₂;
R³² is hydrogen, G^{A}, or an electron withdrawing group,
Lg is OH, CN, or a leaving group, e.g., F, Cl, etc.
R³³ and R³⁴ are independently hydrogen or optionally substituted C₁₋₄ alkyl;
R³⁵ is G^{B}, OG^{B}, NH(G^{B}), N(G^{B})(G^{B}), and
R³⁶ is hydrogen or optionally substituted C₁₋₄ alkyl or an electron withdrawing group,
wherein G^{B} at each occurrence is independently hydrogen, an optionally substituted C₁₋₆ alkyl, optionally substituted C₂₋₆ alkenyl, optionally substituted C₂₋₆ alkynyl, or an optionally substituted ring structure having 4-10 ring atoms; and
wherein G^{A} at each occurrence is independently an optionally substituted C₁₋₆ alkyl, optionally substituted C₂₋₆ alkenyl, optionally substituted C₂₋₆ alkynyl, optionally substituted C₁₋₆ heteroalkyl, or an optionally substituted ring structure having 4-10 ring atoms.

47. The compound of claim 46, or a pharmaceutically acceptable salt thereof, characterized as having a formula of Formula Y-1, Y-2, Y-2-E, Y-2-F, Y-3, Y-3A, Y-3B, Y-3C, Y-4, Y-5, Y-6, or Y-7: 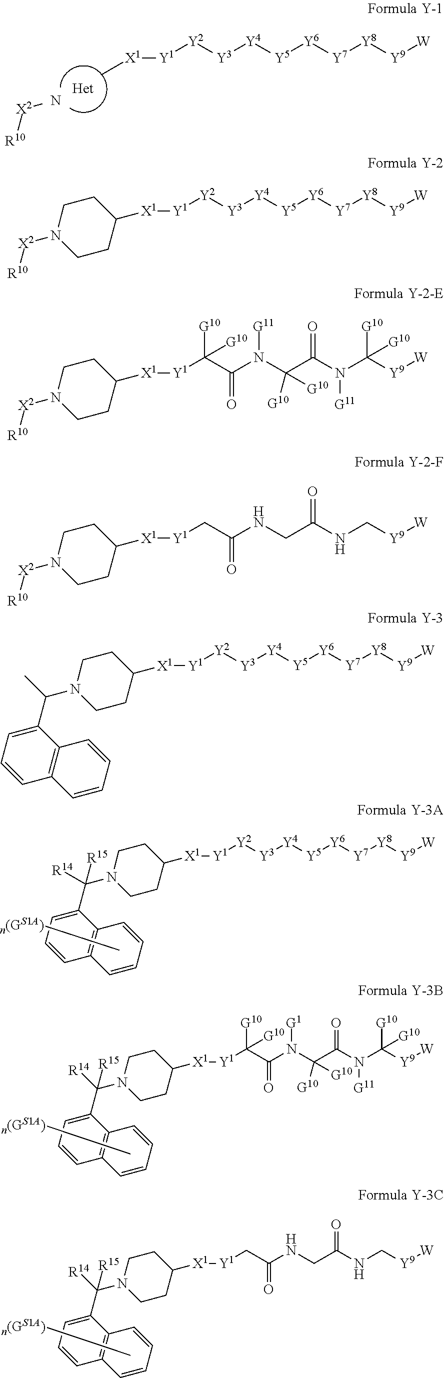 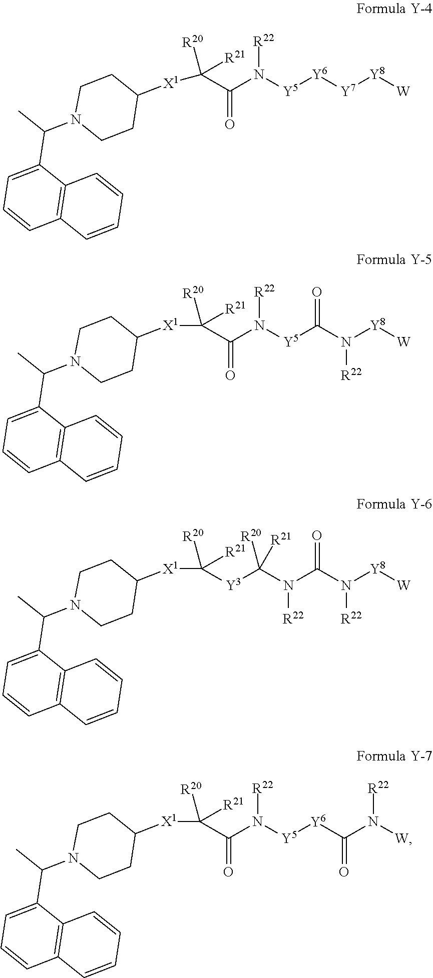
wherein Het represents an optionally substituted 4-10 membered heterocyclic ring, which is bonded with X² through the ring nitrogen atom as drawn, and is bonded with X¹ through another ring atom, wherein the 4-10 membered heterocyclic ring has 0-3 ring heteroatoms each independently O, S, or N, in addition to the as drawn ring nitrogen atom,
wherein in Formula Y-2-E or Y-3B, each G¹⁰ is independently hydrogen, deuterium, methyl, or two G¹⁰ together with the carbon they are both attached to form a cyclopropylene, 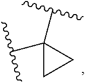 [SMILES: *C1(*)CC1]
and each G¹¹ is hydrogen or methyl, provided that out of all instances of G¹⁰ and G¹¹ combined, at most four instances are not hydrogen or deuterium, preferably, at most three instances are not hydrogen or deuterium, more preferably, only one or two instances are not hydrogen or deuterium;
wherein in Formula Y-3A, Y-3B or Y-3C, (i) R¹⁴ and R¹⁵ are each independently hydrogen, an optionally substituted C₁₋₄ alkyl, optionally substituted C₂₋₄ alkenyl, optionally substituted C₂₋₄ alkynyl, or an optionally substituted ring structure having 4-10 ring atoms; (ii) one of R¹⁴ and R¹⁵ is COOH, or an ester or amide thereof, and the other of R¹⁴ and R¹⁵ is defined in (i); or (iii) R¹⁴ and R¹⁵ together with the carbon they are both attached to form a 3-6 membered ring which is optionally substituted;
wherein n is 0, 1, 2, 3, or 4, preferably, 0, 1, or 2, and
G^{S1A} at each occurrence is independently halogen (preferably F or Cl), OH, C₁₋₄ alkyl optionally substituted with 1-3 F (e.g., methyl, CF₃), C₁₋₄ alkoxy optionally substituted with 1-3 F (e.g., methoxy); or an optionally substituted 3-7 membered ring.

48. The compound of claim 46 or 47, or a pharmaceutically acceptable salt thereof, wherein X1 is NR.

49. The compound of claim 48, or a pharmaceutically acceptable salt thereof, wherein (i) R¹³ is hydrogen, optionally substituted C₁₋₆ alkyl, COG^{A} or SO₂G^{A}; or (ii) R¹³ is an optionally substituted C₁₋₆ alkyl, optionally substituted C₂₋₆ alkenyl or optionally substituted C₂₋₆ alkynyl, such as 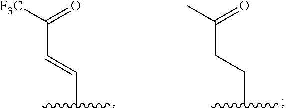
or (iii) R¹³ is as defined in any of claims 16-22.

50. The compound of claim 48, or a pharmaceutically acceptable salt thereof, wherein R¹³ is selected from:

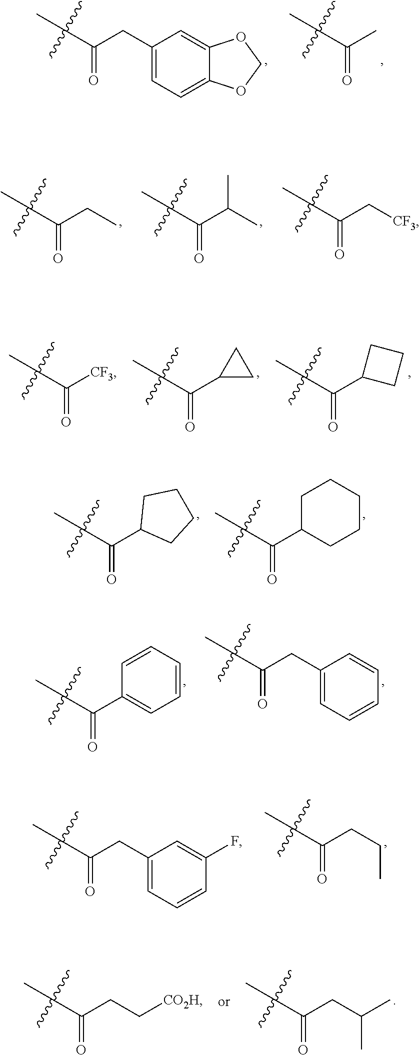

51. The compound of claim 48, or a pharmaceutically acceptable salt thereof, wherein R¹¹ is selected from:

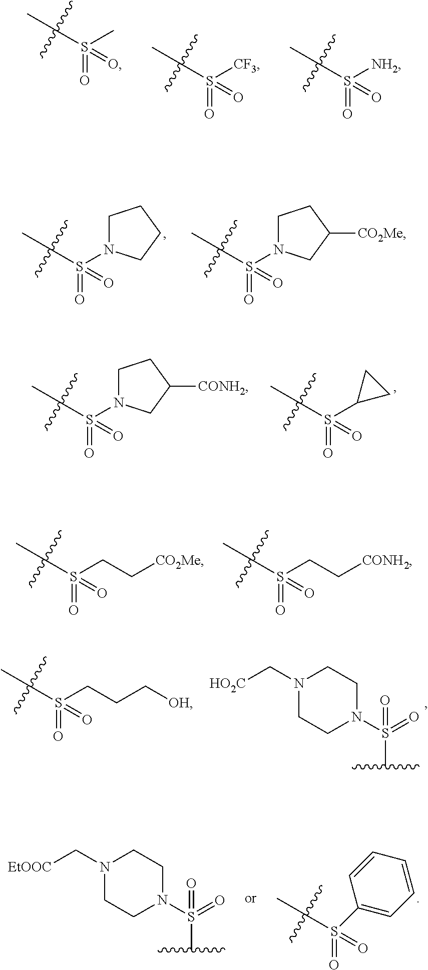

52. The compound of any one of claims 46-51, or a pharmaceutically acceptable salt thereof, wherein (i) R²⁰ and R²¹ at each occurrence are each independently hydrogen or optionally substituted C₁₋₄ alkyl (e.g., CF₃), or (ii) R²⁰ and R²¹ at each occurrence are each independently hydrogen, deuterium, OH, or optionally substituted C₁₋₄ alkyl (e.g., CF₃).

53. The compound of any one of claims 46-52, or a pharmaceutically acceptable salt thereof, wherein R²² at each occurrence is independently hydrogen or optionally substituted C₁₋₄ alkyl.

54. The compound of any one of claims 46-53, or a pharmaceutically acceptable salt thereof, wherein (i) Y² is CH₂ or CH(C₁₋₄ alkyl), such as CH(CH₃), wherein the C₁₋₄ alkyl is optionally substituted, such as with substituents independently selected from F, OH, and NH₂, (ii) Y² is absent, or (iii) Y² is CH(CH₃), C(CH₃)₂, CD₂ or

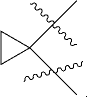 [SMILES: *C1(*)CC1]

55. The compound of any one of claims 46-54, or a pharmaceutically acceptable salt thereof, wherein (i) Y³ is C(O), (ii) Y³ is absent, or (iii) Y³ is CH(OH) or CH(C₁₋₄ alkyl), wherein the C₁₋₄ alkyl is optionally substituted, such as with substituents independently selected from F, OH, and NH₂, for example, Y³ is CH(CF₃).

56. The compound of any one of claims 46-55, or a pharmaceutically acceptable salt thereof, wherein (i) Y⁴ is NH, CH₂ or CH(C₁₋₄ alkyl), (ii) Y⁴ is absent, or (iii) Y⁴ is N(C₁₋₄ alkyl), such as NCH₃, wherein each of the C₁₋₄ alkyl in (i) and (iii) is optionally substituted, such as with substituents independently selected from F, OH, and NH₂.

57. The compound of any one of claims 46-56, or a pharmaceutically acceptable salt thereof, wherein (i) Y⁵ is NH, CH₂, CH(C₁₋₄ alkyl), C(C₁₋₄ alkyl)(C₁₋₄ alkyl), or 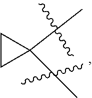 [SMILES: *C1(*)CC1]
(ii) Y⁵ is absent, or (iii) Y⁵ is CH(CH₃), C(CH₃)₂, CD₂ or

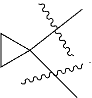 [SMILES: *C1(*)CC1]

58. The compound of any one of claims 46-57, or a pharmaceutically acceptable salt thereof, wherein (i) Y⁶ is C(O), CH₂ or CH(C₁₋₄ alkyl), (ii) Y⁶ is absent, or (iii) Y⁶ is CH(OH) or CH(C₁₋₄ alkyl), wherein the C₁₋₄ alkyl is optionally substituted, such as with substituents independently selected from F, OH, and NH₂, for example, Y⁶ is CH(CF₃).

59. The compound of any one of claims 46-58, or a pharmaceutically acceptable salt thereof, wherein (i) Y⁷ is NH, CH₂ or CH(C₁₋₄ alkyl), (ii) Y⁷ is absent, or (iii) Y⁷ is N(C₁₋₄ alkyl), such as NCH₃, CH(CH₃), C(CH₃)₂, or CD₂, wherein each of the C₁₋₄ alkyl in (i) and (iii) is optionally substituted, such as with substituents independently selected from F, OH, and NH₂.

60. The compound of any one of claims 46-58, or a pharmaceutically acceptable salt thereof, wherein Y⁷ is C(O).

61. The compound of any one of claims 46-60, or a pharmaceutically acceptable salt thereof, wherein Y⁸ is NH or Y⁸ is O or N(C₁₋₄ alkyl), such as NCH₃, wherein the C₁₋₄ alkyl is optionally substituted, such as with substituents independently selected from F, OH, and NH₂.

62. The compound of any one of claims 46-60, or a pharmaceutically acceptable salt thereof, wherein (i) Y⁸ is CH₂ or CH(C₁₋₄ alkyl), (ii) Y⁸ is CO; or (iii)^{Y} is CH(CH₃), C(CH₃)₂, CD₂ or

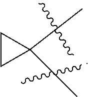 [SMILES: *C1(*)CC1]

63. The compound of any one of claims 46-60, or a pharmaceutically acceptable salt thereof, wherein Y⁸ is absent.

64. The compound of any one of claims 46-63, or a pharmaceutically acceptable salt thereof, wherein (i) Y⁹ is absent, an optionally substituted C₁₋₄ alkylene, such as CH₂ or CD₂, or an optionally substituted 4-8 membered heterocyclic ring having 1 or 2 ring heteroatoms independently O, S, or N, (ii) Y⁹ is a C₃₋₆ membered carbocyclic ring, such as 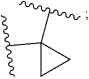 [SMILES: *C1(*)CC1]
or (iii) Y⁹ is a C₁₋₄ heteroalkylene, which is optionally substituted, such as with oxo, F, CF₃, etc.

65. The compound of any one of claims 46-64, or a pharmaceutically acceptable salt thereof, wherein (i) Y¹ is absent, (ii) Y¹ is an optionally substituted C₁₋₄ alkylene, such as CH₂ or CD₂, or (iii) Y¹ is an optionally substituted 4-8 membered heterocyclic ring having 1-3 ring heteroatoms independently O, S, or N, such as or

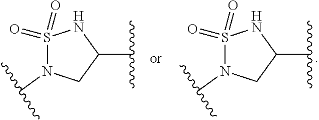 [SMILES: *C1CN(*)S(=O)(=O)N1 ; *C1CN(*)S(=O)(=O)N1]

66. The compound of any one of claims 46-65, or a pharmaceutically acceptable salt thereof, wherein W is 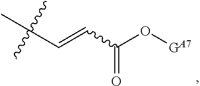
C(O)R³⁵, or 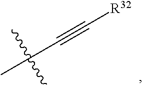
wherein G is an optionally substituted C₁₋₆ alkyl, R³⁵ is hydrogen or a C₁₋₄ alkyl, and R³² is hydrogen or a C₁₋₄ alkyl.

67. The compound of any one of claims 46-65, or a pharmaceutically acceptable salt thereof, wherein W is 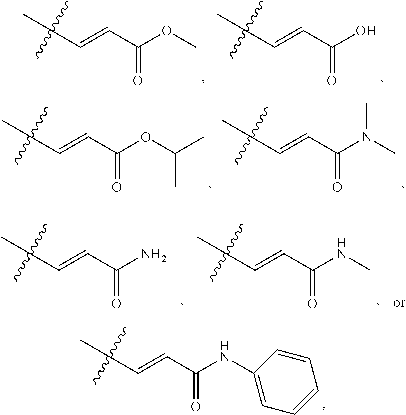
or W is

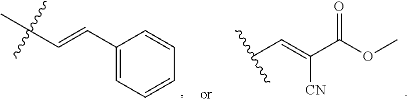

68. The compound of any one of claims 46-65, or a pharmaceutically acceptable salt thereof, wherein W is

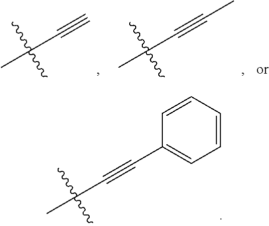

69. The compound of any one of claims 46-65, or a pharmaceutically acceptable salt thereof, wherein Y⁹—W is —(C₁₋₄ alkylene)-CN, CN,

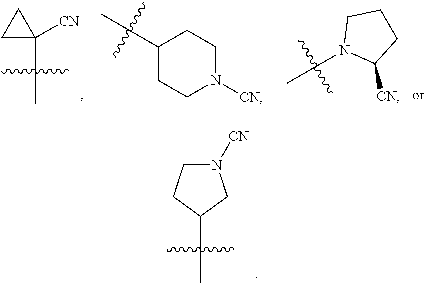

70. The compound of any one of claims 46-65, or a pharmaceutically acceptable salt thereof, wherein W is C(O)H, 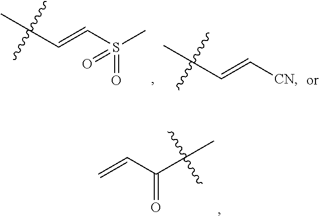
or W is 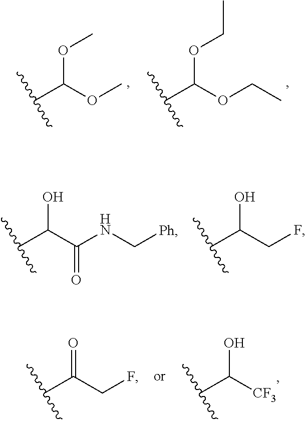
or W is

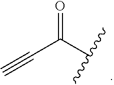 [SMILES: *C(=O)C#C]

71. A compound selected from Compound Nos 1-177 or the compounds shown in Table A herein, or a pharmaceutically acceptable salt thereof.

72. A pharmaceutical composition comprising the compound according to any one of claims 1-71 or a pharmaceutically acceptable salt thereof, and optionally a pharmaceutically acceptable excipient.

73. The pharmaceutical composition of claim 72, further comprising one or more other antiviral agents.

74. A method of treating or preventing viral infection, the method comprising administering to a subject in need thereof an effective amount of the compound according to any one of claims 1-71 or a pharmaceutically acceptable salt thereof or the pharmaceutical composition of claims 72 or 73.

75. The method of claim 74, further comprising administering to the subject one or more other antiviral agents.

76. The method of claim 74 or 75, wherein the viral infection is caused by a virus selected from SARS-CoV, HCoV-229E, HCoV-OC43, HCoV-NL63, MERS-CoV, HCoV-HKU1, and SARS-CoV-2.

77. A method of inhibiting papain-like protease (PLpro) in a subject in need thereof, the method comprising administering to the subject an effective amount of the compound according to any one of claims 1-71 or a pharmaceutically acceptable salt thereof or the pharmaceutical composition of claims 72 or 73.

78. The method of claim 77, wherein the subject suffers from viral infection caused by a virus selected from SARS-CoV, HCoV-229E, HCoV-OC43, HCoV-NL63, MERS-CoV, HCoV-HKU1, and SARS-CoV-2.
